(19) United States
(12) Patent Application Publication
(10) Pub. No.: US 2024/0336914 A1
(43) Pub. Date: Oct. 10, 2024

(54) 2'-MODIFIED NUCLEOSIDE BASED OLIGONUCLEOTIDE PRODRUGS

(71) Applicant: ALNYLAM PHARMACEUTICALS, INC., Cambridge, MA (US)

(72) Inventors: Shigeo MATSUDA, Cambridge, MA (US); Jayaprakash K. NAIR, Cambridge, MA (US); Martin A. MAIER, Cambridge, MA (US); Michelle H. JUNG, Cambridge, MA (US); Anna BISBE, Cambridge, MA (US); Ivan ZLATEV, Cambridge, MA (US); Mark K. SCHLEGEL, Cambridge, MA (US); Christopher BROWN, Cambridge, MA (US); Vasant R. JADHAV, Cambridge, MA (US)

(21) Appl. No.: 18/259,887

(22) PCT Filed: Dec. 30, 2021
(86) PCT No.: PCT/US2021/065650

§ 371 Date: Jun. 29, 2023

Related U.S. Application Data
(60) Provisional application No. 63/132,545, filed on Dec. 31, 2020.

(51) Int. Cl. C12N 15/113

(52) U.S. Cl. CPC C12N 15/113; C12N 15/1137; C12N 2310/14; C12N 2310/315; C12N 2310/321; C12N 2310/322; C12N 2310/351

(57) ABSTRACT

This invention relates to an oligonucleotide comprising one or more 2′-modified nucleosides, wherein the 2′-position of the nucleoside has a structure of formula (I). The invention also relates to a pharmaceutical composition comprising the oligonucleotide described herein and a method of reducing or inhibiting the expression of a target gene by administering to the subject a therapeutically effective amount of the oligonucleotide described herein. The invention also relates to a method of bioactivating an oligonucleotide comprising one or more 2′-modified nucleosides, wherein the 2′-position of the nucleoside is modified by a bio-cleavable linking group, wherein the bio-cleavable linking group comprises acetal, disulfide, carbamate, amide, sulfonamide, a biocleavable carbohydrate linker, or combinations thereof, said method comprising the step of: exposing the oligonucleotide to a physiological condition that causes the bio-cleavable linking group to be cleaved from the 2′-modified nucleoside, thereby regenerating the 2′-OH group of the nucleoside.

### CROSS-REFERENCE TO RELATED APPLICATIONS

This application claims benefit of priority to U.S. Provisional Application No. 63/132,545 filed Dec. 31, 2020, which is herein incorporated by reference in its entirety.

### FIELD OF INVENTION

This invention generally relates to the field of 2′-modified nucleoside based oligonucleotide prodrugs.

### BACKGROUND

Oligonucleotides have been studied for their therapeutic applications for over thirty years. Many chemical modifications have been permanently introduced into oligonucleotides to improve their stability, enhance cell penetration, and increase gene-silencing activities. For instance, permanent modifications at 2′-position of the nucleoside, such as, 2′-O-modifications (2′-F, 2′-OMe) have been developed to increase the nuclease resistance of oligonucleotides.

Alternatively, prodrug approaches have been researched to introduce transient modifications that can be reverted upon an in vivo stimuli. A prodrug is an agent that is administered in an inactive or significantly less active form, and that undergoes chemical or enzymatic transformations in vivo to yield the active parent drug under different stimuli. The prodrug approach can offer a number of advantages over their unmodified counterparts including, e.g., enhancing cell penetration and avoiding or minimizing degradation in serum via cellular sequestration. Compared to more permanent modifications, these transient modifications in oligonucleotide prodrugs offer the opportunity to regulate the oligonucleotide activity using a cellular stimuli as a switch, as the temporarily modified oligonucleotide prodrug is not active until it is triggered to release an unmodified oligonucleotide. Nevertheless, most of them have decreased gene silencing potential.

However, the prodrug approach still has substantial challenges, partially because it is difficult to choose the best transient modifying group (e.g., 2′-position masking/protecting group). For instance, cellular cleavage of the transient modifying groups can often generate products which are viewed as disadvantageous or even toxic. Moreover, the transient modifying groups must strike a balance between allowing absorption in the intestines and allowing cleavage in the blood or target cell.

Thus, there is a continuing need for developing new and improved 2′-modified nucleoside based prodrugs for masking/protecting 2′ position of a nucleoside in an oligonucleotide, in order to make effective and efficient oligonucleotide-based drugs, for efficient in vivo delivery and improved in vivo efficacy of oligonucleotides.

### SUMMARY

One aspect of the invention relates to an oligonucleotide comprising one or more 2′-modified nucleosides, wherein the 2′-position of the nucleoside is modified by a bio-cleavable linking group optionally connected to a ligand, which, upon bioactivation at physiological condition, is being cleaved from the nucleoside to regenerate the 2′—OH group of the nucleoside, wherein the bio-cleavable linking group comprises acetal, disulfide, carbamate, amide, sulfonamide, a biocleavable carbohydrate linker, or combinations thereof.

Another aspect of the invention relates to an oligonucleotide (e.g. a single-stranded iRNA agent or a double-stranded iRNA agent) comprising one or more 2′-modified nucleosides. The 2′-position of the nucleoside has a structure of formula (I):

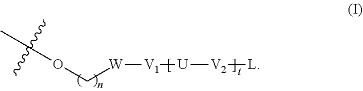

W is O, S-S, N(R^{N}), C(O)N(R^{N}), N(R^{N})C(O), N(R^{N})C(O)O, N(R^{N})S(O)₂, or a biocleavable carbohydrate linker;
V₁ and V₂ are each independently for each occurrence absent, alkylene, arylene, heteroarylene, a divalent cycloalkyl, or a divalent heterocyclyl, each of which can be optionally substituted by one or more R²⁰ groups;
U is independently for each occurrence absent, W, O—W, W—O, or a phosphate or modified phosphate;
R^{N} is H, alkyl, or S(O)₂-aryl, each of which can be optionally substituted by one or more R²⁰ groups;
L is H or one or more ligands;
R²⁰ is independently for each occurrence halo, haloalkyl, hydroxyl, alkoxy, methoxyalkoxy, alkylthio, amino, alkylamino, alkynyl, aminoalkyl, aminoalkoxy, nitro, acetyl, or cyano;
n is an integer of 0-4 (e.g., n is 0 or 1);
t is an integer of 1-3 representing the number of presence for unit [U-V₂], which can be the same or different for each occurrence; and
[symbol] indicates the attachment of the formula (I) to the 2′-position of the nucleoside.

In some embodiments, V₁ is not absent, i.e., V₁ is alkylene, arylene, heteroarylene, a divalent cycloalkyl, or a divalent heterocyclyl, each of which can be optionally substituted by one or more R²⁰ groups.

In some embodiments, the 2′-modified nucleoside has the structure of formula (II):

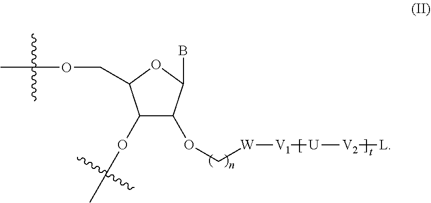

The variables W, V₁, V₂, U, L, t, and n are the same as those defined above in formula (I). B is modified or unmodified nucleobase. Each [symbol] indicates the attachment of the formula (II) to an adjacent nucleotide of the oligonucleotide, or hydrogen, or together with the oxygen to which it is attached forms a phosphate, or a modified phosphate, wherein at least one [symbol] attaches to an adjacent nucleotide.

In some embodiments, B is independently for each occurrence A, A^{Bz}, C, C^{Ac}, C^{Bz}, 5-Me-C, 5-Me-C^{Ac}, G, G^{iBu}, I, U, T, 2-thiouridine, 4-thiouridine, a C5-modified pyrimidine, C2-modified purine, N8-modifed purine, phenoxazine, G-clamp, non-canonical mono-, bi-, and tri-cyclic heterocycles, a pseudouracil, isoC, isoG, 2,6-diamninopurine, a pseudocytosine, 2-aminopurine, xanthosine, N6-alkyl-A, or O6-alkyl-G.

In some embodiments, the 2′-modified nucleoside has the structure of formula (IIa) or (IIb):

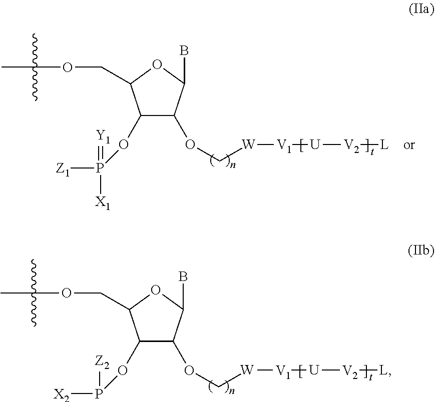

or a salt thereof. In these formulas:
X₁ and Z₁ are each independently H, OH, OM, OR¹³, SH, SM, SR¹³, C(O)H, S(O)H, or alkyl, each of which can be optionally substituted with one or more R^{sub} groups, N(R′)(R″), B(R¹³)₃, BH₃⁻, Se; or D-Q, wherein D is independently for each occurrence absent, O, S, N(R′), alkylene, each of which can be optionally substituted with one or more R^{sub} groups, and Q is independently for each occurrence a nucleoside or oligonucleotide;
X₂ and Z₂ are each independently N(R′)(R″), OR¹⁸, or D-Q, wherein D is independently for each occurrence absent, O, S, N, N(R′), alkylene, each of which can be optionally substituted with one or more R^{sub} groups, and Q is independently for each occurrence a nucleoside or oligonucleotide;
Y₁ is S, O, or N(R′);
M is an organic or inorganic cation;
R¹³ is independently for each occurrence H, alkyl, alkenyl, alkynyl, cycloalkyl, aryl, aralkyl, ω-amino alkyl, ω-hydroxy alkyl, or ω-hydroxy alkenyl, an oxygen protecting group, each of which can be optionally substituted with one or more R^{sub} groups;
R′ and R″ are each independently H, alkyl, alkenyl, alkynyl, aryl, hydroxy, alkyloxy, ω-amino alkyl, ω-hydroxy alkyl, ω-hydroxy alkenyl, or ω-hydroxy alkynyl, each of which can be optionally substituted with one or more R^{sub} groups;
R¹⁸ is H or alkyl, optionally substituted with one or more R^{sub} groups; and
R^{sub} is independently for each occurrence halo, haloalkyl, alkyl, alkaryl, aryl, aralkyl, hydroxy, alkyloxy, aryloxy, oxo, nitro, amino, acylamino, alkylcarbamoyl, arylcarbamoyl, alkylamino, aminoalkyl, alkoxycarbonyl, carboxy, hydroxyalkyl, alkanesulfonyl, arenesulfonyl, alkanesulfonamido, arenesulfonamido, aralkylsulfonamido, alkylcarbonyl, arylcarbonyl, acyloxy, cyano, or ureido. The variables B, W, V₁, V₂, U, L, t, and n are the same as those defined above in formula (II). [symbol] indicates the attachment of the formula (IIa) or (IIb) to the adjacent nucleotide of the oligonucleotide, hydrogen, or together with the oxygen to which it is attached forms a phosphate, or a modified phosphate.

In some embodiments, X₁ in formula (IIa) or (IIb) is D-Q. In one embodiment, X₁ is O-Q. In some embodiments, X₂ in formula (IIa) or (IIb) is D-Q. In one embodiment, X₂ is O-Q.

In some embodiments, the 2′-modified nucleoside has the structure of formula:

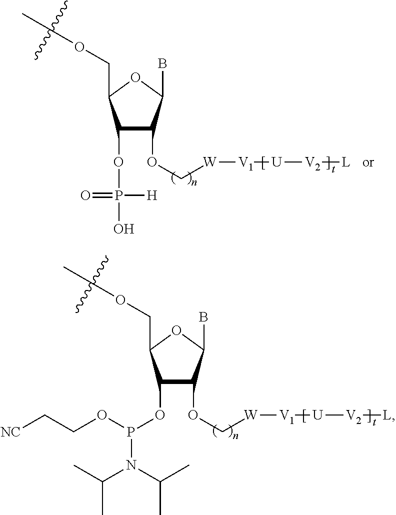

or a salt thereof. The variables B, W, V₁, V₂, U, L, t, and n are the same as those defined above in formula (II).

In one embodiment, the 2′-modified nucleoside has the structure of formula:

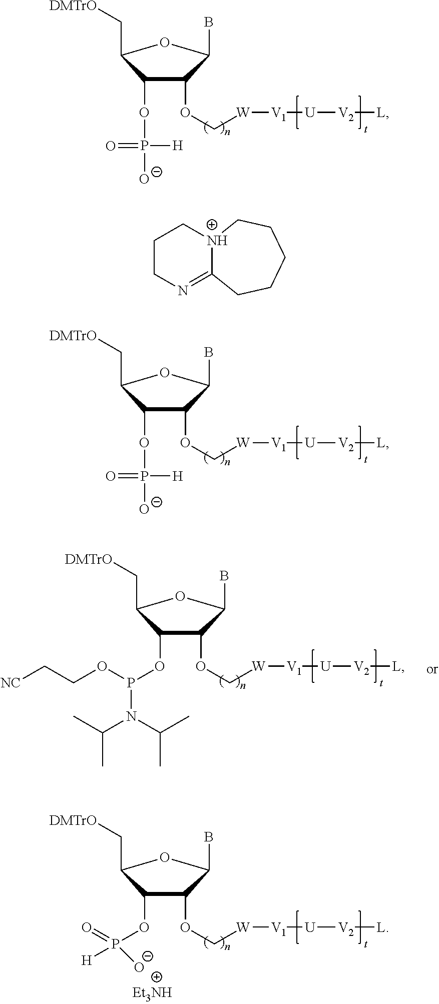

The variables B, W, V₁, V₂, U, L, t, and n are the same as those defined above in formula (II).

In some embodiments, the 2′-modified nucleoside has a modified, abasic sugar and has a structure of (IIIa) or (IIIb):

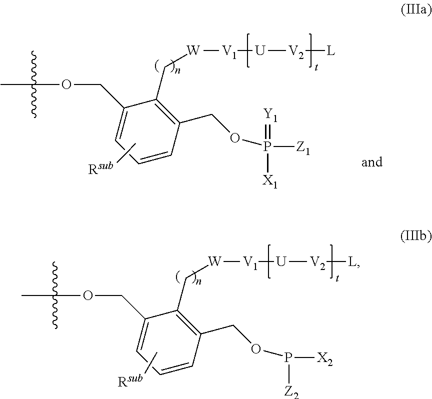

or a salt thereof. In these formulas, the variables X₁, X₂, Y₁, Z₁, Z₂, and R^{sub} are the same as those defined above in formulas (IIa) and (IIb). The variables W, V₁, V₂, U, L, t, and n are the same as those defined above in formula (II).

In some embodiments, the 2′-modified nucleoside has a structure of

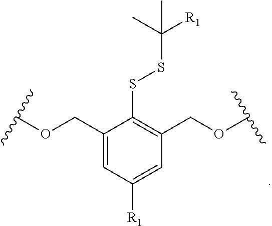

In some embodiments, the 2′-modified nucleoside has a structure of:

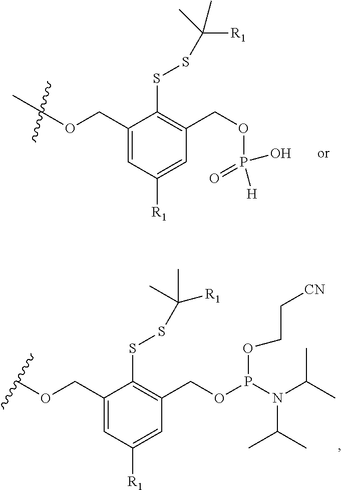

or a salt thereof. In these embodiments, R₁ is independently for each occurrence H, linear or branched C1-C24 alkyl, aryl, hydroxyl, alkoxy, halo, methoxyalkoxy, alkylthio, amino, alkylamino, alkynyl, aminoalkyl, or aminoalkoxy.

In one embodiment, the 2′-modified nucleoside has a structure of:

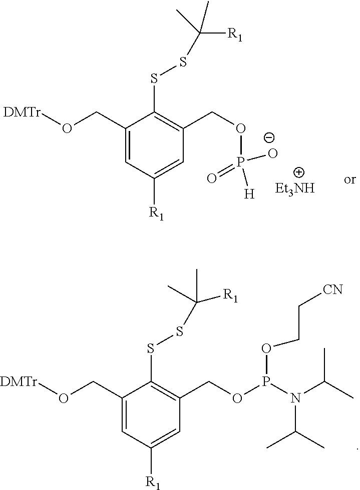

R₁ is independently for each occurrence H, linear or branched C₁-C₂₄ alkyl, aryl, hydroxyl, alkoxy, halo, methoxyalkoxy, alkylthio, amino, alkylamino, alkynyl, aminoalkyl, or aminoalkoxy.

In some embodiments, n is 0 or 1.

In some embodiments, n is 0. W is C(O)N(R^{N}) or a biocleavable carbohydrate linker.

In one embodiment, n is 0, and the W-V₁-[U-V₂]t-L has the structure of:

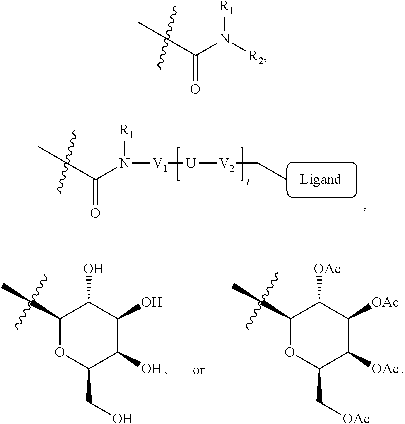

In these formulas:
each R₁ is independently for each occurrence H, linear or branched C₁-C₂₄ alkyl, aryl, hydroxyl, alkoxy, halo, methoxyalkoxy, alkylthio, amino, alkylamino, alkynyl, aminoalkyl, or aminoalkoxy;
R₂ is selected from the group consisting of:

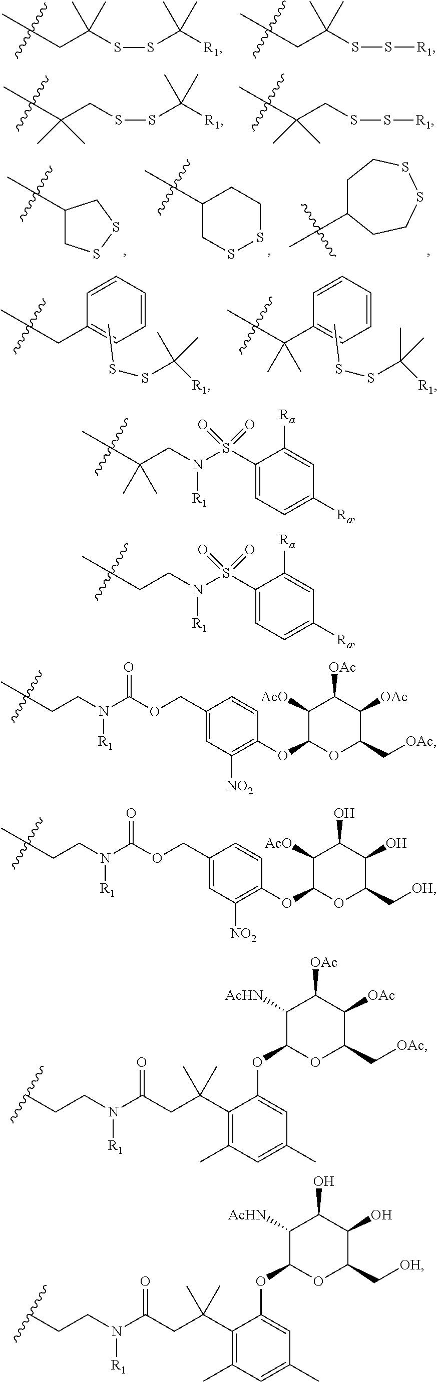

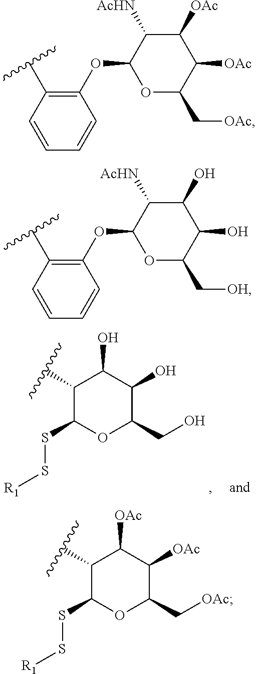

or R₁ and R₂ together from

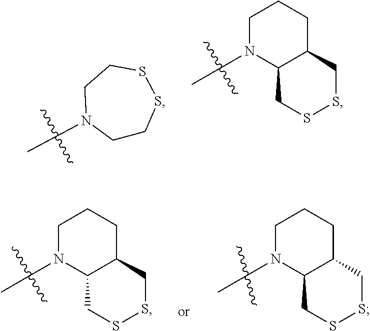 [SMILES: *N1CCSSCC1 ; *N1CCC[C@H]2CSSC[C@H]21 ; *N1CCC[C@H]2CSSC[C@@H]21 ; *N1CCC[C@@H]2CSSC[C@H]21]

and
Rₐ is independently for each occurrence H, NO₂, CF₃, C(O)CH₃, and CN.

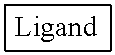

represents a ligand. The variables V₁, V₂, U, and t are the same as those defined above in formula (II).

In some embodiments, n is 1. W is O, S-S, N(R^{N}), C(O)N(R^{N}), N(R^{N})C(O), N(R^{N})C(O)O, or N(R^{N})S(O)₂.

In one embodiment, n is 1, and the W-V₁-[U-V₂]ₜ-L has the structure of

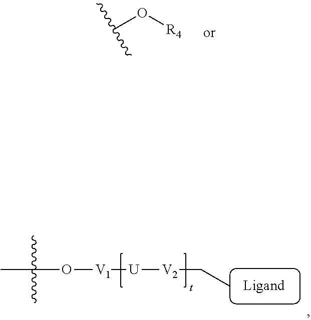

wherein:
R⁴ is

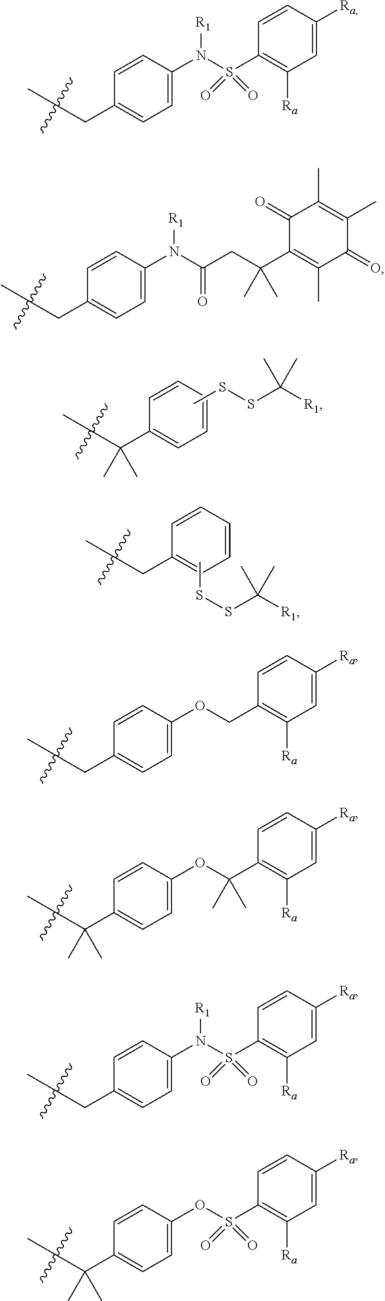

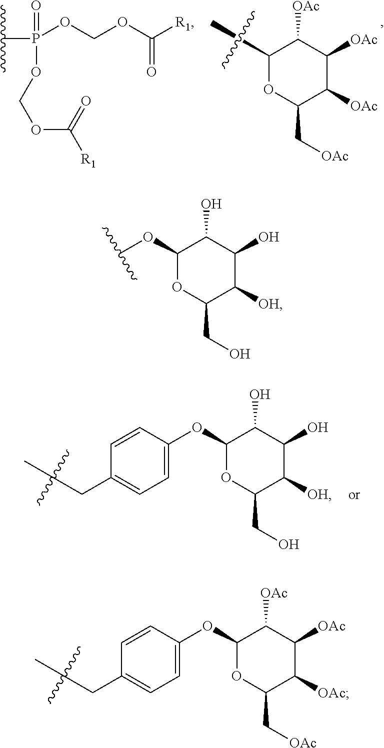

R₁ is independently for each occurrence H, linear or branched C₁-C₂₄ alkyl, aryl, hydroxyl, alkoxy, halo, methoxyalkoxy, alkylthio, amino, alkylamino, alkynyl, aminoalkyl, or aminoalkoxy; and
Rₐ is independently for each occurrence H, NO₂, CF₃, C(O)CH₃, and CN.

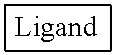

represents a ligand. The variables V₁, V₂, U, and t are the same as those defined above in formula (II).

In an exemplary embodiment, n is 1, and the W-V₁-[U-V₂]ₜ-L has the structure of:

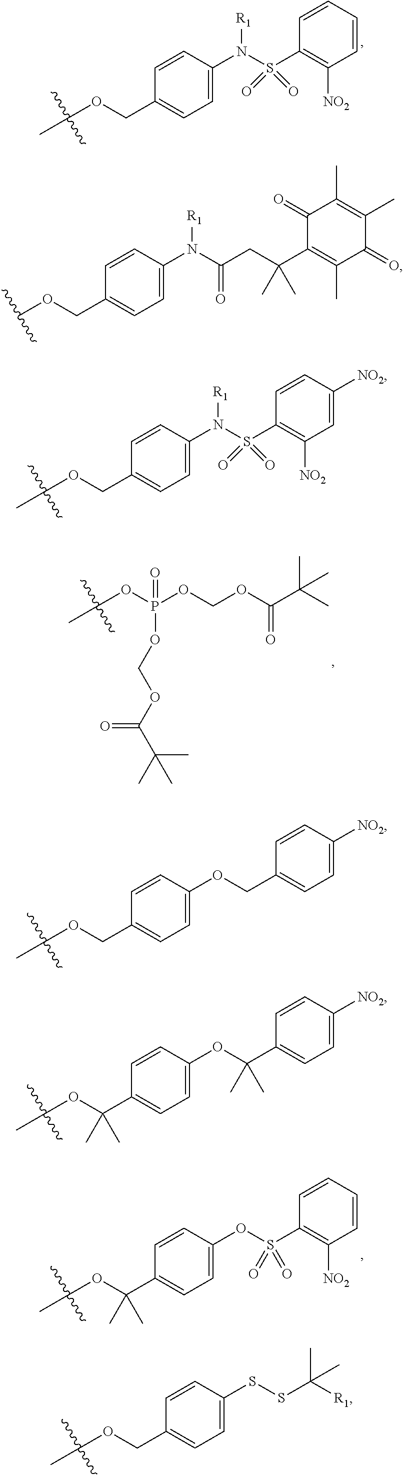

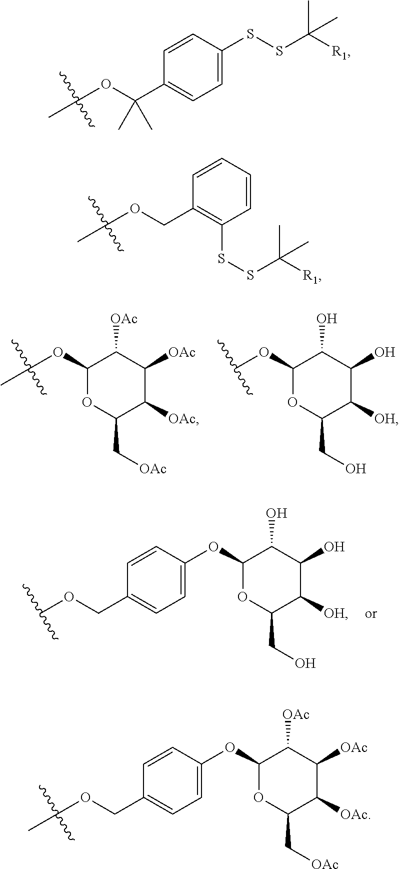

In some embodiments, n is 1, and the W-V₁-[U-V₂]ₜ-L has the structure of:

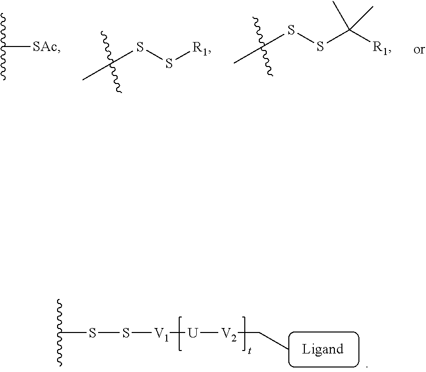

R₁ is independently for each occurrence H, linear or branched C₁-C₂₄ alkyl, aryl, hydroxyl, alkoxy, halo, methoxyalkoxy, alkylthio, amino, alkylamino, alkynyl, aminoalkyl, or aminoalkoxy.

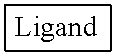

represents a ligand. The variables V₁, V₂, U, and t are the same as those defined above in formula (II).

In an exemplary embodiment, n is 1, and the W-V₁-[U-V₂]ₜ-L has the structure of:

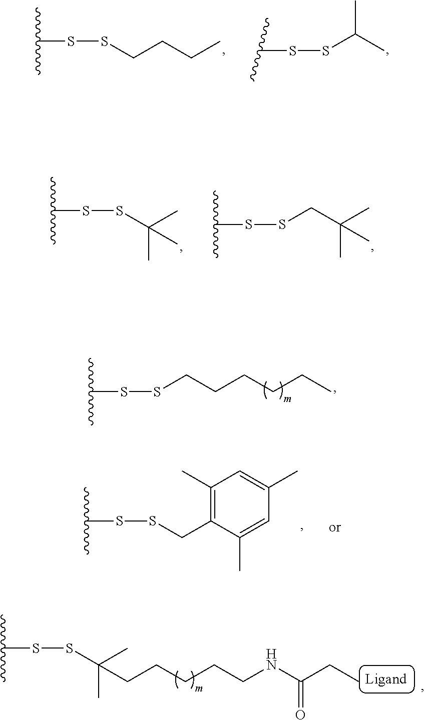

wherein m is an integer of 0-18.

In some embodiments, n is 1, and the W-V₁-[U-V₂]ₜ-L has the structure of:

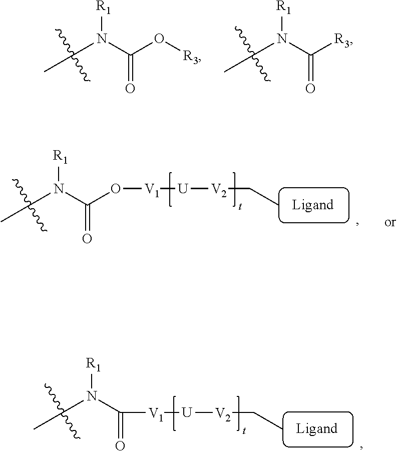

wherein:
R₃ is linear or branched alkyl, aryl,

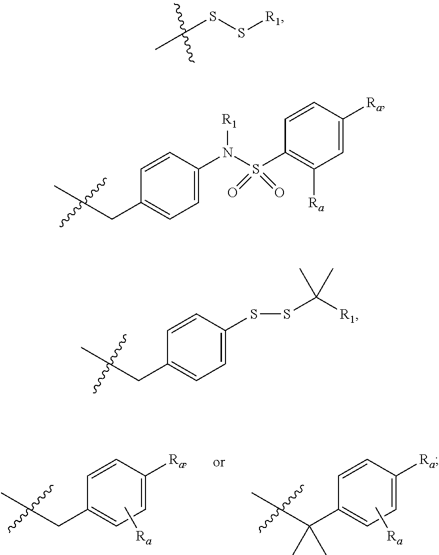

R₁ is independently for each occurrence H, linear or branched C₁-C₂₄ alkyl, aryl, hydroxyl, alkoxy, halo, methoxyalkoxy, alkylthio, amino, alkylamino, alkynyl, aminoalkyl, or aminoalkoxy; and
each Rₐ is independently for each occurrence H, NO₂, CF₃, C(O)CH₃, and CN.

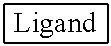

represents a ligand. The variables V₁, V₂, U, and t are the same as those defined above in formula (II).

In an exemplary embodiment, n is 1, and the W-V₁-[U-V₂]ₜ-L has the structure of:

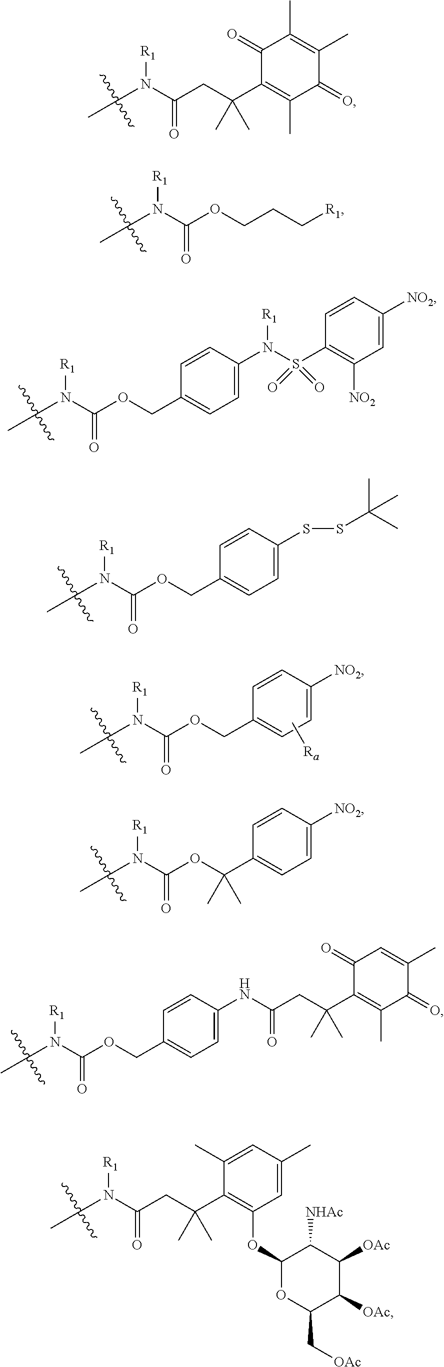

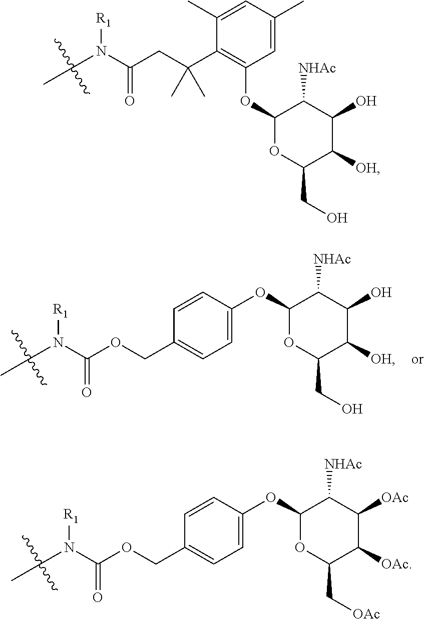

In some embodiments, n is 1, and the W-V₁-U-V₂-L has the structure of

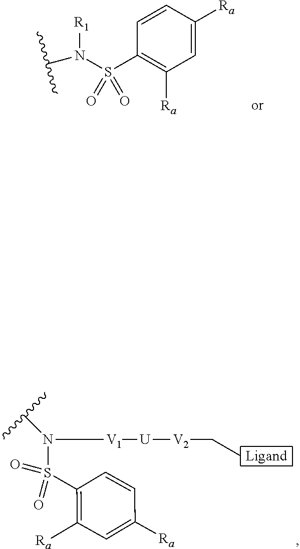

wherein:
R₁ is independently for each occurrence H, linear or branched C₁-C₂₄ alkyl, aryl, hydroxyl, alkoxy, halo, methoxyalkoxy, alkylthio, amino, alkylamino, alkynyl, aminoalkyl, or aminoalkoxy; and
Rₐ is independently for each occurrence H, NO₂, CF₃, C(O)CH₃, and CN.

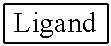

represents a ligand. The variables V₁, V₂, U, and t are the same as those defined above in formula (II).

In an exemplary embodiment, n is 1, and the W-V₁-U-V₂-L has the structure of:

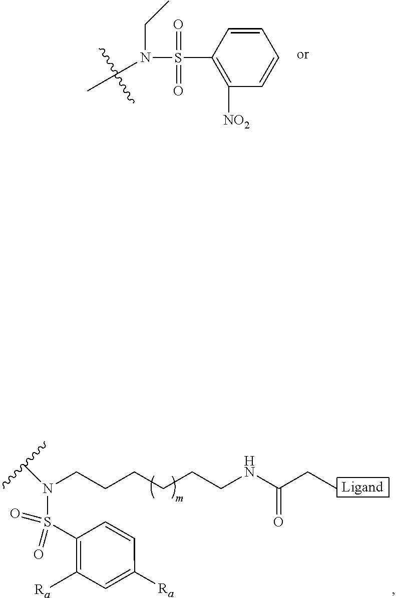

wherein m is an integer of 0-18.

In some embodiments, n is 1, and the W-V₁-U-V₂-L has the structure of:

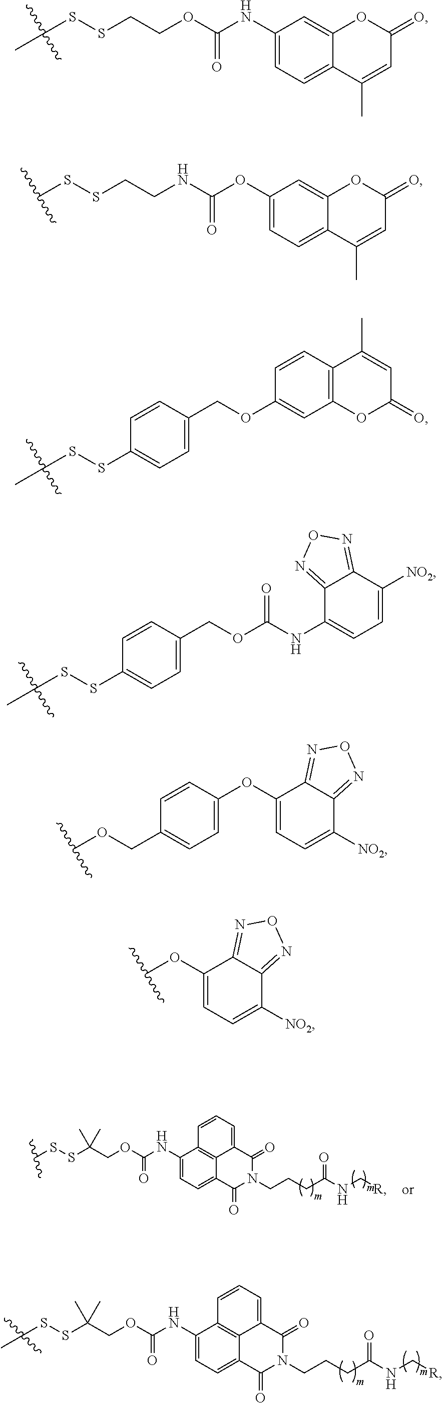

wherein:
R is

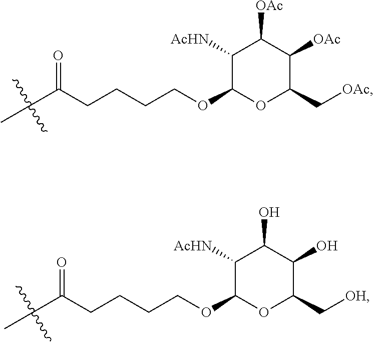 [SMILES: *C(=O)CCCCO[C@@H]1O[C@H](COC(C)=O)[C@H](OC(C)=O)[C@H](OC(C)=O)[C@H]1NC(C)=O ; *C(=O)CCCCO[C@@H]1O[C@H](CO)[C@H](O)[C@H](O)[C@H]1NC(C)=O]

a peptide, a small molecular ligand, GalNAc or multivalent GalNAc, folate or a lipophilic moiety; and
m is independently for each occurrence an integer of 0-18.

In some embodiments, the 2′-modified nucleoside has a structure selected from the group consisting of:

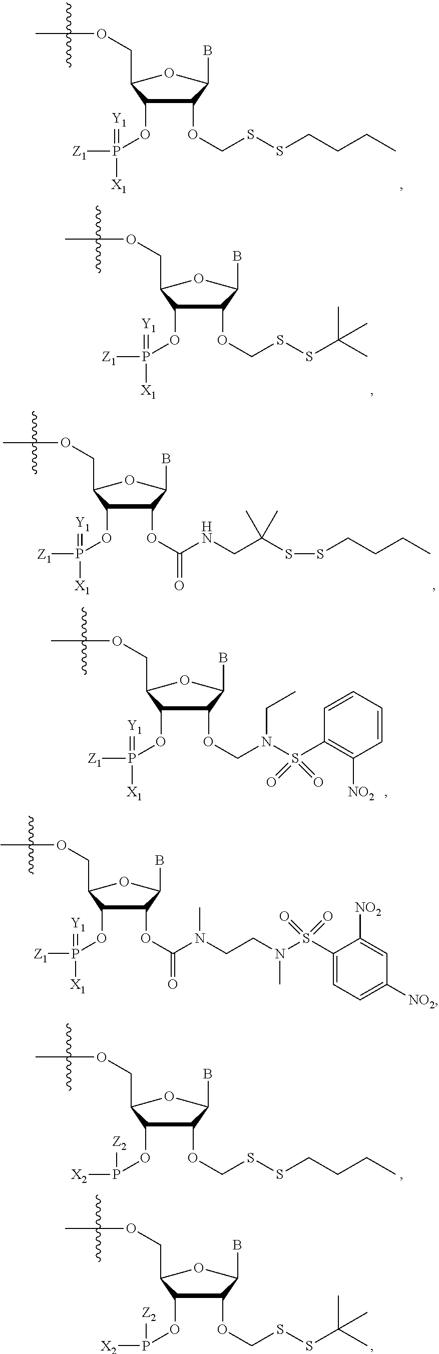

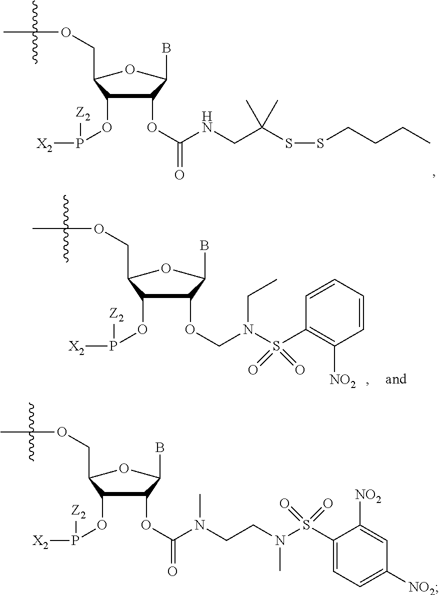

or a salt thereof.

In some embodiment, the 2′-modified nucleoside has a structure selected from the group consisting of:

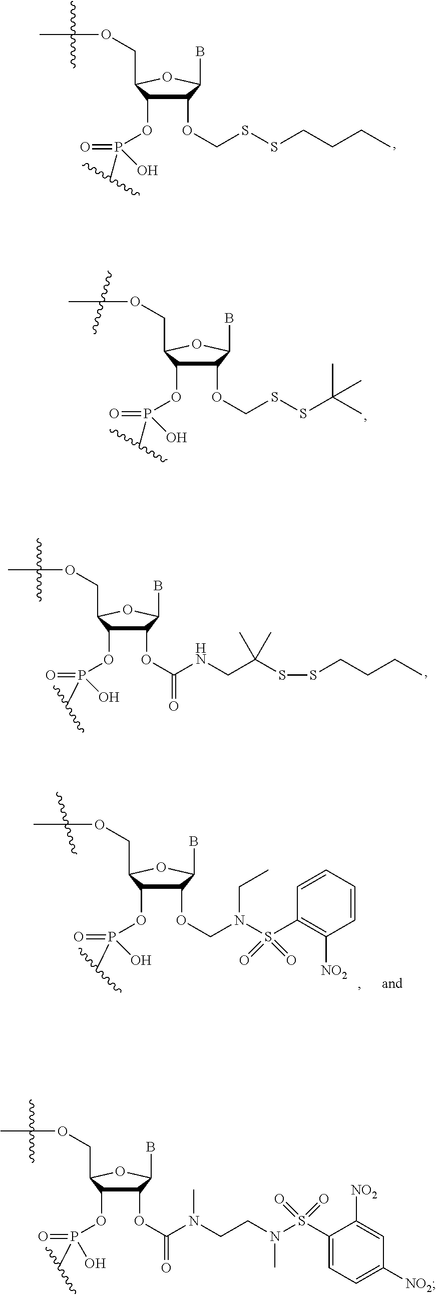

or salt thereof.

Another aspect of the invention relates to a compound of the formula (A), (B), (C), or (D):

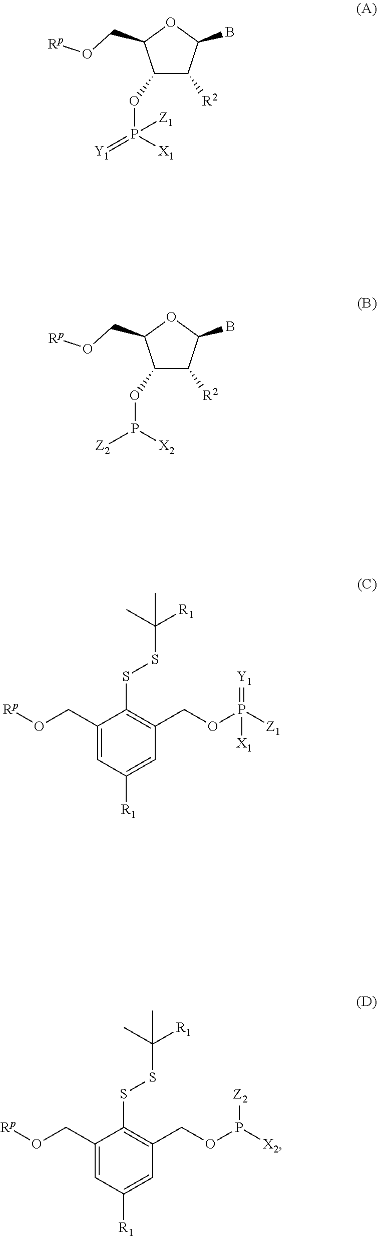

or a salt thereof. In these formulas:
R^{P} is an oxygen protecting group;
B is an optionally modified nucleobase;
R² has a structure of formula (I):

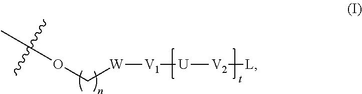

wherein:
[symbol] indicates the attachment of the formula (I) to the 2′-position of the nucleoside;
W is O, S-S, N(R^{N}), C(O)N(R^{N}), N(R^{N})C(O), N(R^{N})C(O)O, N(R^{N})S(O)₂, or a biocleavable carbohydrate linker;
V₁ and V₂ are each independently for each occurrence absent, alkylene, arylene, heteroarylene, a divalent cycloalkyl, or a divalent heterocyclyl, each of which can be optionally substituted by one or more R²⁰ groups;
U is independently for each occurrence absent, W, O-W, W-O, or a phosphate or modified phosphate;
R^{N} is H, alkyl, or S(O)₂-aryl, each of which can be optionally substituted by one or more R²⁰ groups;
L is H or one or more ligands;
R²⁰ is independently for each occurrence halo, haloalkyl, hydroxyl, alkoxy, methoxyalkoxy, alkylthio, amino, alkylamino, alkynyl, aminoalkyl, aminoalkoxy, nitro, acetyl, or cyano;
n is an integer of 0-4 (e.g., n is 0 or 1);
t is an integer of 1-3 representing the number of presence for unit [U-V₂], which can be the same or different for each occurrence; and
X₁ and Z₁ are each independently H, OH, OM, OR¹³, SH, SM, SR¹³, C(O)H, S(O)H, or alkyl, each of which can be optionally substituted with one or more R^{sub} groups, N(R′)(R″), B(R¹³)₃, BH₃⁻, or Se;
or X₁ and Z₁ taken together with the phosphorus atom to which they are attached forms a group of the formula,

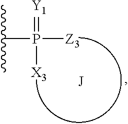

wherein ring J is a optionally substituted monocyclic or bicyclic heterocyclic ring; and X₃ and Z₃ are each independently S, O, or N(R′);
X₂ and Z₂ are each independently N(R′)(R″), or OR¹⁸;
or X₂ and Z₂ taken together with the phosphorus atom to which they are attached forms a group of the formula,

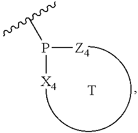

wherein ring T is a optionally substituted monocyclic or bicyclic heterocyclic ring; and X₄ and Z₄ are each independently S, O, or N(R′);
Y₁ is S, O, or N(R′),
M is an organic or inorganic cation;
R¹³ is independently for each occurrence H, alkyl, alkenyl, alkynyl, cycloalkyl, aryl, aralkyl, ω-amino alkyl, ω-hydroxy alkyl, or ω-hydroxy alkenyl, an oxygen protecting group, each of which can be optionally substituted with one or more R^{sub} groups;
R′ and R″ are each independently H, alkyl, alkenyl, alkynyl, aryl, hydroxy, alkyloxy, ω-amino alkyl, ω-hydroxy alkyl, ω-hydroxy alkenyl, or ω-hydroxy alkynyl, each of which can be optionally substituted with one or more R^{sub} groups;
R¹⁸ is H or alkyl, optionally substituted with one or more R^{sub} groups; and
R^{sub} is independently for each occurrence halo, haloalkyl, alkyl, alkaryl, aryl, aralkyl, hydroxy, alkyloxy, aryloxy, oxo, nitro, amino, acylamino, alkylcarbamoyl, arylcarbamoyl, alkylamino, aminoalkyl, alkoxycarbonyl, carboxy, hydroxyalkyl, alkanesulfonyl, arenesulfonyl, alkanesulfonamido, arenesulfonamido, aralkylsulfonamido, alkylcarbonyl, arylcarbonyl, acyloxy, cyano, or ureido.

In some embodiments, V₁ is not absent, i.e., V₁ is alkylene, arylene, heteroarylene, a divalent cycloalkyl, or a divalent heterocyclyl, each of which can be optionally substituted by one or more R²⁰ groups.

In some embodiments,

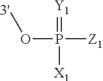

(e.g., in formula (A) or (C)) is

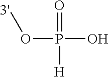

or a salt thereof. In some embodiments, the salt is an optionally substituted pyridinium salt (e.g., pyridinium or 2,6-di-tert-butylpyridinium). In one embodiment, the salt is a trialkylammonium salt (e.g., triethyammoium or N,N-diisopropylethylammoium). In one embodiment, the salt is a cyclic ammonium salt (e.g., a 1,8-diazabicyclo[5.4.0]undec-7-ene (DBU) salt or a 1,5-diazabicyclo(4.3.0)non-5-ene (DBN) salt).

In some embodiments,

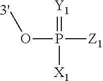

(e.g., in formula (A) or (C)) is

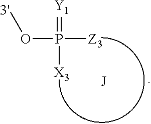

In some embodiments, Yᵢ is S. In one embodiment,

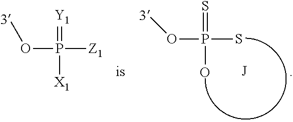

Exemplary compounds for

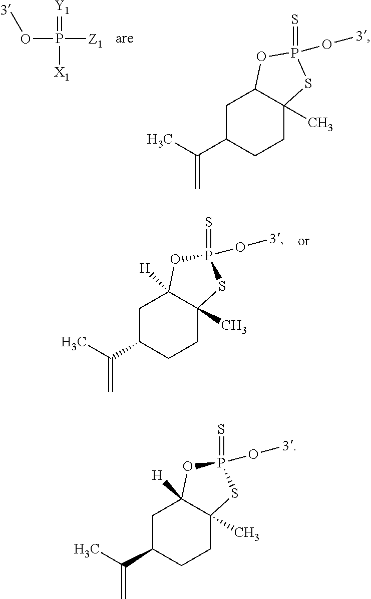

In some embodiments, X₂ in formula (B) or (D) is selected from the group consisting of —OCH₃, —OCH₂CH₃, —OCH₂CH₂CH₃, —OCH₂CH(CH₃)₂, —OCH₂CH₂CN, —OCH₂CH₂Si(CH₃)₃, —OCH₂CH₂Si(CH₂CH₃)₃, —OC(H)═CH₂, —OCH₂C(H)═CH₂,

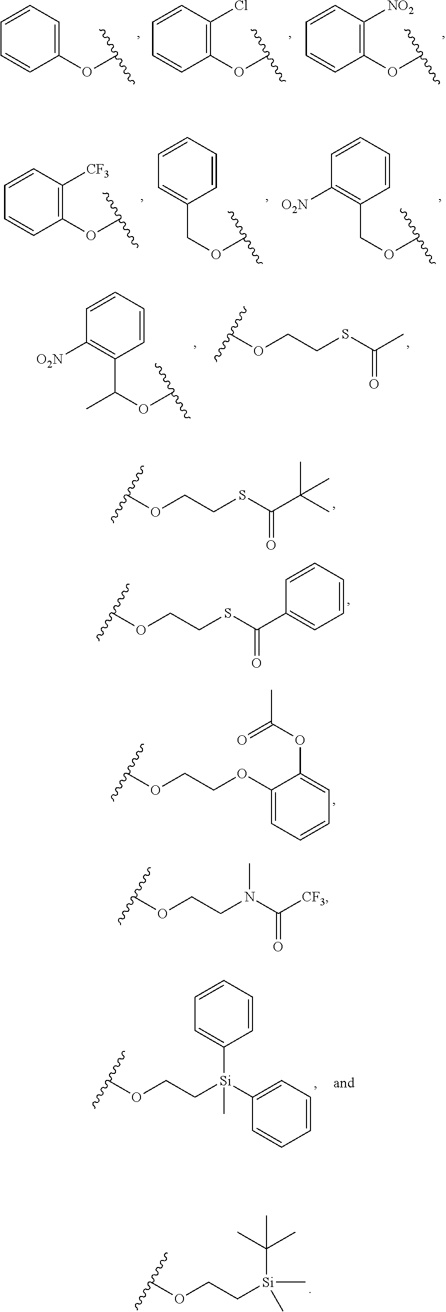

In some embodiments, Z₂ in formula (B) or (D) is selected from the group consisting of

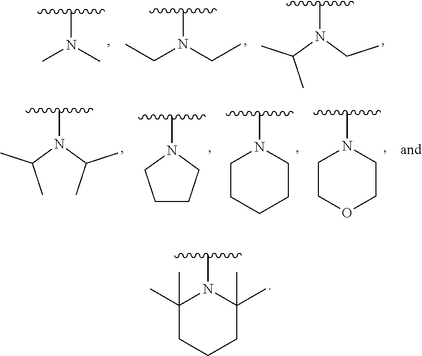

In some embodiments, in formula (B) or (D), X₂ and Z₂ taken together with the phosphorus atom to which they are attached form a cyclic structure selected from the group consisting of

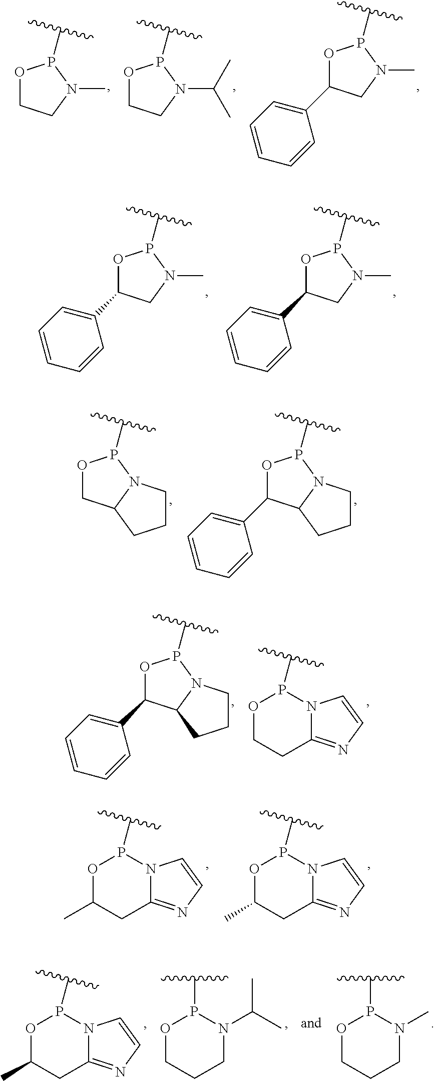

In some embodiments,

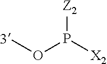

in formula (B) or (D) is

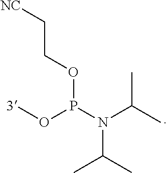

In some embodiments, n is 0 or 1.

In some embodiments, n is 0, and W is C(O)N(R^{N}) or a biocleavable carbohydrate linker.

In some embodiments, the W-V₁-[U-V₂]ₜ-L has the structure of

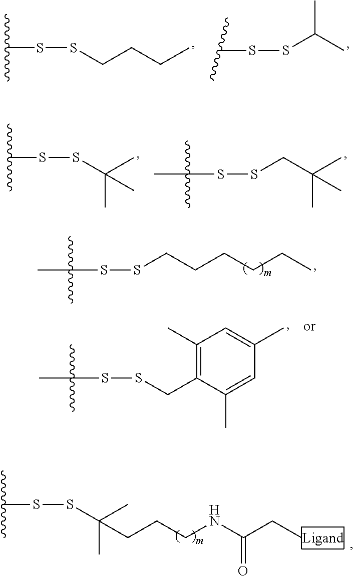

wherein m is an integer of 0-18.

In some embodiments, the W-V₁-[U-V₂]ₜ-L has the structure of

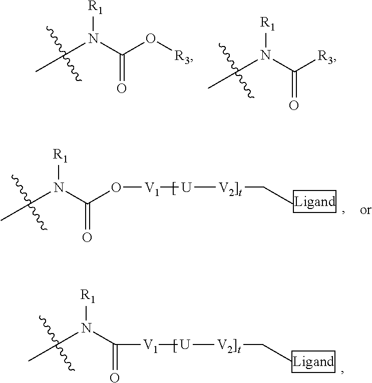

wherein:
R₃ is linear or branched alkyl, aryl,

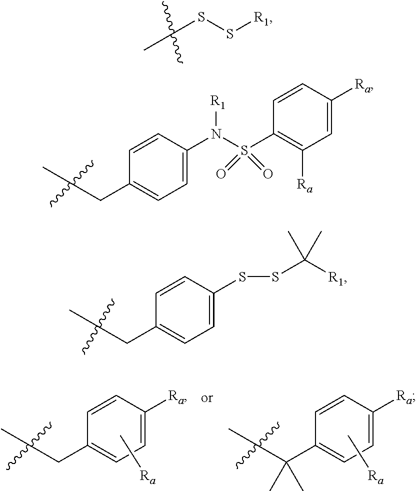

R₁ is independently for each occurrence H, linear or branched C₁-C₂₄ alkyl, aryl, hydroxyl, alkoxy, halo, methoxyalkoxy, alkylthio, amino, alkylamino, alkynyl, aminoalkyl, or aminoalkoxy; and
each Rₐ is independently for each occurrence H, NO₂, CF₃, C(O)CH₃, and CN.

In some embodiments, the W-V₁-[U-V₂]ₜ-L has the structure of

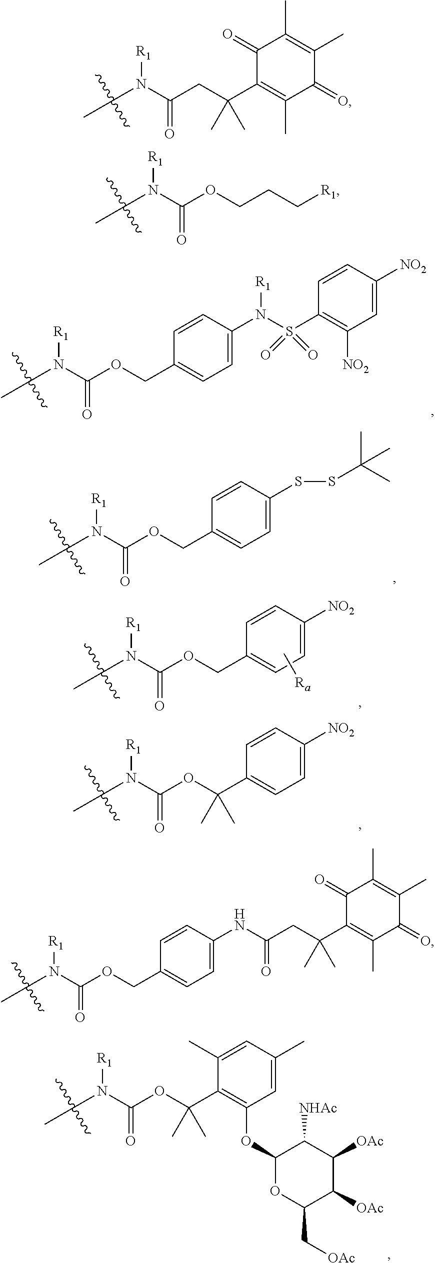

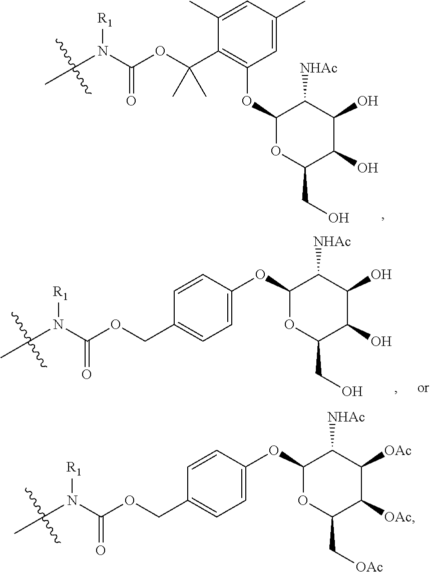

wherein R₁ is independently for each occurrence H, linear or branched C₁-C₂₄ alkyl, aryl, hydroxyl, alkoxy, halo, methoxyalkoxy, alkylthio, amino, alkylamino, alkynyl, aminoalkyl, or aminoalkoxy; and Ac is acetyl.

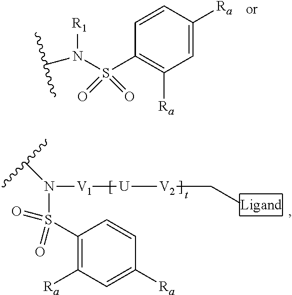

In some embodiments, the W-V₁-U-V₂-L has the structure of

wherein:
R₁ is independently for each occurrence H, linear or branched C₁-C₂₄ alkyl, aryl, hydroxyl, alkoxy, halo, methoxyalkoxy, alkylthio, amino, alkylamino, alkynyl, aminoalkyl, or aminoalkoxy; and
Rₐ is independently for each occurrence H, NO₂, CF₃, C(O)CH₃, and CN.

In some embodiments, the W-V₁-U-V₂-L has the structure of

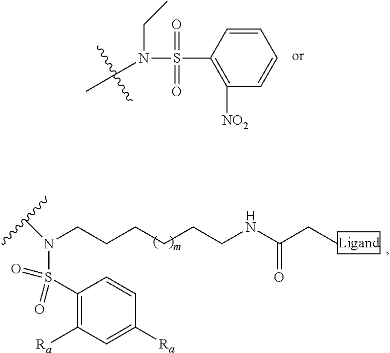

wherein Rₐ is independently for each occurrence H, NO₂, CF₃, C(O)CH₃, and CN; and m is an integer of 0-18.

In some embodiments, n is 1, and the W-V₁-U-V₂-L has the structure of

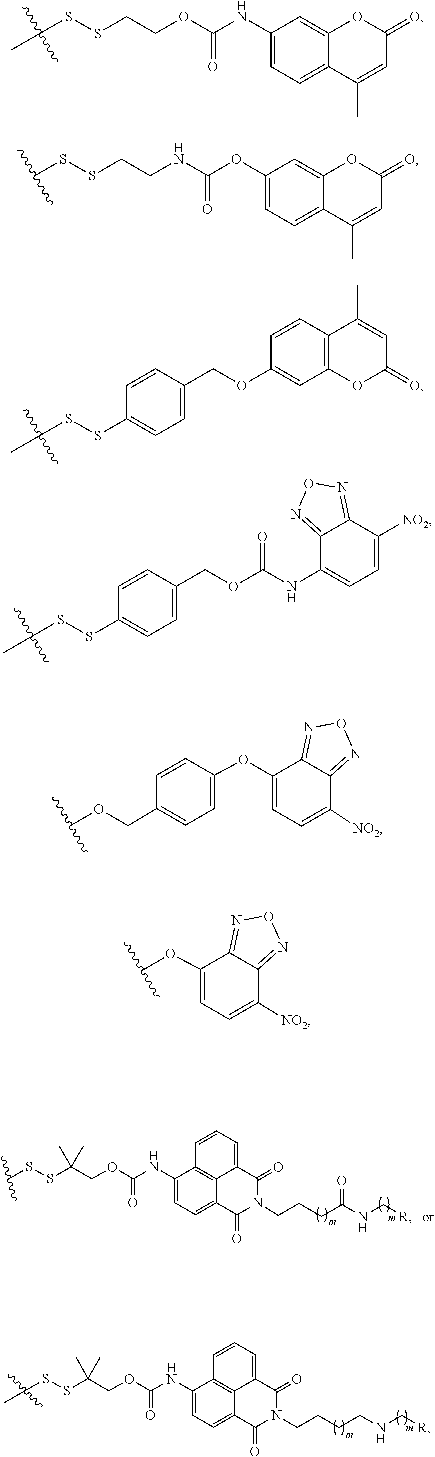

wherein:
R is

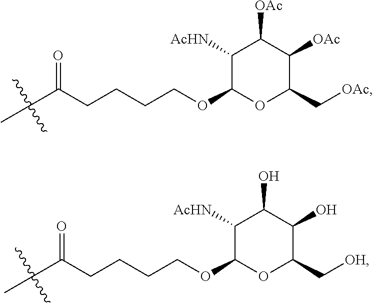 [SMILES: *C(=O)CCCCO[C@@H]1O[C@H](COC(C)=O)[C@H](OC(C)=O)[C@H](OC(C)=O)[C@H]1NC(C)=O ; *C(=O)CCCCO[C@@H]1O[C@H](CO)[C@H](O)[C@H](O)[C@H]1NC(C)=O]

a peptide, a small molecular ligand, GalNAc or multivalent GalNAc, folate or a lipophilic moiety;
Ac is acetyl, and
m is independently for each occurrence an integer of 0-18.

In some embodiments, in formula (A), (B), (C), or (D), R^{P} is an acyl group or an optionally substituted trityl group. In some embodiments, R^{P} is acetyl, pivaloyl, or optionally substituted benzoyl. In some embodiments, R is an optionally substituted trityl group. In one embodiment, R^{P} is 4,4′-dimethoxytrityl (DMTr).

In some embodiments, the compound has a structure selected from the group consisting of

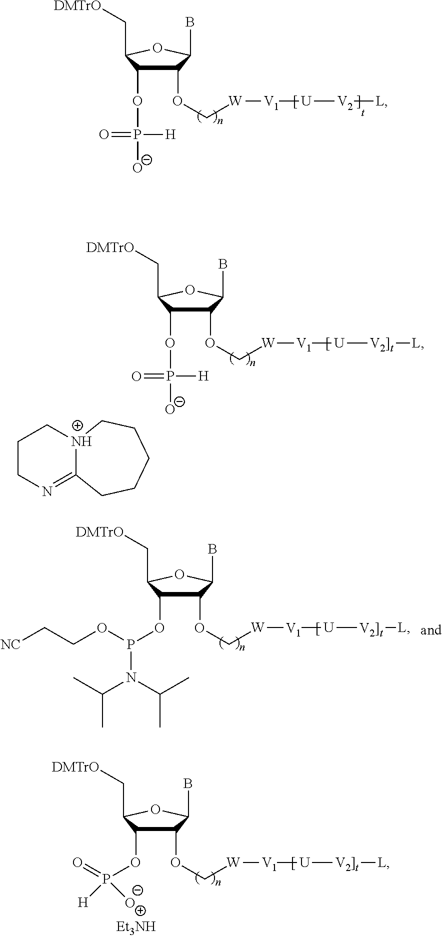

wherein DMTr is 4,4′-dimethoxytrityl.

In some embodiments, the compound has a structure of

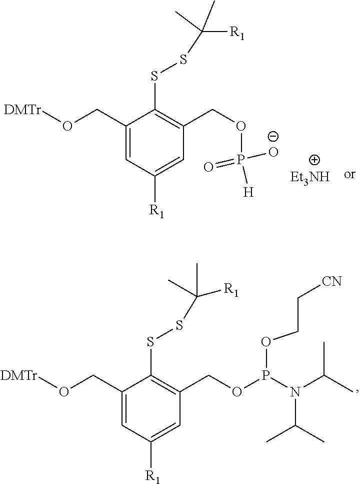

wherein DMT is 4,4′-dimethoxytrityl.

In some embodiments, the L group in all the above formulas in all the above aspects of the invention is one or more ligands, optionally connected via one or more linkers.

In some embodiments, the ligand is a targeting ligand selected from the group consisting of an antibody, a ligand-binding portion of a receptor, a ligand for a receptor, an aptamer, a carbohydrate-based ligand, a fatty acid, a lipoprotein, folate, thyrotropin, melanotropin, surfactant protein A, mucin, glycosylated polyaminoacids, transferrin, bisphosphonate, polyglutamate, polyaspartate, a lipophilic moiety (e.g., a lipophilic moiety that enhances plasma protein binding), a cholesterol, a steroid, bile acid, vitamin B12, biotin, a fluorophore, and a peptide.

In some embodiments, at least one targeting ligand targets a liver tissue. In some embodiments, the targeting ligand is a carbohydrate-based ligand. In one embodiment, the carbohydrate-based ligand is selected from the group consisting of galactose, multivalent galactose, N-acetyl-galactosamine (GalNAc), multivalent GalNAc, mannose, multivalent mannose, lactose, multivalent lactose, N-acetyl-glucosamine (GlcNAc), multivalent GlcNAc, glucose, multivalent glucose, fucose, and multivalent fucose. In one embodiment, the targeting ligand is a GalNAc conjugate. For instance, the GalNAc conjugate is one or more GalNAc derivatives attached through a bivalent or trivalent branched linker, such as:

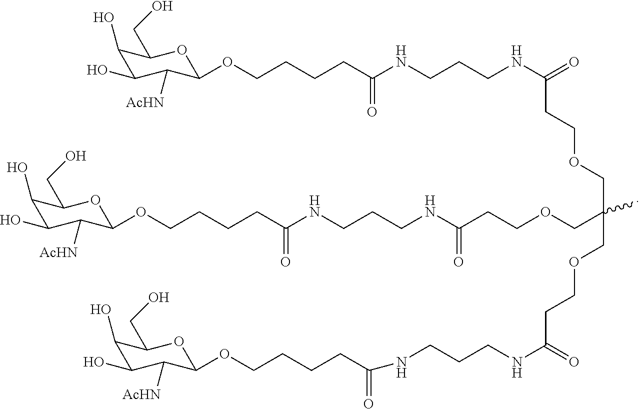 [SMILES: CC(=O)NC1C(OCCCCC(=O)NCCCNC(=O)CCOCC(C)(COCCC(=O)NCCCNC(=O)CCCCOC2OC(CO)C(O)C(O)C2NC(C)=O)COCCC(=O)NCCCNC(=O)CCCCOC2OC(CO)C(O)C(O)C2NC(C)=O)OC(CO)C(O)C1O]

In some embodiments, at least one targeting ligand is a lipophilic moiety. In one embodiment, the lipophilicity of the lipophilic moiety, measured by logK_{ow}, exceeds 0, or the hydrophobicity of the compound, measured by the unbound fraction in the plasma protein binding assay of the compound, exceeds 0.2.

In one embodiment, the lipophilic moiety contains a saturated or unsaturated C₄-C₃₀ hydrocarbon chain, and an optional functional group selected from the group consisting of hydroxy, amine, carboxylic acid, sulfonate, phosphate, thiol, azide, and alkyne. For instance, the lipophilic moiety contains a saturated or unsaturated C₆-C₁₅ hydrocarbon chain.

In some embodiments, at least one targeting ligand targets a receptor which mediates delivery to a specific CNS tissue. In one embodiment, the targeting ligand is selected from the group consisting of Angiopep-2, lipoprotein receptor related protein (LRP) ligand, bEnd.3 cell binding ligand, transferrin receptor (TfR) ligand, manose receptor ligand, glucose transporter protein, and LDL receptor ligand.

In some embodiments, at least one targeting ligand targets a receptor which mediates delivery to an ocular tissue. In one embodiment, the targeting ligand is selected from the group consisting of trans-retinol, RGD peptide, LDL receptor ligand, and carbohydrate-based ligands. In one embodiment, the targeting ligand is a RGD peptide, such as H-Gly-Arg-Gly-Asp-Ser-Pro-Lys-Cys-OH or Cyclo(-Arg-Gly-Asp-D-Phe-Cys).

In some embodiments, the oligonucleotide is a single-stranded oligonucleotide.

In some embodiments, the oligonucleotide is a double-stranded oligonucleotide comprising a sense strand and an antisense strand.

In some embodiments, the oligonucleotide contains at least one 2′-modified nucleoside at the antisense strand, sense strand, or both strands of the oligonucleotide.

In some embodiments, the oligonucleotide contains at least one 2′-modified nucleoside at the first two positions of the 5′-end of the oligonucleotide (single stranded, or sense or antisense strand for a double-stranded).

In some embodiments, the oligonucleotide contains at least one 2′-modified nucleoside at the first two positions of the 3′-end of the oligonucleotide (single stranded, or sense or antisense strand for a double-stranded).

In some embodiments, the oligonucleotide contains at least one 2′-modified nucleoside at the first two positions of the 5′-end of the oligonucleotide (single stranded, or sense or antisense strand for a double-stranded), and at least one 2′-modified nucleoside at the first two positions of the 3′-end of the oligonucleotide (single stranded, or sense or antisense strand for a double-stranded).

In some embodiments, the oligonucleotide contains at least one 2′-modified nucleoside at an internal position of the oligonucleotide (single stranded, or sense or antisense strand for a double-stranded).

In some embodiments, the antisense strand contains at least one 2′-modified nucleoside at the first two positions of the 5′-end of the antisense strand. In one embodiment, the antisense strand contains a 2′-modified nucleoside at position 1 of the 5′-end of the antisense strand. In one embodiment, the antisense strand contains a 2′-modified nucleoside at position 2 of the 5′-end of the antisense strand. In certain embodiments, the antisense strand contains one or more 2′-modified nucleosides at one or more positions selected from the group consisting of positions 2, 6, 8, 9, 14, and 16 of the 5′-end of the antisense strand. In one embodiment, the antisense strand contains 2′-modified nucleosides at position 2, position 14, or positions 2 and 14 of the 5′-end of the antisense strand.

In some embodiments, the sense and antisense strands are each 15 to 30 nucleotides in length. In one embodiment, the sense and antisense strands are each 18 to 30 nucleotides in length. In one embodiment, the sense and antisense strands are each 19 to 25 nucleotides in length. In one embodiment, the sense and antisense strands are each 21 to 23 nucleotides in length.

In some embodiments, the sense strand has a length of 18 to 30 nucleotides (e.g., 19-25 nucleotides, 21-23 nucleotides). The sense strand contains at least one 2′-modified nucleoside at positions 7, 8, 9, 10, 11, 12, or 13, counting from 5′-end of the sense strand. In one embodiment, the sense strand contains at least two 2′-modified nucleosides at positions 7, 8, 9, 10, 11, 12, or 13, counting from 5′-end of the sense strand. In one embodiment, the sense strand contains one or two 2′-modified nucleosides at positions 9, 10, or 11, counting from 5′-end of the sense strand.

In some embodiments, the sense strand further comprises one or two 2′-deoxy modifications within positions 7, 8, 9, 10, 11, 12, or 13, counting from 5′-end of the sense strand. In one embodiment, the one or two 2′-deoxy modifications are within positions 9, 10, or 11, counting from 5′-end of the sense strand.

In one embodiment, each of positions 9 and 11 of the sense strand, counting from 5′-end of the sense strand, are a 2′-modified nucleoside or a 2′-deoxy modification.

In some embodiments, the oligonucleotide comprises a single-stranded overhang on at least one of the termini, e.g., 3′ and/or 5′ overhang(s) of 1-10 nucleotides in length, for instance, an overhang having 1, 2, 3, 4, 5, or 6 nucleotides in length. In some embodiments, both strands have at least one stretch of 1-5 (e.g., 1, 2, 3, 4, or 5) single-stranded nucleotides in the double stranded region. In one embodiment, the single-stranded overhang is 1, 2, or 3 nucleotides in length, optionally on at least one of the termini.

In some embodiments, the oligonucleotide may also have a blunt end, located at the 5′-end of the antisense strand (or the 3′-end of the sense strand), or vice versa. In one embodiment, the oligonucleotide comprises a 3′ overhang at the 3′-end of the antisense strand, and optionally a blunt end at the 5′-end of the antisense strand. In one embodiment, the oligonucleotide has a 5′ overhang at the 5′-end of the sense strand, and optionally a blunt end at the 5′-end of the antisense strand. In one embodiment, the oligonucleotide has two blunt ends at both ends of a double-stranded iRNA duplex.

In one embodiment, the sense strand of the oligonucleotide is 21-nucleotide in length, and the antisense strand is 23-nucleotide in length, wherein the strands form a double-stranded region of 21 consecutive base pairs having a 2-nucleotide long single-stranded overhangs at the 3′-end.

In one embodiment, the sense strand contains at least one 2′-modified nucleoside of formula (I). In one embodiment, the antisense strand contains at least one 2′-modified nucleoside of formula (I). In one embodiment, both the sense strand and the antisense strand each contain at least one 2′-modified nucleoside of formula (I).

In some embodiments, the 2′-modified nucleoside of formula (I) has a structure selected from the group consisting of:

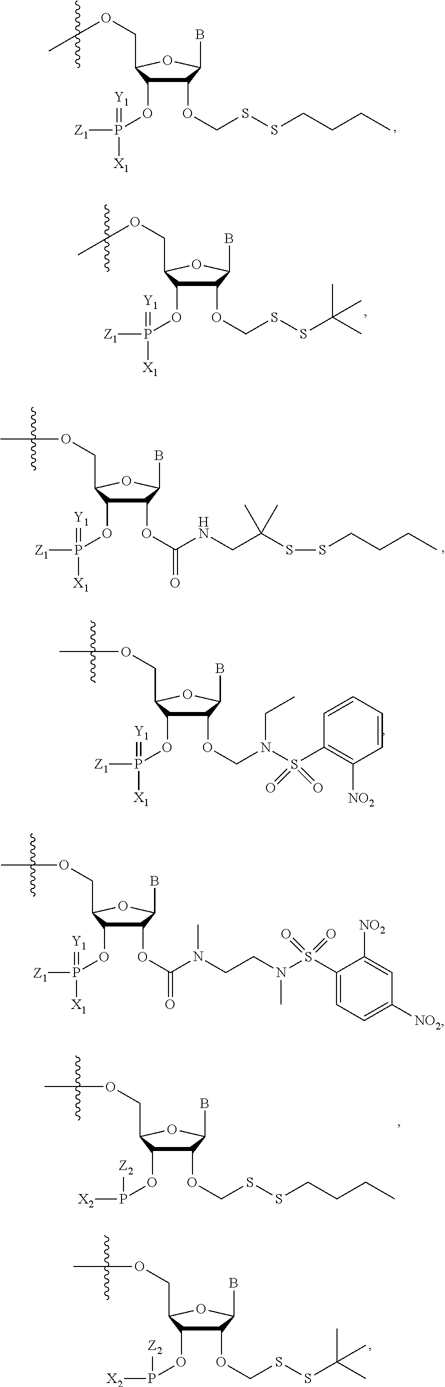

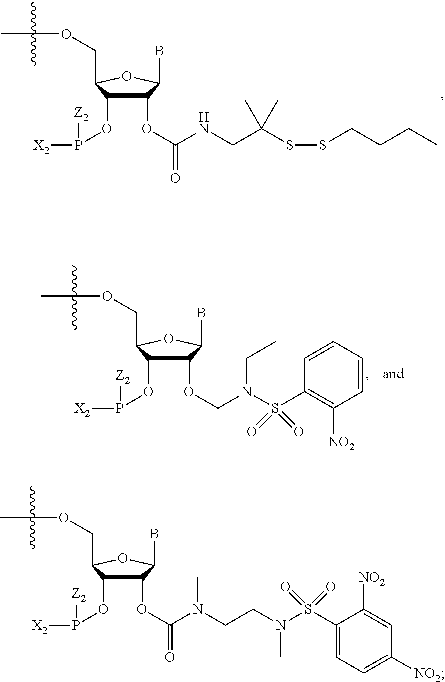

or a salt thereof.

In one embodiment, the 2′-modified nucleoside has a structure selected from the group consisting of:

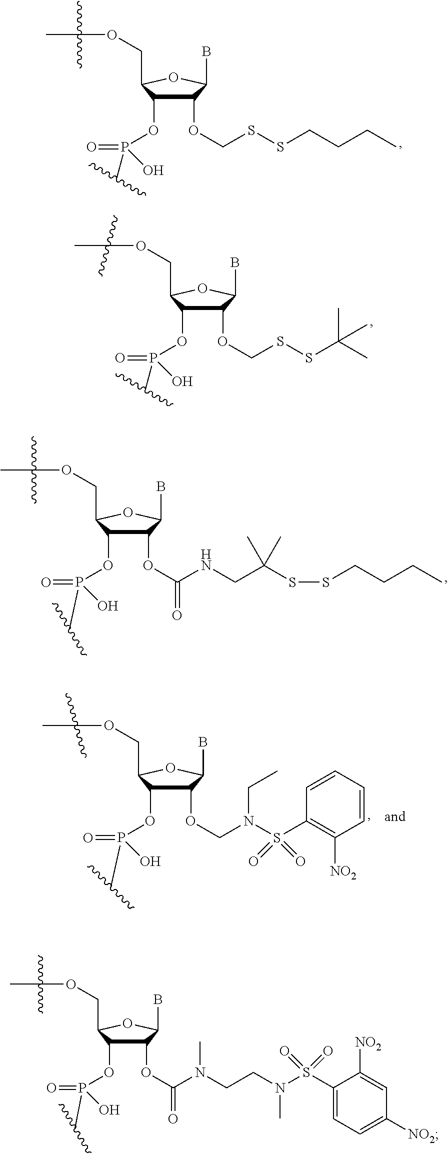

or a salt thereof.

In some embodiments, the 2′-position of the nucleoside has the structure of

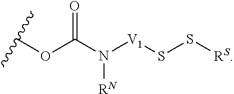

R⁵ is —V₂-[-U-V₂-]ₜ-L or -[-U-V₂-]ₜ-L. The variables R^{N}, V₁, V₂, U, L, and t are the same as those defined above in formula (I). In one embodiment, t is 1-2. In some embodiments, V₁ is alkylene optionally substituted by one or more R²⁰ groups. In some embodiments, V₁ is ethylene or propylene optionally substituted by one or more R²⁰ groups. In one embodiment, V₁ is —CH₂—C(R²⁰)₂—, for instance, V₁ is —CH₂—C(CH₃)₂—. In one embodiment, R^{N} is hydrogen or alkyl.

In some embodiments, the 2′-position of the nucleoside has the structure of

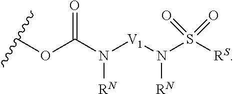

R⁵ is —V₂-[-U-V₂-]ₜ-L or -[-U-V₂-]ₜ-L. The variables R^{N}, V₁, V₂, U, L, and t are the same as those defined above in formula (I). In one embodiment, t is 1-2. In some embodiments, V₁ is alkylene optionally substituted by one or more R²⁰ groups. In some embodiments, V₁ is ethylene or propylene optionally substituted by one or more R²⁰ groups. In one embodiment, V₁ is —CH₂—C(R²⁰)₂—, for instance, V₁ is —CH₂—C(CH₃)₂—. In one embodiment, R^{N} is hydrogen or alkyl.

In some embodiments, the 2′-position of the nucleoside has the structure of

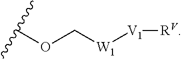

W₁ is —S-S—, —N(R^{N})C(O)O—*, or —N(R^{N})SO₂—*, wherein * represents the bond to V₁. R^{V} is -[-U-V₂-]ₜ-L. The variables R^{N}, V₁, V₂, U, L, and t are the same as those defined above in formula (I). In one embodiment, t is 1-2. In one embodiments, W₁ is —S—S—. In one embodiment, W₁ is —N(R^{N})C(O)O—*. In one embodiment, W₁ is —N(R^{N})SO₂—*. In some embodiments, V₁ is alkylene optionally substituted by one or more R²⁰ groups. In some embodiments, V₁ is ethylene or propylene optionally substituted by one or more R²⁰ groups. In one embodiment, V₁ is —CH₂—C(R²⁰)₂—, for instance, V₁ is —CH₂—C(CH₃)₂—. In one embodiment, R^{N} is hydrogen or alkyl.

In some embodiments, the oligonucleotide further comprises at least one ligand at the 3′-end of the sense strand.

In some embodiments, the sense strand further comprises at least one phosphorothioate linkage at the 3′-end. In some embodiments, the sense strand comprises at least two phosphorothioate linkages at the 3′-end.

In some embodiments, the sense strand further comprises at least one phosphorothioate linkage at the 5′-end. In some embodiments, the sense strand comprises at least two phosphorothioate linkages at the 5′-end.

In some embodiments, the antisense strand further comprises at least one phosphorothioate linkage at the 3′-end. In some embodiments, the antisense strand comprises at least two phosphorothioate linkages at the 3′-end.

In some embodiments, the oligonucleotide further comprises a phosphate or phosphate mimic at the 5′-end of the antisense strand. In one embodiment, the phosphate mimic is a 5′-vinyl phosphonate (VP).

In some embodiments, the 5′-end of the antisense strand does not contain a 5′-vinyl phosphonate (VP).

In some embodiments, the oligonucleotide further comprises at least one terminal, chiral phosphorus atom.

A site specific, chiral modification to the internucleotide linkage may occur at the 5′ end, 3′ end, or both the 5′ end and 3′ end of a strand. This is being referred to herein as a “terminal” chiral modification. The terminal modification may occur at a 3′ or 5′ terminal position in a terminal region, e.g., at a position on a terminal nucleotide or within the last 2, 3, 4, 5, 6, 7, 8, 9 or 10 nucleotides of a strand. A chiral modification may occur on the sense strand, antisense strand, or both the sense strand and antisense strand. Each of the chiral pure phosphorus atoms may be in either Rp configuration or Sp configuration, and combination thereof. More details regarding chiral modifications and chirally-modified dsRNA agents can be found in PCT/US18/67103, entitled “Chirally-Modified Double-Stranded RNA Agents,” filed Dec. 21, 2018, which is incorporated herein by reference in its entirety.

In some embodiments, the oligonucleotide further comprises a terminal, chiral modification occurring at the first internucleotide linkage at the 3′ end of the antisense strand, having the linkage phosphorus atom in Sp configuration; a terminal, chiral modification occurring at the first internucleotide linkage at the 5′ end of the antisense strand, having the linkage phosphorus atom in Rp configuration; and a terminal, chiral modification occurring at the first internucleotide linkage at the 5′ end of the sense strand, having the linkage phosphorus atom in either Rp configuration or Sp configuration.

In one embodiment, the oligonucleotide further comprises a terminal, chiral modification occurring at the first and second internucleotide linkages at the 3′ end of the antisense strand, having the linkage phosphorus atom in Sp configuration; a terminal, chiral modification occurring at the first internucleotide linkage at the 5′ end of the antisense strand, having the linkage phosphorus atom in Rp configuration; and a terminal, chiral modification occurring at the first internucleotide linkage at the 5′ end of the sense strand, having the linkage phosphorus atom in either Rp or Sp configuration.

In one embodiment, the oligonucleotide further comprises a terminal, chiral modification occurring at the first, second, and third internucleotide linkages at the 3′ end of the antisense strand, having the linkage phosphorus atom in Sp configuration; a terminal, chiral modification occurring at the first internucleotide linkage at the 5′ end of the antisense strand, having the linkage phosphorus atom in Rp configuration; and a terminal, chiral modification occurring at the first internucleotide linkage at the 5′ end of the sense strand, having the linkage phosphorus atom in either Rp or Sp configuration.

In one embodiment, the oligonucleotide further comprises a terminal, chiral modification occurring at the first and second internucleotide linkages at the 3′ end of the antisense strand, having the linkage phosphorus atom in Sp configuration; a terminal, chiral modification occurring at the third internucleotide linkages at the 3′ end of the antisense strand, having the linkage phosphorus atom in Rp configuration; a terminal, chiral modification occurring at the first internucleotide linkage at the 5′ end of the antisense strand, having the linkage phosphorus atom in Rp configuration; and a terminal, chiral modification occurring at the first internucleotide linkage at the 5′ end of the sense strand, having the linkage phosphorus atom in either Rp or Sp configuration.

In one embodiment, the oligonucleotide further comprises a terminal, chiral modification occurring at the first and second internucleotide linkages at the 3′ end of the antisense strand, having the linkage phosphorus atom in Sp configuration; a terminal, chiral modification occurring at the first, and second internucleotide linkages at the 5′ end of the antisense strand, having the linkage phosphorus atom in Rp configuration; and a terminal, chiral modification occurring at the first internucleotide linkage at the 5′ end of the sense strand, having the linkage phosphorus atom in either Rp or Sp configuration.

In some embodiments, the oligonucleotide has at least two phosphorothioate internucleotide linkages at the first five nucleotides on the antisense strand (counting from the 5′ end).

In some embodiments, the antisense strand comprises two blocks of one, two, or three phosphorothioate internucleotide linkages separated by 1, 2, 3, 4, 5, 6, 7, 8, 9, 10, 11, 12, 13, 14, 15, 16, 17, or 18 phosphate internucleotide linkages.

In some embodiments, 100%, 95%, 90%, 85%, 80%, 75%, 70%, 65%, 60%, 55%, 50%, 45%, 40%, 35% or 30% of the antisense and sense strand of the oligonucleotide is modified. For example, when 50% of the oligonucleotide is modified, 50% of all nucleotides present in the oligonucleotide contain a modification as described herein.

In some embodiments, the antisense and sense strands of the oligonucleotide comprise at least 30%, 35%, 40%, 45%, 50%, 55%, 60%, 65%, 70%, 75%, 80%, 85%, 90%, 95%, or virtually 100% 2′-O-methyl modified nucleotides.

In one embodiment, the oligonucleotide is a double-stranded dsRNA agent, and at least 50% of the nucleotides of the double-stranded dsRNA agent is independently modified with 2′-O-methyl, 2′-O-allyl, 2′-deoxy, or 2′-fluoro.

In one embodiment, the oligonucleotide is an antisense, and at least 50% of the nucleotides of the antisense is independently modified with LNA, CeNA, 2′-methoxyethyl, or 2′-deoxy.

In some embodiments, the sense and antisense strands comprise 12 or less, 10 or less, 8 or less, 6 or less, 4 or less, 2 or less, or no 2′-F modified nucleotides. In some embodiments, the oligonucleotide has 12 or less, 10 or less, 8 or less, 6 or less, 4 or less, 2 or less, or no 2′-F modifications on the sense strand. In some embodiments, the oligonucleotide has 12 or less, 10 or less, 8 or less, 6 or less, 4 or less, 2 or less, or no 2′-F modifications on the antisense strand. In one embodiment, the sense and the antisense strands comprise no more than ten 2′-fluoro modified nucleotides. In one embodiment, the sense strand comprises no 2′-fluoro modified nucleotides.

In some embodiments, the oligonucleotide contains one or more 2′-0 modifications selected from the group consisting of 2′-deoxy, 2′-O-methoxyalkyl, 2′-O-methyl, 2′-O-allyl, 2′-C-allyl, 2′-fluoro, 2′-O—N-methylacetamido (2′-O-NMA), 2′-O-dimethylaminoethoxyethyl (2′-O-DMAEOE), 2′-O-aminopropyl (2′-O-AP), and 2′-ara-F.

In some embodiments, the oligonucleotide has one or more 2′-F modifications on any position of the sense strand or antisense strand.

In some embodiments, the oligonucleotide has less than 20%, less than 15%, less than 10%, less than 5% non-natural nucleotide, or substantially no non-natural nucleotide. Examples of non-natural nucleotide include acyclic nucleotides, LNA, HNA, CeNA, 2′-O-methoxyalkyl (e.g., 2′-O-methoxymethyl, 2′-O-methoxyethyl, or 2′-O-2-methoxypropanyl), 2′-O-allyl, 2′-C-allyl, 2′-fluoro, 2′-O—N-methylacetamido (2′-O-NMA), a 2′-O-dimethylaminoethoxyethyl (2′-O-DMAEOE), 2′-O-aminopropyl (2′-O-AP), 2′-ara-F, L-nucleoside modification (such as 2′-modified L-nucleoside, e.g., 2′-deoxy-L-nucleoside), BNA abasic sugar, abasic cyclic and open-chain alkyl.

In some embodiments, the oligonucleotide has greater than 80%, greater than 85%, greater than 90%, greater than 95%, or virtually 100% natural nucleotides. For the purpose of these embodiments, natural nucleotides can include those having 2′-OH, 2′-deoxy, and 2′-OMe.

In some embodiments, the antisense strand contains at least one unlocked nucleic acids (UNA) or glycerol nucleic acid (GNA) modification, e.g., at the seed region of the antisense strand. In one embodiment, the seed region is at positions 2-8 (or positions 5-7) of the 5′-end of the antisense strand.

In one embodiment, the oligonucleotide comprises a sense strand and antisense strand each having a length of 15-30 nucleotides; at least two phosphorothioate internucleotide linkages at the first five nucleotides on the antisense strand (counting from the 5′ end); wherein the duplex region is between 19 to 25 base pairs (preferably 19, 20, 21 or 22); wherein the oligonucleotide has less than 20%, less than 15%, less than 10%, less than 5% non-natural nucleotide, or substantially no non-natural nucleotide.

In one embodiment, the oligonucleotide comprises a sense strand and antisense strand each having a length of 15-30 nucleotides; at least two phosphorothioate internucleotide linkages at the first five nucleotides on the antisense strand (counting from the 5′ end); wherein the duplex region is between 19 to 25 base pairs (preferably 19, 20, 21 or 22); wherein the oligonucleotide has greater than 80%, greater than 85%, greater than 95%, or virtually 100% natural nucleotides, such as those having 2′-OH, 2′-deoxy, or 2′-OMe.

One aspect of the invention provides an oligonucleotide comprising a sense strand and an antisense strand, each strand independently having a length of 15 to 35 nucleotides; at least two phosphorothioate internucleotide linkages between the first five nucleotides counting from the 5′ end of the antisense strand; at least three, four, five, or six 2′-deoxy modifications on the sense and/or antisense strands; wherein the oligonucleotide has a double stranded (duplex) region of between 19 to 25 base pairs; wherein the oligonucleotide comprises a ligand.

In one embodiment, the sense strand does not comprise a glycol nucleic acid (GNA).

It is understood that the antisense strand has sufficient complementarity to a target sequence to mediate RNA interference. In other words, the oligonucleotide is capable of inhibiting the expression of a target gene.

In one embodiment, the oligonucleotide comprises at least three 2′-deoxy modifications. The 2′-deoxy modifications are at positions 2 and 14 of the antisense strand, counting from 5′-end of the antisense strand, and at position 11 of the sense strand, counting from 5′-end of the sense strand.

In one embodiment, the oligonucleotide comprises at least five 2′-deoxy modifications. The 2′-deoxy modifications are at positions 2, 12 and 14 of the antisense strand, counting from 5′-end of the antisense strand, and at positions 9 and 11 of the sense strand, counting from 5′-end of the sense strand.

In one embodiment, the oligonucleotide comprises at least seven 2′-deoxy modifications. The 2′-deoxy modifications are at positions 2, 5, 7, 12 and 14 of the antisense strand, counting from 5′-end of the antisense strand, and at positions 9 and 11 of the sense strand, counting from 5′-end of the sense strand.

In one embodiment, the antisense strand comprises at least five 2′-deoxy modifications at positions 2, 5, 7, 12 and 14, counting from 5′-end of the antisense strand. The antisense strand has a length of 18-25 nucleotides, or a length of 18-23 nucleotides.

In one embodiment, the oligonucleotide comprises less than 20%, e.g., less than 15%, less than 10%, or less than 5% non-natural nucleotides, or comprises no non-natural nucleotides.

In one embodiment, the sense strand does not comprise a glycol nucleic acid (GNA); and wherein the oligonucleotide comprises less than 20%, e.g., less than 15%, less than 10%, or less than 5% non-natural nucleotides or comprises all natural nucleotides.

In one embodiment, at least one the sense and antisense strands comprises at least one, e.g., at least two, at least three, at least four, at least five, at least six, or at least seven or more, 2′-deoxy modifications in a central region of the sense or antisense strand.

In one embodiment, the sense strand and/or the antisense strand comprises at least one, e.g., at least two, at least three, at least four, at least five, at least six, or at least seven or more, 2′-deoxy modifications in a central region of the sense strand and/or the antisense strand.

In some embodiment, the sense strand has a length of 18 to 30 nucleotides and comprises at least two 2′-deoxy modifications in the central region of the sense strand. For example, the sense strand has a length of 18 to 30 nucleotides and comprises at least two 2′-deoxy modifications within positions 7, 8, 9, 10, 11, 12, and 13, counting from 5′-end of the sense strand.

In one embodiment, the antisense strand has a length of 18 to 30 nucleotides and comprises at least two 2′-deoxy modifications in the central region of the antisense strand. For example, the antisense strand has length of 18 to 30 nucleotides and comprises at least two 2′-deoxy modifications within positions 10, 11, 12, 13, 14, 15 and 16, counting from 5′-end of the antisense strand.

In one embodiment, the oligonucleotide comprises a sense strand and an antisense strand; wherein the sense strand has a length of 17-30 nucleotide and comprises at least one 2′-deoxy modification in the central region of the sense strand; and wherein the antisense strand independently has a length of 17-30 nucleotides and comprises at least two 2′-deoxy modifications in the central region of the antisense strand.

In one embodiment, the oligonucleotide comprises a sense strand and an antisense strand; wherein the sense strand has a length of 17-30 nucleotide and comprises at least two 2′-deoxy modifications in the central region of the sense strand; and wherein the antisense strand independently has a length of 17-30 nucleotides and comprises at least one 2′-deoxy modification in the central region of the antisense strand.

In one embodiment, the sense strand comprises at least one, e.g., at least two, at least three, at least four, at least five, at least six, at least seven or more, 2′-deoxy modifications in a central region of the sense strand.

In one embodiment, the antisense strand comprises at least one, e.g., at least two, at least three, at least four, at least five, at least six, at least seven or more, 2′-deoxy modifications in a central region of the antisense strand.

In one embodiment, the oligonucleotide comprises less than 20%, e.g., less than 15%, less than 10%, or less than 5% non-natural nucleotides or the oligonucleotide comprises all natural nucleotides; and wherein the sense strand and/or the antisense strand comprises at least one, e.g., at least two, at least three, at least four, at least five, at least six, at least seven or more, 2′-deoxy modifications in a central region of the sense strand and/or the antisense strand.

In one embodiment, the oligonucleotide comprises less than 20%, e.g., less than 15%, less than 10%, or less than 5% non-natural nucleotides or the oligonucleotide comprises all natural nucleotides; and wherein the sense strand comprises at least one, e.g., at least two, at least three, at least four, at least five, at least six, at least seven or more, 2′-deoxy modifications in a central region of the sense strand.

In one embodiment, the oligonucleotide comprises less than 20%, e.g., less than 15%, less than 10%, or less than 5% non-natural nucleotides or the oligonucleotide comprises all natural nucleotides; and wherein the antisense strand comprises at least one, e.g., at least two, at least three, at least four, at least five, at least six, at least seven or more, 2′-deoxy modifications in a central region of the antisense strand.

In one embodiment, when the oligonucleotide comprises less than 8 non-2′OMe nucleotides, the antisense stand comprises at least one DNA. For example, in any one of the embodiments of the invention when the oligonucleotide comprises less than 8 non-2′OMe nucleotides, the antisense stand comprises at least one DNA.

In one embodiment, when the antisense comprises two deoxy nucleotides and said nucleotides are at positions 2 and 14, counting from the 5′-end of the antisense strand, the oligonucleotide comprises 8 or less (e.g., 8, 7, 6, 5, 4, 3, 2, 1 or 0) non-2′OMe nucleotides. For example, in any one of the embodiments of the invention when the antisense comprises two deoxy nucleotides and said nucleotides are at positions 2 and 14, counting from the 5′-end of the antisense strand, the oligonucleotide comprises 0, 1, 2, 3, 4, 5, 6, 7 or 8 non 2′-OMe nucleotides.

Another aspect of the invention relates to a pharmaceutical composition comprising the oligonucleotide described herein, and a pharmaceutically acceptable excipient.

All the above embodiments relating to the oligonucleotide in the above aspect of the invention relating to the oligonucleotide are suitable in this aspect of the invention relating to the pharmaceutical composition.

In another aspect, the invention further provides a method for delivering the oligonucleotide of the invention to a specific target in a subject by subcutaneous or intravenous administration. The invention further provides the oligonucleotide of the invention for use in a method for delivering said agents to a specific target in a subject by subcutaneous or intravenous administration.

Another aspect of the invention relates to a method of reducing or inhibiting the expression of a target gene in a subject, comprising administering to the subject the oligonucleotide described herein above.

All the above embodiments relating to the oligonucleotide in the above aspect of the invention relating to the oligonucleotide are suitable in this aspect of the invention relating to a method of reducing the expression of a target gene in a subject.

Another aspect of the invention relates to a method of bioactivating an oligonucleotide that comprises one or more 2′-modified nucleosides, wherein the 2′-position of the nucleoside is modified by a bio-cleavable linking group optionally connected to a ligand. The method comprises the step of: exposing the oligonucleotide to a physiological condition that causes the bio-cleavable linking group to be cleaved from the 2′-modified nucleoside, thereby regenerating the 2′—OH group of the nucleoside. The bio-cleavable linking group comprises acetal, disulfide, carbamate, amide, sulfonamide, a biocleavable carbohydrate linker, or combinations thereof.

In some embodiments, the physiological condition comprises an oxidative and/or reductive conditions. In one embodiment, the physiological condition comprises a cellular enzyme. In one embodiment, the physiological condition comprises a glutathione.

In some embodiments, the bio-cleavable linking group is connected one or more ligands, selected from the group consisting of an antibody, a ligand-binding portion of a receptor, a ligand for a receptor, an aptamer, a carbohydrate-based ligand, a fatty acid, a lipoprotein, folate, thyrotropin, melanotropin, surfactant protein A, mucin, glycosylated polyaminoacids, transferrin, bisphosphonate, polyglutamate, polyaspartate, a lipophilic moiety that enhances plasma protein binding, a cholesterol, a steroid, bile acid, vitamin B12, biotin, a fluorophore, and a peptide..

In one embodiment, at least one ligand is a carbohydrate-based ligand targeting a liver tissue.

In one embodiment, at least one ligand is a lipophilic moiety.

In one embodiment, at least one ligand targets a receptor which mediates delivery to a CNS tissue or an ocular tissue.

In one embodiment, at least one ligand is a fluorophore.

In some embodiments, in the oligonucleotide that comprises one or more 2′-modified nucleosides, the 2′-position of the nucleoside has a structure of formula (I):

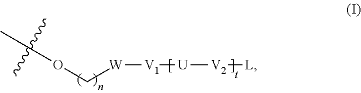

wherein:
W is O, S—S, N(R^{N}), C(O)N(R^{N}), N(R^{N})C(O), N(R^{N})C(O)O, N(R^{N})S(O)₂, or a biocleavable carbohydrate linker;
V₁ and V₂ are each independently for each occurrence absent, alkylene, arylene, heteroarylene, a divalent cycloalkyl, or a divalent heterocyclyl, each of which can be optionally substituted by one or more R²⁰ groups;
U is independently for each occurrence absent, W, O—W, W—O, or a phosphate or modified phosphate;
R^{N} is H, alkyl, or S(O)₂-aryl, each of which can be optionally substituted by one or more R²⁰ groups;
L is H or one or more ligands;
R²⁰ is independently for each occurrence halo, haloalkyl, hydroxyl, alkoxy, methoxyalkoxy, alkylthio, amino, alkylamino, alkynyl, aminoalkyl, aminoalkoxy, nitro, acetyl, or cyano;
n is an integer of 0-4;
t is an integer of 1-3 representing the number of presence for unit [U-V₂], which can be the same or different for each occurrence; and
[symbol] indicates the attachment of the formula (I) to the 2′-position of the nucleoside.

All the above embodiments relating to the oligonucleotide and structural formula at the 2′-position of the nucleoside in the above aspect of the invention relating to the oligonucleotide are suitable in this aspect of the invention relating to a method of bioactivating an oligonucleotide.

### BRIEF DESCRIPTION OF THE DRAWINGS

FIG. 1 is a schematic description of an siRNA containing the 2′-modified nucleoside prodrug designed for intracellular delivery.

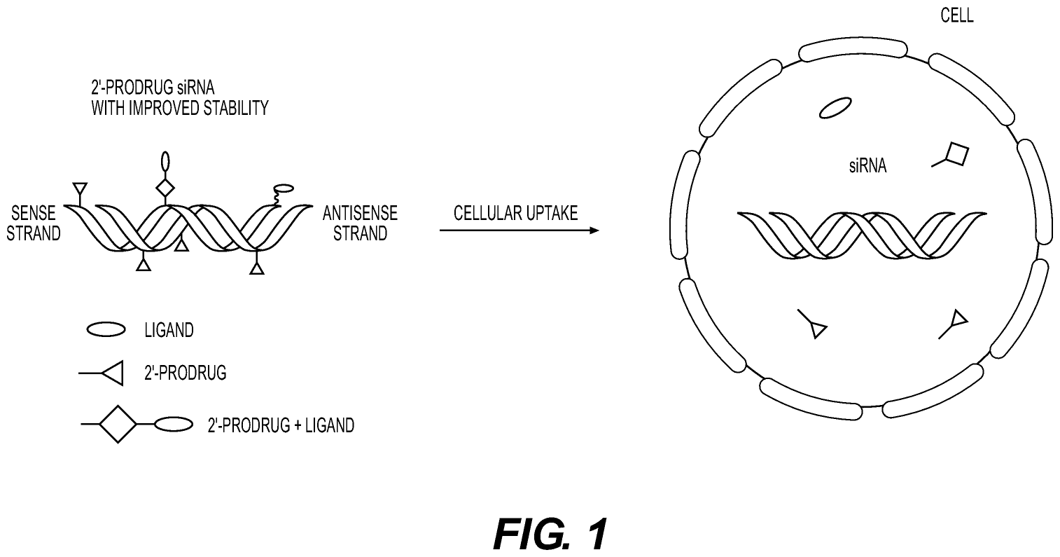

FIG. 2 is a graph depicting the cleavage profiles of different single-stranded oligonucleotides, containing different chemical modification at the 2′-position to protect 2′-OH group, after being treated with glutathione and followed by HPLC over 25 hours.

FIG. 3 is a graph depicting the relative mTTR protein in circulation by ELISA in mice following subcutaneous administration of modified TTR siRNAs, containing the 2′-modified nucleoside prodrug at N1 of the antisense strand, at single dose 0.75 mg/kg compared to PBS control.

FIG. 4 is a graph depicting the relative mTTR protein in circulation by ELISA in mice following subcutaneous administration of modified TTR siRNAs, containing the 2′-modified nucleoside prodrug at N2 of the antisense strand, at single dose 0.75 mg/kg compared to PBS control.

FIG. 5 is a graph depicting in vitro gene silencing activities of TTR mRNA-targeting siRNAs containing the 2′-modified nucleoside prodrug via free uptake in primary mouse hepatocytes at 1, 10, and 100 nm concentrations in cell culture medium.

FIG. 6 is a graph depicting in vitro gene silencing activities of TTR mRNA-targeting siRNAs containing the 2′-modified nucleoside prodrug via transfection with RNAiMAX in primary mouse hepatocytes at 0.1, 1, and 10 nm concentrations.

FIG. 7 is a graph depicting the relative mTTR protein in circulation by ELISA in mice following subcutaneous administration of modified TTR siRNAs containing 2′-prodrug Y87 at position 9 or 11 of the sense strand, at a single dose 0.75 mg/kg.

FIG. 8 is a graph depicting the relative mTTR protein in circulation by ELISA in mice following subcutaneous administration of modified TTR siRNAs containing 2′-prodrug Y137 at position 9 or 11 of the sense strand, at a single dose 0.75 mg/kg.

FIG. 9 is a graph depicting the relative mTTR protein in circulation by ELISA in mice following subcutaneous administration of modified TTR siRNAs containing 2′-prodrug Y₁₃₉ at position 9 or 11 of the sense strand, at a single dose 0.75 mg/kg.

FIG. 10 is a graph depicting the relative mTTR protein in circulation by ELISA in mice following subcutaneous administration of modified TTR siRNAs containing 2′-prodrug Y87 at positions 9 and 11 of the sense strand, or 2′-prodrug Y87 at position 2 of the antisense strand, at a single dose 0.75 mg/kg.

FIG. 11 is a graph depicting the relative mSOD1 mRNA by qPCR compared to aCSF in mice brain following ICV administration of modified SOD1 siRNAs containing 2′-prodrug at a single dose of 100 μg of siRNA at day 8.

### DETAILED DESCRIPTION

The inventors have discovered novel concept of temporarily masking the 2′-position of the nucleoside of an oligonucleotide (e.g., a single-stranded iRNA agent a double-stranded iRNA agent) using bio-cleavable linkers. Under specific cellular environments and conditions such as oxidative and/or reductive conditions, enzyme-triggered degradation can unmask and release the bio-cleavable linkers form the 2′-position of the nucleoside and regenerate 2′-hydroxy group at specific position of the oligonucleotide. The bio-cleavable linkers possess structural units at which specific cellular enzyme can react as its substrate. The linkers are also connected to self-immolative linker units, which are also released spontaneously at physiological conditions.

### The 2′-Modified Nucleoside Based Oligonucleotide Prodrug

One aspect of the invention relates to an oligonucleotide (e.g. a single-stranded iRNA agent or a double-stranded iRNA agent) comprising one or more 2′-modified nucleosides. The 2′-position of the nucleoside has a structure of formula (I):

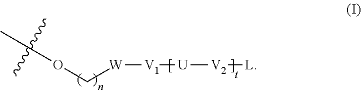

In formula (I):
W is O, S—S, N(R^{N}), C(O)N(R^{N}), N(R^{N})C(O), N(R^{N})C(O)O, N(R^{N})S(O)₂, or a biocleavable carbohydrate linker;
V₁ and V₂ are each independently for each occurrence absent, alkylene, arylene, heteroarylene, a divalent cycloalkyl, or a divalent heterocyclyl, each of which can be optionally substituted by one or more R²⁰ groups;
U is independently for each occurrence absent, W, O-W, W-O, or a phosphate or modified phosphate;
R^{N} is H, alkyl, or S(O)₂-aryl, each of which can be optionally substituted by one or more R²⁰ groups;
L is H or one or more ligands;
R²⁰ is independently for each occurrence halo, haloalkyl, hydroxyl, alkoxy, methoxyalkoxy, alkylthio, amino, alkylamino, alkynyl, aminoalkyl, aminoalkoxy, nitro, acetyl, or cyano;
n is an integer of 0-4 (e.g., n is 0 or 1);
t is an integer of 1-3 representing the number of presence for unit [U-V₂], which can be the same or different for each occurrence; and
[symbol] indicates the attachment of the formula (I) to the 2′-position of the nucleoside.

The 2′-modified nucleoside can have the structure of formula (II):

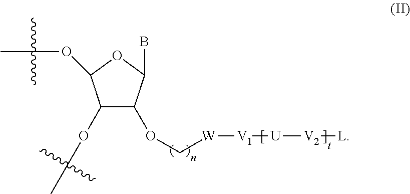

The variables W, V₁, V₂, U, L, t, and n are the same as those defined above in formula (I). B is modified or unmodified nucleobase. Each [symbol] indicates the attachment of the formula (II) to an adjacent nucleotide of the oligonucleotide, or hydrogen, or together with the oxygen to which it is attached forms a phosphate, or a modified phosphate, wherein at least one [symbol] attaches to an adjacent nucleotide.

For instance, the 2′-modified nucleoside can have the structure of formula (IIa) or (IIb):

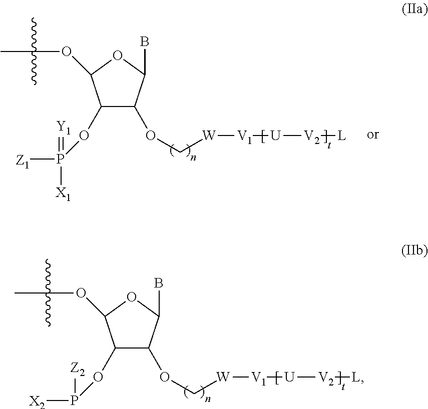

or a salt thereof. In formulas (IIa) and (IIb):

X₁ and Z₁ are each independently H, OH, OM, OR¹³, SH, SM, SR¹³, C(O)H, S(O)H, or alkyl, each of which can be optionally substituted with one or more R^{sub} groups, N(R′)(R″), B(R¹³)₃, BH₃, Se; or D-Q, wherein D is independently for each occurrence absent, O, S, N(R′), alkylene, each of which can be optionally substituted with one or more R^{sub} groups, and Q is independently for each occurrence a nucleoside or oligonucleotide;
X₂ and Z₂ are each independently N(R′)(R″), OR¹⁸, or D-Q, wherein D is independently for each occurrence absent, O, S, N, N(R′), alkylene, each of which can be optionally substituted with one or more R^{sub} groups, and Q is independently for each occurrence a nucleoside or oligonucleotide;
Y₁ is S, O, or N(R′);
M is an organic or inorganic cation;
R¹³ is independently for each occurrence H, alkyl, alkenyl, alkynyl, cycloalkyl, aryl, aralkyl, ω-amino alkyl, ω-hydroxy alkyl, or ω-hydroxy alkenyl, an oxygen protecting group, each of which can be optionally substituted with one or more R^{sub} groups;
R′ and R″ are each independently H, alkyl, alkenyl, alkynyl, aryl, hydroxy, alkyloxy, ω-amino alkyl, ω-hydroxy alkyl, ω-hydroxy alkenyl, or ω-hydroxy alkynyl, each of which can be optionally substituted with one or more R^{sub} groups;
R¹⁸ is H or alkyl, optionally substituted with one or more R^{sub} groups; and
R^{sub} is independently for each occurrence halo, haloalkyl, alkyl, alkaryl, aryl, aralkyl, hydroxy, alkyloxy, aryloxy, oxo, nitro, amino, acylamino, alkylcarbamoyl, arylcarbamoyl, alkylamino, aminoalkyl, alkoxycarbonyl, carboxy, hydroxyalkyl, alkanesulfonyl, arenesulfonyl, alkanesulfonamido, arenesulfonamido, aralkylsulfonamido, alkylcarbonyl, arylcarbonyl, acyloxy, cyano, or ureido. The variables B, W, V₁, V₂, U, L, t, and n are the same as those defined above in formula (II). [symbol] indicates the attachment of the formula (IIa) or (IIb) to the adjacent nucleotide of the oligonucleotide, hydrogen, or together with the oxygen to which it is attached forms a phosphate, or a modified phosphate.

In some embodiments, V₁ is not absent, i.e., V₁ is alkylene, arylene, heteroarylene, a divalent cycloalkyl, or a divalent heterocyclyl, each of which can be optionally substituted by one or more R²⁰ groups.

In some embodiments, the 2′-modified nucleoside has the structure of formula:

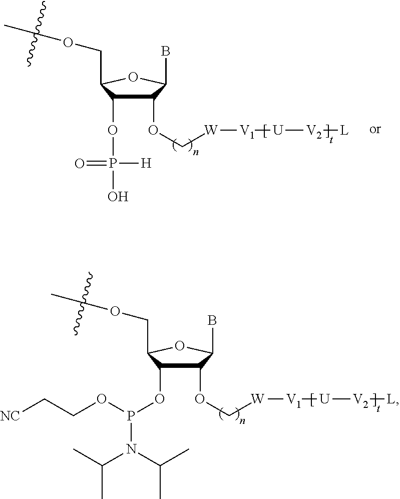

or a salt thereof. The variables B, W, V₁, V₂, U, L, t, and n are the same as those defined above in formula (II).

Exemplary structures for the 2′-modified nucleoside are:

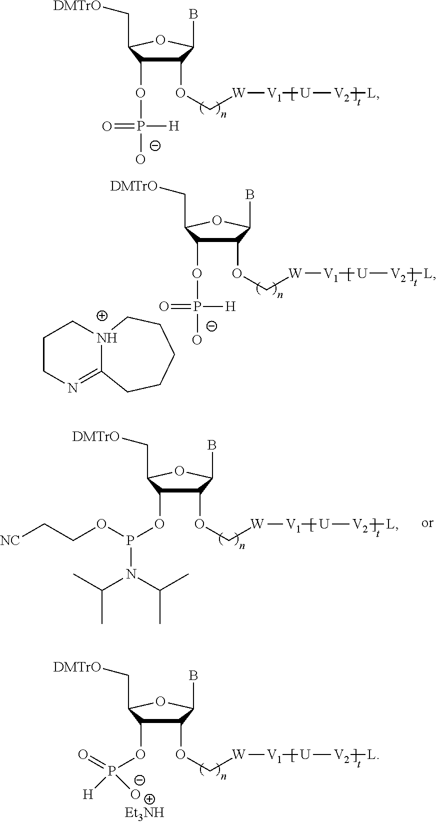

The variables B, W, V₁, V₂, U, L, t, and n are the same as those defined above in formula (II).

In all the formulas for the 2′-modified nucleoside, B is modified or unmodified nucleobase. For instance, B is independently for each occurrence A, A^{Bz}, C, C^{Ac}, C^{Bz}, 5-Me-C, 5-Me-C^{Ac}, G, G^{iBu}, I, U, T, 2-thiouridine, 4-thiouridine, a C5-modified pyrimidine, C2-modified purine, N8-modifed purine, phenoxazine, G-clamp, non-canonical mono-, bi-, and tri-cyclic heterocycles, a pseudouracil, isoC, isoG, 2,6-diamninopurine, a pseudocytosine, 2-aminopurine, xanthosine, N6-alkyl-A, or O6-alkyl-G.

In some embodiments, n is 0 or 1.

In some embodiments, n is 0. W is C(O)N(R^{N}) or a biocleavable carbohydrate linker, as described herein.

In one embodiment, n is 0 and W is C(O)N(R^{N}) (e.g., 2′-carbamate linker). The W-V₁-[U-V₂]ₜ-L has the structure of:

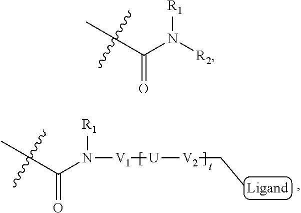

or a biocleavable carbohydrate linker such as

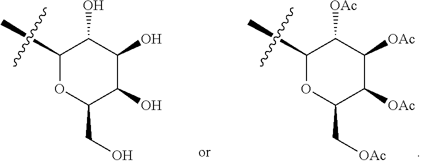

In these formulas:
each R₁ is independently for each occurrence H, linear or branched C₁-C₂₄ alkyl, aryl, hydroxyl, alkoxy, halo, methoxyalkoxy, alkylthio, amino, alkylamino, alkynyl, aminoalkyl, or aminoalkoxy;
   R₂ is selected from the group consisting of:

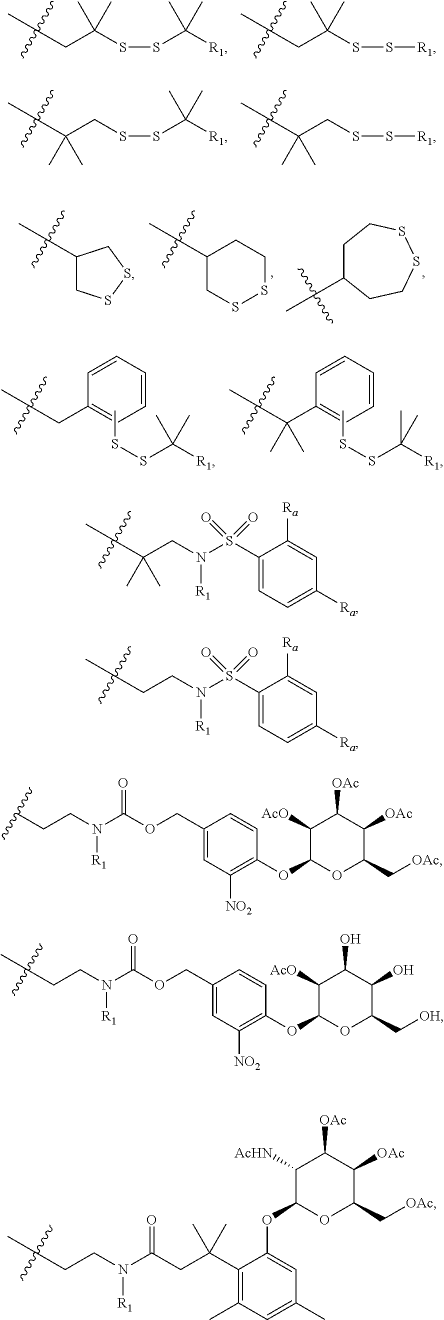

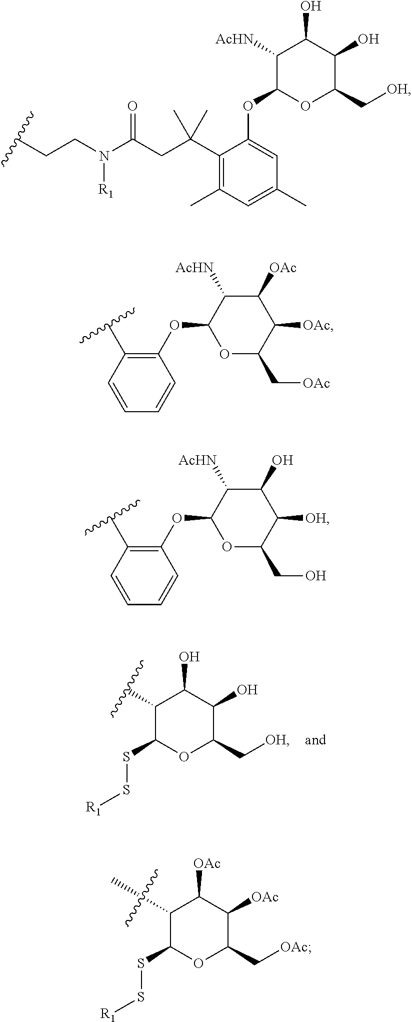

or R₁ and R₂ together form a heterocyclic ring, such as

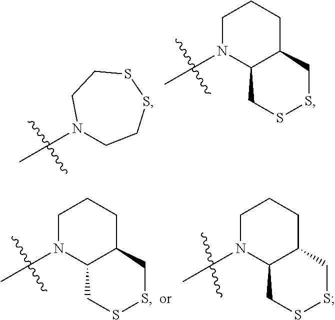 [SMILES: *N1CCSSCC1 ; *N1CCC[C@H]2CSSC[C@H]21 ; *N1CCC[C@H]2CSSC[C@@H]21 ; *N1CCC[C@@H]2CSSC[C@H]21]

and
Rₐ is independently for each occurrence H, NO₂, CF₃, C(O)CH₃, and CN. In the R₂ group definition, the carbohydrate moiety in any of the groups can be replaced with any biocleavable carbohydrate linker as described herein.

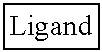

represents a ligand. The variables V₁, V₂, U, and t are the same as those defined above in formula (II).

Exemplary 2′-modified nucleosides include:

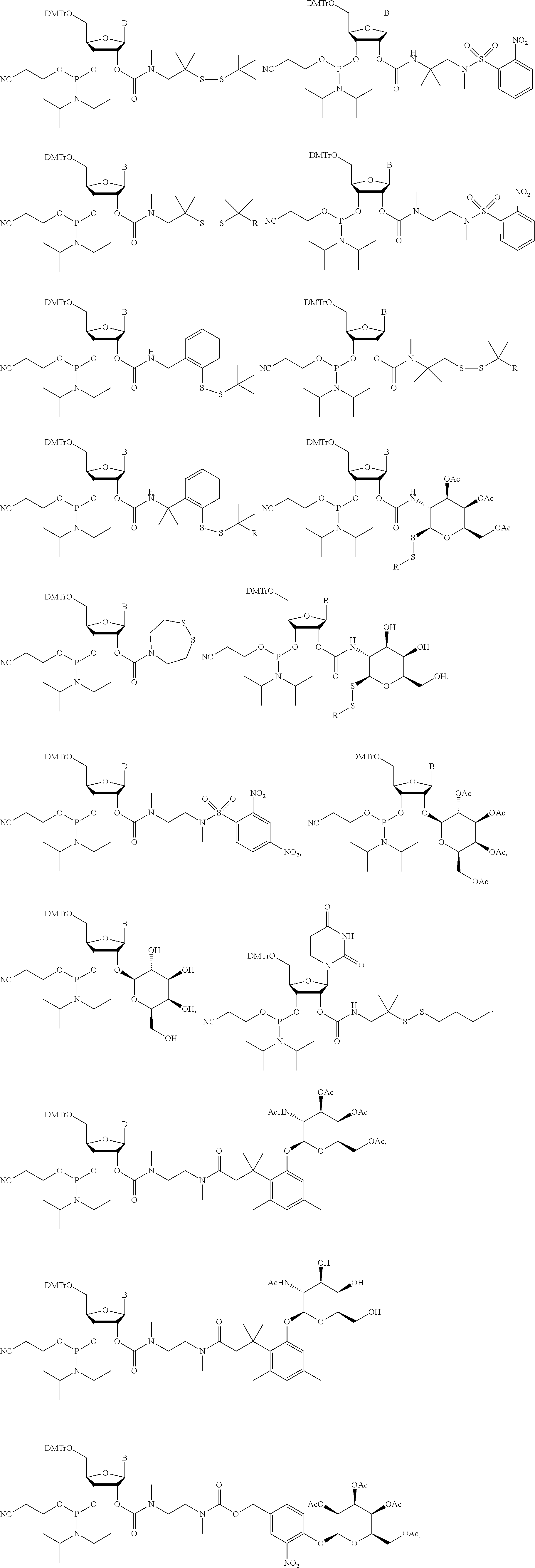

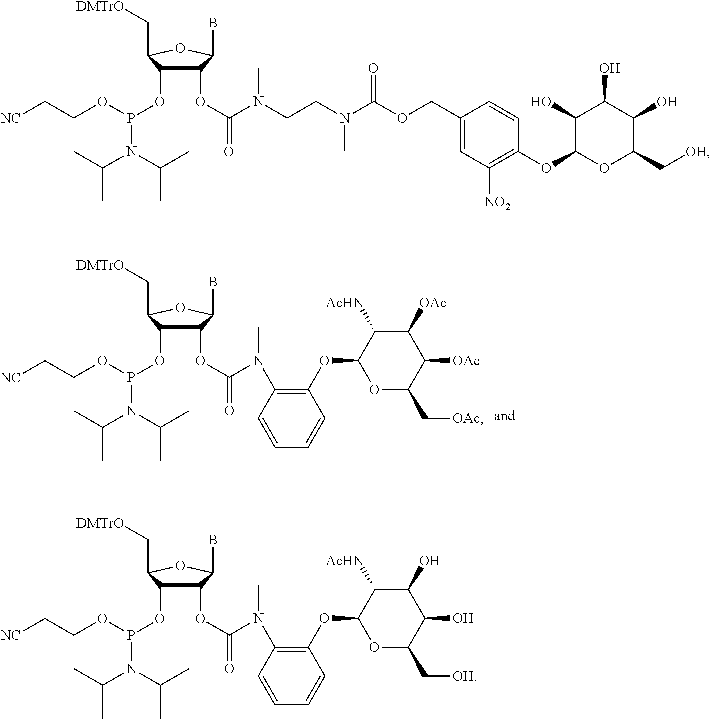

R is independently, for each occurrence, H, linear or branched C₁-C₂₄ alkyl (e.g., Me), C≡CH, OH, O-alkyl (e.g., OMe, O-nPr), O-alkylamino, O—(CH₂)₂OMe, SMe, Cl, F, NMe₂, or NH₂. B is independently, for each occurrence, A^{Bz}; C^{Ac}; 5-Me-C^{Ac}; G^{iBu}; I; U; T; 2-thiouridine; 4-thiouridine; C5-modified pyrimidines; C2-modified purines; N8-modifed purines; phenoxazine; G-clamp; non-canonical mono, bi and tricyclic heterocycles; pseudouracil; isoC; isoG; 2,6-diamninopurine; pseudocytosine; 2-aminopurine; xanthosine; N6-alkyl-A; O6-alkyl-G; or unnatural nucleobase. The carbohydrate moiety in any of these exemplary formulas can be replaced with any biocleavable carbohydrate linker as described herein.

In some embodiments, n is 1. W is O, S—S, N(R^{N}), C(O)N(R^{N}), N(R^{N})C(O), N(R^{N})C(O)O, or N(R^{N})S(O)₂.

In one embodiment, n is 1 and W is 0 (e.g., 2′-acetal linker). The W-V₁-[U-V₂]ₜ-L has the structure of:

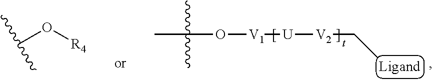

wherein:

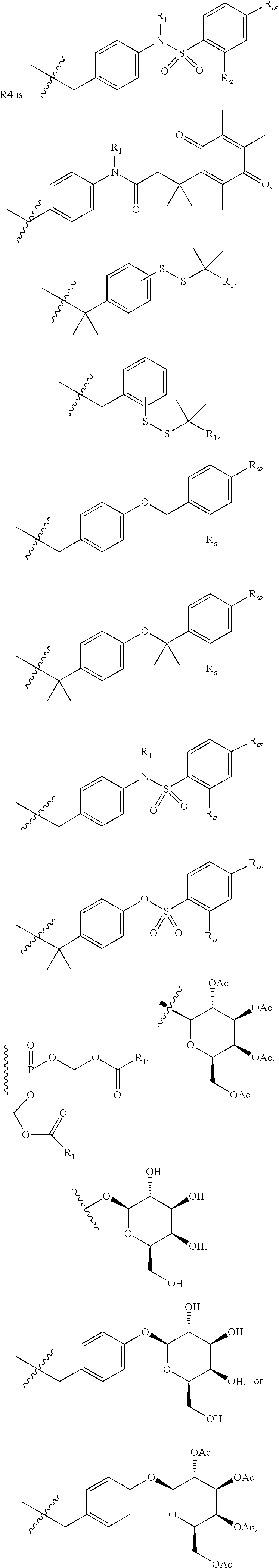

R¹ is independently for each occurrence H, linear or branched C₁-C₂₄ alkyl, aryl, hydroxyl, alkoxy, halo, methoxyalkoxy, alkylthio, amino, alkylamino, alkynyl, aminoalkyl, or aminoalkoxy; and
Rₐ is independently for each occurrence H, NO₂, CF₃, C(O)CH₃, and CN. In the R₄ group definition, the carbohydrate moiety in any of the groups can be replaced with any biocleavable carbohydrate linker as described herein.

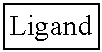

represents a ligand. The variables V₁, V₂, U, and t are the same as those defined above in formula (II).

In an exemplary embodiment, n is 1 and the W-V₁-[U-V₂]ₜ-L has the structure of:

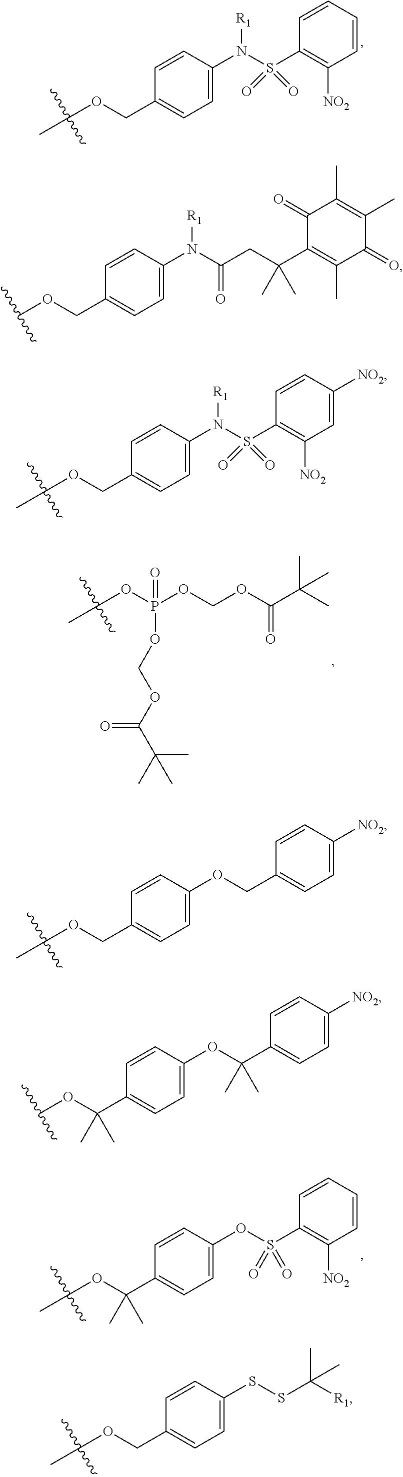

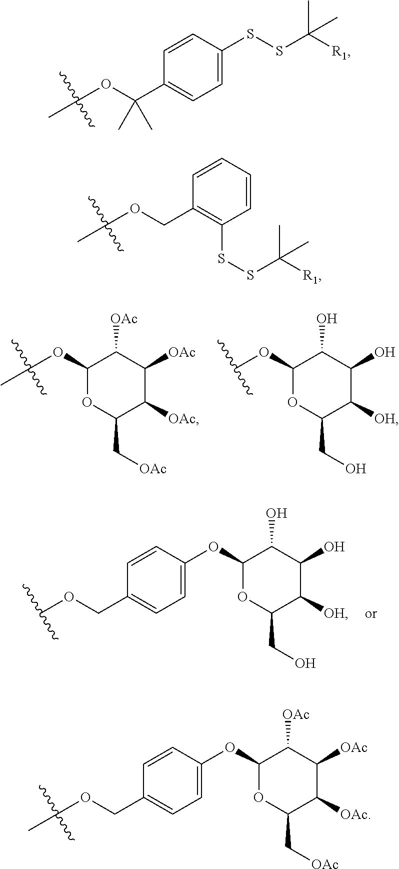

The carbohydrate moiety in any of these exemplary formulas can be replaced with any biocleavable carbohydrate linker as described herein.

Exemplary 2′-modified nucleosides include:

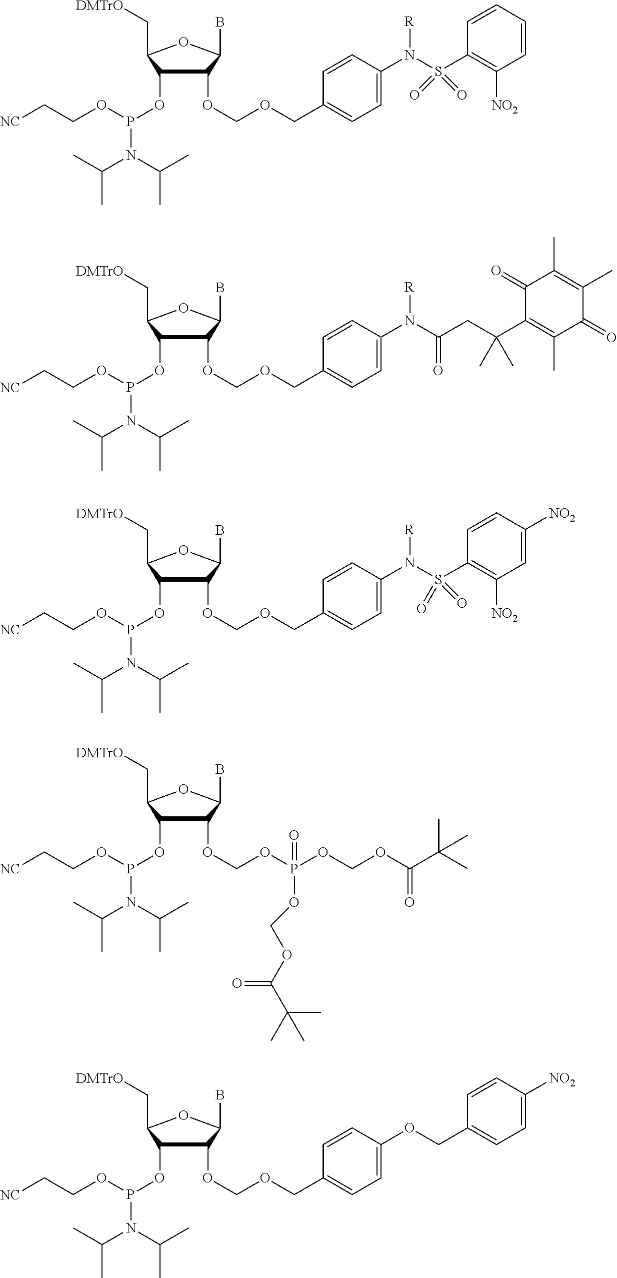

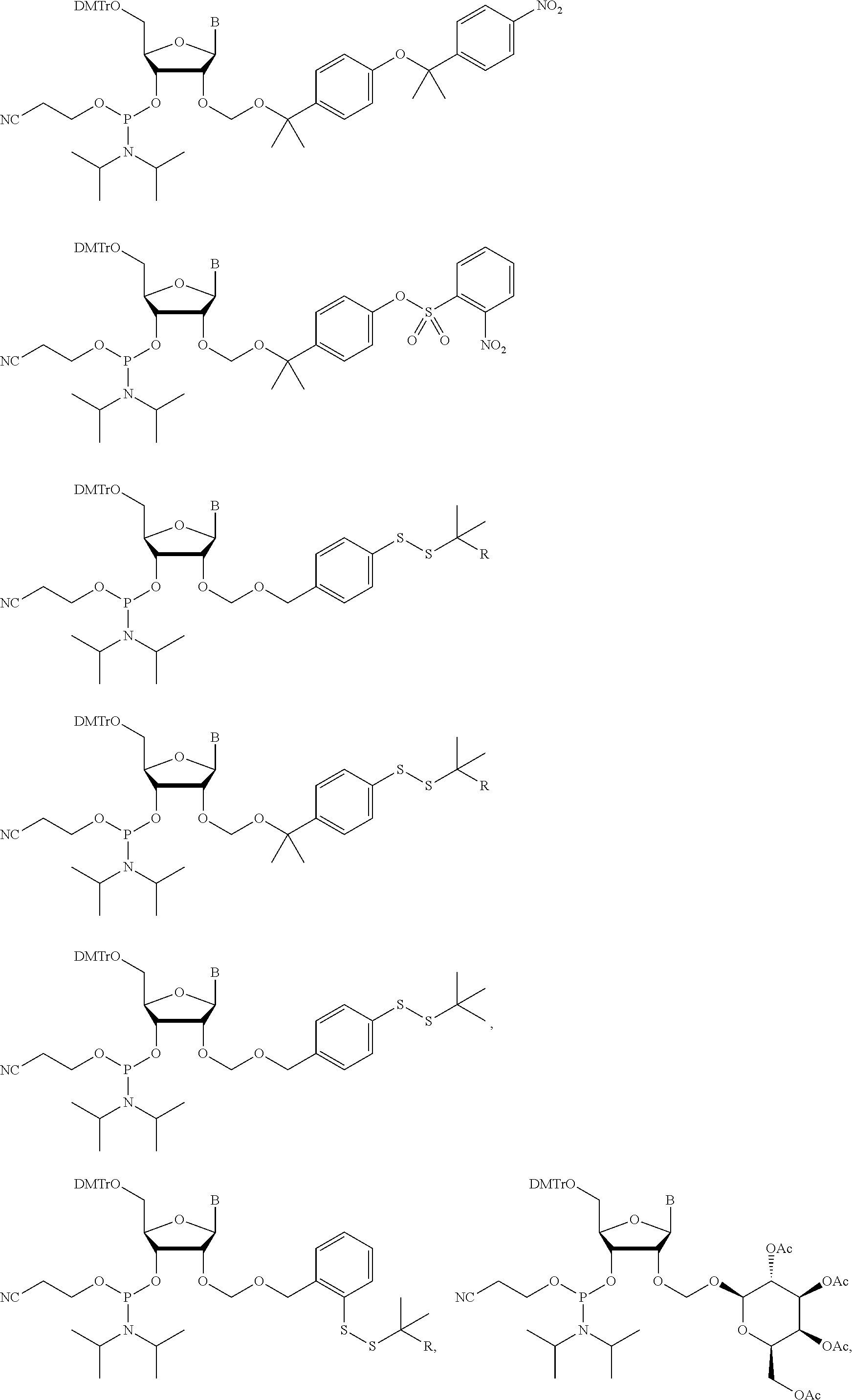

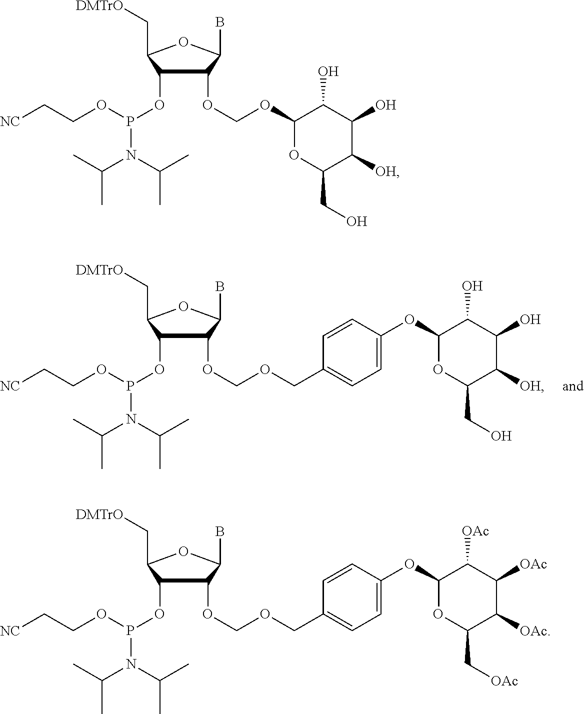

In these formulas, R is independently, for each occurrence, H, linear or branched C₁-C₂₄ alkyl (e.g., Me), C≡CH, OH, O-alkyl (e.g., OMe, 0-nPr), O-alkylamino, O—(CH₂)₂OMe, SMe, Cl, F, NMe₂, or NH₂. B is independently, for each occurrence, A^{Bz}, C^{Ac}; 5-Me-C^{Ac}. G^{iBu}; I; U; T; 2-thiouridine; 4-thiouridine; C5-modified pyrimidines; C2-modified purines; N8-modifed purines; phenoxazine; G-clamp; non-canonical mono, bi and tricyclic heterocycles; pseudouracil; isoC; isoG; 2,6-diamninopurine; pseudocytosine; 2-aminopurine; xanthosine; N6-alkyl-A; O6-alkyl-G; or unnatural nucleobase. The carbohydrate moiety in any of these exemplary formulas can be replaced with any biocleavable carbohydrate linker as described herein.

In some embodiments, n is 1 and W is S—S(e.g., 2′-methylene disulfide linker) The W-V₁-[U-V₂]ₜ-L has the structure of:

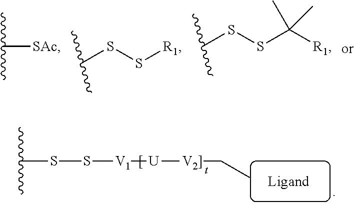

R₁ is independently for each occurrence H, linear or branched C₁-C₂₄ alkyl, aryl, hydroxyl, alkoxy, halo, methoxyalkoxy, alkylthio, amino, alkylamino, alkynyl, aminoalkyl, or aminoalkoxy.

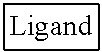

represents a ligand. The variables V₁, V₂, U, and t are the same as those defined above in formula (11).

In an exemplary embodiment, n is 1 and the W-V₁-[U-V₂]c-L has the structure of:

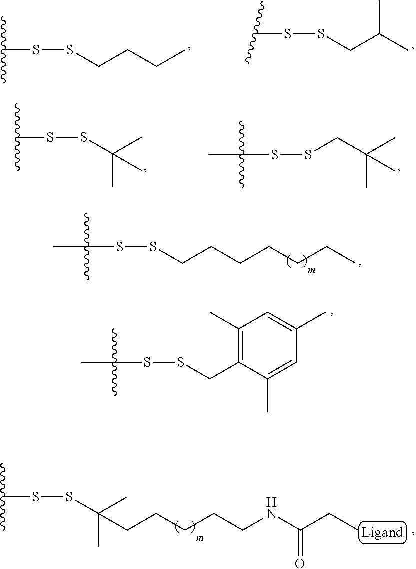

wherein m is an integer of 0-18.

Exemplary 2′-modified nucleosides include:

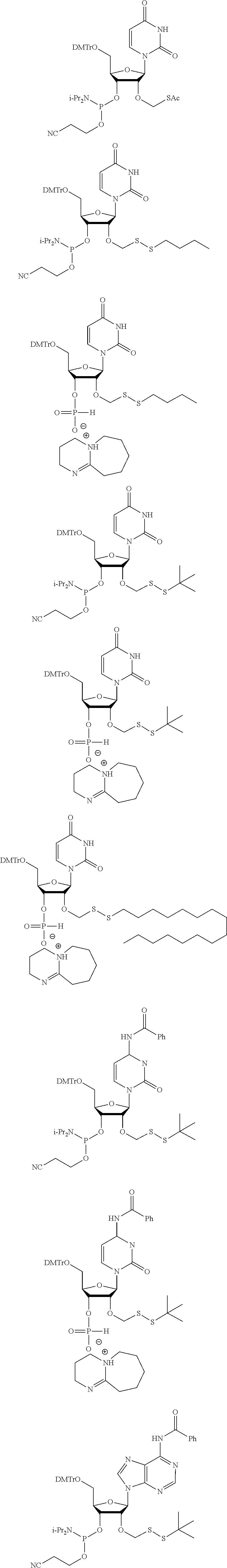

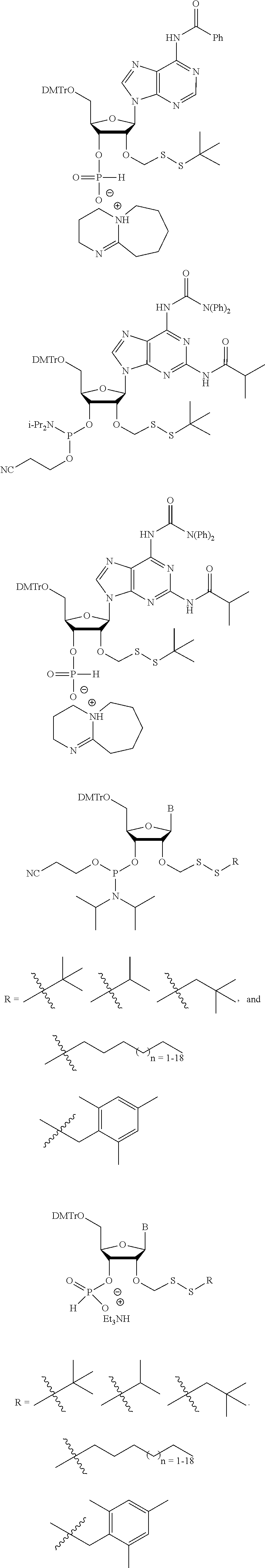

The nucleobase moiety in any of these exemplary formulas can be replaced with any nucleobase B. B is independently, for each occurrence, A^{Bz}; C^{Ac}; 5-Me-C^{Ac}; G^{iBu}; I; U; T; 2-thiouridine; 4-thiouridine; C5-modified pyrimidines; C2-modified purines; N8-modifed purines; phenoxazine; G-clamp; non-canonical mono, bi and tricyclic heterocycles; pseudouracil; isoC; isoG; 2,6-diamninopurine; pseudocytosine; 2-aminopurine; xanthosine; N6-alkyl-A; O6-alkyl-G; or unnatural nucleobase.

In some embodiments, n is 1 and W is N(R^{N})C(O) or N(R^{N})C(O)O (e.g., 2′-methylene amide linker or 2′-methylene carbamate linker). The W-V₁-[U-V₂]ₜ-L has the structure of:

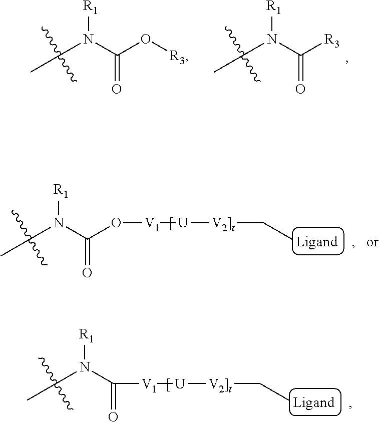

wherein:

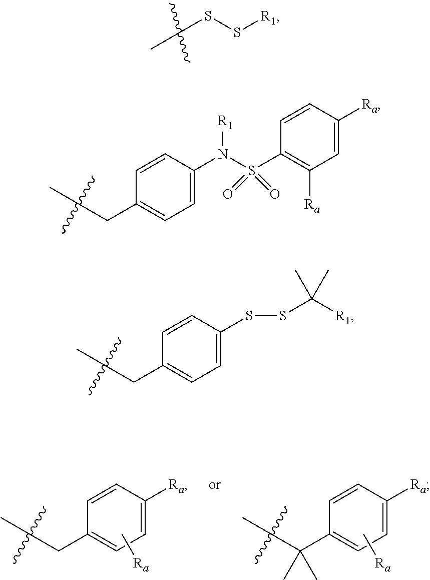

R₃ is linear or branched alkyl, aryl,
R₁ is independently for each occurrence H, linear or branched C₁-C₂₄ alkyl, aryl, hydroxyl, alkoxy, halo, methoxyalkoxy, alkylthio, amino, alkylamino, alkynyl, aminoalkyl, or aminoalkoxy; and
each Rₐ is independently for each occurrence H, NO₂, CF₃, C(O)CH₃, and CN.

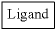

represents a ligand. The variables V₁, V₂, U, and t are the same as those defined above in formula (II).

In an exemplary embodiment, n is 1 and the W-V₁-[U-V₂]ₜ-L has the structure of:

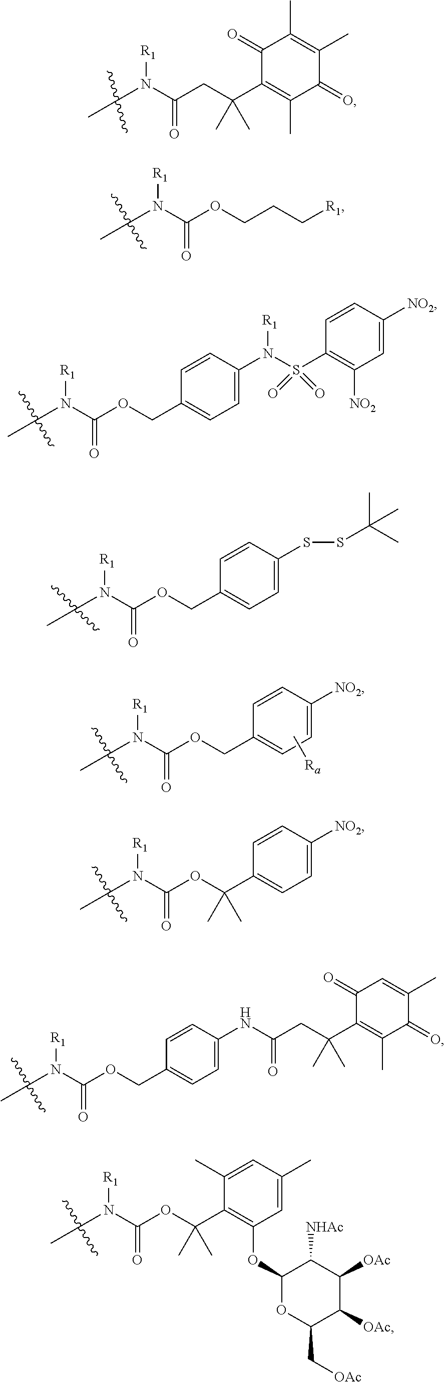

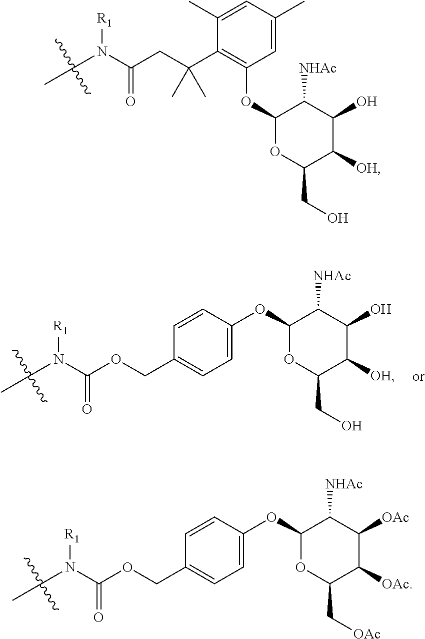

The carbohydrate moiety in any of these formulas can be replaced with any biocleavable carbohydrate linker as described herein.

Exemplary 2′-modified nucleosides include:

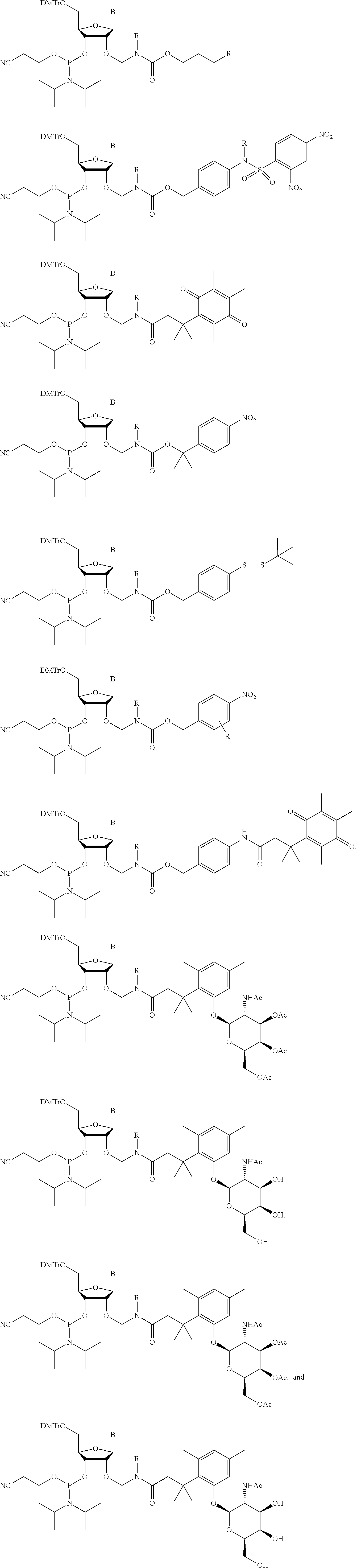

In these formulas, R is independently, for each occurrence, H, linear or branched C₁-C₂₄ alkyl (e.g., Me), C≡CH, OH, O-alkyl (e.g., OMe, O-nPr), O-alkylamino, O—(CH₂)₂OMe, SMe, Cl, F, NMe₂, or NH₂. B is independently, for each occurrence, A^{Bz}; C^{Ac}; 5-Me-C^{Ac}; G^{iBu}; I; U; T; 2-thiouridine; 4-thiouridine; C5-modified pyrimidines; C2-modified purines; N8-modifed purines; phenoxazine; G-clamp; non-canonical mono, bi and tricyclic heterocycles; pseudouracil; isoC; isoG; 2,6-diamninopurine; pseudocytosine; 2-aminopurine; xanthosine; N6-alkyl-A; O6-alkyl-G; or unnatural nucleobase. The carbohydrate moiety in any of these exemplary formulas can be replaced with any biocleavable carbohydrate linker as described herein.

In some embodiments, n is 1 and W is N(R^{N})S(O)₂ (e.g., 2′-methylene sulfonamide linker). The W-V₁-U-V₂-L has the structure of:

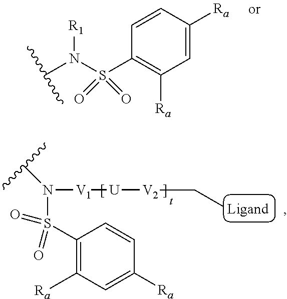

wherein:
R₁ is independently for each occurrence H, linear or branched C₁-C₂₄ alkyl, aryl, hydroxyl, alkoxy, halo, methoxyalkoxy, alkylthio, amino, alkylamino, alkynyl, aminoalkyl, or aminoalkoxy; and
Rₐ is independently for each occurrence H, NO₂, CF₃, C(O)CH₃, and CN.

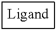

represents a ligand. The variables V₁, V₂, U, and t are the same as those defined above in formula (II).

In an exemplary embodiment, n is 1 and the W-V₁-U-V₂-L has the structure of:

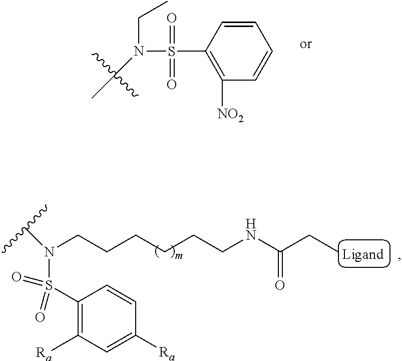

wherein m is an integer of 0-18.

Exemplary 2′-modified nucleosides include:

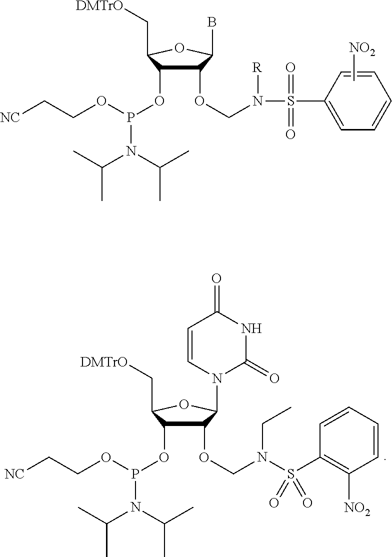

R is independently, for each occurrence, H, linear or branched C₁-C₂₄ alkyl (e.g., Me), C≡CH, OH, O-alkyl (e.g., OMe, O-nPr), O-alkylamino, O—(CH₂)₂OMe, SMe, Cl, F, NMe₂, or NH₂. The nucleobase moiety in the above formula can be replaced with any nucleobase B. B is independently, for each occurrence, A^{Bz}; C^{Ac}; 5-Me-C^{Ac}; G^{iBu}; I; U; T; 2-thiouridine; 4-thiouridine; C5-modified pyrimidines; C2-modified purines; N8-modifed purines; phenoxazine; G-clamp; non-canonical mono, bi and tricyclic heterocycles; pseudouracil; isoC; isoG; 2,6-diamninopurine; pseudocytosine; 2-aminopurine; xanthosine; N6-alkyl-A; O6-alkyl-G; or unnatural nucleobase.

In some embodiments, the W-V₁-U-V₂-L contains a biocleavable carbohydrate linker. Exemplary structures for the W-V₁-[U-V₂]ₜ-L include:

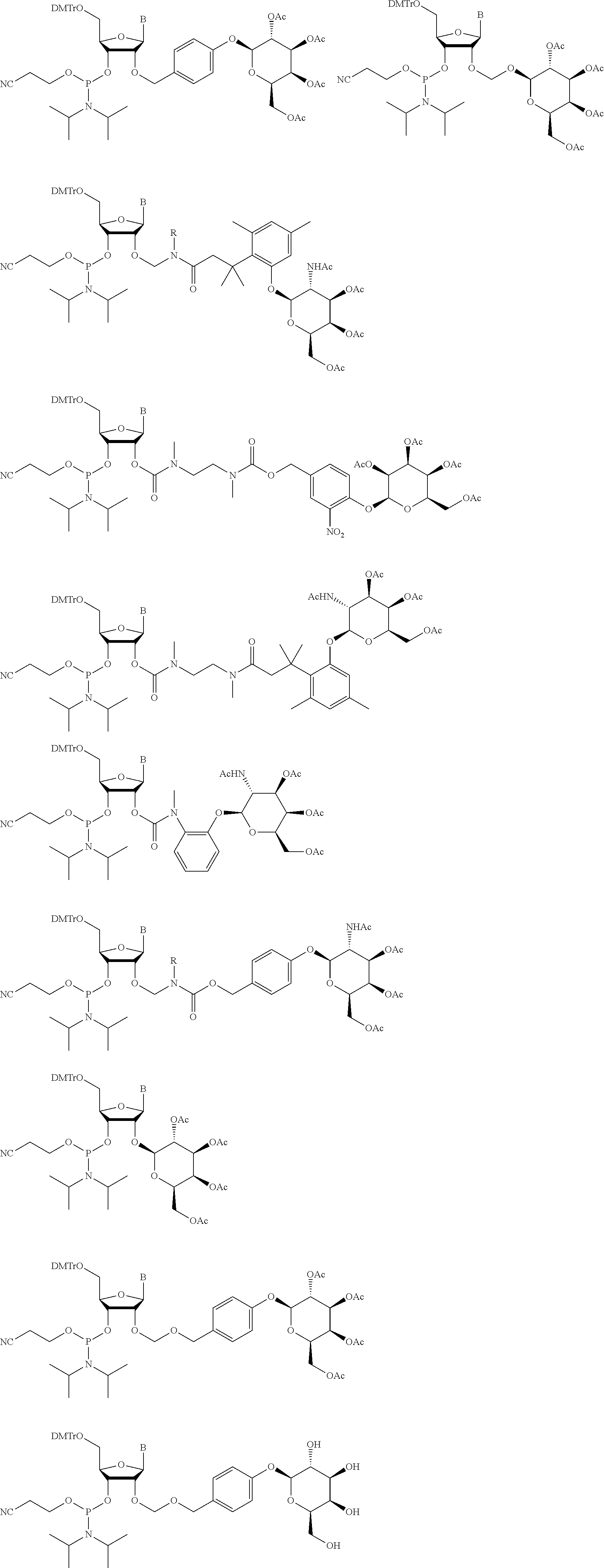

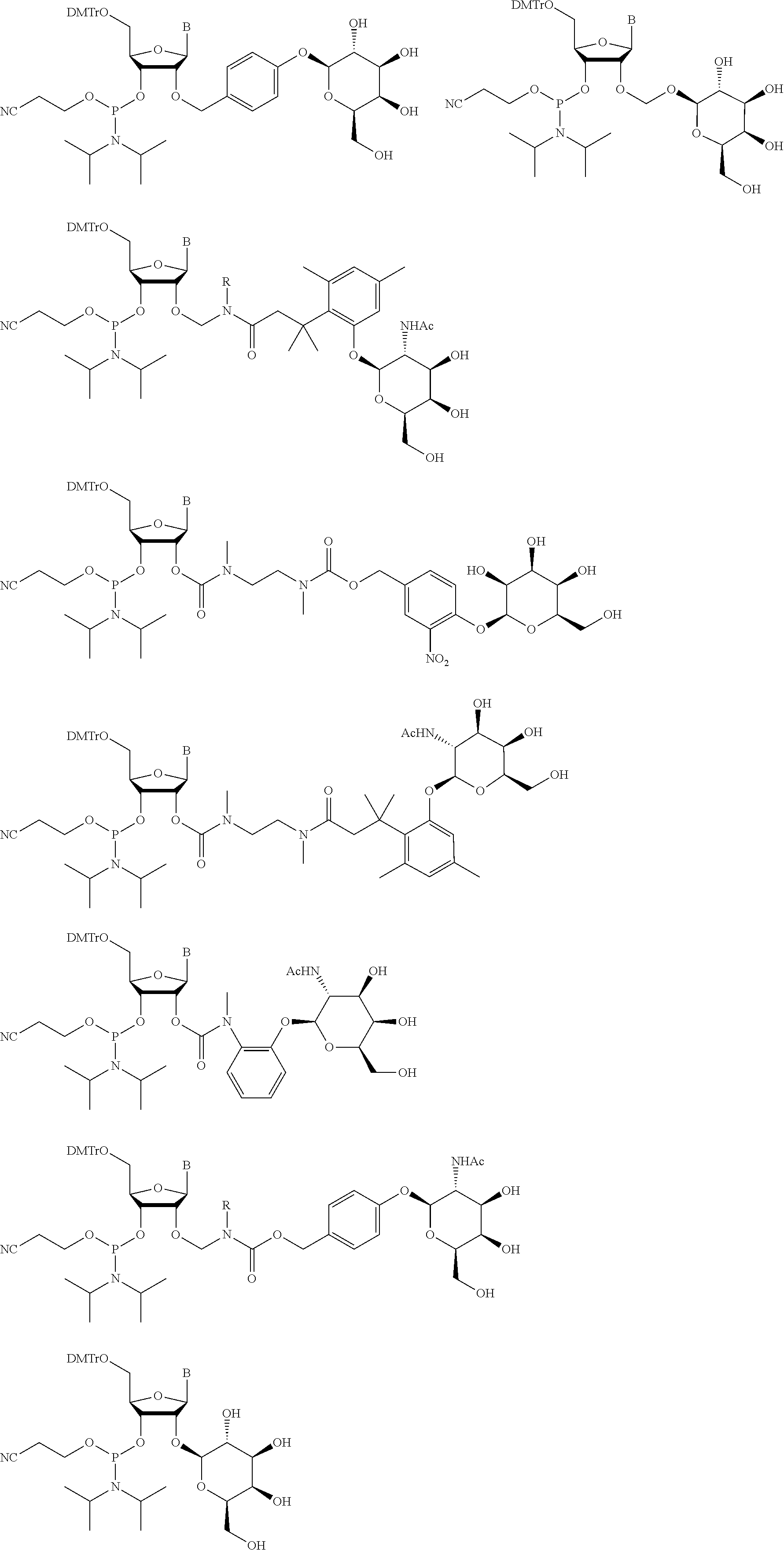

In these formulas, R is independently, for each occurrence, H, linear or branched C₁-C₂₄ alkyl (e.g., Me), C≡CH, OH, O-alkyl (e.g., OMe, O-nPr), O-alkylamino, O—(CH₂)₂OMe, SMe, Cl, F, NMe₂, or NH₂. B is independently, for each occurrence, A^{Bz}; C^{Ac}; 5-Me-C^{Ac}; G^{iBu}; I; U; T; 2-thiouridine; 4-thiouridine; C5-modified pyrimidines; C2-modified purines; N8-modifed purines; phenoxazine; G-clamp; non-canonical mono, bi and tricyclic heterocycles; pseudouracil; isoC; isoG; 2,6-diamninopurine; pseudocytosine; 2-aminopurine; xanthosine; N6-alkyl-A; O6-alkyl-G; or unnatural nucleobase. The carbohydrate moiety in any of these exemplary formulas can be replaced with any biocleavable carbohydrate linker as described herein.

In some embodiments, n is 1, and the W-V₁-U-V₂-L has the structure of:

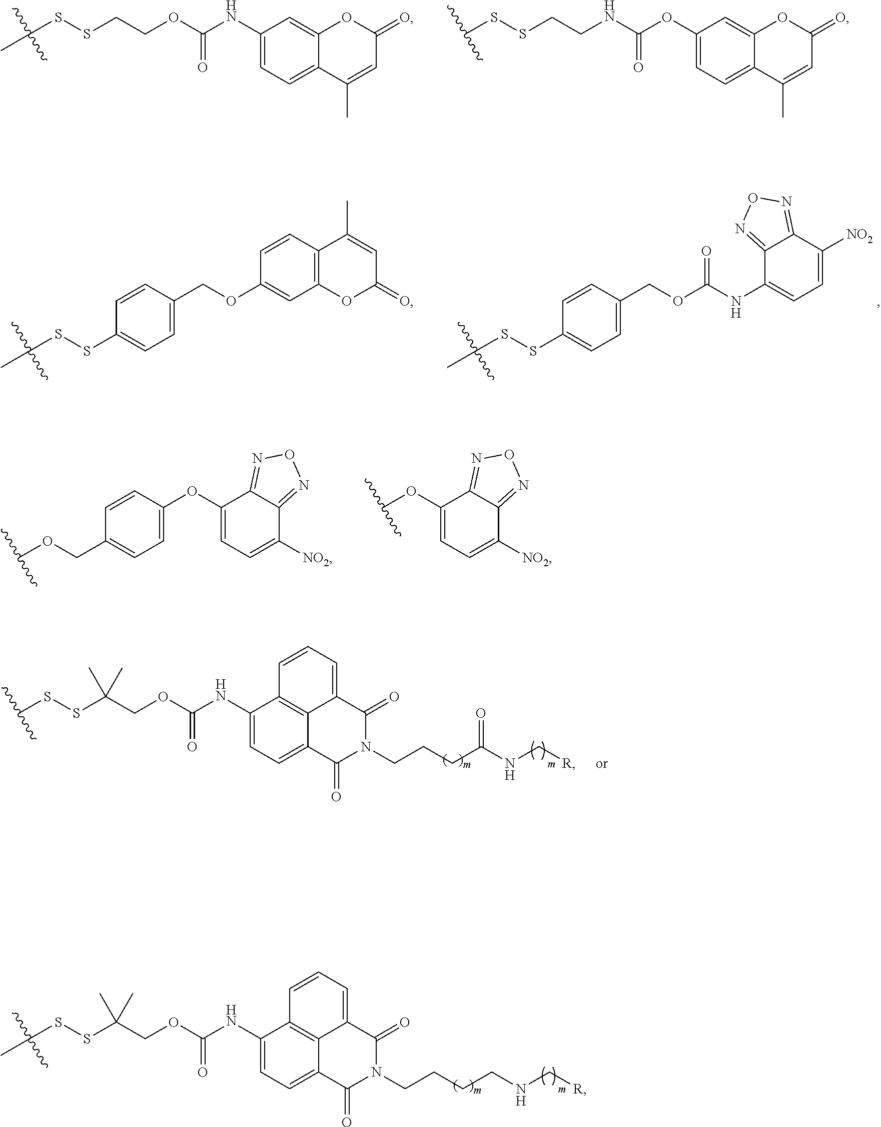

wherein:
R is

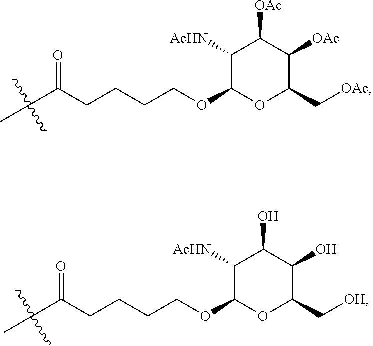 [SMILES: *C(=O)CCCCO[C@@H]1O[C@H](COC(C)=O)[C@H](OC(C)=O)[C@H](OC(C)=O)[C@H]1NC(C)=O ; *C(=O)CCCCO[C@@H]1O[C@H](CO)[C@H](O)[C@H](O)[C@H]1NC(C)=O]

a peptide, a small molecular ligand, GalNAc or multivalent GalNAc, folate or a lipophilic moiety; and
m is independently for each occurrence an integer of 0-18.

In some embodiments, the 2′-modified nucleoside has a structure selected from the group consisting of:

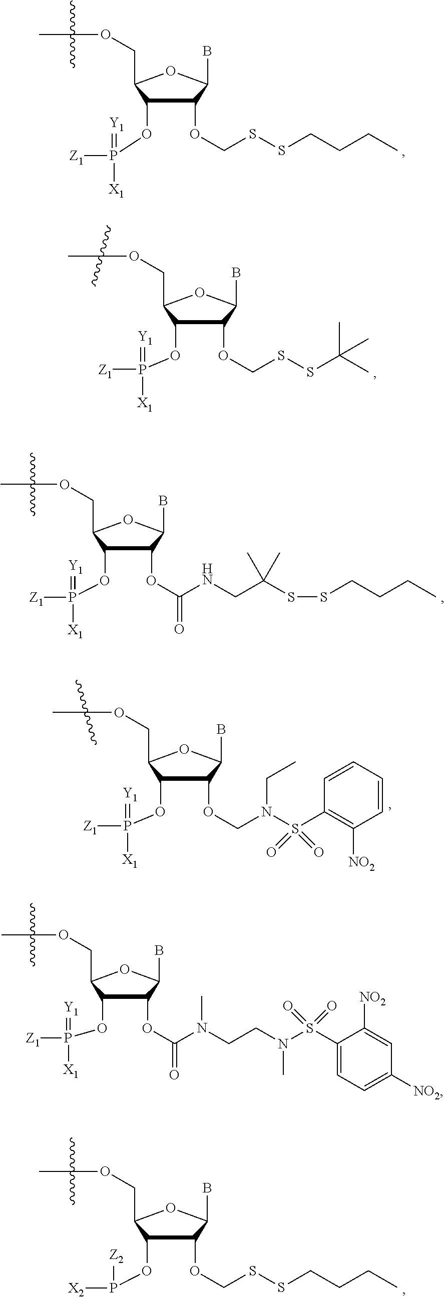

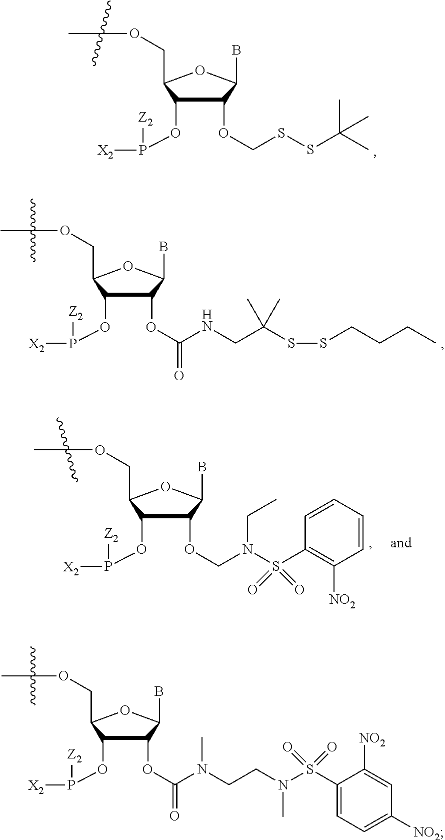

or a salt thereof.

Exemplary 2′-modified nucleosides include:

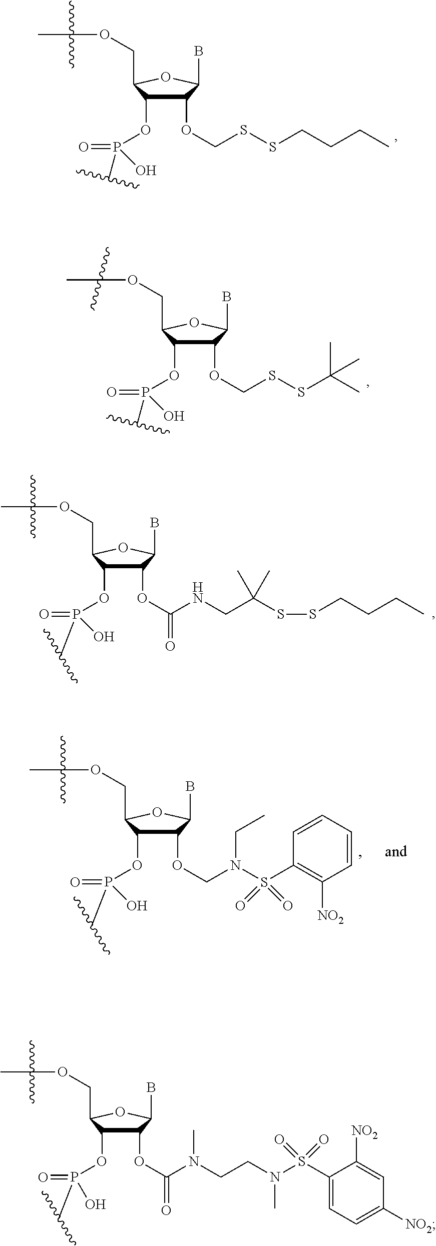

or a salt thereof.

In some embodiments, the 2′-modified nucleoside has a modified, abasic sugar and has a structure of (IIIa) or (IIIb):

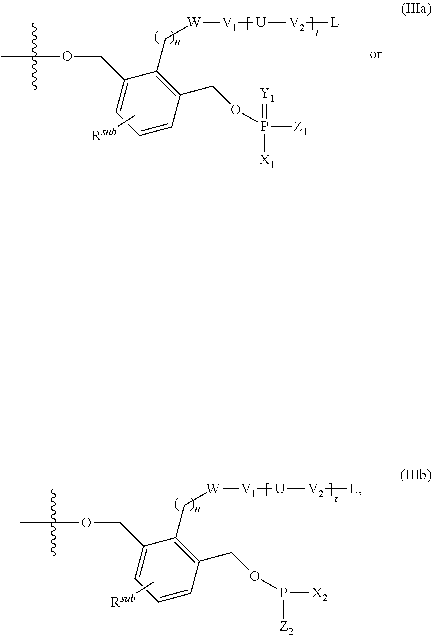

or a salt thereof. In these formulas, the variables X₁, X₂, Y₁, Z₁, Z₂, and R^{sub} are the same as those defined above in formulas (IIa) and (IIb). The variables W, V₁, V₂, U, L, t, and n are the same as those defined above in formula (II).

In some embodiments, the 2′-modified nucleoside has a structure of

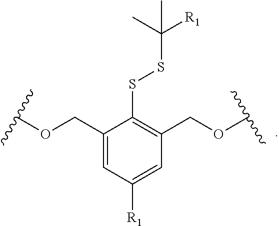

In some embodiments, the 2′-modified nucleoside has a structure of:

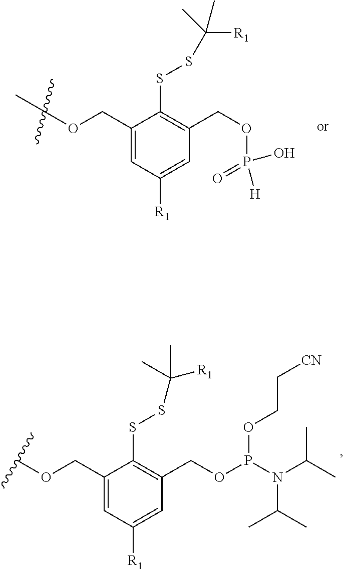

or a salt thereof. In these embodiments, R₁ is independently for each occurrence H, linear or branched C₁-C₂₄ alkyl, aryl, hydroxyl, alkoxy, halo, methoxyalkoxy, alkylthio, amino, alkylamino, alkynyl, aminoalkyl, or aminoalkoxy.

In one embodiment, the 2′-modified nucleoside has a structure of:

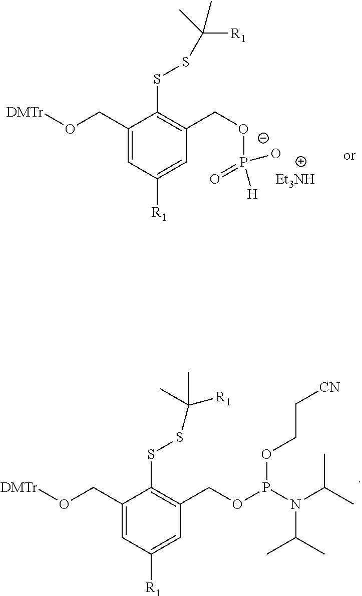

R₁ is independently for each occurrence H, linear or branched C₁-C₂₄ alkyl, aryl, hydroxyl, alkoxy, halo, methoxyalkoxy, alkylthio, amino, alkylamino, alkynyl, aminoalkyl, or aminoalkoxy.

In some embodiments, the oligonucleotide is a single-stranded oligonucleotide, such as a single-stranded iRNA agent (e.g., single-stranded siRNA).

In some embodiments, the oligonucleotide is a double-stranded oligonucleotide, such as a double-stranded iRNA agent (e.g., double-stranded siRNA), comprising a sense strand and an antisense strand.

In some embodiments, the oligonucleotide contains at least one 2′-modified nucleoside at the antisense strand, sense strand, or both strands of the oligonucleotide.

In some embodiments, the oligonucleotide contains at least one 2′-modified nucleoside at the first two positions of the 5′-end of the oligonucleotide.

In some embodiments, the oligonucleotide contains at least one 2′-modified nucleoside at the first two positions of the 3′-end of the oligonucleotide.

In some embodiments, the oligonucleotide contains at least one 2′-modified nucleoside at the first two positions of the 5′-end of the oligonucleotide, and at least one 2′-modified nucleoside at the first two positions of the 3′-end of the oligonucleotide.

In some embodiments, the oligonucleotide contains at least one 2′-modified nucleoside at an internal position of the oligonucleotide.

In one embodiment, the sense strand contains at least one2′-modified nucleoside of formula (I). In one embodiment, the antisense strand contains at least 2′-modified nucleoside of formula (I). In one embodiment, both the sense strand and the antisense strand each contain at least one 2′-modified nucleoside of formula (I).

Introduction of the 2′-modified nucleoside on either the sense or antisense strand or both the sense and antisense strands, are illustrated in Schemes 18-20 in Example 8 below.

Certain terms are abbreviated within chemical structures throughout the application as would be familiar to those skilled in the art, including, e.g., methyl (Me), benzoyl (Bz), acetyl (Ac), isobutyl (iBu), isopropyl (iPr), n-propyl (nPr), ethyl (Et), phenyl (Ph), pivaloyl (Piv), 4,4-dimethoxytrityl (DMTr).

The term “halo” or “halogen” refers to any radical of fluorine, chlorine, bromine or iodine.

The term “aliphatic” or “aliphatic group”, as used herein, means a straight-chain or branched, substituted or unsubstituted hydrocarbon chain that is saturated or contains one or more units of unsaturation, or a monocyclic hydrocarbon or bicyclic or polycyclic hydrocarbon that is saturated or contains one or more units of unsaturation, but is not aromatic, that has a single point of attachment to the rest of the molecule. In some embodiments, aliphatic groups contain 1-50 aliphatic carbon atoms, for instance, 1-10 aliphatic carbon atoms, 1-6 aliphatic carbon atoms, 1-5 aliphatic carbon atoms, 1-4 aliphatic carbon atoms, 1-3 aliphatic carbon atoms, or 1-2 aliphatic carbon atoms. In some embodiments, “cycloaliphatic” refers to a monocyclic or bicyclic C₃-C₁₀ hydrocarbon (e.g., a monocyclic C₃-C₆ hydrocarbon) that is saturated or contains one or more units of unsaturation, but is not aromatic, that has a single point of attachment to the rest of the molecule. Suitable aliphatic groups include, but are not limited to, linear or branched, substituted or unsubstituted alkyl, alkenyl, alkynyl groups and hybrids thereof such as (cycloalkyl)alkyl, (cycloalkenyl)alkyl, or (cycloalkyl)alkenyl.

The term “alkyl” refers to a hydrocarbon chain that may be a straight chain or branched chain, containing the indicated number of carbon atoms. For example, C₁-C₁₂ alkyl indicates that the group may have from 1 to 12 (inclusive) carbon atoms in it. Unless otherwise indicated, “alkyl” generally refers to C₁-C₂₄ alkyl (e.g., C₁-C₁₂ alkyl, C₁-C₈ alkyl, or C₁-C₄ alkyl). The term “haloalkyl” refers to an alkyl in which one or more hydrogen atoms are replaced by halo, and includes alkyl moieties in which all hydrogens have been replaced by halo (e.g., perfluoroalkyl). Alkyl and haloalkyl groups may be optionally inserted with O, N, or S. The terms “aralkyl” refers to an alkyl moiety in which an alkyl hydrogen atom is replaced by an aryl group. Aralkyl includes groups in which more than one hydrogen atom has been replaced by an aryl group. Examples of “aralkyl” include benzyl, 9-fluorenyl, benzhydryl, and trityl groups.

The term “alkenyl” refers to a straight or branched hydrocarbon chain containing 2-8 carbon atoms and characterized in having one or more double bonds. Unless otherwise indicated, “alkenyl” generally refers to C₂-C₈ alkenyl (e.g., C₂-C₆ alkenyl, C₂-C₄ alkenyl, or C₂-C₃ alkenyl). Examples of a typical alkenyl include, but not limited to, allyl, propenyl, 2-butenyl, 3-hexenyl and 3-octenyl groups. The term “alkynyl” refers to a straight or branched hydrocarbon chain containing 2-8 carbon atoms and characterized in having one or more triple bonds. Unless otherwise indicated, “alkynyl” generally refers to C₂-C₈ alkynyl (e.g., C₂-C₆ alkynyl, C₂-C₄ alkynyl, or C₂-C₃ alkynyl). Some examples of a typical alkynyl are ethynyl, 2-propynyl, and 3-methylbutynyl, and propargyl. The sp² and sp³ carbons may optionally serve as the point of attachment of the alkenyl and alkynyl groups, respectively.

The term “alkoxy” refers to an —O-alkyl radical. The term “alkylene” refers to a divalent alkyl (i.e., —R—). The term “aminoalkyl” refers to an alkyl substituted with an amino. The term “mercapto” refers to an —SH radical. The term “thioalkoxy” refers to an —S-alkyl radical.

The term “alkylene” refers to a divalent alkyl group. An “alkylene chain” is a polymethylene group, i.e., —(CH₂)ₙ—, wherein n is a positive integer, preferably from 1 to 6, from 1 to 4, from 1 to 3, from 1 to 2, or from 2 to 3.

The term “alkenylene” refers to a divalent alkenyl group. A substituted alkenylene chain is a polymethylene group containing at least one double bond in which one or more hydrogen atoms are replaced with a substituent.

The term “aryl” refers to a 6-carbon monocyclic or 10-carbon bicyclic aromatic ring system wherein 0, 1, 2, 3, or 4 atoms of each ring may be substituted by a substituent. The term “aryl” may be used interchangeably with the term “aryl ring.” Examples of aryl groups include phenyl, biphenyl, naphthyl, anthracyl, and the like, which may bear one or more substituents. Also included within the scope of the term “aryl,” as it is used herein, is a group in which an aromatic ring is fused to one or more non-aromatic rings, such as indanyl, phthalimidyl, naphthimidyl, phenanthridinyl, or tetrahydronaphthyl, and the like. The term “arylalkyl” or the term “aralkyl” refers to alkyl substituted with an aryl. The term “arylalkoxy” refers to an alkoxy substituted with aryl.

The term “arylene” refers to a divalent aryl group.

The term “heteroaryl” or “heteroar-” refers to an aromatic 5-8 membered monocyclic, 8-12 membered bicyclic, or 11-14 membered tricyclic ring system having 1-3 heteroatoms if monocyclic, 1-6 heteroatoms if bicyclic, or 1-9 heteroatoms if tricyclic, said heteroatoms selected from O, N, or S (e.g., carbon atoms and 1-3, 1-6, or 1-9 heteroatoms of N, O, or S if monocyclic, bicyclic, or tricyclic, respectively), wherein 0, 1, 2, 3, or 4 atoms of each ring may be substituted by a substituent. The term also include groups in which a heteroaromatic ring is fused to one or more aryl, cycloalkyl, or heterocyclyl rings, where the radical or point of attachment is on the heteroaromatic ring. Examples of heteroaryl groups include pyrrolyl, pyridyl, pyridazinyl, pyrazolyl, triazolyl, tetrazolyl, oxazolyl, isoxazolyl, oxadiazolyl, furanyl, imidazolyl, benzimidazolyl, pyrimidinyl, pyrazinyl, indolizinyl, thiophenyl or thienyl, quinolinyl, indolyl, thiazolyl, isothiazolyl, thiadiazolyl, purinyl, naphthyridinyl, pteridinyl, isoindolyl, benzothienyl, benzofuranyl, dibenzofuranyl, indazolyl, benzthiazolyl, quinolyl, isoquinolyl, cinnolinyl, phthalazinyl, quinazolinyl, quinoxalinyl, 4H-quinolizinyl, carbazolyl, acridinyl, phenazinyl, phenothiazinyl, phenoxazinyl, tetrahydroquinolinyl, tetrahydroisoquinolinyl, and pyrido[2,3-b]-1,4-oxazin-3(4H)-one and the like. The term “heteroarylalkyl” or the term “heteroaralkyl” refers to an alkyl substituted with a heteroaryl. The term “heteroarylalkoxy” refers to an alkoxy substituted with heteroaryl.

The term “heteroarylene” refers to a divalent heteroaryl group.

The term “cycloalkyl” or “cyclyl” as employed herein includes saturated and partially unsaturated, but not aromatic, cyclic hydrocarbon groups having 3 to 12 carbons, for example, 3 to 8 carbons, and, for example, 3 to 6 carbons, wherein the cycloalkyl group additionally may be optionally substituted. Cycloalkyl groups include, without limitation, cyclopropyl, cyclobutyl, cyclopentyl, cyclopentenyl, cyclohexyl, cyclohexenyl, cycloheptyl, and cyclooctyl.

The term “heterocyclyl,” “heterocycle,” “heterocyclic radical,” or “heterocyclic ring” refers to a nonaromatic 5-8 membered monocyclic, 8-12 membered bicyclic, or 11-14 membered tricyclic ring system having 1-3 heteroatoms if monocyclic, 1-6 heteroatoms if bicyclic, or 1-9 heteroatoms if tricyclic, said heteroatoms selected from O, N, or S (e.g., carbon atoms and 1-3, 1-6, or 1-9 heteroatoms of N, O, or S if monocyclic, bicyclic, or tricyclic, respectively), wherein 0, 1, 2 or 3 atoms of each ring may be substituted by a substituent. When used in reference to a ring atom of a heterocycle, the term “nitrogen” includes a substituted nitrogen. As an example, in a saturated or partially unsaturated ring having 0-3 heteroatoms selected from oxygen, sulfur or nitrogen, the nitrogen may be N (as in 3,4-dihydro-2H-pyrrolyl), NH (as in pyrrolidinyl), or +NR (as in N-substituted pyrrolidinyl). Examples of heterocyclyl groups include trizolyl, tetrazolyl, piperazinyl, pyrrolidinyl, dioxanyl, dioxolanyl, diazepinyl, oxazepinyl, thiazepinyl, morpholinyl, tetrahydrofuranyl, tetrahydrothiophenyl pyrrolidinyl, piperidinyl, pyrrolinyl, tetrahydroquinolinyl, tetrahydroisoquinolinyl, decahydroquinolinyl, oxazolidinyl, quinuclidinyl, and the like. The term “heterocyclylalkyl” refers to an alkyl group substituted by a heterocyclyl, wherein the alkyl and heterocyclyl portions independently are optionally substituted.

A divalent radical of an alkyl, alkenyl, aryl, heteroaryl, cycloalkyl, heterocyclyl is formed by removal of a hydrogen atom from an alkyl, alkenyl, aryl, heteroaryl, cycloalkyl, and heterocyclyl radical, respectively (or by removal of two hydrogen atoms from an alkane, alkene, arene, heteroarene, cycloalkane, or heterocycle, respectively).

The term “oxo” refers to an oxygen atom, which forms a carbonyl when attached to carbon, an N-oxide when attached to nitrogen, and a sulfoxide or sulfone when attached to sulfur.

The term “acyl” refers to an alkylcarbonyl, cycloalkylcarbonyl, arylcarbonyl, heterocyclylcarbonyl, or heteroarylcarbonyl substituent, any of which may be further substituted by substituents.

The term “substituted” refers to the replacement of one or more hydrogen radicals in a given structure with the radical of a specified substituent including, but not limited to: halo, alkyl, alkenyl, alkynyl, aryl, heterocyclyl, thiol, alkylthio, arylthio, alkylthioalkyl, arylthioalkyl, alkylsulfonyl, alkylsulfonylalkyl, arylsulfonylalkyl, alkoxy, aryloxy, aralkoxy, aminocarbonyl, alkylaminocarbonyl, arylaminocarbonyl, alkoxycarbonyl, aryloxycarbonyl, haloalkyl, amino, trifluoromethyl, cyano, nitro, alkylamino, arylamino, alkylaminoalkyl, arylaminoalkyl, aminoalkylamino, hydroxy, alkoxyalkyl, carboxyalkyl, alkoxycarbonylalkyl, aminocarbonylalkyl, acyl, aralkoxycarbonyl, carboxylic acid, sulfonic acid, sulfonyl, phosphonic acid, aryl, heteroaryl, heterocyclic, and aliphatic. It is understood that the substituent can be further substituted.

Suitable divalent substituents on a saturated carbon atom of an “optionally substituted” group include the following: ═O, ═S, ═NNR*₂, ═NNHC(O)R*, ═NNHC(O)OR*, ═NNHS(O)₂R*, ═NR*, ═NOR*, —O(C(R*₂))₂₋₃O—, or S(C(R*₂))₂₋₃S—, wherein each independent occurrence of R* is selected from hydrogen, C₁₋₆ aliphatic which may be substituted as defined below, or an unsubstituted 5-6-membered saturated, partially unsaturated, or aryl ring having 0-4 heteroatoms independently selected from nitrogen, oxygen, or sulfur. Suitable divalent substituents that are bound to vicinal substitutable carbons of an “optionally substituted” group include: —O(CR*₂)₂₋₃O—, wherein each independent occurrence of R* is selected from hydrogen, C₁₋₆ aliphatic which may be substituted as defined below, or an unsubstituted 5-6-membered saturated, partially unsaturated, or aryl ring having 0-4 heteroatoms independently selected from nitrogen, oxygen, or sulfur.

The term “protecting group,” is meant that a particular functional moiety, e.g., 0, S, or N, is temporarily blocked so that a reaction can be carried out selectively at another reactive site in a multifunctional compound. In certain embodiments, a protecting group reacts selectively in good yield to give a protected substrate that is stable to the projected reactions; the protecting group should be selectively removable in good yield by readily available, preferably non-toxic reagents that do not attack the other functional groups; the protecting group forms an easily separable derivative (more preferably without the generation of new stereogenic centers); and the protecting group has a minimum of additional functionality to avoid further sites of reaction. As detailed herein, oxygen, sulfur, nitrogen, and carbon protecting groups may be utilized.

An “oxygen protecting group” or “hydroxyl protecting group” may include methyl, methoxylmethyl (MOM), methylthiomethyl (MTM), t-butylthiomethyl, (phenyldimethylsilyl)methoxymethyl (SMOM), benzyloxymethyl (BOM), p-methoxybenzyloxymethyl (PMBM), (4-methoxyphenoxy)methyl (p-AOM), guaiacolmethyl (GUM), pbutoxymethyl, 4-pentenyloxymethyl (POM), siloxymethyl, 2-methoxyethoxymethyl (MEM), 2,2,2-trichloroethoxymethyl, bis(2-chloroethoxy)methyl, 2-(trimethylsilyl)ethoxymethyl (SEMOR), tetrahydropyranyl (THP), 3-bromotetrahydropyranyl, tetrahydrothiopyranyl, 1-methoxycyclohexyl, 4-methoxytetrahydropyranyl (MTHP), 4-methoxytetrahydrothiopyranyl, 4-methoxytetrahydrothiopyranyl S,S-dioxide, l-[(2-chloro-4-methyl)phenyl]-4-methoxypiperidin-4-yl (CTMP), 1,4-dioxan-2-yl, tetrahydrofuranyl, tetrahydrothiofuranyl, 2,3,3a,4,5,6,7,7a-octahydro-7,8,8-trimethyl-4,7-methanobenzofuran-2-yl, 1-ethoxyethyl, 1-(2-chloroethoxy)ethyl, 1-methyl-1-methoxyethyl, 1-methyl-1-benzyloxyethyl, 1-methyl-1-benzyloxy-2-fluoroethyl, 2,2,2-trichloroethyl, 2-trimethylsilylethyl, 2-(phenylselenyl)ethyl, t-butyl, allyl, p-chlorophenyl, p-methoxyphenyl, 2,4-dinitrophenyl, benzyl, p-methoxybenzyl, 3,4-dimethoxybenzyl, o-nitrobenzyl, p-nitrobenzyl, p-halobenzyl, 2,6-dichlorobenzyl, p-cyanobenzyl, p-phenylbenzyl, 2-picolyl, 4-picolyl, 3-methyl-2-picolyl N-oxido, diphenylmethyl, p,p′-dinitrobenzhydryl, 5-dibenzosuberyl, triphenylmethyl, a-naphthyldiphenylmethyl, p-methoxyphenyldiphenyl methyl, di(p-methoxyphenyl)phenylmethyl, tri(p-methoxyphenyl)methyl, 4-(4′-bromophenacyloxyphenyl)diphenylmethyl, 4,4′,4″-tris(4,5-dichlorophthalimidophenyl)methyl, 4,4′,4″-tris(levulinoyloxyphenyl)methyl, 4,4′,4″-tris(benzoyloxyphenyl)methyl, 3-(imidazol-1-yl)bis(4′,4″-dimethoxyphenyl)methyl, 1,1-bis(4-methoxyphenyl)-1′-pyrenylmethyl, 9-anthryl, 9-(9-phenyl)xanthenyl, 9-(9-phenyl-10-oxo)anthryl, 1,3-benzodithiolan-2-yl, benzisothiazolyl S,S-dioxido, trimethylsilyl (TMS), triethylsilyl (TES), triisopropylsilyl (TIPS), dimethylisopropylsilyl (IPDMS), diethylisopropylsilyl (DEIPS), dimethylthexylsilyl, t-butyldimethylsilyl (TBDMS), t-butyldiphenylsilyl (TBDPS), tribenzylsilyl, tri-p-xylylsilyl, triphenylsilyl, diphenylmethylsilyl (DPMS), t-butylmethoxyphenylsilyl (TBMPS), formate, benzoylformate, acetate, chloroacetate, dichloroacetate, trichloroacetate, trifluoroacetate, methoxyacetate, triphenylmethoxyacetate, phenoxyacetate, p-chlorophenoxyacetate, 3-phenylpropionate, 4-oxopentanoate (levulinate), 4,4-(ethylenedithio)pentanoate (levulinoyldithioacetal), pivaloate, adamantoate, crotonate, 4-methoxycrotonate, benzoate, p-pheny]benzoate, 2,4,6-trimethylbenzoate (mesitoate), alkyl methyl carbonate, 9-fluorenylmethyl carbonate (Fmoc), alkyl ethyl carbonate, alkyl 2,2,2-trichloroethyl carbonate (Troc), 2-(trimethylsilyl)ethyl carbonate (TMSEC), 2-(phenylsulfonyl) ethyl carbonate (Psec), 2-(triphenylphosphonio) ethyl carbonate (Peoc), alkyl isobutyl carbonate, alkyl vinyl carbonate alkyl allyl carbonate, alkyl p-nitrophenyl carbonate, alkyl benzyl carbonate, alkyl p-methoxybenzyl carbonate, alkyl 3,4-dimethoxybenzyl carbonate, alkyl o-nitrobenzyl carbonate, alkyl p-nitrobenzyl carbonate, alkyl 5-benzyl thiocarbonate, 4-ethoxy-1-napththyl carbonate, methyl dithiocarbonate, 2-iodobenzoate, 4-azidobutyrate, 4-nitro-4-methylpentanoate, o-(dibromomethyl)benzoate, 2-formylbenzenesulfonate, 2-(methylthiomethoxy)ethyl, 4-(methylthiomethoxy)butyrate, 2-(methylthiomethoxymethyl)benzoate, 2,6-dichloro-4-methylphenoxyacetate, 2,6-dichloro-4-(1,1,3,3-tetramethylbutyl)phenoxyacetate, 2,4-bis(1,1-dimethylpropyl)phenoxyacetate, chlorodiphenylacetate, isobutyrate, monosuccinoate, (E)-2-methyl-2-butcnoatc, o-(methoxycarbonyl)benzoate, a-naphthoate, nitrate, alkyl N,N,N′,N′-tetramethylphosphorodiamidate, alkyl N-phcnylcarbamate, borate, dimethylphosphinothioyl, alkyl 2,4-dinitrophenylsulfenate, sulfate, methanesulfonate (mesylate), benzylsulfonate, and tosylate (Ts). For protecting 1,2- or 1,3-diols, the protecting groups include methylene acetal, ethylidene acetal, 1-i-butylethylidene ketal, 1-phenylethylidene ketal, (4-methoxyphenyl)ethylidene acetal, 2,2,2-trichloroethylidene acetal, acetonide, cyclopentylidene ketal, cyclohexylidene ketal, cycloheptylidene ketal, benzylidene acetal, p-methoxybenzylidene acetal, 2,4-dimethoxybenzylidene ketal, 3,4-dimethoxybenzylidene acetal, 2-nitrobenzylidene acetal, methoxymethylene acetal, ethoxymethylene acetal, dimethoxymethylene ortho ester, 1-methoxyethylidene ortho ester, 1-ethoxyethylidine ortho ester, 1,2-dimethoxyethylidene ortho ester, a-methoxybenzylidene ortho ester, l-(N,N-dimethylamino)ethylidene derivative, a-(N,N′-dimethylamino)benzylidene derivative, 2-oxacyclopentylidene ortho ester, di-t-butylsilylene group (DTBS), 1,3-(1,1,3,3-tetraisopropyldisiloxanylidene) derivative (TIPDS), tetra-t-butoxydisiloxane-1,3-diylidene derivative (TBDS), cyclic carbonates, cyclic boronates, ethyl boronate, and phenyl boronate. Additionally, a variety of protecting groups are described in Protective Groups in Organic Synthesis, Third Ed. Greene, T. W. and Wuts, P. G., Eds., John Wiley & Sons, New York: 1999, the entire contents of which are hereby incorporated by reference.

### Oligonucleotide Definitions and Designs

Unless specific definitions are provided, the nomenclature utilized in connection with, and the procedures and techniques of, analytical chemistry, synthetic organic chemistry, and medicinal and pharmaceutical chemistry described herein are those well known and commonly used in the art. Standard techniques may be used for chemical synthesis, and chemical analysis. Certain such techniques and procedures may be found for example in “Carbohydrate Modifications in Antisense Research” Edited by Sangvi and Cook, American Chemical Society, Washington D.C., 1994; “Remington's Pharmaceutical Sciences,” Mack Publishing Co., Easton, Pa., 18th edition, 1990; and “Antisense Drug Technology, Principles, Strategies, and Applications” Edited by Stanley T. Crooke, CRC Press, Boca Raton, Fla.; and Sambrook et al., “Molecular Cloning, A laboratory Manual,” 2d Edition, Cold Spring Harbor Laboratory Press, 1989, which are hereby incorporated by reference for any purpose. Where permitted, all patents, applications, published applications and other publications and other data referred to throughout in the disclosure herein are incorporated by reference in their entirety.

As used herein, the term “target nucleic acid” refers to any nucleic acid molecule the expression or activity of which is capable of being modulated by an siRNA compound. Target nucleic acids include, but are not limited to, RNA (including, but not limited to pre-mRNA and mRNA or portions thereof) transcribed from DNA encoding a target protein, and also cDNA derived from such RNA, and miRNA. For example, the target nucleic acid can be a cellular gene (or mRNA transcribed from the gene) whose expression is associated with a particular disorder or disease state. In some embodiments, a target nucleic acid can be a nucleic acid molecule from an infectious agent.

As used herein, the term “iRNA” refers to an agent that mediates the targeted cleavage of an RNA transcript. These agents associate with a cytoplasmic multi-protein complex known as RNAi-induced silencing complex (RISC). Agents that are effective in inducing RNA interference are also referred to as siRNA, RNAi agent, or iRNA agent, herein. Thus, these terms can be used interchangeably herein. As used herein, the term iRNA includes microRNAs and pre-microRNAs. Moreover, the “compound” or “compounds” of the invention as used herein, also refers to the iRNA agent, and can be used interchangeably with the iRNA agent.

The iRNA agent should include a region of sufficient homology to the target gene, and be of sufficient length in terms of nucleotides, such that the iRNA agent, or a fragment thereof, can mediate downregulation of the target gene. (For ease of exposition the term nucleotide or ribonucleotide is sometimes used herein in reference to one or more monomeric subunits of an iRNA agent. It will be understood herein that the usage of the term “ribonucleotide” or “nucleotide”, herein can, in the case of a modified RNA or nucleotide surrogate, also refer to a modified nucleotide, or surrogate replacement moiety at one or more positions.) Thus, the iRNA agent is or includes a region which is at least partially, and in some embodiments fully, complementary to the target RNA. It is not necessary that there be perfect complementarity between the iRNA agent and the target, but the correspondence must be sufficient to enable the iRNA agent, or a cleavage product thereof, to direct sequence specific silencing, e.g., by RNAi cleavage of the target RNA, e.g., mRNA. Complementarity, or degree of homology with the target strand, is most critical in the antisense strand. While perfect complementarity, particularly in the antisense strand, is often desired some embodiments can include, particularly in the antisense strand, one or more, or for example, 6, 5, 4, 3, 2, or fewer mismatches (with respect to the target RNA). The sense strand need only be sufficiently complementary with the antisense strand to maintain the overall double stranded character of the molecule.

iRNA agents include: molecules that are long enough to trigger the interferon response (which can be cleaved by Dicer (Bernstein et al. 2001. Nature, 409:363-366) and enter a RISC (RNAi-induced silencing complex)); and, molecules which are sufficiently short that they do not trigger the interferon response (which molecules can also be cleaved by Dicer and/or enter a RISC), e.g., molecules which are of a size which allows entry into a RISC, e.g., molecules which resemble Dicer-cleavage products. Molecules that are short enough that they do not trigger an interferon response are termed siRNA agents or shorter iRNA agents herein. “siRNA agent or shorter iRNA agent” as used herein, refers to an iRNA agent, e.g., a double stranded RNA agent or single strand agent, that is sufficiently short that it does not induce a deleterious interferon response in a human cell, e.g., it has a duplexed region of less than 60, 50, 40, or 30 nucleotide pairs. The siRNA agent, or a cleavage product thereof, can down regulate a target gene, e.g., by inducing RNAi with respect to a target RNA, wherein the target may comprise an endogenous or pathogen target RNA.

A “single strand iRNA agent” as used herein, is an iRNA agent which is made up of a single molecule. It may include a duplexed region, formed by intra-strand pairing, e.g., it may be, or include, a hairpin or pan-handle structure. Single strand iRNA agents may be antisense with regard to the target molecule. A single strand iRNA agent may be sufficiently long that it can enter the RISC and participate in RISC mediated cleavage of a target mRNA. A single strand iRNA agent is at least 14, and in other embodiments at least 15, 20, 25, 29, 35, 40, or 50 nucleotides in length. In certain embodiments, it is less than 200, 100, or 60 nucleotides in length.

A loop refers to a region of an iRNA strand that is unpaired with the opposing nucleotide in the duplex when a section of the iRNA strand forms base pairs with another strand or with another section of the same strand.

Hairpin iRNA agents will have a duplex region equal to or at least 17, 18, 19, 29, 21, 22, 23, 24, or 25 nucleotide pairs. The duplex region will may be equal to or less than 200, 100, or 50, in length. In certain embodiments, ranges for the duplex region are 15-30, 17 to 23, 19 to 23, and 19 to 21 nucleotides pairs in length. The hairpin may have a single strand overhang or terminal unpaired region, in some embodiments at the 3′, and in certain embodiments on the antisense side of the hairpin. In some embodiments, the overhangs are 2-3 nucleotides in length.

A “double stranded (ds) iRNA agent” as used herein, is an iRNA agent which includes more than one, and in some cases two, strands in which interchain hybridization can form a region of duplex structure.

As used herein, the terms “siRNA activity” and “RNAi activity” refer to gene silencing by an siRNA.

As used herein, “gene silencing” by a RNA interference molecule refers to a decrease in the mRNA level in a cell for a target gene by at least about 5%, at least about 10%, at least about 20%, at least about 30%, at least about 40%, at least about 50%, at least about 60%, at least about 70%, at least about 80%, at least about 90%, at least about 95%, at least about 99% up to and including 100%, and any integer in between of the mRNA level found in the cell without the presence of the miRNA or RNA interference molecule. In one preferred embodiment, the mRNA levels are decreased by at least about 70%, at least about 80%, at least about 90%, at least about 95%, at least about 99%, up to and including 100% and any integer in between 5% and 100%.”

As used herein the term “modulate gene expression” means that expression of the gene, or level of RNA molecule or equivalent RNA molecules encoding one or more proteins or protein subunits is up regulated or down regulated, such that expression, level, or activity is greater than or less than that observed in the absence of the modulator. For example, the term “modulate” can mean “inhibit,” but the use of the word “modulate” is not limited to this definition.

As used herein, gene expression modulation happens when the expression of the gene, or level of RNA molecule or equivalent RNA molecules encoding one or more proteins or protein subunits is at least 5%, 10%, 20%, 30%, 40%, 50%, 60%, 70%, 80%, 90%, 100%, 2-fold, 3-fold, 4-fold, 5-fold or more different from that observed in the absence of the siRNA, e.g., RNAi agent. The % and/or fold difference can be calculated relative to the control or the non-control, for example,

$$\% difference = \frac{[ expression with siRNA - expression without siRNA ]}{expression without siRNA} \\ or \\ \% difference = \frac{[ expression with siRNA - expression without siRNA ]}{expression without siRNA}$$

As used herein, the term “inhibit”, “down-regulate”, or “reduce” in relation to gene expression, means that the expression of the gene, or level of RNA molecules or equivalent RNA molecules encoding one or more proteins or protein subunits, or activity of one or more proteins or protein subunits, is reduced below that observed in the absence of modulator. The gene expression is down-regulated when expression of the gene, or level of RNA molecules or equivalent RNA molecules encoding one or more proteins or protein subunits, or activity of one or more proteins or protein subunits, is reduced at least 10% lower relative to a corresponding non-modulated control, and preferably at least 10%, 20%, 30%, 40%, 50%, 60%, 70%, 80%, 90%, 95%, 98%, 99% or most preferably, 100% (i.e., no gene expression).

As used herein, the term “increase” or “up-regulate” in relation to gene expression means that the expression of the gene, or level of RNA molecules or equivalent RNA molecules encoding one or more proteins or protein subunits, or activity of one or more proteins or protein subunits, is increased above that observed in the absence of modulator. The gene expression is up-regulated when expression of the gene, or level of RNA molecules or equivalent RNA molecules encoding one or more proteins or protein subunits, or activity of one or more proteins or protein subunits, is increased at least 10% relative to a corresponding non-modulated control, and preferably at least 10%, 20%, 30%, 40%, 50%, 60%, 70%, 80%, 90%, 95%, 98%, 100%, 1.1-fold, 1.25-fold, 1.5-fold, 1.75-fold, 2-fold, 3-fold, 4-fold, 5-fold, 10-fold, 50-fold, 100-fold or more.

The term “increased” or “increase” as used herein generally means an increase by a statically significant amount; for the avoidance of any doubt, “increased” means an increase of at least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, or any increase between 2-fold and 10-fold or greater as compared to a reference level.

The term “reduced” or “reduce” as used herein generally means a decrease by a statistically significant amount. However, for avoidance of doubt, “reduced” means a decrease by at least 10% as compared to a reference level, for example a decrease by at least about 20%, or at least about 30%, or at least about 40%, or at least about 50%, or at least about 60%, or at least about 70%, or at least about 80%, or at least about 90% or up to and including a 100% decrease (i.e. absent level as compared to a reference sample), or any decrease between 10-100% as compared to a reference level.

The double-stranded iRNAs comprise two oligonucleotide strands that are sufficiently complementary to hybridize to form a duplex structure. Generally, the duplex structure is between 15 and 30, more generally between 18 and 25, yet more generally between 19 and 24, and most generally between 19 and 21 base pairs in length. In some embodiments, longer double-stranded iRNAs of between 25 and 30 base pairs in length are preferred. In some embodiments, shorter double-stranded iRNAs of between 10 and 15 base pairs in length are preferred. In another embodiment, the double-stranded iRNA is at least 21 nucleotides long.

In some embodiments, the double-stranded iRNA comprises a sense strand and an antisense strand, wherein the antisense RNA strand has a region of complementarity which is complementary to at least a part of a target sequence, and the duplex region is 14-30 nucleotides in length. Similarly, the region of complementarity to the target sequence is between 14 and 30, more generally between 18 and 25, yet more generally between 19 and 24, and most generally between 19 and 21 nucleotides in length.

The phrase “antisense strand” as used herein, refers to an oligonucleotide strand that is substantially or 100% complementary to a target sequence of interest. The phrase “antisense strand” includes the antisense region of both oligonucleotide strands that are formed from two separate strands, as well as unimolecular oligonucleotide strands that are capable of forming hairpin or dumbbell type structures. The terms “antisense strand” and “guide strand” are used interchangeably herein.

The phrase “sense strand” refers to an oligonucleotide strand that has the same nucleoside sequence, in whole or in part, as a target sequence such as a messenger RNA or a sequence of DNA. The terms “sense strand” and “passenger strand” are used interchangeably herein.

By “specifically hybridizable” and “complementary” is meant that a nucleic acid can form hydrogen bond(s) with another nucleic acid sequence by either traditional Watson-Crick or other non-traditional types. In reference to the nucleic molecules of the present invention, the binding free energy for a nucleic acid molecule with its complementary sequence is sufficient to allow the relevant function of the nucleic acid to proceed, e.g., RNAi activity. Determination of binding free energies for nucleic acid molecules is well known in the art (see, e.g., Turner et al, 1987, CSH Symp. Quant. Biol. LII pp. 123-133; Frier et al., 1986, Proc. Nat. Acad. Sci. USA 83:9373-9377; Turner et al., 1987, I. Am. Chem. Soc. 109:3783-3785). A percent complementarity indicates the percentage of contiguous residues in a nucleic acid molecule that can form hydrogen bonds (e.g., Watson-Crick base pairing) with a second nucleic acid sequence (e.g., 5, 6, 7, 8, 9,10 out of 10 being 50%, 60%, 70%, 80%, 90%, and 100% complementary). “Perfectly complementary” or 100% complementarity means that all the contiguous residues of a nucleic acid sequence will hydrogen bond with the same number of contiguous residues in a second nucleic acid sequence. Less than perfect complementarity refers to the situation in which some, but not all, nucleoside units of two strands can hydrogen bond with each other. “Substantial complementarity” refers to polynucleotide strands exhibiting 90% or greater complementarity, excluding regions of the polynucleotide strands, such as overhangs, that are selected so as to be noncomplementary. Specific binding requires a sufficient degree of complementarity to avoid non-specific binding of the oligonucleotide to non-target sequences under conditions in which specific binding is desired, i.e., under physiological conditions in the case of in vivo assays or therapeutic treatment, or in the case of in vitro assays, under conditions in which the assays are performed. The non-target sequences typically differ by at least 5 nucleotides.

In some embodiments, the double-stranded region is equal to or at least, 10, 11, 12, 13, 14, 15, 16, 17, 18, 19, 20, 21, 22, 23, 23, 24, 25, 26, 27, 28, 29, 30 or more nucleotide pairs in length.

In some embodiments, the antisense strand is equal to or at least 14, 15, 16, 17, 18, 19, 20, 21, 22, 23, 23, 24, 25, 26, 27, 28, 29, or 30 nucleotides in length.

In some embodiments, the sense strand is equal to or at least 10, 11, 12, 13, 14, 15, 16, 17, 18, 19, 20, 21, 22, 23, 23, 24, 25, 26, 27, 28, 29, or 30 nucleotides in length.

In one embodiment, the sense and antisense strands are each 15 to 30 nucleotides in length. In one embodiment, the sense and antisense strands are each 19 to 25 nucleotides in length. In one embodiment, the sense and antisense strands are each 21 to 23 nucleotides in length.

In some embodiments, one strand has at least one stretch of 1-5 single-stranded nucleotides in the double-stranded region. By “stretch of single-stranded nucleotides in the double-stranded region” is meant that there is present at least one nucleotide base pair at both ends of the single-stranded stretch. In some embodiments, both strands have at least one stretch of 1-5 (e.g., 1, 2, 3, 4, or 5) single-stranded nucleotides in the double stranded region. When both strands have a stretch of 1-5 (e.g., 1, 2, 3, 4, or 5) single-stranded nucleotides in the double stranded region, such single-stranded nucleotides can be opposite to each other (e.g., a stretch of mismatches) or they can be located such that the second strand has no single-stranded nucleotides opposite to the single-stranded iRNAs of the first strand and vice versa (e.g., a single-stranded loop). In some embodiments, the single-stranded nucleotides are present within 8 nucleotides from either end, for example 8, 7, 6, 5, 4, 3, or 2 nucleotide from either the 5′ or 3′ end of the region of complementarity between the two strands.

In one embodiment, the oligonucleotide comprises a single-stranded overhang on at least one of the termini. In one embodiment, the single-stranded overhang is 1, 2, or 3 nucleotides in length.

In one embodiment, the sense strand of the iRNA agent is 21-nucleotides in length, and the antisense strand is 23-nucleotides in length, wherein the strands form a double-stranded region of 21 consecutive base pairs having a 2-nucleotide long single-stranded overhangs at the 3′-end.

In some embodiments, each strand of the double-stranded iRNA has a ZXY structure, such as is described in PCT Publication No. 2004080406, which is hereby incorporated by reference in its entirety.

In certain embodiment, the two strands of double-stranded oligonucleotide can be linked together. The two strands can be linked to each other at both ends, or at one end only. By linking at one end is meant that 5′-end of first strand is linked to the 3′-end of the second strand or 3′-end of first strand is linked to 5′-end of the second strand. When the two strands are linked to each other at both ends, 5′-end of first strand is linked to 3′-end of second strand and 3′-end of first strand is linked to 5′-end of second strand. The two strands can be linked together by an oligonucleotide linker including, but not limited to, (N)n; wherein N is independently a modified or unmodified nucleotide and n is 3-23. In some embodiments, n is 3-10, e.g., 3, 4, 5, 6, 7, 8, 9, or 10. In some embodiments, the oligonucleotide linker is selected from the group consisting of GNRA, (G)₄, (U)₄, and (dT)₄, wherein N is a modified or unmodified nucleotide and R is a modified or unmodified purine nucleotide. Some of the nucleotides in the linker can be involved in base-pair interactions with other nucleotides in the linker. The two strands can also be linked together by a non-nucleosidic linker, e.g. a linker described herein. It will be appreciated by one of skill in the art that any oligonucleotide chemical modifications or variations describe herein can be used in the oligonucleotide linker.

Hairpin and dumbbell type oligonucleotide will have a duplex region equal to or at least 14, 15, 15, 16, 17, 18, 19, 29, 21, 22, 23, 24, or 25 nucleotide pairs. The duplex region can be equal to or less than 200, 100, or 50, in length. In some embodiments, ranges for the duplex region are 15-30, 17 to 23, 19 to 23, and 19 to 21 nucleotides pairs in length.

The hairpin oligonucleotide can have a single strand overhang or terminal unpaired region, in some embodiments at the 3′, and in some embodiments on the antisense side of the hairpin. In some embodiments, the overhangs are 1-4, more generally 2-3 nucleotides in length. The hairpin oligonucleotide s that can induce RNA interference are also referred to as “shRNA” herein.

In certain embodiments, two oligonucleotide strands specifically hybridize when there is a sufficient degree of complementarity to avoid non-specific binding of the antisense strand to non-target nucleic acid sequences under conditions in which specific binding is desired, i.e., under physiological conditions in the case of in vivo assays or therapeutic treatment, and under conditions in which assays are performed in the case of in vitro assays.

As used herein, “stringent hybridization conditions” or “stringent conditions” refers to conditions under which an antisense strand will hybridize to its target sequence, but to a minimal number of other sequences. Stringent conditions are sequence-dependent and will be different in different circumstances, and “stringent conditions” under which antisense strand hybridize to a target sequence are determined by the nature and composition of the antisense strand and the assays in which they are being investigated.

It is understood in the art that incorporation of nucleotide affinity modifications may allow for a greater number of mismatches compared to an unmodified oligonucleotide. Similarly, certain oligonucleotide sequences may be more tolerant to mismatches than other oligonucleotide sequences. One of ordinary skill in the art is capable of determining an appropriate number of mismatches between oligonucleotides, or between an oligonucleotide and a target nucleic acid, such as by determining melting temperature (Tm). Tm or ATm can be calculated by techniques that are familiar to one of ordinary skill in the art. For example, techniques described in Freier et al. (Nucleic Acids Research, 1997, 25, 22: 4429-4443) allow one of ordinary skill in the art to evaluate nucleotide modifications for their ability to increase the melting temperature of an RNA:DNA duplex.

Additional dsRNA Design

In one embodiment, the iRNA agent is a double ended bluntmer of 19 nt in length, wherein the sense strand contains at least one motif of three 2′-F modifications on three consecutive nucleotides at positions 7, 8, 9 from the 5′end. The antisense strand contains at least one motif of three 2′-O-methyl modifications on three consecutive nucleotides at positions 11, 12, 13 from the 5′end.

In one embodiment, the iRNA agent is a double ended bluntmer of 20 nt in length, wherein the sense strand contains at least one motif of three 2′-F modifications on three consecutive nucleotides at positions 8, 9, 10 from the 5′end. The antisense strand contains at least one motif of three 2′-O-methyl modifications on three consecutive nucleotides at positions 11, 12, 13 from the 5′end.

In one embodiment, the iRNA agent is a double ended bluntmer of 21 nt in length, wherein the sense strand contains at least one motif of three 2′-F modifications on three consecutive nucleotides at positions 9, 10, 11 from the 5′end. The antisense strand contains at least one motif of three 2′-O-methyl modifications on three consecutive nucleotides at positions 11, 12, 13 from the 5′end.

In one embodiment, the iRNA agent comprises a 21 nucleotides (nt) sense strand and a 23 nucleotides (nt) antisense, wherein the sense strand contains at least one motif of three 2′-F modifications on three consecutive nucleotides at positions 9, 10, 11 from the 5′end; the antisense strand contains at least one motif of three 2′-O-methyl modifications on three consecutive nucleotides at positions 11, 12, 13 from the 5′end, wherein one end of the iRNA is blunt, while the other end is comprises a 2 nt overhang. Preferably, the 2 nt overhang is at the 3′-end of the antisense. Optionally, the iRNA agent further comprises a ligand (e.g., GalNAc₃).

In one embodiment, the iRNA agent comprises a sense and antisense strands, wherein: the sense strand is 25-30 nucleotide residues in length, wherein starting from the 5′ terminal nucleotide (position 1) positions 1 to 23 of said first strand comprise at least 8 ribonucleotides; antisense strand is 36-66 nucleotide residues in length and, starting from the 3′ terminal nucleotide, comprises at least 8 ribonucleotides in the positions paired with positions 1-23 of sense strand to form a duplex; wherein at least the 3′ terminal nucleotide of antisense strand is unpaired with sense strand, and up to 6 consecutive 3′ terminal nucleotides are unpaired with sense strand, thereby forming a 3′ single stranded overhang of 1-6 nucleotides; wherein the 5′ terminus of antisense strand comprises from 10-30 consecutive nucleotides which are unpaired with sense strand, thereby forming a 10-30 nucleotide single stranded 5′ overhang; wherein at least the sense strand 5′ terminal and 3′ terminal nucleotides are base paired with nucleotides of antisense strand when sense and antisense strands are aligned for maximum complementarity, thereby forming a substantially duplexed region between sense and antisense strands; and antisense strand is sufficiently complementary to a target RNA along at least 19 ribonucleotides of antisense strand length to reduce target gene expression when said double stranded nucleic acid is introduced into a mammalian cell; and wherein the sense strand contains at least one motif of three 2′-F modifications on three consecutive nucleotides, where at least one of the motifs occurs at or near the cleavage site. The antisense strand contains at least one motif of three 2′-O-methyl modifications on three consecutive nucleotides at or near the cleavage site.

In one embodiment, the iRNA agent comprises a sense and antisense strands, wherein said iRNA agent comprises a first strand having a length which is at least 25 and at most 29 nucleotides and a second strand having a length which is at most 30 nucleotides with at least one motif of three 2′-O-methyl modifications on three consecutive nucleotides at position 11, 12, 13 from the 5′ end; wherein said 3′ end of said first strand and said 5′ end of said second strand form a blunt end and said second strand is 1-4 nucleotides longer at its 3′ end than the first strand, wherein the duplex region which is at least 25 nucleotides in length, and said second strand is sufficiently complementary to a target mRNA along at least 19 nt of said second strand length to reduce target gene expression when said iRNA agent is introduced into a mammalian cell, and wherein dicer cleavage of said iRNA preferentially results in an siRNA comprising said 3′ end of said second strand, thereby reducing expression of the target gene in the mammal. Optionally, the iRNA agent further comprises a ligand (e.g., GalNAc₃).

In one embodiment, the sense strand contains at least one motif of three identical modifications on three consecutive nucleotides, where one of the motifs occurs at the cleavage site in the sense strand. For instance, the sense strand can contain at least one motif of three 2′-F modifications on three consecutive nucleotides within 7-15 positions from the 5′end.

In one embodiment, the antisense strand can also contain at least one motif of three identical modifications on three consecutive nucleotides, where one of the motifs occurs at or near the cleavage site in the antisense strand. For instance, the antisense strand can contain at least one motif of three 2′-O-methyl modifications on three consecutive nucleotides within 9-15 positions from the 5′end.

For iRNA agent having a duplex region of 17-23 nt in length, the cleavage site of the antisense strand is typically around the 10, 11 and 12 positions from the 5′-end. Thus the motifs of three identical modifications may occur at the 9, 10, 11 positions; 10, 11, 12 positions; 11, 12, 13 positions; 12, 13, 14 positions; or 13, 14, 15 positions of the antisense strand, the count starting from the 1^{st} nucleotide from the 5′-end of the antisense strand, or, the count starting from the 1^{st} paired nucleotide within the duplex region from the 5′-end of the antisense strand. The cleavage site in the antisense strand may also change according to the length of the duplex region of the iRNA from the 5′-end.

In some embodiments, the iRNA agent comprises a sense strand and antisense strand each having 14 to 30 nucleotides, wherein the sense strand contains at least two motifs of three identical modifications on three consecutive nucleotides, where at least one of the motifs occurs at or near the cleavage site within the strand and at least one of the motifs occurs at another portion of the strand that is separated from the motif at the cleavage site by at least one nucleotide. In one embodiment, the antisense strand also contains at least one motif of three identical modifications on three consecutive nucleotides, where at least one of the motifs occurs at or near the cleavage site within the strand. The modification in the motif occurring at or near the cleavage site in the sense strand is different than the modification in the motif occurring at or near the cleavage site in the antisense strand.

In some embodiments, the iRNA agent comprises a sense strand and antisense strand each having 14 to 30 nucleotides, wherein the sense strand contains at least one motif of three 2′-F modifications on three consecutive nucleotides, where at least one of the motifs occurs at or near the cleavage site in the strand. In one embodiment, the antisense strand also contains at least one motif of three 2′-O-methyl modifications on three consecutive nucleotides at or near the cleavage site.

In some embodiments, the iRNA agent comprises a sense strand and antisense strand each having 14 to 30 nucleotides, wherein the sense strand contains at least one motif of three 2′-F modifications on three consecutive nucleotides at positions 9, 10, 11 from the 5′end, and wherein the antisense strand contains at least one motif of three 2′-O-methyl modifications on three consecutive nucleotides at positions 11, 12, 13 from the 5′end.

In one embodiment, the iRNA agent comprises mismatch(es) with the target, within the duplex, or combinations thereof. The mismatch can occur in the overhang region or the duplex region. The base pair can be ranked on the basis of their propensity to promote dissociation or melting (e.g., on the free energy of association or dissociation of a particular pairing, the simplest approach is to examine the pairs on an individual pair basis, though next neighbor or similar analysis can also be used). In terms of promoting dissociation: A:U is preferred over G:C; G:U is preferred over G:C; and I:C is preferred over G:C (I═inosine). Mismatches, e.g., non-canonical or other than canonical pairings (as described elsewhere herein) are preferred over canonical (A:T, A:U, G:C) pairings; and pairings which include a universal base are preferred over canonical pairings.

In one embodiment, the iRNA agent comprises at least one of the first 1, 2, 3, 4, or 5 base pairs within the duplex regions from the 5′-end of the antisense strand can be chosen independently from the group of: A:U, G:U, I:C, and mismatched pairs, e.g., non-canonical or other than canonical pairings or pairings which include a universal base, to promote the dissociation of the antisense strand at the 5′-end of the duplex.

In one embodiment, the nucleotide at the 1 position within the duplex region from the 5′-end in the antisense strand is selected from the group consisting of A, dA, dU, U, and dT. Alternatively, at least one of the first 1, 2 or 3 base pair within the duplex region from the 5′-end of the antisense strand is an AU base pair. For example, the first base pair within the duplex region from the 5′-end of the antisense strand is an AU base pair.

In one embodiment, 100%, 95%, 90%, 85%, 80%, 75%, 70%, 65%, 60%, 55%, 50%, 45%, 40%, 35% or 30% of the dsRNA agent is modified. For example, when 50% of the dsRNA agent is modified, 50% of all nucleotides present in the dsRNA agent contain a modification as described herein.

In some embodiments, the dsRNA agent further comprises one or more 2′-O modifications selected from the group consisting of 2′-deoxy, 2′-O-methoxyalkyl, 2′-O-methyl, 2′-O-allyl, 2′-C-allyl, 2′-fluoro, 2′-O—N-methylacetamido (2′-O-NMA), 2′-O-dimethylaminoethoxyethyl (2′-O-DMAEOE), 2′-O-aminopropyl (2′-O-AP), and 2′-ara-F.

In one embodiment, each of the sense and antisense strands is independently modified with non-natural nucleotides such as acyclic nucleotides, LNA, HNA, CeNA, 2′-methoxyethyl, 2′-O-methyl, 2′-O-allyl, 2′-C-allyl, 2′-deoxy, 2′-fluoro, 2′-O—N-methylacetamido (2′-O-NMA), a 2′-O-dimethylaminoethoxyethyl (2′-O-DMAEOE), 2′-O-aminopropyl (2′-O-AP), or 2′-ara-F.

In one embodiment, each of the sense and antisense strands of the dsRNA agent contains at least two different modifications.

In some embodiments, the oligonucleotide contains one, two, three, four, five, six, seven, eight, nine, ten, eleven or twelve 2′-F modification(s). In one example, oligonucleotide contains nine or ten 2′-F modifications.

In one embodiment, the oligonucleotide does not contain any 2′-F modification.

The iRNA agent may further comprise at least one phosphorothioate or methylphosphonate internucleotide linkage. The phosphorothioate or methylphosphonate internucleotide linkage modification may occur on any nucleotide of the sense strand or antisense strand or both in any position of the strand. For instance, the internucleotide linkage modification may occur on every nucleotide on the sense strand or antisense strand; each internucleotide linkage modification may occur in an alternating pattern on the sense strand or antisense strand; or the sense strand or antisense strand may contain both internucleotide linkage modifications in an alternating pattern. The alternating pattern of the internucleotide linkage modification on the sense strand may be the same or different from the antisense strand, and the alternating pattern of the internucleotide linkage modification on the sense strand may have a shift relative to the alternating pattern of the internucleotide linkage modification on the antisense strand.

In one embodiment, the iRNA comprises the phosphorothioate or methylphosphonate internucleotide linkage modification in the overhang region. For example, the overhang region may contain two nucleotides having a phosphorothioate or methylphosphonate internucleotide linkage between the two nucleotides. Internucleotide linkage modifications also may be made to link the overhang nucleotides with the terminal paired nucleotides within duplex region. For example, at least 2, 3, 4, or all the overhang nucleotides may be linked through phosphorothioate or methylphosphonate internucleotide linkage, and optionally, there may be additional phosphorothioate or methylphosphonate internucleotide linkages linking the overhang nucleotide with a paired nucleotide that is next to the overhang nucleotide. For instance, there may be at least two phosphorothioate internucleotide linkages between the terminal three nucleotides, in which two of the three nucleotides are overhang nucleotides, and the third is a paired nucleotide next to the overhang nucleotide. Preferably, these terminal three nucleotides may be at the 3′-end of the antisense strand.

In one embodiment, the sense strand and/or antisense strand comprises one or more blocks of phosphorothioate or methylphosphonate internucleotide linkages. In one example, the sense strand comprises one block of two phosphorothioate or methylphosphonate internucleotide linkages. In one example, the antisense strand comprises two blocks of two phosphorothioate or methylphosphonate internucleotide linkages. For example, the two blocks of phosphorothioate or methylphosphonate internucleotide linkages are separated by 16-18 phosphate internucleotide linkages.

In one embodiment, each of the sense and antisense strands has 15-30 nucleotides. In one example, the sense strand has 19-22 nucleotides, and the antisense strand has 19-25 nucleotides. In another example, the sense strand has 21 nucleotides, and the antisense strand has 23 nucleotides.

In one embodiment, the nucleotide at position 1 of the 5′-end of the antisense strand in the duplex is selected from the group consisting of A, dA, dU, U, and dT. In one embodiment, at least one of the first, second, and third base pair from the 5′-end of the antisense strand is an AU base pair.

In one embodiment, the antisense strand of the dsRNA agent is 100% complementary to a target RNA to hybridize thereto and inhibits its expression through RNA interference. In another embodiment, the antisense strand of the dsRNA agent is at least 95%, at least 90%, at least 85%, at least 80%, at least 75%, at least 70%, at least 65%, at least 60%, at least 55%, or at least 50% complementary to a target RNA.

In one aspect, the invention relates to a dsRNA agent as defined herein capable of inhibiting the expression of a target gene. The dsRNA agent comprises a sense strand and an antisense strand, each strand having 14 to 40 nucleotides. The sense strand contains at least one thermally destabilizing nucleotide, wherein at least one of said thermally destabilizing nucleotide occurs at or near the site that is opposite to the seed region of the antisense strand (i.e. at position 2-8 of the 5′-end of the antisense strand).

The thermally destabilizing nucleotide can occur, for example, between positions 14-17 of the 5′-end of the sense strand when the sense strand is 21 nucleotides in length. The antisense strand contains at least two modified nucleic acids that are smaller than a sterically demanding 2′-OMe modification. Preferably, the two modified nucleic acids that are smaller than a sterically demanding 2′-OMe are separated by 11 nucleotides in length. For example, the two modified nucleic acids are at positions 2 and 14 of the 5′end of the antisense strand.

In one embodiment, the dsRNA agents comprise:
(a) a sense strand having:
   (i) a length of 18-23 nucleotides;
   (ii) three consecutive 2′-F modifications at positions 7-15; and
(b) an antisense strand having:
   (i) a length of 18-23 nucleotides;
   (ii) at least 2′-F modifications anywhere on the strand; and
   (iii) at least two phosphorothioate internucleotide linkages at the first five nucleotides (counting from the 5′ end);
wherein the dsRNA agents have one or more lipophilic monomers containing one or more lipophilic moieties conjugated to one or more positions on at least one strand; and either have two nucleotides overhang at the 3′-end of the antisense strand, and a blunt end at the 5′-end of the antisense strand; or blunt end both ends of the duplex.

In one embodiment, the dsRNA agents comprise:
(a) a sense strand having:
   (i) a length of 18-23 nucleotides;
   (ii) less than four 2′-F modifications;
(b) an antisense strand having:
   (i) a length of 18-23 nucleotides;
   (ii) at less than twelve 2′-F modification; and
   (iii) at least two phosphorothioate internucleotide linkages at the first five nucleotides (counting from the 5′ end);
wherein the dsRNA agents have one or more lipophilic monomers containing one or more lipophilic moieties conjugated to one or more positions on at least one strand; and either have two nucleotides overhang at the 3′-end of the antisense strand, and a blunt end at the 5′-end of the antisense strand; or blunt end both ends of the duplex.

In one embodiment, the dsRNA agents comprise:
(a) a sense strand having:
   (i) a length of 19-35 nucleotides;
   (ii) less than four 2′-F modifications;
(b) an antisense strand having:
   (i) a length of 19-35 nucleotides;
   (ii) at less than twelve 2′-F modification; and
   (iii) at least two phosphorothioate internucleotide linkages at the first five nucleotides (counting from the 5′ end);
wherein the duplex region is between 19 to 25 base pairs (preferably 19, 20, 21 or 22); and wherein the dsRNA agents have one or more lipophilic monomers containing one or more lipophilic moieties conjugated to one or more positions on at least one strand; and either have two nucleotides overhang at the 3′-end of the antisense strand, and a blunt end at the 5′-end of the antisense strand; or blunt end both ends of the duplex.

In one embodiment, the dsRNA agents comprise a sense strand and antisense strands having a length of 15-30 nucleotides; at least two phosphorothioate internucleotide linkages at the first five nucleotides on the antisense strand (counting from the 5′ end); wherein the duplex region is between 19 to 25 base pairs (preferably 19, 20, 21 or 22); wherein the dsRNA agents have one or more lipophilic monomers containing one or more lipophilic moieties conjugated to one or more positions on at least one strand; and wherein the dsRNA agents have less than 20%, less than 15% and less than 10% non-natural nucleotide.

In one embodiment, the dsRNA agents comprise a sense strand and antisense strands having a length of 15-30 nucleotides; at least two phosphorothioate internucleotide linkages at the first five nucleotides on the antisense strand (counting from the 5′ end); wherein the duplex region is between 19 to 25 base pairs (preferably 19, 20, 21 or 22); wherein the dsRNA agents have one or more lipophilic monomers containing one or more lipophilic moieties conjugated to one or more positions on at least one strand; and wherein the dsRNA agents have greater than 80%, greater than 85% and greater than 90% natural nucleotide, such as 2′-OH, 2′-deoxy and 2′-OMe are natural nucleotides.

In one embodiment, the dsRNA agents comprise a sense strand and antisense strands having a length of 15-30 nucleotides; at least two phosphorothioate internucleotide linkages at the first five nucleotides on the antisense strand (counting from the 5′ end); wherein the duplex region is between 19 to 25 base pairs (preferably 19, 20, 21 or 22); wherein the dsRNA agents have one or more lipophilic monomers containing one or more lipophilic moieties conjugated to one or more positions on at least one strand; and wherein the dsRNA agents have 100% natural nucleotide, such as 2′-OH, 2′-deoxy and 2′-OMe are natural nucleotides.

In one embodiment, the dsRNA agents comprises a sense strand and an antisense strand, each strand having 14 to 30 nucleotides, wherein the sense strand sequence is represented by formula (I):

$${5}^{′} {n}_{p} - {N}_{a} - {( \begin{matrix}X & X & X\end{matrix} )}_{i} - {N}_{b} - \begin{matrix}Y & Y & Y\end{matrix} - {N}_{b} - {( \begin{matrix}Z & Z & Z\end{matrix} )}_{j} - {N}_{a} - {n}_{q} {3}^{′} \qquad ( I )$$

wherein:
i and j are each independently 0 or 1;
p and q are each independently 0-6;
each Nₐ independently represents an oligonucleotide sequence comprising 0-25 modified nucleotides, each sequence comprising at least two differently modified nucleotides;
each N_{b} independently represents an oligonucleotide sequence comprising 1, 2, 3, 4, 5, or 6 modified nucleotides;
each nₚ and n_{q} independently represent an overhang nucleotide;
wherein N_{b} and Y do not have the same modification;
wherein XXX, YYY and ZZZ each independently represent one motif of three identical modifications on three consecutive nucleotides;
wherein the dsRNA agents have one or more lipophilic monomers containing one or more lipophilic moieties conjugated to one or more positions on at least one strand; and
wherein the antisense strand of the dsRNA comprises two blocks of one, two or three phosphorothioate internucleotide linkages separated by 1, 2, 3, 4, 5, 6, 7, 8, 9, 10, 11, 12, 13, 14, 15, 16, 17, or 18 phosphate internucleotide linkages.

Various publications described multimeric siRNA and can all be used with the iRNA of the invention. Such publications include WO2007/091269, U.S. Pat. No. 7,858,769, WO2010/141511, WO2007/117686, WO2009/014887 and WO2011/031520, which are hereby incorporated by reference in their entirety.

In some embodiments, the antisense strand is 100% complementary to a target RNA to hybridize thereto and inhibits its expression through RNA interference. In another embodiment, the antisense strand is at least 95%, at least 90%, at least 85%, at least 80%, at least 75%, at least 70%, at least 65%, at least 60%, at least 55%, or at least 50% complementary to a target RNA.

### Nucleic Acid Modifications

In some embodiments, the oligonucleotide comprises at least one nucleic acid modification described herein. For example, at least one modification selected from the group consisting of modified internucleoside linkage, modified nucleobase, modified sugar, and any combinations thereof. Without limitations, such a modification can be present anywhere in the oligonucleotide. For example, the modification can be present in one of the RNA molecules.

### Nucleic Acid Modifications (Nucleobases)

The naturally occurring base portion of a nucleoside is typically a heterocyclic base. The two most common classes of such heterocyclic bases are the purines and the pyrimidines. For those nucleosides that include a pentofuranosyl sugar, a phosphate group can be linked to the 2′, 3′ or 5′ hydroxyl moiety of the sugar. In forming oligonucleotides, those phosphate groups covalently link adjacent nucleosides to one another to form a linear polymeric compound. Within oligonucleotides, the phosphate groups are commonly referred to as forming the internucleoside backbone of the oligonucleotide. The naturally occurring linkage or backbone of RNA and of DNA is a 3′ to 5′ phosphodiester linkage.

In addition to “unmodified” or “natural” nucleobases such as the purine nucleobases adenine (A) and guanine (G), and the pyrimidine nucleobases thymine (T), cytosine (C) and uracil (U), many modified nucleobases or nucleobase mimetics known to those skilled in the art are amenable with the oligonucleotides described herein. The unmodified or natural nucleobases can be modified or replaced to provide iRNAs having improved properties. For example, nuclease resistant oligonucleotides can be prepared with these bases or with synthetic and natural nucleobases (e.g., inosine, xanthine, hypoxanthine, nubularine, isoguanisine, or tubercidine) and any one of the oligomer modifications described herein. Alternatively, substituted or modified analogs of any of the above bases and “universal bases” can be employed. When a natural base is replaced by a non-natural and/or universal base, the nucleotide is said to comprise a modified nucleobase and/or a nucleobase modification herein. Modified nucleobase and/or nucleobase modifications also include natural, non-natural and universal bases, which comprise conjugated moieties, e.g. a ligand described herein. Preferred conjugate moieties for conjugation with nucleobases include cationic amino groups which can be conjugated to the nucleobase via an appropriate alkyl, alkenyl or a linker with an amide linkage.

An oligonucleotide described herein can also include nucleobase (often referred to in the art simply as “base”) modifications or substitutions. As used herein, “unmodified” or “natural” nucleobases include the purine bases adenine (A) and guanine (G), and the pyrimidine bases thymine (T), cytosine (C) and uracil (U). Exemplary modified nucleobases include, but are not limited to, other synthetic and natural nucleobases such as inosine, xanthine, hypoxanthine, nubularine, isoguanisine, tubercidine, 2-(halo)adenine, 2-(alkyl)adenine, 2-(propyl)adenine, 2-(amino)adenine, 2-(aminoalkyl)adenine, 2-(aminopropyl)adenine, 2-(methylthio)-N⁶-(isopentenyl)adenine, 6-(alkyl)adenine, 6-(methyl)adenine, 7-(deaza)adenine, 8-(alkenyl)adenine, 8-(alkyl)adenine, 8-(alkynyl)adenine, 8-(amino)adenine, 8-(halo)adenine, 8-(hydroxyl)adenine, 8-(thioalkyl)adenine, 8-(thiol)adenine, N⁶-(isopentyl)adenine, N⁶-(methyl)adenine, N⁶, N⁶-(dimethyl)adenine, 2-(alkyl)guanine,2-(propyl)guanine, 6-(alkyl)guanine, 6-(methyl)guanine, 7-(alkyl)guanine, 7-(methyl)guanine, 7-(deaza)guanine, 8-(alkyl)guanine, 8-(alkenyl)guanine, 8-(alkynyl)guanine, 8-(amino)guanine, 8-(halo)guanine, 8-(hydroxyl)guanine, 8-(thioalkyl)guanine, 8-(thiol)guanine, N-(methyl)guanine, 2-(thio)cytosine, 3-(deaza)-5-(aza)cytosine, 3-(alkyl)cytosine, 3-(methyl)cytosine, 5-(alkyl)cytosine, 5-(alkynyl)cytosine, 5-(halo)cytosine, 5-(methyl)cytosine, 5-(propynyl)cytosine, 5-(propynyl)cytosine, 5-(trifluoromethyl)cytosine, 6-(azo)cytosine, N⁴-(acetyl)cytosine, 3-(3-amino-3-carboxypropyl)uracil, 2-(thio)uracil, 5-(methyl)-2-(thio)uracil, 5-(methylaminomethyl)-2-(thio)uracil, 4-(thio)uracil, 5-(methyl)-4-(thio)uracil, 5-(methylaminomethyl)-4-(thio)uracil, 5-(methyl)-2,4-(dithio)uracil, 5-(methylaminomethyl)-2,4-(dithio)uracil, 5-(2-aminopropyl)uracil, 5-(alkyl)uracil, 5-(alkynyl)uracil, 5-(allylamino)uracil, 5-(aminoallyl)uracil, 5-(aminoalkyl)uracil, 5-(guanidiniumalkyl)uracil, 5-(1,3-diazole-1-alkyl)uracil, 5-(cyanoalkyl)uracil, 5-(dialkylaminoalkyl)uracil, 5-(dimethylaminoalkyl)uracil, 5-(halo)uracil, 5-(methoxy)uracil, uracil-5-oxyacetic acid, 5-(methoxycarbonylmethyl)-2-(thio)uracil, 5-(methoxycarbonyl-methyl)uracil, 5-(propynyl)uracil, 5-(propynyl)uracil, 5-(trifluoromethyl)uracil, 6-(azo)uracil, dihydrouracil, N³-(methyl)uracil, 5-uracil (i.e., pseudouracil), 2-(thio)pseudouracil,4-(thio)pseudouracil,2,4-(dithio)psuedouracil,5-(alkyl)pseudouracil, 5-(methyl)pseudouracil, 5-(alkyl)-2-(thio)pseudouracil, 5-(methyl)-2-(thio)pseudouracil, 5-(alkyl)-4-(thio)pseudouracil, 5-(methyl)-4-(thio)pseudouracil, 5-(alkyl)-2,4-(dithio)pseudouracil, 5-(methyl)-2,4-(dithio)pseudouracil, 1-substituted pseudouracil, 1-substituted 2(thio)-pseudouracil, 1-substituted 4-(thio)pseudouracil, 1-substituted 2,4-(dithio)pseudouracil, 1-(aminocarbonylethylenyl)-pseudouracil, 1-(aminocarbonylethylenyl)-2(thio)-pseudouracil, 1-(aminocarbonylethylenyl)-4-(thio)pseudouracil, 1-(aminocarbonylethylenyl)-2,4-(dithio)pseudouracil, 1-(aminoalkylaminocarbonylethylenyl)-pseudouracil, 1-(aminoalkylamino-carbonylethylenyl)-2(thio)-pseudouracil, 1-(aminoalkylaminocarbonylethylenyl)-4-(thio)pseudouracil, 1-(aminoalkylaminocarbonylethylenyl)-2,4-(dithio)pseudouracil, 1,3-(diaza)-2-(oxo)-phenoxazin-1-yl, 1-(aza)-2-(thio)-3-(aza)-phenoxazin-1-yl, 1,3-(diaza)-2-(oxo)-phenthiazin-1-yl, 1-(aza)-2-(thio)-3-(aza)-phenthiazin-1-yl, 7-substituted 1,3-(diaza)-2-(oxo)-phenoxazin-1-yl, 7-substituted 1-(aza)-2-(thio)-3-(aza)-phenoxazin-1-yl, 7-substituted 1,3-(diaza)-2-(oxo)-phenthiazin-1-yl, 7-substituted 1-(aza)-2-(thio)-3-(aza)-phenthiazin-1-yl, 7-(aminoalkylhydroxy)-1,3-(diaza)-2-(oxo)-phenoxazin-1-yl, 7-(aminoalkylhydroxy)-1-(aza)-2-(thio)-3-(aza)-phenoxazin-1-yl, 7-(aminoalkylhydroxy)-1,3-(diaza)-2-(oxo)-phenthiazin-1-yl, 7-(aminoalkylhydroxy)-1-(aza)-2-(thio)-3-(aza)-phenthiazin-1-yl, 7-(guanidiniumalkylhydroxy)-1,3-(diaza)-2-(oxo)-phenoxazin-1-yl, 7-(guanidiniumalkylhydroxy)-1-(aza)-2-(thio)-3-(aza)-phenoxazin-1-yl, 7-(guanidiniumalkyl-hydroxy)-1,3-(diaza)-2-(oxo)-phenthiazin-1-yl, 7-(guanidiniumalkylhydroxy)-1-(aza)-2-(thio)-3-(aza)-phenthiazin-1-yl, 1,3,5-(triaza)-2,6-(dioxa)-naphthalene, inosine, xanthine, hypoxanthine, nubularine, tubercidine, isoguanisine, inosinyl, 2-aza-inosinyl, 7-deaza-inosinyl, nitroimidazolyl, nitropyrazolyl, nitrobenzimidazolyl, nitroindazolyl, aminoindolyl, pyrrolopyrimidinyl, 3-(methyl)isocarbostyrilyl, 5-(methyl)isocarbostyrilyl, 3-(methyl)-7-(propynyl)isocarbostyrilyl, 7-(aza)indolyl, 6-(methyl)-7-(aza)indolyl, imidizopyridinyl, 9-(methyl)-imidizopyridinyl, pyrrolopyrizinyl, isocarbostyrilyl, 7-(propynyl)isocarbostyrilyl, propynyl-7-(aza)indolyl, 2,4,5-(trimethyl)phenyl, 4-(methyl)indolyl, 4,6-(dimethyl)indolyl, phenyl, napthalenyl, anthracenyl, phenanthracenyl, pyrenyl, stilbenyl, tetracenyl, pentacenyl, difluorotolyl, 4-(fluoro)-6-(methyl)benzimidazole, 4-(methyl)benzimidazole, 6-(azo)thymine, 2-pyridinone, 5-nitroindole, 3-nitropyrrole, 6-(aza)pyrimidine, 2-(amino)purine, 2,6-(diamino)purine, 5-substituted pyrimidines, N²-substituted purines, N⁶-substituted purines, O⁶-substituted purines, substituted 1,2,4-triazoles, pyrrolo-pyrimidin-2-on-3-yl, 6-phenyl-pyrrolo-pyrimidin-2-on-3-yl, para-substituted-6-phenyl-pyrrolo-pyrimidin-2-on-3-yl, ortho-substituted-6-phenyl-pyrrolo-pyrimidin-2-on-3-yl, bis-ortho-substituted-6-phenyl-pyrrolo-pyrimidin-2-on-3-yl, para-(aminoalkylhydroxy)-6-phenyl-pyrrolo-pyrimidin-2-on-3-yl, ortho-(aminoalkylhydroxy)-6-phenyl-pyrrolo-pyrimidin-2-on-3-yl, bis-ortho--(aminoalkylhydroxy)-6-phenyl-pyrrolo-pyrimidin-2-on-3-yl, pyridopyrimidin-3-yl, 2-oxo-7-amino-pyridopyrimidin-3-yl, 2-oxo-pyridopyrimidine-3-yl, or any O-alkylated or N-alkylated derivatives thereof. Alternatively, substituted or modified analogs of any of the above bases and “universal bases” can be employed.

As used herein, a universal nucleobase is any nucleobase that can base pair with all of the four naturally occurring nucleobases without substantially affecting the melting behavior, recognition by intracellular enzymes or activity of the iRNA duplex. Some exemplary universal nucleobases include, but are not limited to, 2,4-difluorotoluene, nitropyrrolyl, nitroindolyl, 8-aza-7-deazaadenine, 4-fluoro-6-methylbenzimidazle, 4-methylbenzimidazle, 3-methyl isocarbostyrilyl, 5-methyl isocarbostyrilyl, 3-methyl-7-propynyl isocarbostyrilyl, 7-azaindolyl, 6-methyl-7-azaindolyl, imidizopyridinyl, 9-methyl-imidizopyridinyl, pyrrolopyrizinyl, isocarbostyrilyl, 7-propynyl isocarbostyrilyl, propynyl-7-azaindolyl, 2,4,5-trimethylphenyl, 4-methylinolyl, 4,6-dimethylindolyl, phenyl, napthalenyl, anthracenyl, phenanthracenyl, pyrenyl, stilbenyl, tetracenyl, pentacenyl, and structural derivatives thereof (see for example, Loakes, 2001, Nucleic Acids Research, 29, 2437-2447).

Further nucleobases include those disclosed in U.S. Pat. No. 3,687,808; those disclosed in International Application No. PCT/US09/038425, filed Mar. 26, 2009; those disclosed in the Concise Encyclopedia Of Polymer Science And Engineering, pages 858-859, Kroschwitz, J. I., ed. John Wiley & Sons, 1990; those disclosed by English et al., Angewandte Chemie, International Edition, 1991, 30, 613; those disclosed in Modified Nucleosides in Biochemistry, Biotechnology and Medicine, Herdewijin, P.Ed. Wiley-VCH, 2008; and those disclosed by Sanghvi, Y. S., Chapter 15, dsRNA Research and Applications, pages 289-302, Crooke, S. T. and Lebleu, B., Eds., CRC Press, 1993. Contents of all of the above are herein incorporated by reference.

In certain embodiments, a modified nucleobase is a nucleobase that is fairly similar in structure to the parent nucleobase, such as for example a 7-deaza purine, a 5-methyl cytosine, or a G-clamp. In certain embodiments, nucleobase mimetic includes more complicated structures, such as for example a tricyclic phenoxazine nucleobase mimetic. Methods for preparation of the above noted modified nucleobases are well known to those skilled in the art.

### Nucleic Acid Modifications (Sugar)

As discussed above, described herein is the modification of 2′-position of a nucleoside of an oligonucleotide by a biocleavable linking group as a temporary protecting group at the 2′-position of the nucleoside. The sugar moieties of the remaining nucleosides/nucleotides can also be modified using the methods described below.

The oligonucleotide provided herein can comprise one or more (e.g., 1, 2, 3, 4, 5, 6, 7, 8, 9, 10, 11, 12, 13, 14, 15 or more) monomer, including a nucleoside or nucleotide, having a modified sugar moiety. For example, the furanosyl sugar ring of a nucleoside can be modified in a number of ways including, but not limited to, addition of a substituent group, bridging of two non-geminal ring atoms to form a locked nucleic acid or bicyclic nucleic acid. In certain embodiments, oligonucleotides comprise one or more (e.g., 1, 2, 3, 4, 5, 6, 7, 8, 9, 10, 11, 12, 13, 14, 15 or more) monomers that are LNA.

In some embodiments of a locked nucleic acid, the 2′ position of furnaosyl is connected to the 4′ position by a linker selected independently from —[C(R1)(R2)]ₙ—, —[C(R1)(R2)]ₙ—O—, —[C(R1)(R2)]ₙ—N(R1)—, —[C(R1)(R2)]ₙ—N(R1)—O—, [C(R1R2)]ₙ—O—N(R1)—, —C(R1)═C(R2)—O—, —C(R1)═N—, —C(R1)═N—O—, C(═NR1)—, C(═NR1)—O—, C(═O)—, C(═O)O—, C(═S)—, C(═S)O—, C(═S)S—, O, Si(R1)₂—, S(═O)X— and N(R1)—;
wherein:
x is 0, 1, or 2;
n is 1, 2, 3, or 4;
each R¹ and R² is, independently, H, a protecting group, hydroxyl, C1-C12 alkyl, substituted C1-C12 alkyl, C2-C12 alkenyl, substituted C2-C12 alkenyl, C2-C12 alkynyl, substituted C2-C12 alkynyl, C5-C20 aryl, substituted C5-C20 aryl, heterocycle radical, substituted heterocycle radical, heteroaryl, substituted heteroaryl, C5-C7 alicyclic radical, substituted C5-C7 alicyclic radical, halogen, OJ1, NJ1J2, SJ1, N3, COOJ1, acyl (C(═O) H), substituted acyl, CN, sulfonyl (S(═O)2-J1), or sulfoxyl (S(═O)—J1); and
each J1 and J2 is, independently, H, C1-C12 alkyl, substituted C1-C12 alkyl, C2-C12 alkenyl, substituted C2-C12 alkenyl, C2-C12 alkynyl, substituted C2-C12 alkynyl, C5-C20 aryl, substituted C5-C20 aryl, acyl (C(═O)—H), substituted acyl, a heterocycle radical, a substituted heterocycle radical, C1-C12 aminoalkyl, substituted C1-C12 aminoalkyl or a protecting group.

In some embodiments, each of the linkers of the LNA compounds is, independently, [C(R1)(R2)]ₙ—, [C(R1)(R2)]ₙ—O—, C(R1R2)—N(R1)—O or C(R1R2)—O—N(R1)—. In another embodiment, each of said linkers is, independently, 4′-CH₂-2′, 4′—(CH₂)₂-2′, 4′—(CH₂)₃-2′, 4′-CH₂—O—2′, 4′—(CH₂)₂—O—2′, 4′-CH₂—O—N(R1)-2′ and 4′-CH₂—N(R1)—O—2′-wherein each R1 is, independently, H, a protecting group or C1-C12 alkyl.

Certain LNA's have been prepared and disclosed in the patent literature as well as in scientific literature (Singh et al., Chem. Commun., 1998, 4, 455-456; Koshkin et al., Tetrahedron, 1998, 54, 3607-3630; Wahlestedt et al., Proc. Natl. Acad. Sci. U.S.A., 2000, 97, 5633-5638; Kumar et al., Bioorg. Med. Chem. Lett., 1998, 8, 2219-2222; WO 94/14226; WO 2005/021570; Singh et al., J. Org. Chem., 1998, 63, 10035-10039; Examples of issued US patents and published applications that disclose LNA s include, for example, U.S. Pat. Nos. 7,053,207; 6,268,490; 6,770,748; 6,794,499; 7,034,133; and 6,525,191; and U.S. Pre-Grant Publication Nos. 2004-0171570; 2004-0219565; 2004-0014959; 2003-0207841; 2004-0143114; and 20030082807.

Also provided herein are LNAs in which the 2′-hydroxyl group of the ribosyl sugar ring is linked to the 4′ carbon atom of the sugar ring thereby forming a methyleneoxy (4′-CH₂—O—2′) linkage to form the bicyclic sugar moiety (reviewed in Elayadi et al., Curr. Opinion Invens. Drugs, 2001, 2, 558-561; Braasch et al., Chem. Biol., 2001, 8 1-7; and Orum et al., Curr. Opinion Mol. Ther., 2001, 3, 239-243; see also U.S. Pat. Nos. 6,268,490 and 6,670,461). The linkage can be a methylene (—CH₂—) group bridging the 2′ oxygen atom and the 4′ carbon atom, for which the term methyleneoxy (4′-CH₂—O—2′) LNA is used for the bicyclic moiety; in the case of an ethylene group in this position, the term ethyleneoxy (4′-CH₂CH₂—O-2′) LNA is used (Singh et al., Chem. Commun., 1998, 4, 455-456: Morita et al., Bioorganic Medicinal Chemistry, 2003, 11, 2211-2226). Methyleneoxy (4′-CH₂—O—2′) LNA and other bicyclic sugar analogs display very high duplex thermal stabilities with complementary DNA and RNA (Tm=+3 to +100° C.), stability towards 3′-exonucleolytic degradation and good solubility properties. Potent and nontoxic antisense oligonucleotides comprising BNAs have been described (Wahlestedt et al., Proc. Natl. Acad. Sci. U.S.A., 2000, 97, 5633-5638).

An isomer of methyleneoxy (4′-CH₂—O—2′) LNA that has also been discussed is alpha-L-methyleneoxy (4′-CH₂—O—2′) LNA which has been shown to have superior stability against a 3′-exonuclease. The alpha-L-methyleneoxy (4′-CH₂—O—2′) LNA's were incorporated into antisense gapmers and chimeras that showed potent antisense activity (Frieden et al., Nucleic Acids Research, 2003, 21, 6365-6372).

The synthesis and preparation of the methyleneoxy (4′-CH₂—O—2′) LNA monomers adenine, cytosine, guanine, 5-methyl-cytosine, thymine and uracil, along with their oligomerization, and nucleic acid recognition properties have been described (Koshkin et al., Tetrahedron, 1998, 54, 3607-3630). BNAs and preparation thereof are also described in WO 98/39352 and WO 99/14226.

Analogs of methyleneoxy (4′-CH₂—O—2′) LNA, phosphorothioate-methyleneoxy (4′-CH₂—O—2′) LNA and 2′-thio-LNAs, have also been prepared (Kumar et al., Bioorg. Med. Chem. Lett., 1998, 8, 2219-2222). Preparation of locked nucleoside analogs comprising oligodeoxyribonucleotide duplexes as substrates for nucleic acid polymerases has also been described (Wengel et al., WO 99/14226). Furthermore, synthesis of 2′-amino-LNA, a novel comformationally restricted high-affinity oligonucleotide analog has been described in the art (Singh et al., J. Org. Chem., 1998, 63, 10035-10039). In addition, 2′-Amino- and 2′-methylamino-LNA's have been prepared and the thermal stability of their duplexes with complementary RNA and DNA strands has been previously reported.

Modified sugar moieties are well known and can be used to alter, typically increase, the affinity of the antisense compound for its target and/or increase nuclease resistance. A representative list of preferred modified sugars includes but is not limited to bicyclic modified sugars, including methyleneoxy (4′-CH₂—O—2′) LNA and ethyleneoxy (4′—(CH₂)₂—O—2′ bridge) ENA; substituted sugars, especially 2′-substituted sugars having a 2′-F, 2′-OCH₃ or a 2′-O(CH₂)₂—OCH₃ substituent group; and 4′-thio modified sugars. Sugars can also be replaced with sugar mimetic groups among others. Methods for the preparations of modified sugars are well known to those skilled in the art. Some representative patents and publications that teach the preparation of such modified sugars include, but are not limited to, U.S. Pat. Nos. 4,981,957; 5,118,800; 5,319,080; 5,359,044; 5,393,878; 5,446,137; 5,466,786; 5,514,785; 5,519,134; 5,567,811; 5,576,427; 5,591,722; 5,597,909; 5,610,300; 5,627,053; 5,639,873; 5,646,265; 5,658,873; 5,670,633; 5,792,747; 5,700,920; 6,531,584; and 6,600,032; and WO 2005/121371.

Examples of “oxy”-2′ hydroxyl group modifications include alkoxy or aryloxy (OR, e.g., R═H, alkyl, cycloalkyl, aryl, aralkyl, heteroaryl or sugar); polyethyleneglycols (PEG), O(CH₂CH₂O)CH₂CH₂OR, n═1-50; “locked” nucleic acids (LNA) in which the furanose portion of the nucleoside includes a bridge connecting two carbon atoms on the furanose ring, thereby forming a bicyclic ring system; O-AMINE or O—(CH₂)ₙAMINE (n═1-10, AMINE═NH₂; alkylamino, dialkylamino, heterocyclyl, arylamino, diaryl amino, heteroaryl amino, diheteroaryl amino, ethylene diamine or polyamino); and 0-CH₂CH₂(NCH₂CH₂NMe₂)₂.

“Deoxy” modifications include hydrogen (i.e. deoxyribose sugars, which are of particular relevance to the single-strand overhangs); halo (e.g., fluoro); amino (e.g. NH₂; alkylamino, dialkylamino, heterocyclyl, arylamino, diaryl amino, heteroaryl amino, diheteroaryl amino, or amino acid); NH(CH₂CH₂NH),CH₂CH₂-AMINE (AMINE═NH₂; alkylamino, dialkylamino, heterocyclyl, arylamino, diaryl amino, heteroaryl amino, or diheteroaryl amino); —NHC(O)R (R═alkyl, cycloalkyl, aryl, aralkyl, heteroaryl or sugar); cyano; mercapto; alkyl-thio-alkyl; thioalkoxy; thioalkyl; alkyl; cycloalkyl; aryl; alkenyl and alkynyl, which can be optionally substituted with e.g., an amino functionality.

Other suitable 2′-modifications, e.g., modified MOE, are described in U.S. Patent Application Publication No. 20130130378, contents of which are herein incorporated by reference.

A modification at the 2′ position can be present in the arabinose configuration The term “arabinose configuration” refers to the placement of a substituent on the C2′ of ribose in the same configuration as the 2′-OH is in the arabinose.

The sugar can comprise two different modifications at the same carbon in the sugar, e.g., gem modification. The sugar group can also contain one or more carbons that possess the opposite stereochemical configuration than that of the corresponding carbon in ribose. Thus, an oligonucleotide can include one or more monomers containing e.g., arabinose, as the sugar. The monomer can have an alpha linkage at the 1′ position on the sugar, e.g., alpha-nucleosides. The monomer can also have the opposite configuration at the 4′-position, e.g., C5′ and H4′ or substituents replacing them are interchanged with each other. When the C5′ and H4′ or substituents replacing them are interchanged with each other, the sugar is said to be modified at the 4′ position.

The oligonucleotide disclosed herein can also include abasic sugars, i.e., a sugar which lack a nucleobase at C-1′ or has other chemical groups in place of a nucleobase at C1′. See for example U.S. Pat. No. 5,998,203, content of which is herein incorporated in its entirety. These abasic sugars can also be further containing modifications at one or more of the constituent sugar atoms. The oligonucleotide can also contain one or more sugars that are the L isomer, e.g. L-nucleosides. Modification to the sugar group can also include replacement of the 4′-O with a sulfur, optionally substituted nitrogen or CH₂ group. In some embodiments, linkage between C1′ and nucleobase is in a configuration.

Sugar modifications can also include a “acyclic nucleotide,” which refers to any nucleotide having an acyclic ribose sugar, e.g., wherein a C—C bonds between ribose carbons (e.g., C1′-C2′, C2′-C3′, C3′-C4′, C4′-04′, C1′-04′) is absent and/or at least one of ribose carbons or oxygen (e.g., C1′, C2′, C3′, C4′ or 04′) are independently or in combination absent from the nucleotide. In some embodiments, acyclic nucleotide is

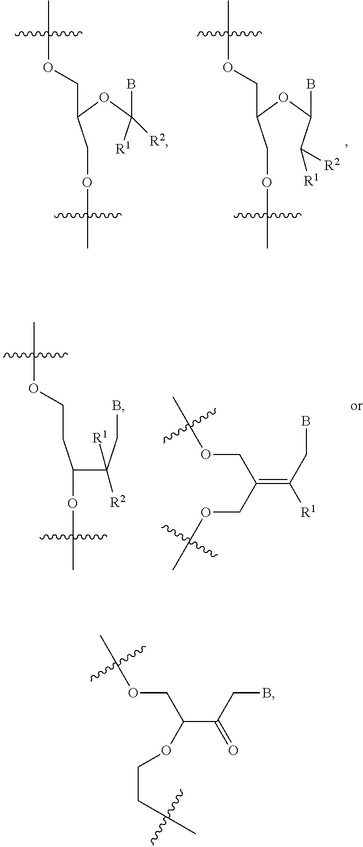

wherein B is a modified or unmodified nucleobase, R₁ and R₂ independently are H, halogen, OR₃, or alkyl; and R₃ is H, alkyl, cycloalkyl, aryl, aralkyl, heteroaryl or sugar).

In some embodiments, sugar modifications are selected from the group consisting of 2′-H, 2′-O-Me (2′-O-methyl), 2′-O-MOE (2′-O-methoxyethyl), 2′-F, 2′-O—[2-(methylamino)-2-oxoethyl] (2′-O-NMA), 2′—S-methyl, 2′-O—CH₂-(4′-C) (LNA), 2′-O—CH₂CH₂-(4′-C) (ENA), 2′-O-aminopropyl (2′-O-AP), 2′-O-dimethylaminoethyl (2′-O-DMAOE), 2′-O-dimethylaminopropyl (2′-O-DMAP), 2′-O-dimethylaminoethyloxyethyl (2′-O-DMAEOE) and gem 2′-OMe/2′F with 2′-O-Me in the arabinose configuration.

It is to be understood that when a particular nucleotide is linked through its 2′-position to the next nucleotide, the sugar modifications described herein can be placed at the 3′-position of the sugar for that particular nucleotide, e.g., the nucleotide that is linked through its 2′-position. A modification at the 3′ position can be present in the xylose configuration The term “xylose configuration” refers to the placement of a substituent on the C3′ of ribose in the same configuration as the 3′-OH is in the xylose sugar.

The hydrogen attached to C4′ and/or C1′ can be replaced by a straight- or branched-optionally substituted alkyl, optionally substituted alkenyl, optionally substituted alkynyl, wherein backbone of the alkyl, alkenyl and alkynyl can contain one or more of O, S, S(O), SO₂, N(R′), C(O), N(R′)C(O)O, OC(O)N(R′), CH(Z′), phosphorous containing linkage, optionally substituted aryl, optionally substituted heteroaryl, optionally substituted heterocyclic or optionally substituted cycloalkyl, where R′ is hydrogen, acyl or optionally substituted aliphatic, Z′ is selected from the group consisting of OR₁₁, COR₁₁, CO₂R₁₁,

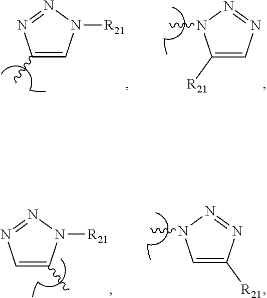

NR₂₁R₃₁, CONR₂₁R₃₁, CON(H)NR₂₁R₃₁, ONR₂₁R₃₁, CON(H)N═CR₄₁R⁵¹, N(R₂₁)C(═NR₃₁)NR₂₁R₃₁, N(R₂₁)C(O)NR₂₁R₃₁, N(R₂₁)C(S)NR₂₁R₃₁, OC(O)NR₂₁R₃₁, SC(O)NR₂₁R₃₁, N(R₂₁)C(S)OR₁₁, N(R₂₁)C(O)OR₁₁, N(R₂₁)C(O)SR₁₁, N(R₂₁)N═CR₄₁R₅₁, ON═CR₄₁R⁵¹, SO₂R₁₁, SOR₁₁, SR₁₁, and substituted or unsubstituted heterocyclic; R₂₁ and R₃₁ for each occurrence are independently hydrogen, acyl, unsubstituted or substituted aliphatic, aryl, heteroaryl, heterocyclic, OR₁₁, COR₁₁, CO₂R₁₁, or NR₁₁R₁₁′; or R₂₁ and R₃₁, taken together with the atoms to which they are attached, form a heterocyclic ring; R₄₁ and R₅₁ for each occurrence are independently hydrogen, acyl, unsubstituted or substituted aliphatic, aryl, heteroaryl, heterocyclic, OR₁₁, COR₁₁, or CO₂R₁₁, or NR₁₁R₁₁′; and R₁₁ and R₁₁′ are independently hydrogen, aliphatic, substituted aliphatic, aryl, heteroaryl, or heterocyclic. In some embodiments, the hydrogen attached to the C4′ of the 5′ terminal nucleotide is replaced.

In some embodiments, C4′ and C5′ together form an optionally substituted heterocyclic, preferably comprising at least one —PX(Y)—, wherein X is H, OH, OM, SH, optionally substituted alkyl, optionally substituted alkoxy, optionally substituted alkylthio, optionally substituted alkylamino or optionally substituted dialkylamino, where M is independently for each occurrence an alkali metal or transition metal with an overall charge of +1; and Y is O, S, or NR′, where R′ is hydrogen, optionally substituted aliphatic. Preferably this modification is at the 5′ terminal of the iRNA.

In certain embodiments, the oligonucleotide comprises at least two regions of at least two contiguous monomers of the above formula. In certain embodiments, the oligonucleotide comprises a gapped motif. In certain embodiments, the oligonucleotide comprises at least one region of from about 8 to about 14 contiguous β-D-2′-deoxyribofuranosyl nucleosides. In certain embodiments, the oligonucleotide comprises at least one region of from about 9 to about 12 contiguous β-D-2′-deoxyribofuranosyl nucleosides.

In certain embodiments, the oligonucleotide comprises at least one (e.g., 1, 2, 3, 4, 5, 6, 7, 8, 9, 10, 11, 12, 13, 14, 15 or more) comprises at least one (S)-cEt monomer of the formula:

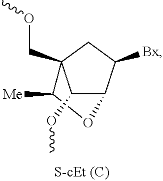

wherein Bx is heterocyclic base moiety.

In certain embodiments, monomers include sugar mimetics. In certain such embodiments, a mimetic is used in place of the sugar or sugar-internucleoside linkage combination, and the nucleobase is maintained for hybridization to a selected target. Representative examples of a sugar mimetics include, but are not limited to, cyclohexenyl or morpholino. Representative examples of a mimetic for a sugar-internucleoside linkage combination include, but are not limited to, peptide nucleic acids (PNA) and morpholino groups linked by uncharged achiral linkages. In some instances a mimetic is used in place of the nucleobase. Representative nucleobase mimetics are well known in the art and include, but are not limited to, tricyclic phenoxazine analogs and universal bases (Berger et al., Nuc Acid Res. 2000, 28:2911-14, incorporated herein by reference). Methods of synthesis of sugar, nucleoside and nucleobase mimetics are well known to those skilled in the art.

### Nucleic Acid Modifications (Intersugar Linkage)

Described herein are linking groups that link monomers (including, but not limited to, modified and unmodified nucleosides and nucleotides) together, thereby forming an oligonucleotide. Such linking groups are also referred to as intersugar linkage. The two main classes of linking groups are defined by the presence or absence of a phosphorus atom. Representative phosphorus containing linkages include, but are not limited to, phosphodiesters (P═O), phosphotriesters, methylphosphonates, phosphoramidate, and phosphorothioates (P═S). Representative non-phosphorus containing linking groups include, but are not limited to, methylenemethylimino (CH₂—N(CH₃)—O CH2—), thiodiester (—O—C(O)—S—), thionocarbamate (—O—C(O)(NH)—S—); siloxane (—O—Si(H)₂—O—); and N,N′-dimethylhydrazine (CH₂—N(CH₃)—N(CH₃)—).

Modified linkages, compared to natural phosphodiester linkages, can be used to alter, typically increase, nuclease resistance of the oligonucleotides. In certain embodiments, linkages having a chiral atom can be prepared as racemic mixtures, as separate enantiomers. Representative chiral linkages include, but are not limited to, alkylphosphonates and phosphorothioates. Methods of preparation of phosphorous-containing and non-phosphorous-containing linkages are well known to those skilled in the art.

The phosphate group in the linking group can be modified by replacing one of the oxygens with a different substituent. One result of this modification can be increased resistance of the oligonucleotide to nucleolytic breakdown. Examples of modified phosphate groups include phosphorothioate, phosphoroselenates, borano phosphates, borano phosphate esters, hydrogen phosphonates, phosphoroamidates, alkyl or aryl phosphonates and phosphotriesters. In some embodiments, one of the non-bridging phosphate oxygen atoms in the linkage can be replaced by any of the following: S, Se, BR₃ (R is hydrogen, alkyl, aryl), C (i.e. an alkyl group, an aryl group, etc. . . . ), H, NR₂ (R is hydrogen, optionally substituted alkyl, aryl), or (R is optionally substituted alkyl or aryl). The phosphorous atom in an unmodified phosphate group is achiral. However, replacement of one of the non-bridging oxygens with one of the above atoms or groups of atoms renders the phosphorous atom chiral; in other words a phosphorous atom in a phosphate group modified in this way is a stereogenic center. The stereogenic phosphorous atom can possess either the “R” configuration (herein Rp) or the “S” configuration (herein Sp).

Phosphorodithioates have both non-bridging oxygens replaced by sulfur. The phosphorus center in the phosphorodithioates is achiral which precludes the formation of oligonucleotides diastereomers. Thus, while not wishing to be bound by theory, modifications to both non-bridging oxygens, which eliminate the chiral center, e.g. phosphorodithioate formation, can be desirable in that they cannot produce diastereomer mixtures. Thus, the non-bridging oxygens can be independently any one of O, S, Se, B, C, H, N, or OR (R is alkyl or aryl).

The phosphate linker can also be modified by replacement of bridging oxygen, (i.e. oxygen that links the phosphate to the sugar of the monomer), with nitrogen (bridged phosphoroamidates), sulfur (bridged phosphorothioates) and carbon (bridged methylenephosphonates). The replacement can occur at the either one of the linking oxygens or at both linking oxygens. When the bridging oxygen is the 3′-oxygen of a nucleoside, replacement with carbon is preferred. When the bridging oxygen is the 5′-oxygen of a nucleoside, replacement with nitrogen is preferred.

Modified phosphate linkages where at least one of the oxygen linked to the phosphate has been replaced or the phosphate group has been replaced by a non-phosphorous group, are also referred to as “non-phosphodiester intersugar linkage” or “non-phosphodiester linker.”

In certain embodiments, the phosphate group can be replaced by non-phosphorus containing connectors, e.g. dephospho linkers. Dephospho linkers are also referred to as non-phosphodiester linkers herein. While not wishing to be bound by theory, it is believed that since the charged phosphodiester group is the reaction center in nucleolytic degradation, its replacement with neutral structural mimics should impart enhanced nuclease stability. Again, while not wishing to be bound by theory, it can be desirable, in some embodiment, to introduce alterations in which the charged phosphate group is replaced by a neutral moiety.

Examples of moieties which can replace the phosphate group include, but are not limited to, amides (for example amide-3 (3′-CH₂—C(═O)—N(H)-5′) and amide-4 (3′-CH₂—N(H)—C(═O)-5′)), hydroxylamino, siloxane (dialkylsiloxane), carboxamide, carbonate, carboxymethyl, carbamate, carboxylate ester, thioether, ethylene oxide linker, sulfide, sulfonate, sulfonamide, sulfonate ester, thioformacetal (3′—S—CH₂—O—5′), formacetal (3′-O—CH₂—O—5′), oxime, methyleneimino, methykenecarbonylamino, methylenemethylimino (MMI, 3′-CH₂—N(CH₃)—O—5′), methylenehydrazo, methylenedimethylhydrazo, methyleneoxymethylimino, ethers (C3′—O—C5′), thioethers (C3′—S—C5′), thioacetamido (C3′—N(H)—C(═O)—CH₂—S—C5′, C3′—O—P(O)—O—SS—C5′, C3′-CH₂—NH—NH—C5′, 3′—NHP(O)(OCH₃)—O—5′ and 3′-NHP(O)(OCH₃)—O—5′ and nonionic linkages containing mixed N, O, S and CH₂ component parts. See for example, Carbohydrate Modifications in Antisense Research; Y. S. Sanghvi and P.D. Cook Eds. ACS Symposium Series 580; Chapters 3 and 4, (pp. 40-65). Preferred embodiments include methylenemethylimino (MMI), methylenecarbonylamino, amides, carbamate and ethylene oxide linker.

One skilled in the art is well aware that in certain instances replacement of a non-bridging oxygen can lead to enhanced cleavage of the intersugar linkage by the neighboring 2′-OH, thus in many instances, a modification of a non-bridging oxygen can necessitate modification of 2′-OH, e.g., a modification that does not participate in cleavage of the neighboring intersugar linkage, e.g., arabinose sugar, 2′-O-alkyl, 2′-F, LNA and ENA.

Preferred non-phosphodiester intersugar linkages include phosphorothioates, phosphorothioates with an at least 1%, 5%, 10%, 20%, 30%, 40%, 50%, 60%, 70%, 80%, 90% 95% or more enantiomeric excess of Sp isomer, phosphorothioates with an at least 1%, 5%, 10%, 20%, 30%, 40%, 50%, 60%, 70%, 80%, 90% 95% or more enantiomeric excess of Rp isomer, phosphorodithioates, phosphotriesters, aminoalkylphosphotriesters, alkyl-phosphonaters (e.g., methyl-phosphonate), selenophosphates, phosphoramidates (e.g., N-alkylphosphoramidate), and boranophosphonates.

In some embodiments, the oligonucleotide further comprises at least one (e.g., 1, 2, 3, 4, 5, 6, 7, 8, 9, 10, 11, 12, 13, 14, 15 or more and up to including all) modified or nonphosphodiester linkages. In some embodiments, the oligonucleotide further comprises at least one (e.g., 1, 2, 3, 4, 5, 6, 7, 8, 9, 10, 11, 12, 13, 14, 15 or more and up to including all) phosphorothioate linkages.

The oligonucleotide can also be constructed wherein the phosphate linker and the sugar are replaced by nuclease resistant nucleoside or nucleotide surrogates. While not wishing to be bound by theory, it is believed that the absence of a repetitively charged backbone diminishes binding to proteins that recognize polyanions (e.g. nucleases). Again, while not wishing to be bound by theory, it can be desirable in some embodiment, to introduce alterations in which the bases are tethered by a neutral surrogate backbone. Examples include the morpholino, cyclobutyl, pyrrolidine, peptide nucleic acid (PNA), aminoethylglycyl PNA (aegPNA) and backbone-extended pyrrolidine PNA (bepPNA) nucleoside surrogates. A preferred surrogate is a PNA surrogate.

The oligonucleotide described herein can contain one or more asymmetric centers and thus give rise to enantiomers, diastereomers, and other stereoisomeric configurations that may be defined, in terms of absolute stereochemistry, as (R) or (S), such as for sugar anomers, or as (D) or (L) such as for amino acids et al. Included in the oligonucleotide are all such possible isomers, as well as their racemic and optically pure forms.

### Nucleic Acid Modifications (Terminal Modifications)

In some embodiments, the oligonucleotide further comprises a phosphate or phosphate mimic at the 5′-end of the antisense strand. In one embodiment, the phosphate mimic is a 5′-vinyl phosphonate (VP).

In some embodiments, the 5′-end of the antisense strand does not contain a 5′-vinyl phosphonate (VP).

Ends of the iRNA agent can be modified. Such modifications can be at one end or both ends. For example, the 3′ and/or 5′ ends of an iRNA can be conjugated to other functional molecular entities such as labeling moieties, e.g., fluorophores (e.g., pyrene, TAMRA, fluorescein, Cy3 or Cy5 dyes) or protecting groups (based e.g., on sulfur, silicon, boron or ester). The functional molecular entities can be attached to the sugar through a phosphate group and/or a linker. The terminal atom of the linker can connect to or replace the linking atom of the phosphate group or the C-3′ or C-5′ O, N, S or C group of the sugar. Alternatively, the linker can connect to or replace the terminal atom of a nucleotide surrogate (e.g., PNAs).

When a linker/phosphate-functional molecular entity-linker/phosphate array is interposed between two strands of a double stranded oligonucleotide, this array can substitute for a hairpin loop in a hairpin-type oligonucleotide.

Terminal modifications useful for modulating activity include modification of the 5′ end of iRNAs with phosphate or phosphate analogs. In certain embodiments, the 5′end of an iRNA is phosphorylated or includes a phosphoryl analog. Exemplary 5′-phosphate modifications include those which are compatible with RISC mediated gene silencing. Modifications at the 5′-terminal end can also be useful in stimulating or inhibiting the immune system of a subject. In some embodiments, the 5′-end of the oligonucleotide comprises the modification

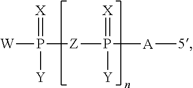

wherein W, X and Y are each independently selected from the group consisting of 0, OR (R is hydrogen, alkyl, aryl), S, Se, BR₃ (R is hydrogen, alkyl, aryl), BH₃⁻, C (i.e. an alkyl group, an aryl group, etc. . . . ), H, NR₂ (R is hydrogen, alkyl, aryl), or OR (R is hydrogen, alkyl or aryl); A and Z are each independently for each occurrence absent, O, S, CH₂, NR (R is hydrogen, alkyl, aryl), or optionally substituted alkylene, wherein backbone of the alkylene can comprise one or more of O, S, SS and NR (R is hydrogen, alkyl, aryl) internally and/or at the end; and n is 0-2. In some embodiments, n is 1 or 2. It is understood that A is replacing the oxygen linked to 5′ carbon of sugar. When n is 0, W and Y together with the P to which they are attached can form an optionally substituted 5-8 membered heterocyclic, wherein W an Y are each independently O, S, NR′ or alkylene. Preferably the heterocyclic is substituted with an aryl or heteroaryl. In some embodiments, one or both hydrogen on C5′ of the 5′-terminal nucleotides are replaced with a halogen, e.g., F.

Exemplary 5′-modifications include, but are not limited to, 5′-monophosphate ((HO)₂(O)P—O—5′); 5′-diphosphate ((HO)₂(O)P—O—P(HO)(O)—O—5′); 5′-triphosphate ((HO)₂(O)P—O—(HO)(O)P—O—P(HO)(O)—O—5′); 5′-monothiophosphate (phosphorothioate; (HO)₂(S)P—O—5′); 5′-monodithiophosphate (phosphorodithioate; (HO)(HS)(S)P—O—5′), 5′-phosphorothiolate ((HO)₂(O)P—S—5′); 5′-alpha-thiotriphosphate; 5′-beta-thiotriphosphate; 5′-gamma-thiotriphosphate; 5′-phosphoramidates ((HO)₂(O)P—NH-5′, (HO)(NH₂)(O)P—O—5′). Other 5′-modification include 5′-alkylphosphonates (R(OH)(O)P—O—5′, R═alkyl, e.g., methyl, ethyl, isopropyl, propyl, etc. . . . ), 5′-alkyletherphosphonates (R(OH)(O)P—O—5′, R═alkylether, e.g., methoxymethyl (CH₂OMe), ethoxymethyl, etc. . . . ); 5′-guanosine cap (7-methylated or non-methylated) (7m-G-O—5′—(HO)(O)P—O—(HO)(O)P—O—P(HO)(O)—O—5′); 5′-adenosine cap (Appp), and any modified or unmodified nucleotide cap structure (N—O—5′—(HO)(O)P—O—(HO)(O)P—O—P(HO)(O)—O—5′). Other exemplary 5′-modifications include where Z is optionally substituted alkyl at least once, e.g., ((HO)₂(X)P—O[—(CH₂)ₐ—O—P(X)(OH)—O]_{b}— 5′, ((HO)₂(X)P—O[—(CH2)ₐ—P(X)(OH)—O]_{b}— 5′, ((HO)₂(X)P—[—(CH2)ₐ—O—P(X)(OH)—O]_{b}— 5′; dialkyl terminal phosphates and phosphate mimics: HO[—(CH₂)ₐ—O—P(X)(OH)—O]_{b}— 5′, H₂N[—(CH₂)ₐ—O—P(X)(OH)—O]_{b}— 5′, H[—(CH₂)ₐ—O—P(X)(OH)—O]_{b}— 5′, Me₂N[—(CH₂)ₐ—O—P(X)(OH)—O]_{b}— 5′, HO[—(CH₂)ₐ—P(X)(OH)—O]_{b}— 5′, H₂N[—(CH₂)ₐ—P(X)(OH)—O]_{b}— 5′, H[—(CH₂)ₐ—P(X)(OH)—O]_{b}— 5′, Me₂N[—(CH₂)ₐ—P(X)(OH)—O]_{b}— 5′, wherein a and b are each independently 1-10. Other embodiments, include replacement of oxygen and/or sulfur with BH₃, BH₃⁻ and/or Se.

Terminal modifications can also be useful for monitoring distribution, and in such cases the preferred groups to be added include fluorophores, e.g., fluorescein or an Alexa dye, e.g., Alexa 488. Terminal modifications can also be useful for enhancing uptake, useful modifications for this include targeting ligands. Terminal modifications can also be useful for cross-linking an oligonucleotide to another moiety; modifications useful for this include mitomycin C, psoralen, and derivatives thereof.

### Thermally Destabilizing Modifications

The oligonucleotide, such as iRNAs or dsRNA agents, can be optimized for RNA interference by increasing the propensity of the iRNA duplex to disassociate or melt (decreasing the free energy of duplex association) by introducing a thermally destabilizing modification in the sense strand at a site opposite to the seed region of the antisense strand (i.e., at positions 2-8 of the 5′-end of the antisense strand). This modification can increase the propensity of the duplex to disassociate or melt in the seed region of the antisense strand.

The thermally destabilizing modifications can include abasic modification; mismatch with the opposing nucleotide in the opposing strand; and sugar modification such as 2′-deoxy modification or acyclic nucleotide, e.g., unlocked nucleic acids (UNA) or glycerol nucleic acid (GNA).

Exemplified abasic modifications are:

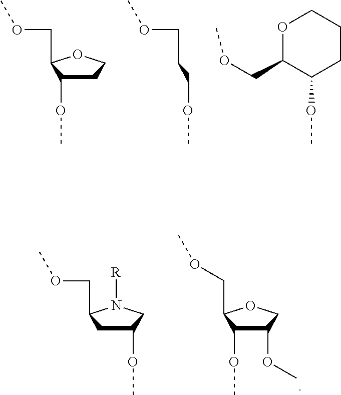

Exemplified sugar modifications are:

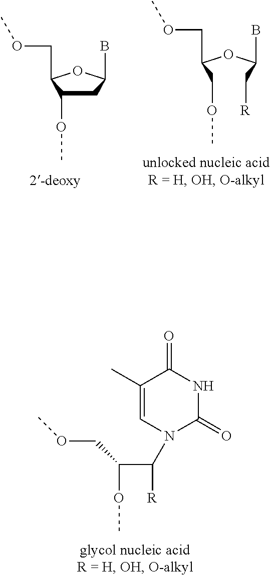

The term “UNA” refers to unlocked acyclic nucleic acid, wherein any of the bonds of the sugar has been removed, forming an unlocked “sugar” residue. In one example, UNA also encompasses monomers with bonds between C1′-C4′ being removed (i.e. the covalent carbon-oxygen-carbon bond between the C1′ and C4′ carbons). In another example, the C2′-C3′ bond (i.e. the covalent carbon-carbon bond between the C2′ and C3′ carbons) of the sugar is removed (see Mikhailov et. al., Tetrahedron Letters, 26 (17): 2059 (1985); and Fluiter et al., Mol. Biosyst., 10: 1039 (2009), which are hereby incorporated by reference in their entirety). The acyclic derivative provides greater backbone flexibility without affecting the Watson-Crick pairings. The acyclic nucleotide can be linked via 2′-5′ or 3′-5′ linkage.

The term ‘GNA’ refers to glycol nucleic acid which is a polymer similar to DNA or RNA but differing in the composition of its “backbone” in that is composed of repeating glycerol units linked by phosphodiester bonds:

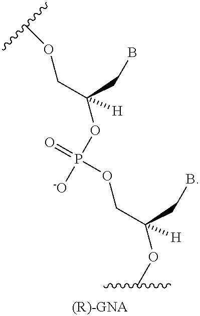

The thermally destabilizing modification can be mismatches (i.e., noncomplementary base pairs) between the thermally destabilizing nucleotide and the opposing nucleotide in the opposite strand within the dsRNA duplex. Exemplary mismatch basepairs include G:G, G:A, G:U, G:T, A:A, A:C, C:C, C:U, C:T, U:U, T:T, U:T, or a combination thereof. Other mismatch base pairings known in the art are also amenable to the present invention. A mismatch can occur between nucleotides that are either naturally occurring nucleotides or modified nucleotides, i.e., the mismatch base pairing can occur between the nucleobases from respective nucleotides independent of the modifications on the ribose sugars of the nucleotides. In certain embodiments, the oligonucleotide, such as siRNA or iRNA agent, contains at least one nucleobase in the mismatch pairing that is a 2′-deoxy nucleobase; e.g., the 2′-deoxy nucleobase is in the sense strand.

More examples of abasic nucleotide, acyclic nucleotide modifications (including UNA and GNA), and mismatch modifications have been described in detail in WO 2011/133876, which is herein incorporated by reference in its entirety.

The thermally destabilizing modifications may also include universal base with reduced or abolished capability to form hydrogen bonds with the opposing bases, and phosphate modifications.

Nucleobase modifications with impaired or completely abolished capability to form hydrogen bonds with bases in the opposite strand have been evaluated for destabilization of the central region of the dsRNA duplex as described in WO 2010/0011895, which is herein incorporated by reference in its entirety. Exemplary nucleobase modifications are:

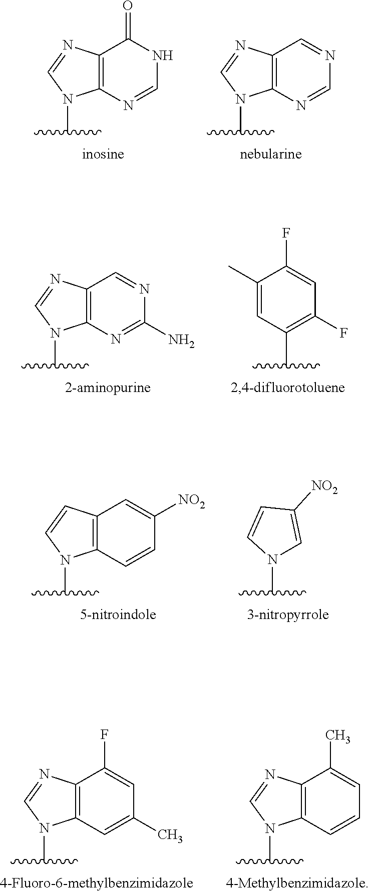

Exemplary phosphate modifications known to decrease the thermal stability of dsRNA duplexes compared to natural phosphodiester linkages are:

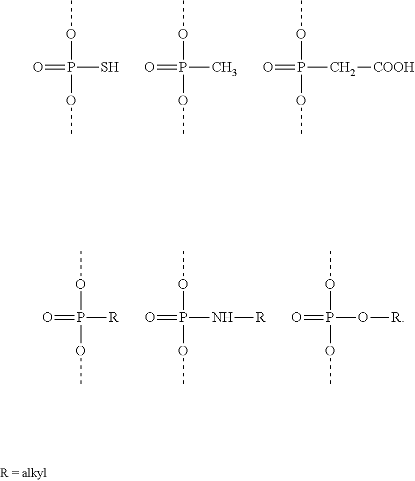

In some embodiments, the oligonucleotide can comprise 2′-5′ linkages (with 2′-H, 2′-OH and 2′-OMe and with P═O or P═S). For example, the 2′-5′ linkages modifications can be used to promote nuclease resistance or to inhibit binding of the sense to the antisense strand, or can be used at the 5′ end of the sense strand to avoid sense strand activation by RISC.

In another embodiment, the oligonucleotide can comprise L sugars (e.g., L ribose, L-arabinose with 2′-H, 2′-OH and 2′-OMe). For example, these L sugar modifications can be used to promote nuclease resistance or to inhibit binding of the sense to the antisense strand, or can be used at the 5′ end of the sense strand to avoid sense strand activation by RISC.

In some embodiments, the L group in the 2′-modified nucleoside of formula (I) is one or more targeting ligands, optionally connected via one or more linkers/tethers.

Introduction of the targeting ligands into an oligonucleotide via a 2′-modified nucleoside of formula (I), on either the sense or antisense strand or both the sense and antisense strands, are illustrated in Schemes 21-22 in Example 9 below. These targeting ligands can be cleaved off from the 2′-position, regenerating 2′—OH group, after the siRNA oligonucleotide enters into cytosol.

In some embodiments, the targeting ligand is selected from the group consisting of an antibody, a ligand-binding portion of a receptor, a ligand for a receptor, an aptamer, a carbohydrate-based ligand, a fatty acid, a lipoprotein, folate, thyrotropin, melanotropin, surfactant protein A, mucin, glycosylated polyaminoacids, transferrin, bisphosphonate, polyglutamate, polyaspartate, a lipophilic moiety that enhances plasma protein binding, a cholesterol, a steroid, bile acid, vitamin B12, biotin, a fluorophore, and a peptide.

In certain embodiments, at least one ligand is a carbohydrate-based ligand targeting a liver tissue. In one embodiment, the carbohydrate-based ligand is selected from the group consisting of galactose, multivalent galactose, N-acetyl-galactosamine (GalNAc), multivalent GalNAc, mannose, multivalent mannose, lactose, multivalent lactose, N-acetyl-glucosamine (GlcNAc), multivalent GlcNAc, glucose, multivalent glucose, fucose, and multivalent fucose.

In certain embodiments, at least one ligand is a lipophilic moiety. In one embodiment, the lipophilicity of the lipophilic moiety, measured by logK_{ow}, exceeds 0, or the hydrophobicity of the compound, measured by the unbound fraction in the plasma protein binding assay of the compound, exceeds 0.2.

In one embodiment, the lipophilic moiety contains a saturated or unsaturated C₄-C₃₀ hydrocarbon chain, and an optional functional group selected from the group consisting of hydroxy, amine, carboxylic acid, sulfonate, phosphate, thiol, azide, and alkyne. For instance, the lipophilic moiety contains a saturated or unsaturated C₆-C₁₈ hydrocarbon chain.

Additional lipophilic moieties and additional details regarding lipophilicity of the lipophilic moiety and hydrophobicity of the oligonucleotide can be found in PCT Application No. PCT/US20/59399, entitled “Extrahepatic Delivery,” filed on Nov. 6, 2020, the content of which is incorporated herein by reference in its entirety.

In certain embodiments, at least one ligand targets a receptor which mediates delivery to a CNS tissue. In one embodiment, the targeting ligand is selected from the group consisting of Angiopep-2, lipoprotein receptor related protein (LRP) ligand, bEnd.3 cell binding ligand, transferrin receptor (TfR) ligand, manose receptor ligand, glucose transporter protein, and LDL receptor ligand.

In certain embodiments, at least one ligand targets a receptor which mediates delivery to an ocular tissue. In one embodiment, the targeting ligand is selected from the group consisting of trans-retinol, RGD peptide, LDL receptor ligand, and carbohydrate based ligands.

The targeting ligands can also be introduced into the oligonucleotide not through the bio-cleavable linker at the 2′-modified nucleoside of formula (I) described herein.

In some embodiments, the oligonucleotide contains at least one targeting ligand at the 5′-end, 3′-end, and/or internal position(s) of the antisense strand.

In some embodiments, the oligonucleotide contains at least one targeting ligand at the 5′-end, 3′-end, and/or internal position(s) of the sense strand.

In some embodiments, the oligonucleotide contains at least one 2′-modified nucleoside of formula (I) at the 5′-end, 3′-end, and/or internal position(s) of the antisense strand, and at least one targeting ligand at the 5′-end, 3′-end, and/or internal position(s) of the sense strand.

In one embodiment, the oligonucleotide contains at least one 2′-modified nucleoside of formula (I) at the 5′-end of the antisense strand, and at least one targeting ligand at the 3′-end of the sense strand.

In some embodiments, one or more targeting ligands are connected to the 2′-modified nucleoside of formula (I) via one or more linkers/tethers, as described below.

In some embodiments, one or more targeting ligands are connected to the oligonucleotide at a position different than the 2′-modified nucleoside of formula (I), via one or more linkers/tethers, as described below.

### Linkers Tethers

Linkers/Tethers are connected to the oligonucleotide at a “tethering attachment point (TAP).” Linkers/Tethers may include any C₁-C₁₀₀ carbon-containing moiety, (e.g. C₁—C₇₅, C₁-C₅₀, C₁-C₂₀, C₁-C₁₀; C₁, C₂, C₃, C₄, C₅, C₆, C₇, C₈, C₉, or C₁₀), and may have at least one nitrogen atom. In certain embodiments, the nitrogen atom forms part of a terminal amino or amido (NHC(O)—) group on the linker/tether, which may serve as a connection point for oligonucleotide. Non-limited examples of linkers/tethers (underlined) include TAP-(CH₂)ₙNH—; TAP-C(O)(CH₂)ₙNH—; TAP-NR″″CH₂)ₙNH—, TAP-C(O)—(CH₂)ₙ—C(O)—; TAP-C(O)—(CH₂)ₙ—C(O)O—; TAP-C(O)—O—; TAP-C(O)—(CH₂)ₙ—NH—C(O)—; TAP-C(O)—(CH₂)ₙ—; TAP-C(O)—NH—; TAP-C(O)—; TAP-(CH₂)ₙ—C(O)—; TAP-(CH₂)ₙ—C(O)O—; TAP-(CH₂)ₙ—; or TAP-(CH₂)ₙ—NH—C(O)—; in which n is 1-20 (e.g., 1, 2, 3, 4, 5, 6, 7, 8, 9, 10, 11, 12, 13, 14, 15, 16, 17, 18, 19, or 20) and R″″ is C₁-C₆ alkyl. Preferably, n is 5, 6, or 11. In other embodiments, the nitrogen may form part of a terminal oxyamino group, e.g., —ONH₂, or hydrazino group, —NHNH₂. The linker/tether may optionally be substituted, e.g., with hydroxy, alkoxy, perhaloalkyl, and/or optionally inserted with one or more additional heteroatoms, e.g., N, O, or S. Preferred tethered ligands may include, e.g., TAP-(CH₂)ₙNH(LIGAND); TAP-C(O)(CH₂)ₙNH(LIGAND); TAP-NR″″(CH₂)ₙNH(LIGAND); TAP-(CH₂)ₙONH(LIGAND); TAP-C(O)(CH₂)ₙONH(LIGAND); TAP-NR″″CH₂)ₙONH(LIGAND); TAP-(CH₂)ₙNHNH₂(LIGAND), TAP-C(O)(CH₂)ₙNHNH2(LIGAND); TAP-NR″″(CH₂)ₙNHNH2(LIGAND); TAP-C(O)—(CH₂)ₙ—C(O)(LIGAND); TAP-C(O)—(CH₂)ₙ—C(O)O(LIGAND); TAP-C(O)—O(LIGAND); TAP-C(O)—(CH₂)ₙ—NH—C(O)(LIGAND); TAP-C(O)—(CH₂)ₙ(LIGAND); TAP-C(O)—NH(LIGAND); TAP-C(O)(LIGAND); TAP-(CH₂)ₙ—C(O) (LIGAND); TAP-(CH₂)ₙ—C(O)O(LIGAND); TAP-(CH₂)ₙ(LIGAND); or TAP-(CH₂)ₙ—NH—C(O)(LIGAND). In some embodiments, amino terminated linkers/tethers (e.g., NH₂, ONH₂, NH₂NH₂) can form an imino bond (i.e., C═N) with the ligand. In some embodiments, amino terminated linkers/tethers (e.g., NH₂, ONH₂, NH₂NH₂) can acylated, e.g., with C(O)CF₃.

In some embodiments, the linker/tether can terminate with a mercapto group (i.e., SH) or an olefin (e.g., CH═CH₂). For example, the tether can be TAP-(CH₂)ₙ—SH, TAP-C(O)(CH₂)ₙSH, TAP-(CH₂)ₙ—(CH═CH₂), or TAP-C(O)(CH₂)ₙ(CH═CH₂), in which n can be as described elsewhere. The tether may optionally be substituted, e.g., with hydroxy, alkoxy, perhaloalkyl, and/or optionally inserted with one or more additional heteroatoms, e.g., N, O, or S. The double bond can be cis or trans or E or Z.

In other embodiments, the linker/tether may include an electrophilic moiety, preferably at the terminal position of the linker/tether. Exemplary electrophilic moieties include, e.g., an aldehyde, alkyl halide, mesylate, tosylate, nosylate, or brosylate, or an activated carboxylic acid ester, e.g. an NHS ester, or a pentafluorophenyl ester. Preferred linkers/tethers (underlined) include TAP-(CH₂)ₙCHO; TAP-C(O)(CH₂)ₙCHO; or TAP-NR″″(CH₂),CHO, in which n is 1-6 and R″″ is C₁-C₆ alkyl; or TAP-(CH₂)ₙC(O)ONHS; TAP-C(O)(CH₂),C(O)ONHS; or TAP-NR″″(CH₂)ₙC(O)ONHS, in which n is 1-6 and R″″ is C₁-C₆ alkyl; TAP-(CH₂)ₙC(O)OC₆F₅; TAP-C(O)(CH₂)ₙC(O) OC₆F₅; or TAP-NR″″(CH₂)ₙC(O) OC₆F₅, in which n is 1-11 and R″″ is C₁-C₆ alkyl; or —(CH₂)ₙCH₂LG; TAP-C(O)(CH₂)ₙCH₂LG; or TAP-NR″″(CH₂)ₙCH₂LG, in which n can be as described elsewhere and R″″ is C₁-C₆ alkyl (LG can be a leaving group, e.g., halide, mesylate, tosylate, nosylate, brosylate). Tethering can be carried out by coupling a nucleophilic group of a ligand, e.g., a thiol or amino group with an electrophilic group on the tether.

In other embodiments, it can be desirable for the monomer to include a phthalimido group (K) at the terminal position of the linker/tether.

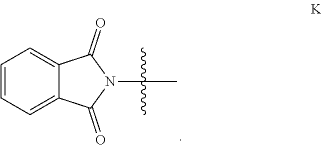 [SMILES: *N1C(=O)c2ccccc2C1=O]

In other embodiments, other protected amino groups can be at the terminal position of the linker/tether, e.g., alloc, monomethoxy trityl (MMT), trifluoroacetyl, Fmoc, or aryl sulfonyl (e.g., the aryl portion can be ortho-nitrophenyl or ortho, para-dinitrophenyl).

Any of the linkers/tethers described herein may further include one or more additional linking groups, e.g., —O—(CH₂)ₙ—, —(CH₂)ₙ—SS—, —(CH₂)ₙ—, or —(CH═CH)—.

### Cleavable Linkers/Tethers

In some embodiments, at least one of the linkers/tethers can be a redox cleavable linker, an acid cleavable linker, an esterase cleavable linker, a phosphatase cleavable linker, or a peptidase cleavable linker.

In one embodiment, at least one of the linkers/tethers can be a reductively cleavable linker (e.g., a disulfide group).

In one embodiment, at least one of the linkers/tethers can be an acid cleavable linker (e.g., a hydrazone group, an ester group, an acetal group, or a ketal group).

In one embodiment, at least one of the linkers/tethers can be an esterase cleavable linker (e.g., an ester group).

In one embodiment, at least one of the linkers/tethers can be a phosphatase cleavable linker (e.g., a phosphate group).

In one embodiment, at least one of the linkers/tethers can be a peptidase cleavable linker (e.g., a peptide bond).

Cleavable linking groups are susceptible to cleavage agents, e.g., pH, redox potential or the presence of degradative molecules. Generally, cleavage agents are more prevalent or found at higher levels or activities inside cells than in serum or blood. Examples of such degradative agents include: redox agents which are selected for particular substrates or which have no substrate specificity, including, e.g., oxidative or reductive enzymes or reductive agents such as mercaptans, present in cells, that can degrade a redox cleavable linking group by reduction; esterases; endosomes or agents that can create an acidic environment, e.g., those that result in a pH of five or lower; enzymes that can hydrolyze or degrade an acid cleavable linking group by acting as a general acid, peptidases (which can be substrate specific), and phosphatases.

A cleavable linkage group, such as a disulfide bond can be susceptible to pH. The pH of human serum is 7.4, while the average intracellular pH is slightly lower, ranging from about 7.1-7.3. Endosomes have a more acidic pH, in the range of 5.5-6.0, and lysosomes have an even more acidic pH at around 5.0. Some tethers will have a linkage group that is cleaved at a preferred pH, thereby releasing the iRNA agent from a ligand (e.g., a targeting or cell-permeable ligand, such as cholesterol) inside the cell, or into the desired compartment of the cell.

A chemical junction (e.g., a linking group) that links a ligand to an iRNA agent can include a disulfide bond. When the iRNA agent/ligand complex is taken up into the cell by endocytosis, the acidic environment of the endosome will cause the disulfide bond to be cleaved, thereby releasing the iRNA agent from the ligand (Quintana et al., Pharm Res. 19:1310-1316, 2002; Patri et al., Curr. Opin. Curr. Biol. 6:466-471, 2002). The ligand can be a targeting ligand or a second therapeutic agent that may complement the therapeutic effects of the iRNA agent.

A tether can include a linking group that is cleavable by a particular enzyme. The type of linking group incorporated into a tether can depend on the cell to be targeted by the iRNA agent. For example, an iRNA agent that targets an mRNA in liver cells can be conjugated to a tether that includes an ester group. Liver cells are rich in esterases, and therefore the tether will be cleaved more efficiently in liver cells than in cell types that are not esterase-rich. Cleavage of the tether releases the iRNA agent from a ligand that is attached to the distal end of the tether, thereby potentially enhancing silencing activity of the iRNA agent. Other cell-types rich in esterases include cells of the lung, renal cortex, and testis.

Tethers that contain peptide bonds can be conjugated to iRNA agents target to cell types rich in peptidases, such as liver cells and synoviocytes. For example, an iRNA agent targeted to synoviocytes, such as for the treatment of an inflammatory disease (e.g., rheumatoid arthritis), can be conjugated to a tether containing a peptide bond.

In general, the suitability of a candidate cleavable linking group can be evaluated by testing the ability of a degradative agent (or condition) to cleave the candidate linking group. It will also be desirable to also test the candidate cleavable linking group for the ability to resist cleavage in the blood or when in contact with other non-target tissue, e.g., tissue the iRNA agent would be exposed to when administered to a subject. Thus one can determine the relative susceptibility to cleavage between a first and a second condition, where the first is selected to be indicative of cleavage in a target cell and the second is selected to be indicative of cleavage in other tissues or biological fluids, e.g., blood or serum. The evaluations can be carried out in cell free systems, in cells, in cell culture, in organ or tissue culture, or in whole animals. It may be useful to make initial evaluations in cell-free or culture conditions and to confirm by further evaluations in whole animals. In preferred embodiments, useful candidate compounds are cleaved at least 2, 4, 10 or 100 times faster in the cell (or under in vitro conditions selected to mimic intracellular conditions) as compared to blood or serum (or under in vitro conditions selected to mimic extracellular conditions).

### Redox Cleavable Linking Groups

One class of cleavable linking groups are redox cleavable linking groups that are cleaved upon reduction or oxidation. An example of reductively cleavable linking group is a disulphide linking group (—S—S—). To determine if a candidate cleavable linking group is a suitable “reductively cleavable linking group,” or for example is suitable for use with a particular iRNA moiety and particular targeting agent one can look to methods described herein. For example, a candidate can be evaluated by incubation with dithiothreitol (DTT), or other reducing agent using reagents know in the art, which mimic the rate of cleavage which would be observed in a cell, e.g., a target cell. The candidates can also be evaluated under conditions which are selected to mimic blood or serum conditions. In a preferred embodiment, candidate compounds are cleaved by at most 10% in the blood. In preferred embodiments, useful candidate compounds are degraded at least 2, 4, 10 or 100 times faster in the cell (or under in vitro conditions selected to mimic intracellular conditions) as compared to blood (or under in vitro conditions selected to mimic extracellular conditions). The rate of cleavage of candidate compounds can be determined using standard enzyme kinetics assays under conditions chosen to mimic intracellular media and compared to conditions chosen to mimic extracellular media.

### Phosphate-Based Cleavable Linking Groups

Phosphate-based linking groups are cleaved by agents that degrade or hydrolyze the phosphate group. An example of an agent that cleaves phosphate groups in cells are enzymes such as phosphatases in cells. Examples of phosphate-based linking groups are O—P(O)(ORk)—O—, —O—P(S)(ORk)—O—, —O—P(S)(SRk)—O—, —S—P(O)(ORk)—O—, —O—P(O)(ORk)—S—, S—P(O)(ORk)—S—, O—P(S)(ORk)—S—, S—P(S)(ORk)—O—, O—P(O)(Rk)—O—, —O—P(S)(Rk)—O—, —S—P(O)(Rk)—O—, —S—P(S)(Rk)—O—, —S—P(O)(Rk)—S—, —O—P(S)(Rk)—S—. Preferred embodiments are —O—P(O)(OH)—O—, —O—P(S)(OH)—O—, —O—P(S)(SH)—O—, —S—P(O)(OH)—O—, —O—P(O)(OH) —S—, —S—P(O)(OH)—S—, —O—P(S)(OH)—S—, —S—P(S)(OH)—O—, —O—P(O)(H)—O—, —O—P(S)(H)—O—, —S—P(O)(H)—O—, —S—P(S)(H)—O—, —S—P(O)(H)—S—, —O—P(S)(H)—S—. A preferred embodiment is —O—P(O)(OH)—O—. These candidates can be evaluated using methods analogous to those described above.

### Acid Cleavable Linking Groups

Acid cleavable linking groups are linking groups that are cleaved under acidic conditions. In preferred embodiments acid cleavable linking groups are cleaved in an acidic environment with a pH of about 6.5 or lower (e.g., about 6.0, 5.5, 5.0, or lower), or by agents such as enzymes that can act as a general acid. In a cell, specific low pH organelles, such as endosomes and lysosomes can provide a cleaving environment for acid cleavable linking groups. Examples of acid cleavable linking groups include but are not limited to hydrazones, ketals, acetals, esters, and esters of amino acids. Acid cleavable groups can have the general formula —C═NN—, C(O)O, or —OC(O). A preferred embodiment is when the carbon attached to the oxygen of the ester (the alkoxy group) is an aryl group, substituted alkyl group, or tertiary alkyl group such as dimethyl pentyl or t-butyl. These candidates can be evaluated using methods analogous to those described above.

### Ester-Based Linking Groups

Ester-based linking groups are cleaved by enzymes such as esterases and amidases in cells. Examples of ester-based cleavable linking groups include but are not limited to esters of alkylene, alkenylene and alkynylene groups. Ester cleavable linking groups have the general formula C(O)O—, or OC(O)—. These candidates can be evaluated using methods analogous to those described above.

### Peptide-Based Cleaving Groups

Peptide-based linking groups are cleaved by enzymes such as peptidases and proteases in cells. Peptide-based cleavable linking groups are peptide bonds formed between amino acids to yield oligopeptides (e.g., dipeptides, tripeptides etc.) and polypeptides. Peptide-based cleavable groups do not include the amide group (—C(O)NH—). The amide group can be formed between any alkylene, alkenylene or alkynelene. A peptide bond is a special type of amide bond formed between amino acids to yield peptides and proteins. The peptide based cleavage group is generally limited to the peptide bond (i.e., the amide bond) formed between amino acids yielding peptides and proteins and does not include the entire amide functional group. Peptide cleavable linking groups have the general formula NHCHR¹C(O)NHCHR²C(O), where R¹ and R² are the R groups of the two adjacent amino acids. These candidates can be evaluated using methods analogous to those described above.

### Biocleavable Linkers/Tethers

The linkers can also include biocleavable linkers that are nucleotide and non-nucleotide linkers or combinations thereof that connect two parts of a molecule, for example, one or both strands of two individual siRNA molecule to generate a bis(siRNA). In some embodiments, mere electrostatic or stacking interaction between two individual siRNAs can represent a linker. The non-nucleotide linkers include tethers or linkers derived from monosaccharides, disaccharides, oligosaccharides, and derivatives thereof, aliphatic, alicyclic, heterocyclic, and combinations thereof.

In some embodiments, at least one of the linkers (tethers) is a bio-cleavable linker selected from the group consisting of DNA, RNA, disulfide, amide, functionalized monosaccharides or oligosaccharides of galactosamine, glucosamine, glucose, galactose, and mannose, and combinations thereof.

In one embodiment, the bio-cleavable carbohydrate linker may have 1 to 10 saccharide units, which have at least one anomeric linkage capable of connecting two siRNA units. When two or more saccharides are present, these units can be linked via 1-3, 1-4, or 1-6 sugar linkages, or via alkyl chains.

Exemplary bio-cleavable carbohydrate linkers include:

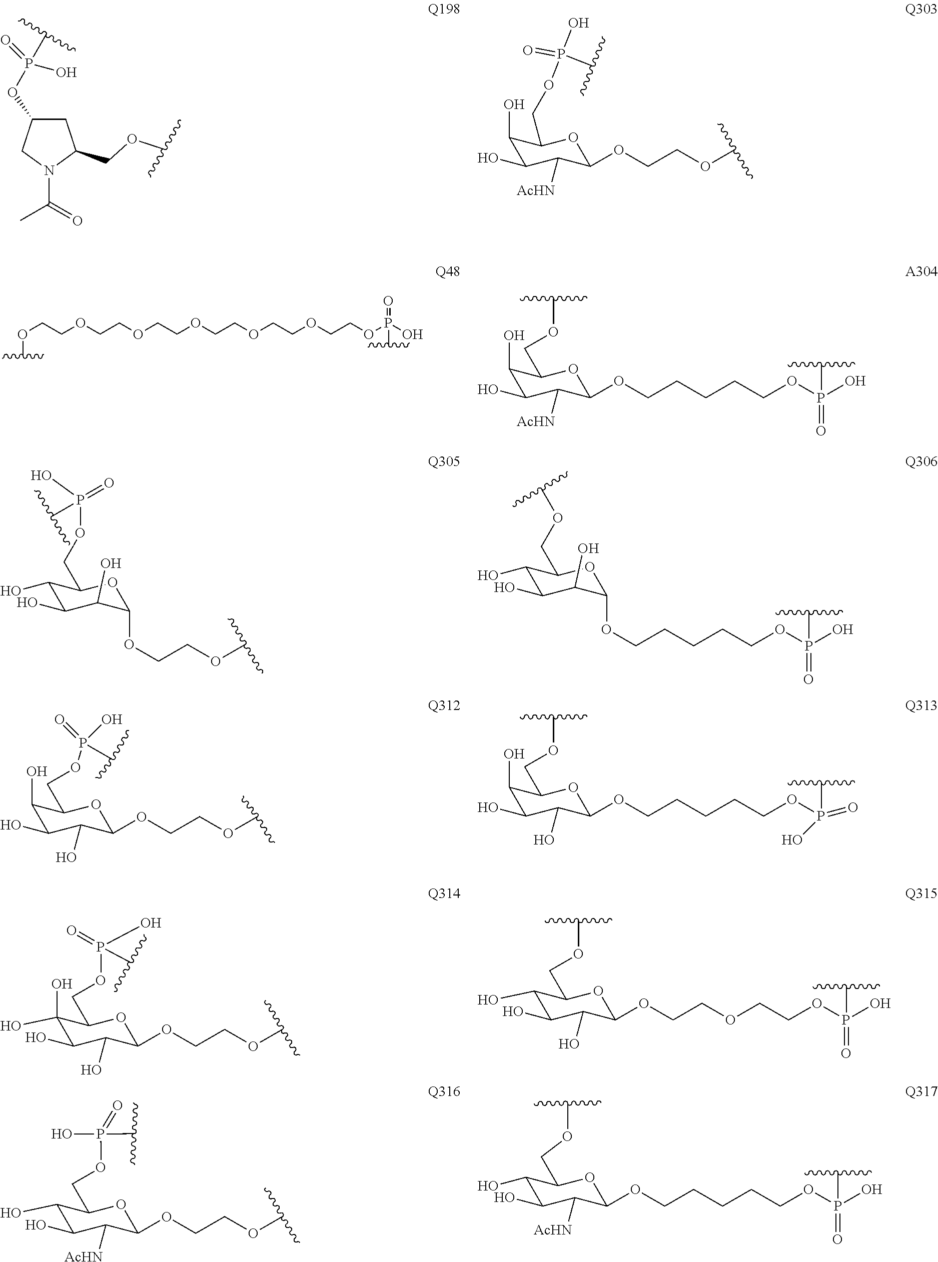

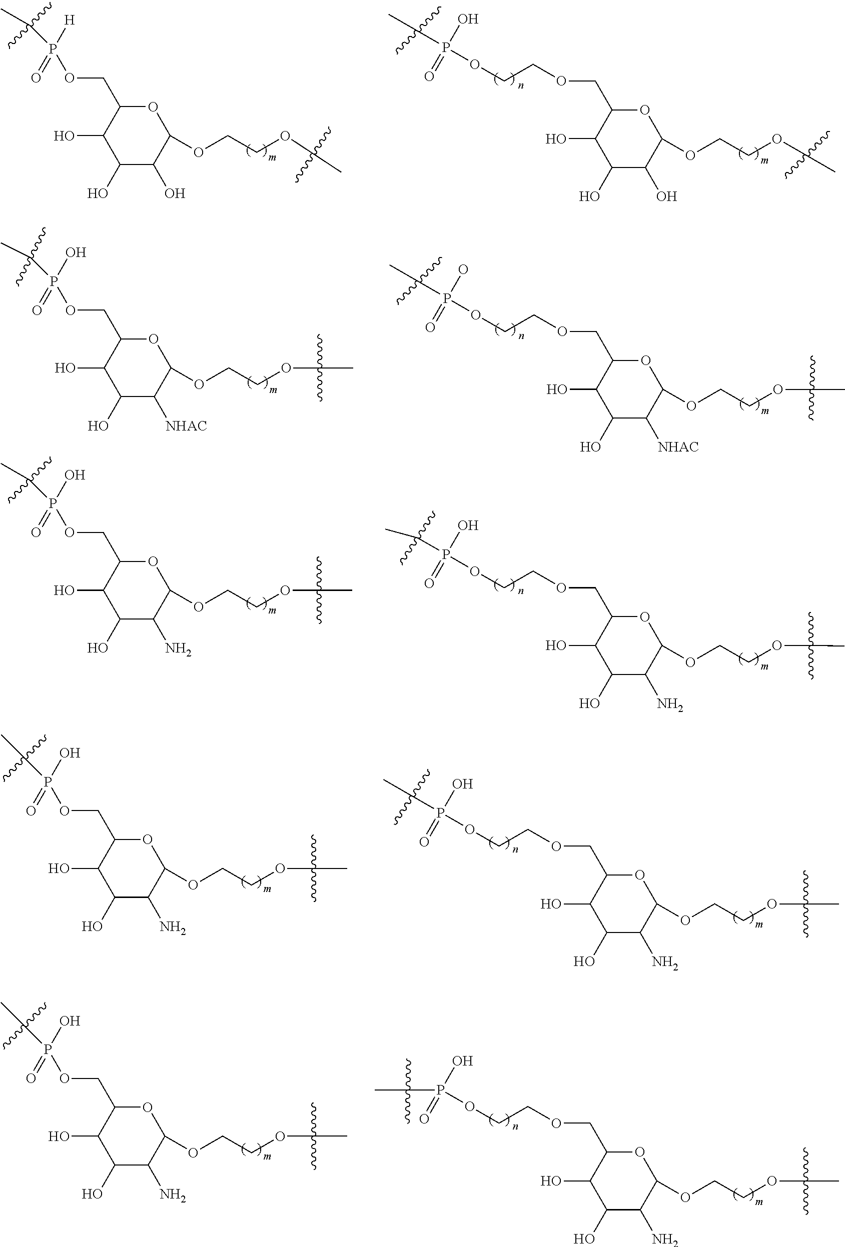

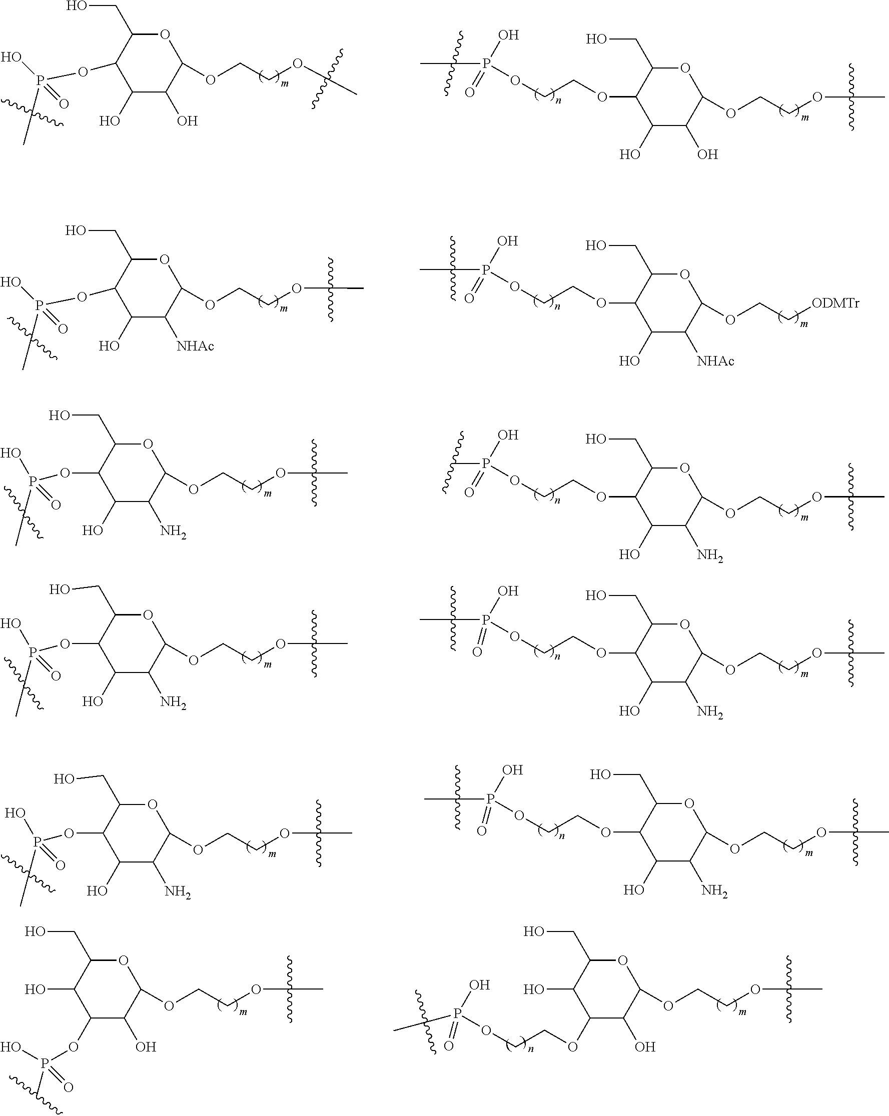

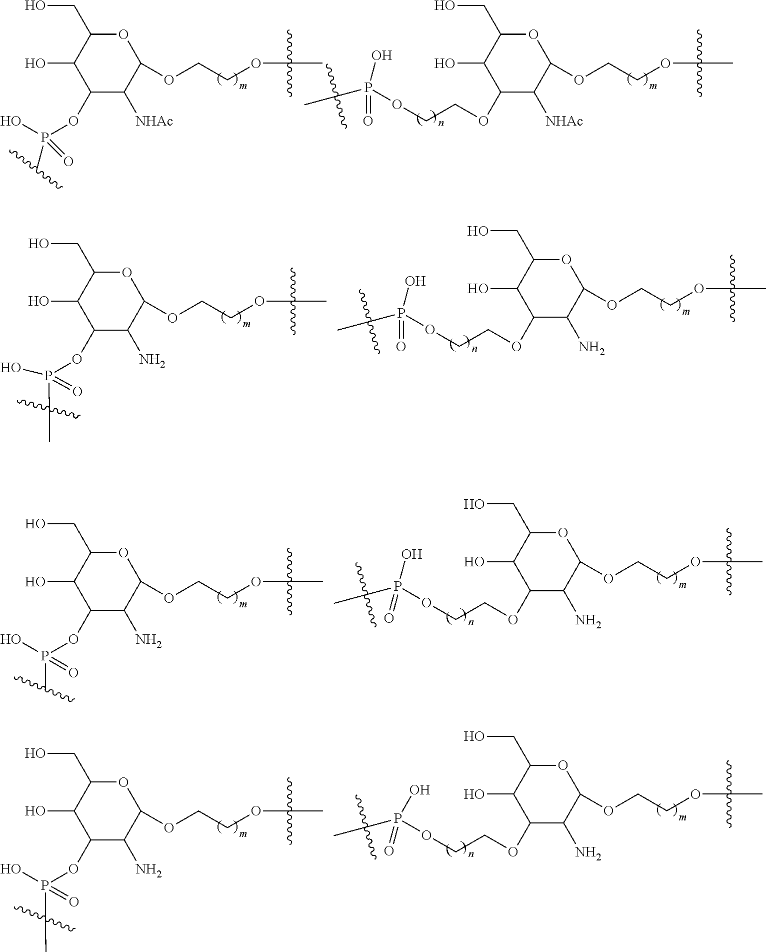

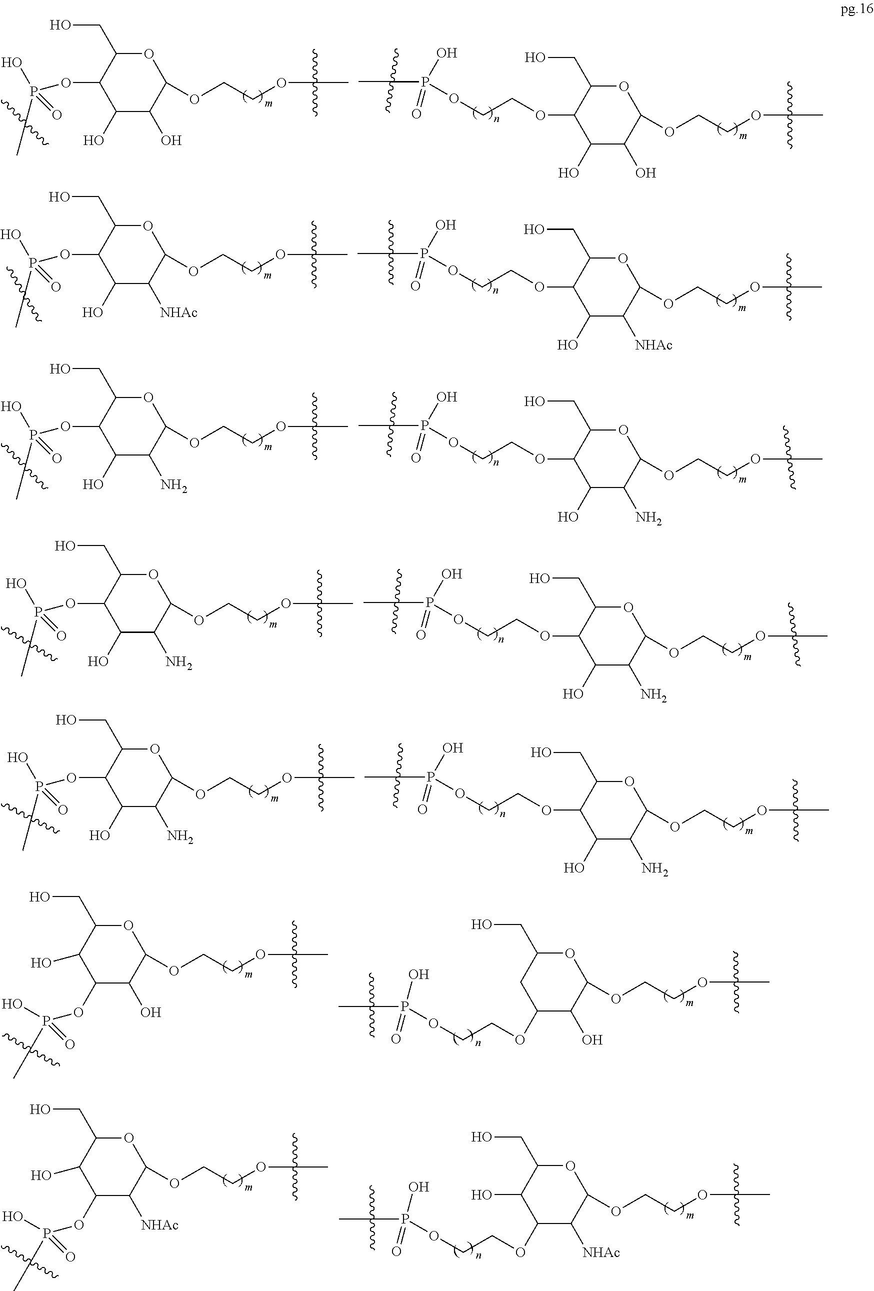

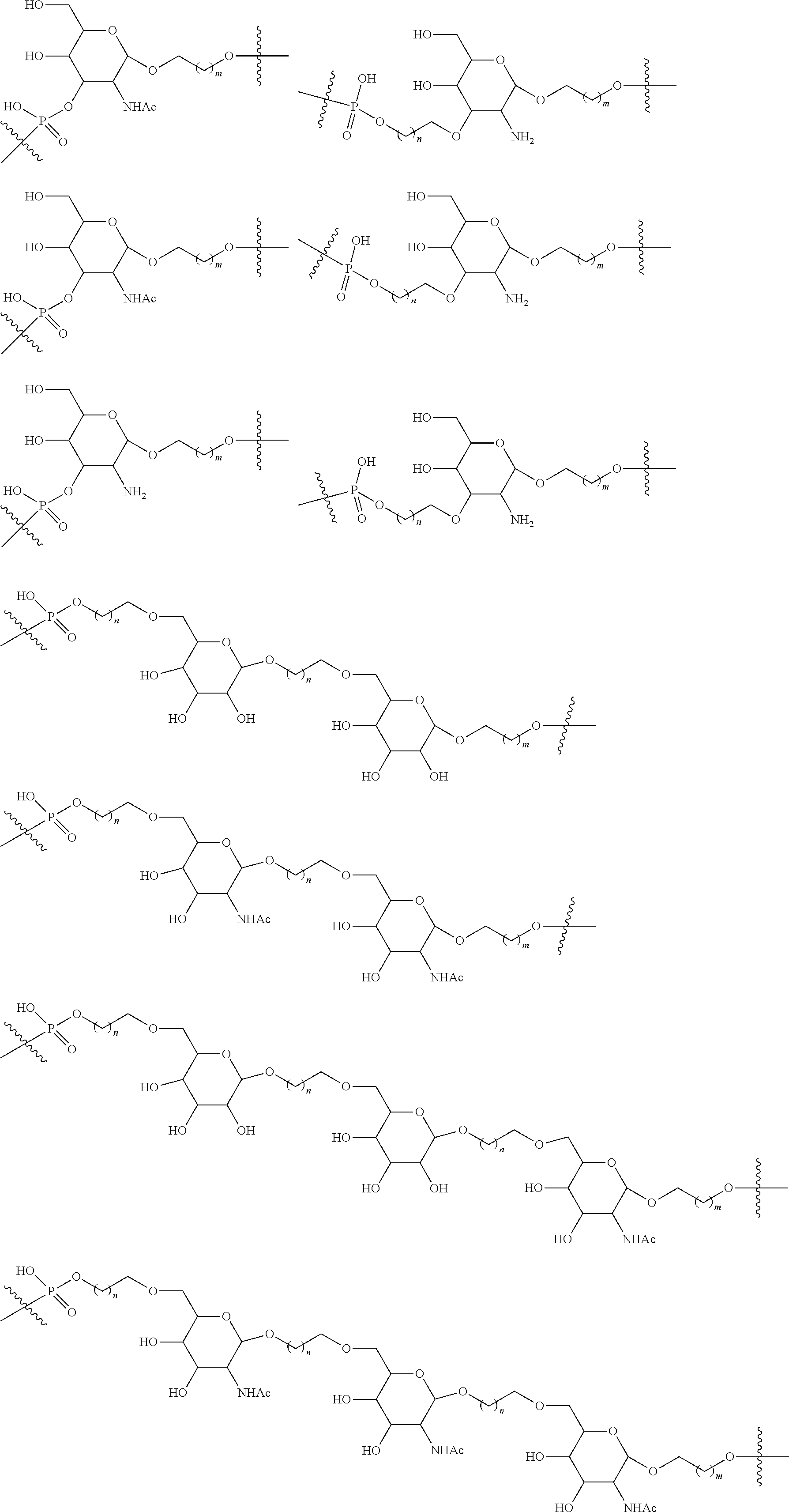

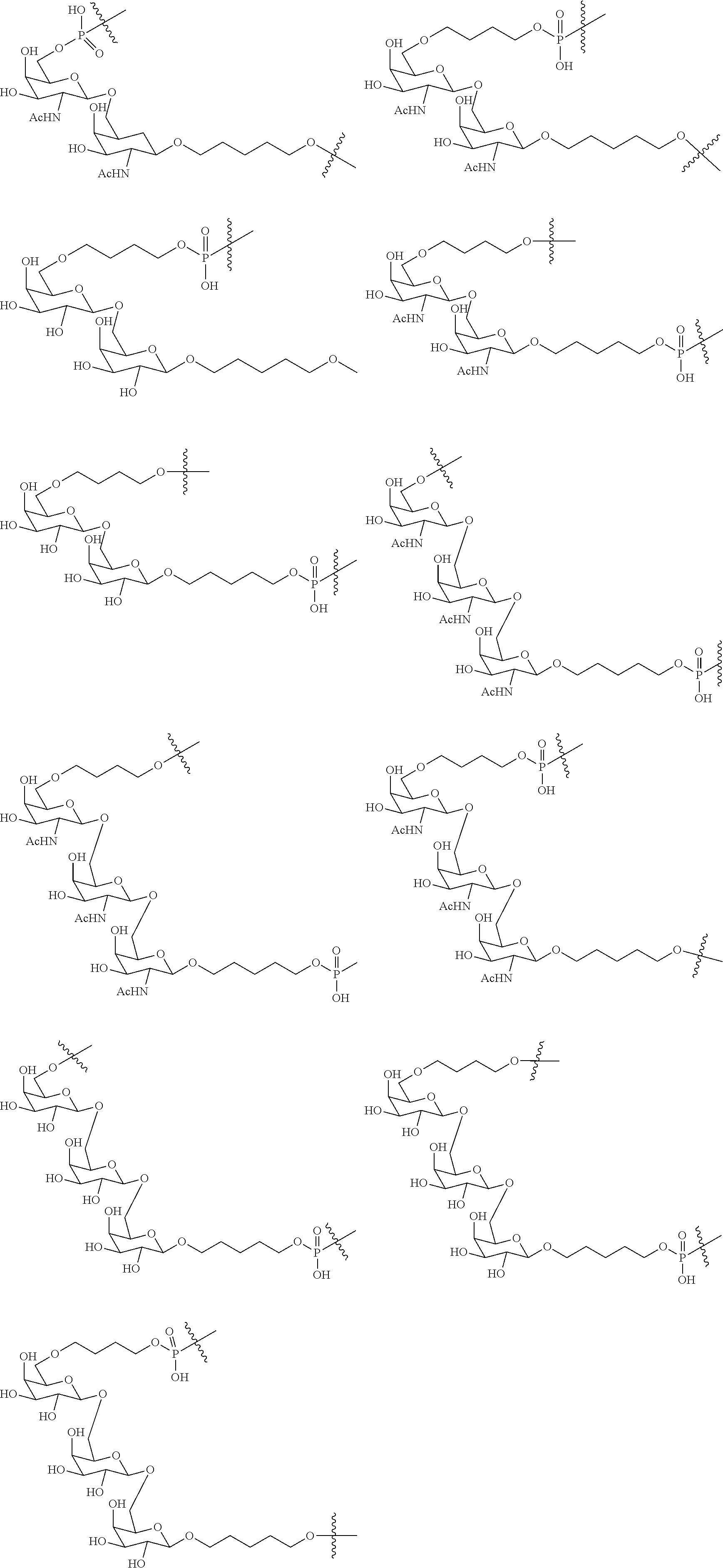

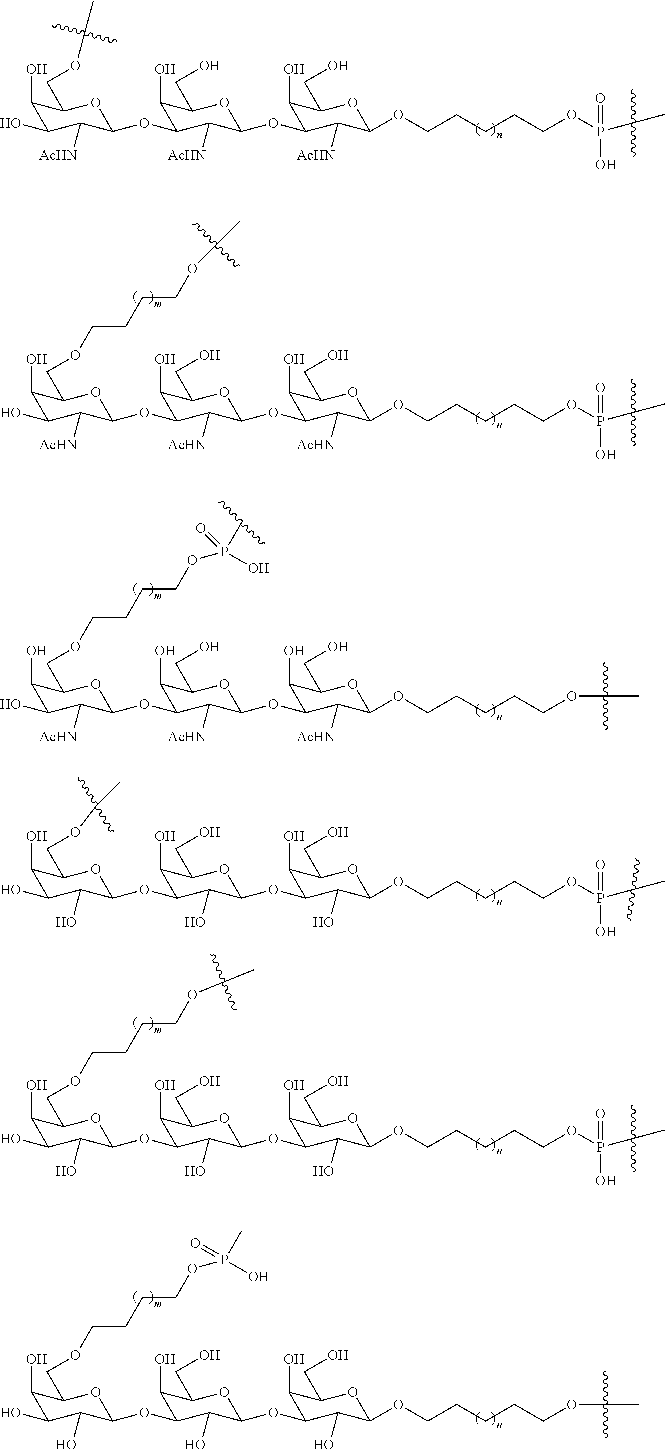

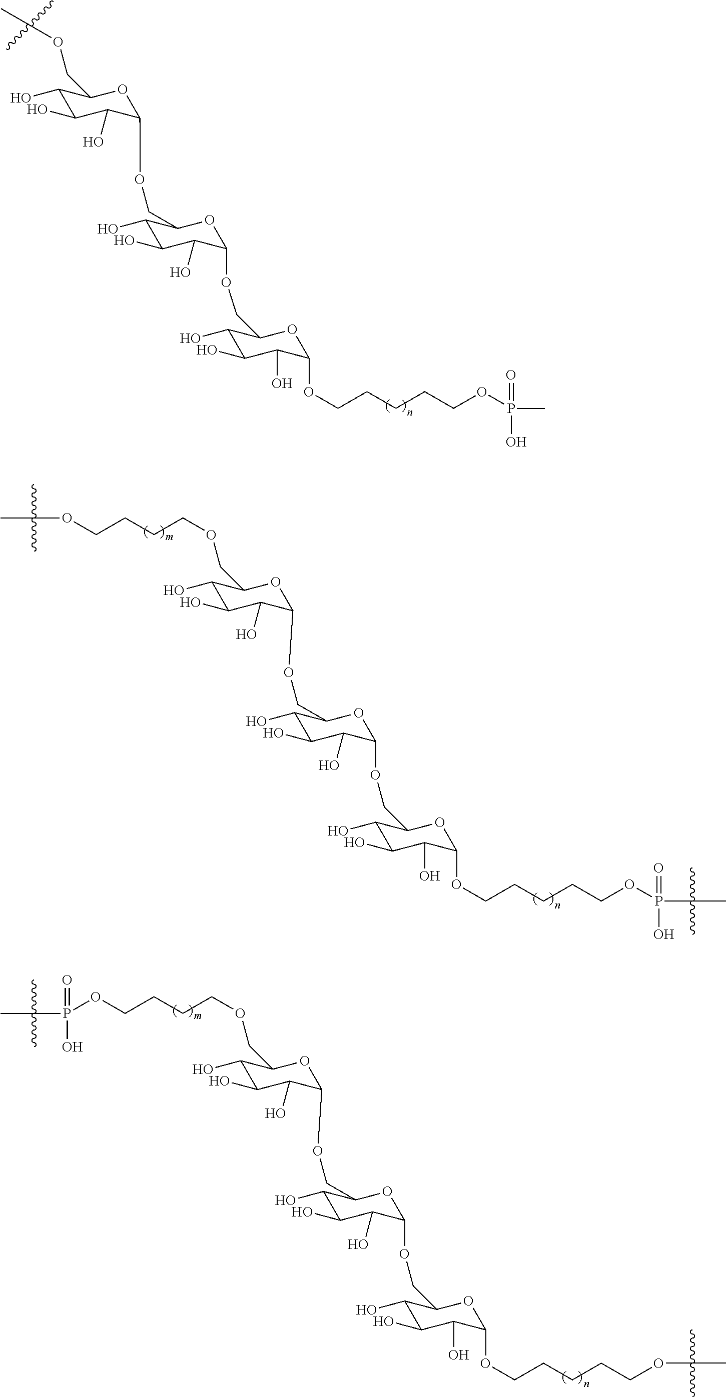

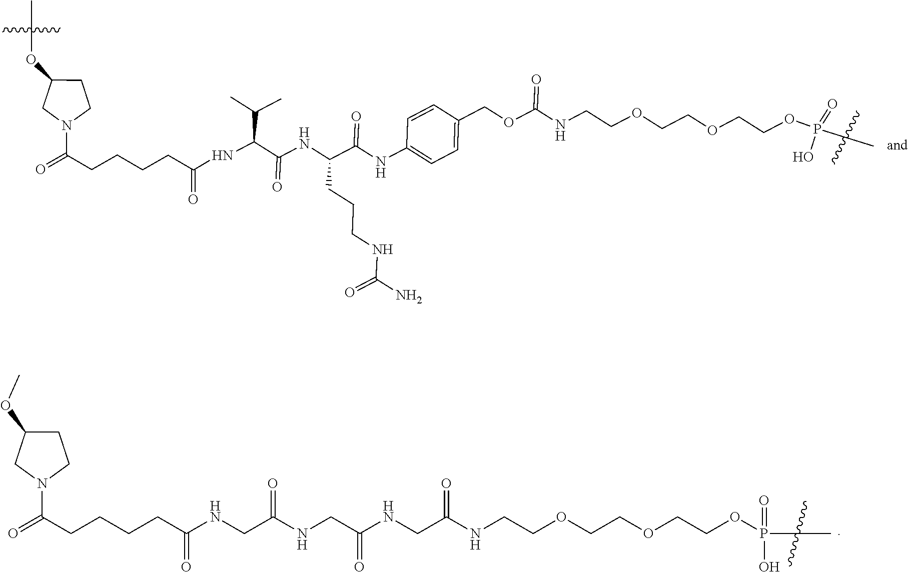

More discussion about the biocleavable linkers may be found in PCT application No. PCT/US18/14213, entitled “Endosomal Cleavable Linkers,” filed on Jan. 18, 2018, the content of which is incorporated herein by reference in its entirety.

### Carriers

In some embodiments, one or more targeting ligands are connected to the 2′-modified nucleoside of formula (I) via one or more carriers, as described herein, and optionally via one or more linkers/tethers, as described above,

In some embodiments, one or more targeting ligands are connected to the oligonucleotide at a position different than the 2′-modified nucleoside of formula (I), via one or more carriers, as described herein, and optionally via one or more linkers/tethers, as described above.

The carrier can be a cyclic group or an acyclic group. In one embodiment, the cyclic group is selected from the group consisting of pyrrolidinyl, pyrazolinyl, pyrazolidinyl, imidazolinyl, imidazolidinyl, piperidinyl, piperazinyl, [1,3]dioxolane, oxazolidinyl, isoxazolidinyl, morpholinyl, thiazolidinyl, isothiazolidinyl, quinoxalinyl, pyridazinonyl, tetrahydrofuryl, and decalin. In one embodiment, the acyclic group is a moiety based on a serinol backbone or a diethanolamine backbone.

The carrier can replace one or more nucleotide(s) of the iRNA agent.

In some embodiments, the carrier replaces one or more nucleotide(s) in the internal position(s) of the iRNA agent.

In other embodiments, the carrier replaces the nucleotides at the terminal end of the sense strand or antisense strand. In one embodiment, the carrier replaces the terminal nucleotide on the 3′ end of the sense strand, thereby functioning as an end cap protecting the 3′ end of the sense strand. In one embodiment, the carrier is a cyclic group having an amine, for instance, the carrier may be pyrrolidinyl, pyrazolinyl, pyrazolidinyl, imidazolinyl, imidazolidinyl, piperidinyl, piperazinyl, [1,3]dioxolanyl, oxazolidinyl, isoxazolidinyl, morpholinyl, thiazolidinyl, isothiazolidinyl, quinoxalinyl, pyridazinonyl, tetrahydrofuranyl, or decalinyl.

A ribonucleotide subunit in which the ribose sugar of the subunit has been so replaced is referred to herein as a ribose replacement modification subunit (RRMS). The carrier can be a cyclic or acyclic moiety and include two “backbone attachment points” (e.g., hydroxyl groups) and a ligand. The targeting ligand can be directly attached to the carrier or indirectly attached to the carrier by an intervening linker/tether, as described above.

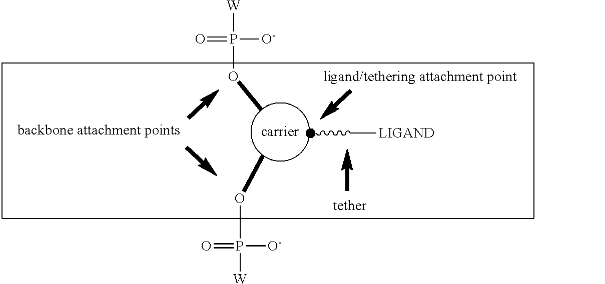

The ligand-conjugated monomer subunit may be the 5′ or 3′ terminal subunit of the iRNA molecule, i.e., one of the two “W” groups may be a hydroxyl group, and the other “W” group may be a chain of two or more unmodified or modified ribonucleotides. Alternatively, the ligand-conjugated monomer subunit may occupy an internal position, and both “W” groups may be one or more unmodified or modified ribonucleotides. More than one ligand-conjugated monomer subunit may be present in an iRNA agent.

### Sugar Replacement-Based Monomers, e.g., Ligand-Conjugated Monomers (Cyclic)

Cyclic sugar replacement-based monomers, e.g., sugar replacement-based ligand-conjugated monomers, are also referred to herein as RRMS monomer compounds. The carriers may have the general formula (LCM-2) provided below (In that structure preferred backbone attachment points can be chosen from R¹ or R²; R³ or R⁴; or R⁹ and R¹⁰ if Y is CR⁹R¹⁰ (two positions are chosen to give two backbone attachment points, e.g., R¹ and R⁴, or R⁴ and R⁹)). Preferred tethering attachment points include R⁷; R⁵ or R⁶ when X is CH₂. The carriers are described below as an entity, which can be incorporated into a strand. Thus, it is understood that the structures also encompass the situations wherein one (in the case of a terminal position) or two (in the case of an internal position) of the attachment points, e.g., R¹ or R²; R³ or R⁴; or R⁹ or R¹⁰ (when Y is CR⁹R¹⁰), is connected to the phosphate, or modified phosphate, e.g., sulfur containing, backbone. E.g., one of the above-named R groups can be —CH₂—, wherein one bond is connected to the carrier and one to a backbone atom, e.g., a linking oxygen or a central phosphorus atom.

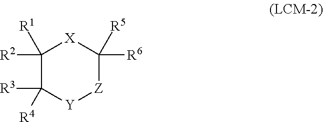

wherein:
X is N(CO)R⁷, NR⁷ or CH₂;
Y is NR⁸, O, S, CR⁹R¹⁰;
Z is CR¹¹R¹² or absent;
Each of R¹, R², R³, R⁴, R⁹, and R¹⁰ is, independently, H, OR^{a}, or (CH₂)ₙOR, provided that at least two of R¹, R², R³, R⁴, R⁹, and R¹⁰ are OR^{a} and/or (CH₂)ₙOR^{b};
Each of R⁵, R⁶, R¹¹, and R¹² is, independently, a ligand, H, C₁-C₆ alkyl optionally substituted with 1-3 R¹³, or C(O)NHR⁷; or R⁵ and R¹¹ together are C₃-C₈ cycloalkyl optionally substituted with R¹⁴;
R⁷ can be a ligand, e.g., R⁷ can be R^{d}, or R⁷ can be a ligand tethered indirectly to the carrier, e.g., through a tethering moiety, e.g., C₁-C₂₀ alkyl substituted with NR^{c}R^{d}; or C₁-C₂₀ alkyl substituted with NHC(O)R^{d};
R⁸ is H or C₁-C₆ alkyl;
R¹³ is hydroxy, C₁-C₄ alkoxy, or halo;
R¹⁴ is NR^{c}R⁷;
R¹⁵ is C₁-C₆ alkyl optionally substituted with cyano, or C₂-C₆ alkenyl;
R¹⁶ is C₁-C₁₀ alkyl;
R¹⁷ is a liquid or solid phase support reagent;
L is —C(O)(CH₂)_{q}C(O)—, or —C(O)(CH₂)_{q}S—;
R^{a} is a protecting group, e.g., CAr₃; (e.g., a dimethoxytrityl group) or
Si(X^{5′})(X^{5″})(X^{5′″}) in which (X^{5′}), (X^{5′″}), and (X^{5′″}) are as described elsewhere.
R^{b} is P(O)(O⁻)H, P(OR¹⁵)N(R¹⁶)₂ or L-R¹⁷;
R^{c} is H or C₁-C₆ alkyl;
R^{d} is H or a ligand;
Each Ar is, independently, C₆-C₁₀ aryl optionally substituted with C₁-C₄ alkoxy;
n is 1-4; and q is 0-4.

Exemplary carriers include those in which, e.g., X is N(CO)R⁷ or NR7, Y is CR⁹R¹⁰, and Z is absent; or X is N(CO)R⁷ or NR⁷, Y is CR⁹R¹⁰, and Z is CR¹¹R¹²; or X is N(CO)R⁷ or NR⁷, Y is NR⁸, and Z is CR¹¹R¹²; or X is N(CO)R⁷ or NR⁷, Y is O, and Z is CR¹¹R¹²; or X is CH₂; Y is CR⁹R¹⁰; Z is CR¹¹R¹², and R⁵ and R¹¹ together form C₆ cycloalkyl (H, z=2), or the indane ring system, e.g., X is CH₂; Y is CR⁹R¹⁰; Z is CR¹¹R¹², and R⁵ and R¹¹ together form C₅ cycloalkyl (H, z=1).

In certain embodiments, the carrier may be based on the pyrroline ring system or the 4-hydroxyproline ring system, e.g., X is N(CO)R⁷ or NR⁷, Y is CR⁹R¹⁰, and Z is absent (D).

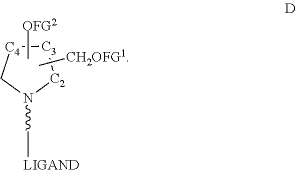

OFG¹ is preferably attached to a primary carbon, e.g., an exocyclic alkylene group, e.g., a methylene group, connected to one of the carbons in the five-membered ring (—CH₂OFG¹ in D). OFG² is preferably attached directly to one of the carbons in the five-membered ring (—OFG² in D). For the pyrroline-based carriers, —CH₂OFG¹ may be attached to C-2 and OFG² may be attached to C-3; or —CH₂OFG¹ may be attached to C-3 and OFG² may be attached to C-4. In certain embodiments, CH₂OFG¹ and OFG² may be geminally substituted to one of the above-referenced carbons. For the 3-hydroxyproline-based carriers, —CH₂OFG¹ may be attached to C-2 and OFG² may be attached to C-4. The pyrroline- and 4-hydroxyproline-based monomers may therefore contain linkages (e.g., carbon-carbon bonds) wherein bond rotation is restricted about that particular linkage, e.g. restriction resulting from the presence of a ring. Thus, CH₂OFG¹ and OFG² may be cis or trans with respect to one another in any of the pairings delineated above. Accordingly, all cis/trans isomers are expressly included. The monomers may also contain one or more asymmetric centers and thus occur as racemates and racemic mixtures, single enantiomers, individual diastereomers and diastereomeric mixtures. All such isomeric forms of the monomers are expressly included (e.g., the centers bearing CH₂OFG¹ and OFG² can both have the R configuration; or both have the S configuration; or one center can have the R configuration and the other center can have the S configuration and vice versa). The tethering attachment point is preferably nitrogen. Preferred examples of carrier D include the following:

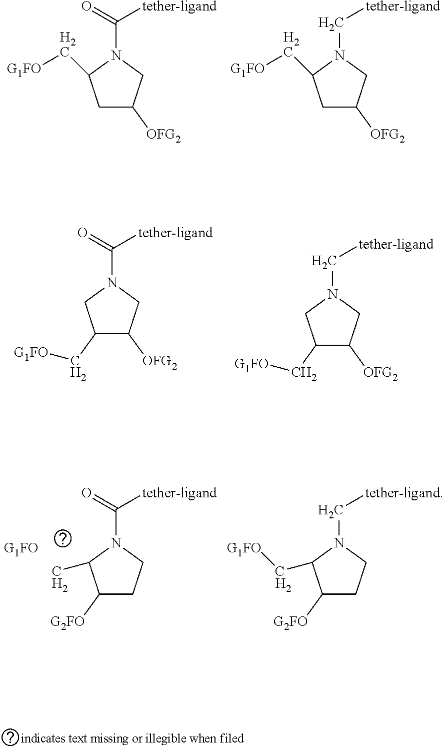

In certain embodiments, the carrier may be based on the piperidine ring system (E), e.g., X is N(CO)R⁷ or NR⁷, Y is CR⁹R¹⁰, and Z is CR¹¹R¹².

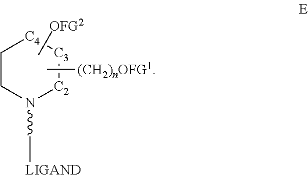

OFG¹ is preferably attached to a primary carbon, e.g., an exocyclic alkylene group, e.g., a methylene group (n=1) or ethylene group (n=2), connected to one of the carbons in the six-membered ring [—(CH₂)ₙOFG¹ in E]. OFG² is preferably attached directly to one of the carbons in the six-membered ring (—OFG² in E). —(CH₂)ₙOFG¹ and OFG² may be disposed in a geminal manner on the ring, i.e., both groups may be attached to the same carbon, e.g., at C-2, C-3, or C-4. Alternatively, —(CH₂)ₙOFG¹ and OFG² may be disposed in a vicinal manner on the ring, i.e., both groups may be attached to adjacent ring carbon atoms, e.g., —(CH₂)ₙOFG¹ may be attached to C-2 and OFG² may be attached to C-3; —(CH₂)ₙOFG¹ may be attached to C-3 and OFG² may be attached to C-2; —(CH₂)ₙOFG¹ may be attached to C-3 and OFG² may be attached to C-4; or —(CH₂)ₙOFG¹ may be attached to C-4 and OFG² may be attached to C-3. The piperidine-based monomers may therefore contain linkages (e.g., carbon-carbon bonds) wherein bond rotation is restricted about that particular linkage, e.g. restriction resulting from the presence of a ring. Thus, —(CH₂)ₙOFG¹ and OFG² may be cis or trans with respect to one another in any of the pairings delineated above. Accordingly, all cis/trans isomers are expressly included. The monomers may also contain one or more asymmetric centers and thus occur as racemates and racemic mixtures, single enantiomers, individual diastereomers and diastereomeric mixtures. All such isomeric forms of the monomers are expressly included (e.g., the centers bearing CH₂OFG¹ and OFG² can both have the R configuration; or both have the S configuration; or one center can have the R configuration and the other center can have the S configuration and vice versa). The tethering attachment point is preferably nitrogen.

In certain embodiments, the carrier may be based on the piperazine ring system (F), e.g., X is N(CO)R⁷ or NR⁷, Y is NR⁸, and Z is CR¹¹R¹², or the morpholine ring system (G), e.g., X is N(CO)R⁷ or NR⁷, Y is O, and Z is CR¹¹R¹².

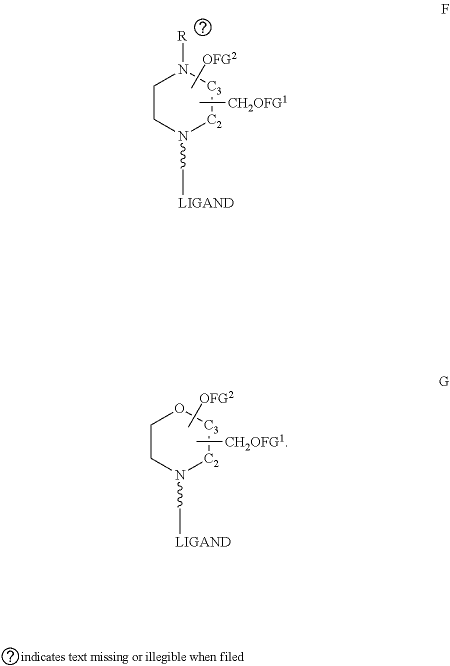

OFG¹ is preferably attached to a primary carbon, e.g., an exocyclic alkylene group, e.g., a methylene group, connected to one of the carbons in the six-membered ring (—CH₂OFG¹ in F or G). OFG² is preferably attached directly to one of the carbons in the six-membered rings (—OFG² in F or G). For both F and G, —CH₂OFG¹ may be attached to C-2 and OFG² may be attached to C-3; or vice versa. In certain embodiments, CH₂OFG¹ and OFG² may be geminally substituted to one of the above-referenced carbons. The piperazine- and morpholine-based monomers may therefore contain linkages (e.g., carbon-carbon bonds) wherein bond rotation is restricted about that particular linkage, e.g. restriction resulting from the presence of a ring. Thus, CH₂OFG¹ and OFG² may be cis or trans with respect to one another in any of the pairings delineated above. Accordingly, all cis/trans isomers are expressly included. The monomers may also contain one or more asymmetric centers and thus occur as racemates and racemic mixtures, single enantiomers, individual diastereomers and diastereomeric mixtures. All such isomeric forms of the monomers are expressly included (e.g., the centers bearing CH₂OFG¹ and OFG² can both have the R configuration; or both have the S configuration; or one center can have the R configuration and the other center can have the S configuration and vice versa). R′″ can be, e.g., C1-C₆ alkyl, preferably CH₃. The tethering attachment point is preferably nitrogen in both F and G.

In certain embodiments, the carrier may be based on the decalin ring system, e.g., X is CH₂; Y is CR⁹R¹⁰; Z is CR¹¹R¹², and R⁵ and R¹¹ together form C₆ cycloalkyl (H, z=2), or the indane ring system, e.g., X is CH₂; Y is CR⁹R¹⁰; Z is CR¹¹R¹², and R⁵ and R¹¹ together form C₅ cycloalkyl (H, z=1).

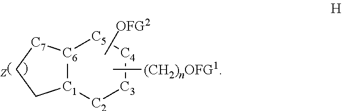

OFG¹ is preferably attached to a primary carbon, e.g., an exocyclic methylene group (n═1) or ethylene group (n═2) connected to one of C-2, C-3, C-4, or C-5 [—(CH₂)ₙOFG¹ in H]. OFG² is preferably attached directly to one of C-2, C-3, C-4, or C-5 (—OFG² in H). —(CH₂)ₙOFG¹ and OFG² may be disposed in a geminal manner on the ring, i.e., both groups may be attached to the same carbon, e.g., at C-2, C-3, C-4, or C-5. Alternatively, —(CH₂)ₙOFG¹ and OFG² may be disposed in a vicinal manner on the ring, i.e., both groups may be attached to adjacent ring carbon atoms, e.g., —(CH₂)ₙOFG¹ may be attached to C-2 and OFG² may be attached to C-3; —(CH₂)ₙOFG¹ may be attached to C-3 and OFG² may be attached to C-2; —(CH₂)ₙOFG¹ may be attached to C-3 and OFG² may be attached to C-4; or —(CH₂)ₙOFG¹ may be attached to C-4 and OFG² may be attached to C-3; —(CH₂)ₙOFG¹ may be attached to C-4 and OFG² may be attached to C-5; or —(CH₂)ₙOFG¹ may be attached to C-5 and OFG² may be attached to C-4. The decalin or indane-based monomers may therefore contain linkages (e.g., carbon-carbon bonds) wherein bond rotation is restricted about that particular linkage, e.g. restriction resulting from the presence of a ring. Thus, —(CH₂)ₙOFG¹ and OFG² may be cis or trans with respect to one another in any of the pairings delineated above. Accordingly, all cis/trans isomers are expressly included. The monomers may also contain one or more asymmetric centers and thus occur as racemates and racemic mixtures, single enantiomers, individual diastereomers and diastereomeric mixtures. All such isomeric forms of the monomers are expressly included (e.g., the centers bearing CH₂OFG¹ and OFG² can both have the R configuration; or both have the S configuration; or one center can have the R configuration and the other center can have the S configuration and vice versa). In a preferred embodiment, the substituents at C-1 and C-6 are trans with respect to one another. The tethering attachment point is preferably C-6 or C-7.

Other carriers may include those based on 3-hydroxyproline (J).

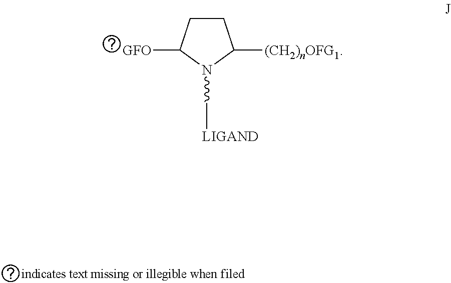

Thus, —(CH₂),OFG¹ and OFG² may be cis or trans with respect to one another. Accordingly, all cis/trans isomers are expressly included. The monomers may also contain one or more asymmetric centers and thus occur as racemates and racemic mixtures, single enantiomers, individual diastereomers and diastereomeric mixtures. All such isomeric forms of the monomers are expressly included (e.g., the centers bearing CH₂OFG¹ and OFG² can both have the R configuration; or both have the S configuration; or one center can have the R configuration and the other center can have the S configuration and vice versa). The tethering attachment point is preferably nitrogen.

Details about more representative cyclic, sugar replacement-based carriers can be found in U.S. Pat. Nos. 7,745,608 and 8,017,762, which are herein incorporated by reference in their entireties.

### Sugar Replacement-Based Monomers (Acyclic)

Acyclic sugar replacement-based monomers, e.g., sugar replacement-based ligand-conjugated monomers, are also referred to herein as ribose replacement monomer subunit (RRMS) monomer compounds. Preferred acyclic carriers can have formula LCM-3 or LCM-4:

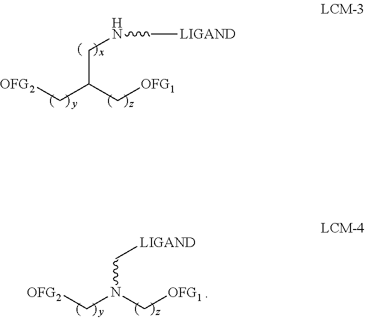

In some embodiments, each of x, y, and z can be, independently of one another, 0, 1, 2, or 3. In formula LCM-3, when y and z are different, then the tertiary carbon can have either the R or S configuration. In preferred embodiments, x is zero and y and z are each 1 in formula LCM-3 (e.g., based on serinol), and y and z are each 1 in formula LCM-3. Each of formula LCM-3 or LCM-4 below can optionally be substituted, e.g., with hydroxy, alkoxy, perhaloalkyl.

Details about more representative acyclic, sugar replacement-based carriers can be found in U.S. Pat. Nos. 7,745,608 and 8,017,762, which are herein incorporated by reference in their entireties.

In some embodiments, the oligonucleotide comprises one or more targeting ligands conjugated to the 5′ end of the sense strand or the 5′ end of the antisense strand, optionally via a carrier and/or linker/tether.

In some embodiments, the oligonucleotide comprises one or more targeting ligands conjugated to the 3′ end of the sense strand or the 3′ end of the antisense strand, optionally via a carrier and/or linker/tether.

In some embodiments, the oligonucleotide comprises one or more targeting ligands conjugated to both ends of the sense strand, optionally via a carrier and/or linker/tether.

In some embodiments, the oligonucleotide comprises one or more targeting ligands conjugated to both ends of the antisense strand, optionally via a carrier and/or linker/tether.

In some embodiments, the oligonucleotide comprises one or more targeting ligands conjugated to internal position(s) of the sense or antisense strand, optionally via a carrier and/or linker/tether.

In some embodiments, one or more targeting ligands are conjugated to the ribose, nucleobase, and/or at the internucleotide linkages. In some embodiments, one or more targeting ligands are conjugated to the ribose at the 2′ position, 3′ position, 4′ position, and/or 5′ position of the ribose. In some embodiments, one or more targeting ligands are conjugated at the nucleobase of natural (such as A, T, G, C, or U) or modified as defined herein. In some embodiments, one or more targeting ligands are conjugated at the phosphate or 2′-modified nucleoside as defined herein.

In some embodiments, the oligonucleotide comprises one or more targeting ligands conjugated to the 5′ end or 3′ end of the sense strand, and one or more same or different targeting ligands conjugated to the 5′ end or 3′ end of the antisense strand,

In some embodiments, at least one targeting ligand is located on one or more terminal positions of the sense strand or antisense strand. In one embodiment, at least one targeting ligand is located on the 3′ end or 5′ end of the sense strand. In one embodiment, at least one targeting ligand is located on the 3′ end or 5′ end of the antisense strand.

In some embodiments, at least one targeting ligand is conjugated to one or more internal positions on at least one strand. Internal positions of a strand refers to the nucleotide on any position of the strand, except the terminal position from the 3′ end and 5′ end of the strand (e.g., excluding 2 positions: position 1 counting from the 3′ end and position 1 counting from the 5′ end).

In one embodiment, at least one targeting ligand is located on one or more internal positions on at least one strand, which include all positions except the terminal two positions from each end of the strand (e.g., excluding 4 positions: positions 1 and 2 counting from the 3′ end and positions 1 and 2 counting from the 5′ end). In one embodiment, the targeting ligand is located on one or more internal positions on at least one strand, which include all positions except the terminal three positions from each end of the strand (e.g., excluding 6 positions: positions 1, 2, and 3 counting from the 3′ end and positions 1, 2, and 3 counting from the 5′ end).

In one embodiment, at least one targeting ligand is located on one or more positions of at least one end of the duplex region, which include all positions within the duplex region, but not include the overhang region or the carrier that replaces the terminal nucleotide on the 3′ end of the sense strand.

In one embodiment, at least one targeting ligand is located on the sense strand within the first five, four, three, two, or first base pairs at the 5′-end of the antisense strand of the duplex region.

In one embodiment, at least one targeting ligand (e.g., a lipophilic moiety) is located on one or more internal positions on at least one strand, except the cleavage site region of the sense strand, for instance, the targeting ligand (e.g., a lipophilic moiety) is not located on positions 9-12 counting from the 5′-end of the sense strand, for example, the targeting ligand (e.g., a lipophilic moiety) is not located on positions 9-11 counting from the 5′-end of the sense strand. Alternatively, the internal positions exclude positions 11-13 counting from the 3′-end of the sense strand.

In one embodiment, at least one targeting ligand (e.g., a lipophilic moiety) is located on one or more internal positions on at least one strand, which exclude the cleavage site region of the antisense strand. For instance, the internal positions exclude positions 12-14 counting from the 5′-end of the antisense strand.

In one embodiment, at least one targeting ligand (e.g., a lipophilic moiety) is located on one or more internal positions on at least one strand, which exclude positions 11-13 on the sense strand, counting from the 3′-end, and positions 12-14 on the antisense strand, counting from the 5′-end.

In one embodiment, one or more targeting ligands (e.g., a lipophilic moiety) are located on one or more of the following internal positions: positions 4-8 and 13-18 on the sense strand, and positions 6-10 and 15-18 on the antisense strand, counting from the 5′end of each strand.

In one embodiment, one or more targeting ligands (e.g., a lipophilic moiety) are located on one or more of the following internal positions: positions 5, 6, 7, 15, and 17 on the sense strand, and positions 15 and 17 on the antisense strand, counting from the 5′end of each strand.

### Target Genes

Without limitations, target genes for siRNAs include, but are not limited to genes promoting unwanted cell proliferation, growth factor gene, growth factor receptor gene, genes expressing kinases, an adaptor protein gene, a gene encoding a G protein super family molecule, a gene encoding a transcription factor, a gene which mediates angiogenesis, a viral gene, a gene required for viral replication, a cellular gene which mediates viral function, a gene of a bacterial pathogen, a gene of an amoebic pathogen, a gene of a parasitic pathogen, a gene of a fungal pathogen, a gene which mediates an unwanted immune response, a gene which mediates the processing of pain, a gene which mediates a neurological disease, an allene gene found in cells characterized by loss of heterozygosity, or one allege gene of a polymorphic gene.

Specific exemplary target genes for the siRNAs include, but are not limited to, PCSK-9, ApoC3, AT3, AGT, ALAS1, TMPR, HAO1, AGT, C5, CCR-5, PDGF beta gene; Erb-B gene, Src gene; CRK gene; GRB2 gene; RAS gene; MEKK gene; INK gene; RAF gene; Erk1/2 gene; PCNA(p21) gene; MYB gene; c-MYC gene; JUN gene; FOS gene; BCL-2 gene; Cyclin D gene; VEGF gene; EGFR gene; Cyclin A gene; Cyclin E gene; WNT-1 gene; beta-catenin gene; c-MET gene; PKC gene; NFKB gene; STAT3 gene; survivin gene; Her2/Neu gene; topoisomerase I gene; topoisomerase II alpha gene; p73 gene; p21(WAF1/CIP1) gene, p27(KIP1) gene; PPM1D gene; caveolin I gene; MIB I gene; MTAI gene; M68 gene; tumor suppressor genes; p53 gene; DN-p63 gene; pRb tumor suppressor gene; APC1 tumor suppressor gene; BRCA1 tumor suppressor gene; PTEN tumor suppressor gene; MLL fusion genes, e.g., MLL-AF9, BCR/ABL fusion gene; TEL/AML1 fusion gene; EWS/FLI1 fusion gene; TLS/FUS1 fusion gene; PAX3/FKHR fusion gene; AML1/ETO fusion gene; alpha v-integrin gene; Flt-1 receptor gene; tubulin gene; Human Papilloma Virus gene, a gene required for Human Papilloma Virus replication, Human Immunodeficiency Virus gene, a gene required for Human Immunodeficiency Virus replication, Hepatitis A Virus gene, a gene required for Hepatitis A Virus replication, Hepatitis B Virus gene, a gene required for Hepatitis B Virus replication, Hepatitis C Virus gene, a gene required for Hepatitis C Virus replication, Hepatitis D Virus gene, a gene required for Hepatitis D Virus replication, Hepatitis E Virus gene, a gene required for Hepatitis E Virus replication, Hepatitis F Virus gene, a gene required for Hepatitis F Virus replication, Hepatitis G Virus gene, a gene required for Hepatitis G Virus replication, Hepatitis H Virus gene, a gene required for Hepatitis H Virus replication, Respiratory Syncytial Virus gene, a gene that is required for Respiratory Syncytial Virus replication, Herpes Simplex Virus gene, a gene that is required for Herpes Simplex Virus replication, herpes Cytomegalovirus gene, a gene that is required for herpes Cytomegalovirus replication, herpes Epstein Barr Virus gene, a gene that is required for herpes Epstein Barr Virus replication, Kaposi's Sarcoma-associated Herpes Virus gene, a gene that is required for Kaposi's Sarcoma-associated Herpes Virus replication, JC Virus gene, human gene that is required for JC Virus replication, myxovirus gene, a gene that is required for myxovirus gene replication, rhinovirus gene, a gene that is required for rhinovirus replication, coronavirus gene, a gene that is required for coronavirus replication, West Nile Virus gene, a gene that is required for West Nile Virus replication, St. Louis Encephalitis gene, a gene that is required for St. Louis Encephalitis replication, Tick-borne encephalitis virus gene, a gene that is required for Tick-borne encephalitis virus replication, Murray Valley encephalitis virus gene, a gene that is required for Murray Valley encephalitis virus replication, dengue virus gene, a gene that is required for dengue virus gene replication, Simian Virus 40 gene, a gene that is required for Simian Virus 40 replication, Human T Cell Lymphotropic Virus gene, a gene that is required for Human T Cell Lymphotropic Virus replication, Moloney-Murine Leukemia Virus gene, a gene that is required for Moloney-Murine Leukemia Virus replication, encephalomyocarditis virus gene, a gene that is required for encephalomyocarditis virus replication, measles virus gene, a gene that is required for measles virus replication, Vericella zoster virus gene, a gene that is required for Vericella zoster virus replication, adenovirus gene, a gene that is required for adenovirus replication, yellow fever virus gene, a gene that is required for yellow fever virus replication, poliovirus gene, a gene that is required for poliovirus replication, poxvirus gene, a gene that is required for poxvirus replication, plasmodium gene, a gene that is required for plasmodium gene replication, Mycobacterium ulcerans gene, a gene that is required for Mycobacterium ulcerans replication, Mycobacterium tuberculosis gene, a gene that is required for Mycobacterium tuberculosis replication, Mycobacterium leprae gene, a gene that is required for Mycobacterium leprae replication, Staphylococcus aureus gene, a gene that is required for Staphylococcus aureus replication, Streptococcus pneumoniae gene, a gene that is required for Streptococcus pneumoniae replication, Streptococcus pyogenes gene, a gene that is required for Streptococcus pyogenes replication, Chlamydia pneumoniae gene, a gene that is required for Chlamydia pneumoniae replication, Mycoplasma pneumoniae gene, a gene that is required for Mycoplasma pneumoniae replication, an integrin gene, a selectin gene, complement system gene, chemokine gene, chemokine receptor gene, GCSF gene, Gro1 gene, Gro2 gene, Gro3 gene, PF4 gene, MIG gene, Pro-Platelet Basic Protein gene, MIP-1I gene, MIP-1J gene, RANTES gene, MCP-1 gene, MCP-2 gene, MCP-3 gene, CMBKR1 gene, CMBKR2 gene, CMBKR3 gene, CMBKR5v, AIF-1 gene, I-309 gene, a gene to a component of an ion channel, a gene to a neurotransmitter receptor, a gene to a neurotransmitter ligand, amyloid-family gene, presenilin gene, HD gene, DRPLA gene, SCA1 gene, SCA2 gene, MJD1 gene, CACNLlA4 gene, SCA7 gene, SCA8 gene, allele gene found in loss of heterozygosity (LOH) cells, one allele gene of a polymorphic gene and combinations thereof.

The loss of heterozygosity (LOH) can result in hemizygosity for sequence, e.g., genes, in the area of LOH. This can result in a significant genetic difference between normal and disease-state cells, e.g., cancer cells, and provides a useful difference between normal and disease-state cells, e.g., cancer cells. This difference can arise because a gene or other sequence is heterozygous in duploid cells but is hemizygous in cells having LOH. The regions of LOH will often include a gene, the loss of which promotes unwanted proliferation, e.g., a tumor suppressor gene, and other sequences including, e.g., other genes, in some cases a gene which is essential for normal function, e.g., growth. Methods of the invention rely, in part, on the specific modulation of one allele of an essential gene with a composition of the invention.

In certain embodiments, the invention provides an olignucleotide that modulates a micro-RNA.

### Targeting CNS

In some embodiments, the invention provides an oligonucleotide that targets APP for Early Onset Familial Alzheimer Disease, ATXN2 for Spinocerebellar Ataxia 2 and ALS, and C9orf72 for Amyotrophic Lateral Sclerosis and Frontotemporal Dementia.

In some embodiments, the invention provides an oligonucleotide that targets TARDBP for ALS, MAPT (Tau) for Frontotemporal Dementia, and HTT for Huntington Disease.

In some embodiments, the invention provides an oligonucleotide that targets SNCA for Parkinson Disease, FUS for ALS, ATXN3 for Spinocerebellar Ataxia 3, ATXN1 for SCA1, genes for SCA7 and SCA8, ATN1 for DRPLA, MeCP2 for XLMR, PRNP for Prion Diseases, recessive CNS disorders: Lafora Disease, DMPK for DM1 (CNS and Skeletal Muscle), and TTR for hATTR (CNS, ocular and systemic).

Spinocerebellar ataxia is an inherited brain-function disorder. Dominantly inherited forms of spinocerebellar ataxias, such as SCA1-8, are devastating disorders with no disease-modifying therapy. Exemplary targets include SCA2, SCA3, and SCA1.

More detailed descriptions about these CNS targeting receptors and related diseases may be found in PCT Application No. PCT/US20/59399, entitled “Extrahepatic Delivery,” filed on Nov. 6, 2020, the content of which is incorporated herein by reference in its entirety.

In some embodiments, the invention provides an oligonucleotide that target genes for diseases including, but are not limited to, age-related macular degeneration (AMD) (dry and wet), birdshot chorioretinopathy, dominant retinitis pigmentosa 4, Fuch's dystrophy, hATTR amyloidosis, hereditary and sporadic glaucoma, and stargardt's disease.

In some embodiments, the oligonucleotide targets VEGF for wet (or exudative) AMD.

In some embodiments, the oligonucleotide targets C3 for dry (or nonexudative) AMD.

In some embodiments, the oligonucleotide targets CFB for dry (or nonexudative) AMD.

In some embodiments, the oligonucleotide targets MYOC for glaucoma.

In some embodiments, the oligonucleotide targets ROCK2 for glaucoma.

In some embodiments, the oligonucleotide targets ADRB2 for glaucoma.

In some embodiments, the oligonucleotide targets CA2 for glaucoma.

In some embodiments, the oligonucleotide targets CRYGC for cataract.

In some embodiments, the oligonucleotide targets PPP3CB for dry eye syndrome.

### Ligands

In certain embodiments, the oligonucleotide is further modified by covalent attachment of one or more conjugate groups. In general, conjugate groups modify one or more properties of the attached compound of the invention including but not limited to pharmacodynamic, pharmacokinetic, binding, absorption, cellular distribution, cellular uptake, charge and clearance. Conjugate groups are routinely used in the chemical arts and are linked directly or via an optional linking moiety or linking group to a parent compound such as an oligonucleotide. A preferred list of conjugate groups includes without limitation, intercalators, reporter molecules, polyamines, polyamides, polyethylene glycols, thioethers, polyethers, cholesterols, thiocholesterols, cholic acid moieties, folate, lipids, phospholipids, biotin, phenazine, phenanthridine, anthraquinone, adamantane, acridine, fluoresceins, rhodamines, coumarins and dyes.

In some embodiments, the oligonucleotide further comprises a targeting ligand that targets a receptor which mediates delivery to a specific CNS tissue. These targeting ligands can also be conjugated in combination with a lipophilic moiety to enable specific intrathecal and systemic delivery.

Exemplary targeting ligands that targets the receptor mediated delivery to a CNS tissue are peptide ligands such as Angiopep-2, lipoprotein receptor related protein (LRP) ligand, bEnd.3 cell binding ligand; transferrin receptor (TfR) ligand (which can utilize iron transport system in brain and cargo transport into the brain parenchyma); manose receptor ligand (which targets olfactory ensheathing cells, glial cells), glucose transporter protein, and LDL receptor ligand.

In some embodiments, the oligonucleotide further comprises a targeting ligand that targets a receptor which mediates delivery to a specific ocular tissue. These targeting ligands can also be conjugated in combination with a lipophilic moiety to enable specific ocular delivery (e.g., intravitreal delivery) and systemic delivery. Exemplary targeting ligands that targets the receptor mediated delivery to a ocular tissue are lipophilic ligands such as all-trans retinol (which targets the retinoic acid receptor); RGD peptide (which targets retinal pigment epithelial cells), such as H-Gly-Arg-Gly-Asp-Ser-Pro-Lys-Cys-OH or Cyclo(-Arg-Gly-Asp-D-Phe-Cys; LDL receptor ligands; and carbohydrate based ligands (which targets endothelial cells in posterior eye).

Preferred conjugate groups amenable to the present invention include lipid moieties such as a cholesterol moiety (Letsinger et al., Proc. Natl. Acad. Sci. USA, 1989, 86, 6553); cholic acid (Manoharan et al., Bioorg. Med. Chem. Lett., 1994, 4, 1053); a thioether, e.g., hexyl-S-tritylthiol (Manoharan et al., Ann. N.Y. Acad. Sci., 1992, 660, 306; Manoharan et al., Bioorg. Med. Chem. Let., 1993, 3, 2765); a thiocholesterol (Oberhauser et al., Nucl. Acids Res., 1992, 20, 533); an aliphatic chain, e.g., dodecandiol or undecyl residues (Saison-Behmoaras et al., EMBO J., 1991, 10, 111; Kabanov et al., FEBS Lett., 1990, 259, 327; Svinarchuk et al., Biochimie, 1993, 75, 49); a phospholipid, e.g., di-hexadecyl-rac-glycerol or triethylammonium-1,2-di-O-hexadecyl-rac-glycero-3-H-phosphonate (Manoharan et al., Tetrahedron Lett., 1995, 36, 3651; Shea et al., Nucl. Acids Res., 1990, 18, 3777); a polyamine or a polyethylene glycol chain (Manoharan et al., Nucleosides & Nucleotides, 1995, 14, 969); adamantane acetic acid (Manoharan et al., Tetrahedron Lett., 1995, 36, 3651); a palmityl moiety (Mishra et al., Biochim. Biophys. Acta, 1995, 1264, 229); or an octadecylamine or hexylamino-carbonyl-oxycholesterol moiety (Crooke et al., J. Pharmacol. Exp. Ther., 1996, 277, 923).

Generally, a wide variety of entities, e.g., ligands, can be coupled to the oligonucleotides described herein. Ligands can include naturally occurring molecules, or recombinant or synthetic molecules. Exemplary ligands include, but are not limited to, polylysine (PLL), poly L-aspartic acid, poly L-glutamic acid, styrene-maleic acid anhydride copolymer, poly(L-lactide-co-glycolied) copolymer, divinyl ether-maleic anhydride copolymer, N-(2-hydroxypropyl)methacrylamide copolymer (HMPA), polyethylene glycol (PEG, e.g., PEG-2K, PEG-5K, PEG-10K, PEG-12K, PEG-15K, PEG-20K, PEG-40K), MPEG, [MPEG]₂, polyvinyl alcohol (PVA), polyurethane, poly(2-ethylacryllic acid), N-isopropylacrylamide polymers, polyphosphazine, polyethylenimine, cationic groups, spermine, spermidine, polyamine, pseudopeptide-polyamine, peptidomimetic polyamine, dendrimer polyamine, arginine, amidine, protamine, cationic lipid, cationic porphyrin, quaternary salt of a polyamine, thyrotropin, melanotropin, lectin, glycoprotein, surfactant protein A, mucin, glycosylated polyaminoacids, transferrin, bisphosphonate, polyglutamate, polyaspartate, aptamer, asialofetuin, hyaluronan, procollagen, immunoglobulins (e.g., antibodies), insulin, transferrin, albumin, sugar-albumin conjugates, intercalating agents (e.g., acridines), cross-linkers (e.g. psoralen, mitomycin C), porphyrins (e.g., TPPC4, texaphyrin, Sapphyrin), polycyclic aromatic hydrocarbons (e.g., phenazine, dihydrophenazine), artificial endonucleases (e.g., EDTA), lipophilic molecules (e.g., steroids, bile acids, cholesterol, cholic acid, adamantane acetic acid, 1-pyrene butyric acid, dihydrotestosterone, 1,3-Bis-O(hexadecyl)glycerol, geranyloxyhexyl group, hexadecylglycerol, borneol, menthol, 1,3-propanediol, heptadecyl group, palmitic acid, myristic acid,03-(oleoyl)lithocholic acid, 03-(oleoyl)cholenic acid, dimethoxytrityl, or phenoxazine), peptides (e.g., an alpha helical peptide, amphipathic peptide, RGD peptide, cell permeation peptide, endosomolytic/fusogenic peptide), alkylating agents, phosphate, amino, mercapto, polyamino, alkyl, substituted alkyl, radiolabeled markers, enzymes, haptens (e.g. biotin), transport/absorption facilitators (e.g., naproxen, aspirin, vitamin E, folic acid), synthetic ribonucleases (e.g., imidazole, bisimidazole, histamine, imidazole clusters, acridine-imidazole conjugates, Eu3+ complexes of tetraazamacrocycles), dinitrophenyl, HRP, AP, antibodies, hormones and hormone receptors, lectins, carbohydrates, multivalent carbohydrates, vitamins (e.g., vitamin A, vitamin E, vitamin K, vitamin B, e.g., folic acid, B12, riboflavin, biotin and pyridoxal), vitamin cofactors, lipopolysaccharide, an activator of p38 MAP kinase, an activator of NF-1B, taxon, vincristine, vinblastine, cytochalasin, nocodazole, japlakinolide, latrunculin A, phalloidin, swinholide A, indanocine, myoservin, tumor necrosis factor alpha (TNFalpha), interleukin-1 beta, gamma interferon, natural or recombinant low density lipoprotein (LDL), natural or recombinant high-density lipoprotein (HDL), and a cell-permeation agent (e.g., a helical cell-permeation agent).

Peptide and peptidomimetic ligands include those having naturally occurring or modified peptides, e.g., D or L peptides; a, 3, or 7 peptides; N-methyl peptides; azapeptides; peptides having one or more amide, i.e., peptide, linkages replaced with one or more urea, thiourea, carbamate, or sulfonyl urea linkages; or cyclic peptides. A peptidomimetic (also referred to herein as an oligopeptidomimetic) is a molecule capable of folding into a defined three-dimensional structure similar to a natural peptide. The peptide or peptidomimetic ligand can be about 5-50 amino acids long, e.g., about 5, 10, 15, 20, 25, 30, 35, 40, 45, or 50 amino acids long.

Exemplary amphipathic peptides include, but are not limited to, cecropins, lycotoxins, paradaxins, buforin, CPF, bombinin-like peptide (BLP), cathelicidins, ceratotoxins, S. clava peptides, hagfish intestinal antimicrobial peptides (HFIAPs), magainines, brevinins-2, dermaseptins, melittins, pleurocidin, H₂A peptides, Xenopus peptides, esculentinis-1, and caerins.

As used herein, the term “endosomolytic ligand” refers to molecules having endosomolytic properties. Endosomolytic ligands promote the lysis of and/or transport of the composition of the invention, or its components, from the cellular compartments such as the endosome, lysosome, endoplasmic reticulum (ER), Golgi apparatus, microtubule, peroxisome, or other vesicular bodies within the cell, to the cytoplasm of the cell. Some exemplary endosomolytic ligands include, but are not limited to, imidazoles, poly or oligoimidazoles, linear or branched polyethyleneimines (PEIs), linear and branched polyamines, e.g. spermine, cationic linear and branched polyamines, polycarboxylates, polycations, masked oligo or poly cations or anions, acetals, polyacetals, ketals/polyketals, orthoesters, linear or branched polymers with masked or unmasked cationic or anionic charges, dendrimers with masked or unmasked cationic or anionic charges, polyanionic peptides, polyanionic peptidomimetics, pH-sensitive peptides, natural and synthetic fusogenic lipids, natural and synthetic cationic lipids.

Exemplary endosomolytic/fusogenic peptides include, but are not limited to,

AALEALAEALEALAEALEALAEAAAAGGC (GALA);
AALAEALAEALAEALAEALAEALAAAAGGC (EALA);
ALEALAEALEALAEA;
GLFEAIEGFIENGWEGMIWDYG (INF-7);
GLFGAIAGFIENGWEGMIDGWYG (Inf HA-2);
GLFEAIEGFIENGWEGMIDGWYGCGLFEAIEGF
IENGWEGMID GWYGC (diINF-7);
GLFEAIEGFIENGWEGMIDGGCGLFEAIEGFIEN
GWEGMIDGGC (diINF-3);
GLFGALAEALAEALAEHLAEALAEALEALAAGGSC (GLF);
GLFEAIEGFIENGWEGLAEALAEALEALAAGGSC (GALA-INF3);
GLF EAI EGFI ENGW EGnI DG K GLF EAI EGFI ENGW
EGnI DG (INF-5, n is norleucine);
LFEALLELLESLWELLLEA (JTS-1);
GLFKALLKLLKSLWKLLLKA (ppTG1);
GLFRALLRLLRSLWRLLLRA (ppTG20);
WEAKLAKALAKALAKHLAKALAKALKACEA (KALA);
GLFFEAIAEFIEGGWEGLIEGC (HA);
GIGAVLKVLTTGLPALISWIKRKRQQ (Melittin);
H5WYG;
and
CHK₆HC.

Without wishing to be bound by theory, fusogenic lipids fuse with and consequently destabilize a membrane. Fusogenic lipids usually have small head groups and unsaturated acyl chains. Exemplary fusogenic lipids include, but are not limited to, 1,2-dileoyl-sn-3-phosphoethanolamine (DOPE), phosphatidylethanolamine (POPE), palmitoyloleoylphosphatidylcholine (POPC), (6Z,9Z,28Z,31Z)-heptatriaconta-6,9,28,31-tetraen-19-ol (Di-Lin), N-methyl(2,2-di((9Z,12Z)-octadeca-9,12-dienyl)-1,3-dioxolan-4-yl)methanamine (DLin-k-DMA) and N-methyl-2-(2,2-di((9Z,12Z)-octadeca-9,12-dienyl)-1,3-dioxolan-4-yl)ethanamine (also referred to as XTC herein).

Synthetic polymers with endosomolytic activity amenable to the present invention are described in U.S. Pat. App. Pub. Nos. 2009/0048410; 2009/0023890; 2008/0287630; 2008/0287628; 2008/0281044; 2008/0281041; 2008/0269450; 2007/0105804; 20070036865; and 2004/0198687, contents of which are hereby incorporated by reference in their entirety.

Exemplary cell permeation peptides include, but are not limited to, RQIKIWFQNRRMKWKK (penetratin); GRKKRRQRRRPPQC (Tat fragment 48-60); GALFLGWLGAAGSTMGAWSQPKKKRKV (signal sequence based peptide); LLIILRRRIRKQAHAHSK (PVEC); GWTLNSAGYLLKINLKALAALAKKIL (transportan); KLALKLALKALKAALKLA (amphiphilic model peptide); RRRRRRRRR (Arg9); KFFKFFKFFK (Bacterial cell wall permeating peptide); LLGDFFRKSKEKIGKEFKRIVQRIKDFLRNLVPRTES (LL-37); SWLSKTAKKLENSAKKRISEGIAIAIQGGPR (cecropin P1); ACYCRIPACIAGERRYGTCIYQGRLWAFCC (a-defensin); DHYNCVSSGGQCLYSACPIFTKIQGTCYRGKAKCCK (0-defensin); RRRPRPPYLPRPRPPPFFPPRLPPRIPPGFPPRFPPRFPGKR-NH2 (PR-39); ILPWKWPWWPWRR-NH2 (indolicidin); AAVALLPAVLLALLAP (RFGF); AALLPVLLAAP (RFGF analogue); and RKCRIVVIRVCR (bactenecin).

Exemplary cationic groups include, but are not limited to, protonated amino groups, derived from e.g., O-AMINE (AMINE═NH₂; alkylamino, dialkylamino, heterocyclyl, arylamino, diaryl amino, heteroaryl amino, or diheteroaryl amino, ethylene diamine, polyamino); aminoalkoxy, e.g., O(CH₂)ₙAMINE, (e.g., AMINE═NH₂; alkylamino, dialkylamino, heterocyclyl, arylamino, diaryl amino, heteroaryl amino, or diheteroaryl amino, ethylene diamine, polyamino); amino (e.g. NH₂; alkylamino, dialkylamino, heterocyclyl, arylamino, diaryl amino, heteroaryl amino, diheteroaryl amino, or amino acid); and NH(CH₂CH₂NH)ₙCH₂CH₂-AMINE (AMINE═NH₂; alkylamino, dialkylamino, heterocyclyl, arylamino, diaryl amino, heteroaryl amino, or diheteroaryl amino).

As used herein the term “targeting ligand” refers to any molecule that provides an enhanced affinity for a selected target, e.g., a cell, cell type, tissue, organ, region of the body, or a compartment, e.g., a cellular, tissue or organ compartment. Some exemplary targeting ligands include, but are not limited to, antibodies, antigens, folates, receptor ligands, carbohydrates, aptamers, integrin receptor ligands, chemokine receptor ligands, transferrin, biotin, serotonin receptor ligands, PSMA, endothelin, GCPII, somatostatin, LDL and HDL ligands.

Carbohydrate based targeting ligands include, but are not limited to, D-galactose, multivalent galactose, N-acetyl-D-galactosamine (GalNAc), multivalent GalNAc, e.g. GalNAc₂ and GalNAc₃ (GalNAc and multivalent GalNAc are collectively referred to herein as GalNAc conjugates); D-mannose, multivalent mannose, multivalent lactose, N-acetyl-glucosamine, Glucose, multivalent Glucose, multivalent fucose, glycosylated polyaminoacids and lectins. The term multivalent indicates that more than one monosaccharide unit is present. Such monosaccharide subunits can be linked to each other through glycosidic linkages or linked to a scaffold molecule.

A number of folate and folate analogs amenable to the present invention as ligands are described in U.S. Pat. Nos. 2,816,110; 5,552,545; 6,335,434 and 7,128,893, contents of which are herein incorporated in their entireties by reference.

As used herein, the terms “PK modulating ligand” and “PK modulator” refers to molecules which can modulate the pharmacokinetics of the composition of the invention. Some exemplary PK modulator include, but are not limited to, lipophilic molecules, bile acids, sterols, phospholipid analogues, peptides, protein binding agents, vitamins, fatty acids, phenoxazine, aspirin, naproxen, ibuprofen, suprofen, ketoprofen, (S)-(+)-pranoprofen, carprofen, PEGs, biotin, and transthyretia-binding ligands (e.g., tetraiidothyroacetic acid, 2, 4, 6-triiodophenol and flufenamic acid). Oligonucleotides that comprise a number of phosphorothioate intersugar linkages are also known to bind to serum protein, thus short oligonucleotides, e.g. oligonucleotides of comprising from about 5 to 30 nucleotides (e.g., 5 to 25 nucleotides, preferably 5 to 20 nucleotides, e.g., 5, 6, 7, 8, 9, 10, 11, 12, 13, 14, 15, 16, 17, 18, 19, or 20 nucleotides), and that comprise a plurality of phosphorothioate linkages in the backbone are also amenable to the present invention as ligands (e.g. as PK modulating ligands). The PK modulating oligonucleotide can comprise at least 3, 4, 5, 6, 7, 8, 9, 10, 11, 12, 13, 14, 15 or more phosphorothioate and/or phosphorodithioate linkages. In some embodiments, all internucleotide linkages in PK modulating oligonucleotide are phosphorothioate and/or phosphorodithioates linkages. In addition, aptamers that bind serum components (e.g. serum proteins) are also amenable to the present invention as PK modulating ligands. Binding to serum components (e.g. serum proteins) can be predicted from albumin binding assays, such as those described in Oravcova, et al., Journal of Chromatography B (1996), 677: 1-27.

When two or more ligands are present, the ligands can all have same properties, all have different properties or some ligands have the same properties while others have different properties. For example, a ligand can have targeting properties, have endosomolytic activity or have PK modulating properties. In a preferred embodiment, all the ligands have different properties.

The ligand or tethered ligand can be present on a monomer when said monomer is incorporated into a component of the compound of the invention (e.g., a compound of the invention or linker). In some embodiments, the ligand can be incorporated via coupling to a “precursor” monomer after said “precursor” monomer has been incorporated into a component of the compound of the invention (e.g., a compound of the invention or linker). For example, a monomer having, e.g., an amino-terminated tether (i.e., having no associated ligand), e.g., monomer-linker-NH₂ can be incorporated into a component of the compounds of the invention (e.g., a compound of the invention or linker). In a subsequent operation, i.e., after incorporation of the precursor monomer into a component of the compounds of the invention (e.g., a compound of the invention or linker), a ligand having an electrophilic group, e.g., a pentafluorophenyl ester or aldehyde group, can subsequently be attached to the precursor monomer by coupling the electrophilic group of the ligand with the terminal nucleophilic group of the precursor monomer's tether.

In another example, a monomer having a chemical group suitable for taking part in Click Chemistry reaction can be incorporated e.g., an azide or alkyne terminated tether/linker. In a subsequent operation, i.e., after incorporation of the precursor monomer into the strand, a ligand having complementary chemical group, e.g. an alkyne or azide can be attached to the precursor monomer by coupling the alkyne and the azide together.

In some embodiments, ligand can be conjugated to nucleobases, sugar moieties, or internucleosidic linkages of the oligonucleotide. Conjugation to purine nucleobases or derivatives thereof can occur at any position including, endocyclic and exocyclic atoms. In some embodiments, the 2-, 6-, 7-, or 8-positions of a purine nucleobase are attached to a conjugate moiety. Conjugation to pyrimidine nucleobases or derivatives thereof can also occur at any position. In some embodiments, the 2-, 5-, and 6-positions of a pyrimidine nucleobase can be substituted with a conjugate moiety. When a ligand is conjugated to a nucleobase, the preferred position is one that does not interfere with hybridization, i.e., does not interfere with the hydrogen bonding interactions needed for base pairing.

Conjugation to sugar moieties of nucleosides can occur at any carbon atom. Exemplary carbon atoms of a sugar moiety that can be attached to a conjugate moiety include the 2′, 3′, and 5′ carbon atoms. The 1′ position can also be attached to a conjugate moiety, such as in an abasic residue. Internucleosidic linkages can also bear conjugate moieties. For phosphorus-containing linkages (e.g., phosphodiester, phosphorothioate, phosphorodithioate, phosphoroamidate, and the like), the conjugate moiety can be attached directly to the phosphorus atom or to an O, N, or S atom bound to the phosphorus atom. For amine- or amide-containing internucleosidic linkages (e.g., PNA), the conjugate moiety can be attached to the nitrogen atom of the amine or amide or to an adjacent carbon atom.

There are numerous methods for preparing conjugates of oligonucleotides. Generally, an oligonucleotide is attached to a conjugate moiety by contacting a reactive group (e.g., OH, SH, amine, carboxyl, aldehyde, and the like) on the oligonucleotide with a reactive group on the conjugate moiety. In some embodiments, one reactive group is electrophilic and the other is nucleophilic.

For example, an electrophilic group can be a carbonyl-containing functionality and a nucleophilic group can be an amine or thiol. Methods for conjugation of nucleic acids and related oligonucleotides with and without linking groups are well described in the literature such as, for example, in Manoharan in Antisense Research and Applications, Crooke and LeBleu, eds., CRC Press, Boca Raton, Fla., 1993, Chapter 17, which is incorporated herein by reference in its entirety.

The ligand can be attached to the oligonucleotide via a linker or a carrier monomer, e.g., a ligand carrier. The carriers include (i) at least one “backbone attachment point,” preferably two “backbone attachment points” and (ii) at least one “tethering attachment point.” A “backbone attachment point” as used herein refers to a functional group, e.g. a hydroxyl group, or generally, a bond available for, and that is suitable for incorporation of the carrier monomer into the backbone, e.g., the phosphate, or modified phosphate, e.g., sulfur containing, backbone, of an oligonucleotide. A “tethering attachment point” (TAP) in refers to an atom of the carrier monomer, e.g., a carbon atom or a heteroatom (distinct from an atom which provides a backbone attachment point), that connects a selected moiety. The selected moiety can be, e.g., a carbohydrate, e.g. monosaccharide, disaccharide, trisaccharide, tetrasaccharide, oligosaccharide and polysaccharide. Optionally, the selected moiety is connected by an intervening tether to the carrier monomer. Thus, the carrier will often include a functional group, e.g., an amino group, or generally, provide a bond, that is suitable for incorporation or tethering of another chemical entity, e.g., a ligand to the constituent atom.

Representative U.S. patents that teach the preparation of conjugates of nucleic acids include, but are not limited to, U.S. Pat. Nos. 4,828,979; 4,948,882; 5,218, 105; 5,525,465; 5,541,313; 5,545,730; 5,552,538; 5,578, 717, 5,580,731; 5,580,731; 5,591,584; 5,109,124; 5,118, 802; 5,138,045; 5,414,077; 5,486,603; 5,512,439; 5,578, 718; 5,608,046; 4,587,044; 4,605,735; 4,667,025; 4,762, 779; 4,789,737; 4,824,941; 4,835,263; 4,876,335; 4,904, 582; 4,958,013; 5,082,830; 5,112,963; 5,214,136; 5,082, 830; 5,112,963; 5,149,782; 5,214,136; 5,245,022; 5,254, 469; 5,258,506; 5,262,536; 5,272,250; 5,292,873; 5,317,098; 5,371,241, 5,391,723; 5,416,203, 5,451,463; 5,510, 475; 5,512,667; 5,514,785; 5,565,552; 5,567,810; 5,574, 142; 5,585,481; 5,587,371; 5,595,726; 5,597,696; 5,599, 923; 5,599,928; 5,672,662; 5,688,941; 5,714,166; 6,153, 737; 6,172,208; 6,300,319; 6,335,434; 6,335,437; 6,395, 437; 6,444,806; 6,486,308; 6,525,031; 6,528,631; 6,559, 279; contents of which are herein incorporated in their entireties by reference.

In some embodiments, the oligonucleotide further comprises a targeting ligand that targets a liver tissue. In some embodiments, the targeting ligand is a carbohydrate-based ligand. In one embodiment, the targeting ligand is a GalNAc conjugate.

Because the ligand can be conjugated to the iRNA agent via a linker or carrier, and because the linker or carrier can contain a branched linker, the iRNA agent can then contain multiple ligands via the same or different backbone attachment points to the carrier, or via the branched linker(s). For instance, the branchpoint of the branched linker may be a bivalent, trivalent, tetravalent, pentavalent, or hexavalent atom, or a group presenting such multiple valences. In certain embodiments, the branchpoint is —N, —N(Q)—C, —O—C, —S—C, —SS—C, —C(O)N(Q)—C, —OC(O)N(Q)—C, —N(Q)C(O)—C, or —N(Q)C(O)O—C; wherein Q is independently for each occurrence H or optionally substituted alkyl. In other embodiment, the branchpoint is glycerol or glycerol derivative.

Evaluation of Candidate iRNAs

One can evaluate a candidate iRNA agent, e.g., a modified RNA, for a selected property by exposing the agent or modified molecule and a control molecule to the appropriate conditions and evaluating for the presence of the selected property. For example, resistance to a degradant can be evaluated as follows. A candidate modified RNA (and a control molecule, usually the unmodified form) can be exposed to degradative conditions, e.g., exposed to a milieu, which includes a degradative agent, e.g., a nuclease. E.g., one can use a biological sample, e.g., one that is similar to a milieu, which might be encountered, in therapeutic use, e.g., blood or a cellular fraction, e.g., a cell-free homogenate or disrupted cells. The candidate and control could then be evaluated for resistance to degradation by any of a number of approaches. For example, the candidate and control could be labeled prior to exposure, with, e.g., a radioactive or enzymatic label, or a fluorescent label, such as Cy3 or Cy5. Control and modified RNA's can be incubated with the degradative agent, and optionally a control, e.g., an inactivated, e.g., heat inactivated, degradative agent. A physical parameter, e.g., size, of the modified and control molecules are then determined. They can be determined by a physical method, e.g., by polyacrylamide gel electrophoresis or a sizing column, to assess whether the molecule has maintained its original length, or assessed functionally. Alternatively, Northern blot analysis can be used to assay the length of an unlabeled modified molecule.

A functional assay can also be used to evaluate the candidate agent. A functional assay can be applied initially or after an earlier non-functional assay, (e.g., assay for resistance to degradation) to determine if the modification alters the ability of the molecule to silence gene expression. For example, a cell, e.g., a mammalian cell, such as a mouse or human cell, can be co-transfected with a plasmid expressing a fluorescent protein, e.g., GFP, and a candidate RNA agent homologous to the transcript encoding the fluorescent protein (see, e.g., WO 00/44914). For example, a modified dsiRNA homologous to the GFP mRNA can be assayed for the ability to inhibit GFP expression by monitoring for a decrease in cell fluorescence, as compared to a control cell, in which the transfection did not include the candidate dsiRNA, e.g., controls with no agent added and/or controls with a non-modified RNA added. Efficacy of the candidate agent on gene expression can be assessed by comparing cell fluorescence in the presence of the modified and unmodified dssiRNAs.

In an alternative functional assay, a candidate dssiRNA homologous to an endogenous mouse gene, for example, a maternally expressed gene, such as c-mos, can be injected into an immature mouse oocyte to assess the ability of the agent to inhibit gene expression in vivo (see, e.g., WO 01/36646). A phenotype of the oocyte, e.g., the ability to maintain arrest in metaphase II, can be monitored as an indicator that the agent is inhibiting expression. For example, cleavage of c-mos mRNA by a dssiRNA would cause the oocyte to exit metaphase arrest and initiate parthenogenetic development (Colledge et al. Nature 370: 65-68, 1994; Hashimoto et al. Nature, 370:68-71, 1994). The effect of the modified agent on target RNA levels can be verified by Northern blot to assay for a decrease in the level of target mRNA, or by Western blot to assay for a decrease in the level of target protein, as compared to a negative control. Controls can include cells in which with no agent is added and/or cells in which a non-modified RNA is added.

### Physiological Effects

The siRNAs described herein can be designed such that determining therapeutic toxicity is made easier by the complementarity of the siRNA with both a human and a non-human animal sequence. By these methods, an siRNA can consist of a sequence that is fully complementary to a nucleic acid sequence from a human and a nucleic acid sequence from at least one non-human animal, e.g., a non-human mammal, such as a rodent, ruminant or primate. For example, the non-human mammal can be a mouse, rat, dog, pig, goat, sheep, cow, monkey, Pan paniscus, Pan troglodytes, Macaca mulatto, or Cynomolgus monkey. The sequence of the siRNA could be complementary to sequences within homologous genes, e.g., oncogenes or tumor suppressor genes, of the non-human mammal and the human. By determining the toxicity of the siRNA in the non-human mammal, one can extrapolate the toxicity of the siRNA in a human. For a more strenuous toxicity test, the siRNA can be complementary to a human and more than one, e.g., two or three or more, non-human animals.

The methods described herein can be used to correlate any physiological effect of an siRNA on a human, e.g., any unwanted effect, such as a toxic effect, or any positive, or desired effect.

Increasing Cellular Uptake of siRNAs

Described herein are various siRNA compositions that contain covalently attached conjugates that increase cellular uptake and/or intracellular targeting of the siRNAs.

Additionally provided are methods of the invention that include administering an siRNA and a drug that affects the uptake of the siRNA into the cell. The drug can be administered before, after, or at the same time that the siRNA is administered. The drug can be covalently or non-covalently linked to the siRNA. The drug can be, for example, a lipopolysaccharide, an activator of p38 MAP kinase, or an activator of NF-κB. The drug can have a transient effect on the cell. The drug can increase the uptake of the siRNA into the cell, for example, by disrupting the cell's cytoskeleton, e.g., by disrupting the cell's microtubules, microfilaments, and/or intermediate filaments. The drug can be, for example, taxon, vincristine, vinblastine, cytochalasin, nocodazole, japlakinolide, latrunculin A, phalloidin, swinholide A, indanocine, or myoservin. The drug can also increase the uptake of the siRNA into a given cell or tissue by activating an inflammatory response, for example. Exemplary drugs that would have such an effect include tumor necrosis factor alpha (TNFalpha), interleukin-1 beta, a CpG motif, gamma interferon or more generally an agent that activates a toll-like receptor.

siRNA Production

An siRNA can be produced, e.g., in bulk, by a variety of methods. Exemplary methods include: organic synthesis and RNA cleavage, e.g., in vitro cleavage.

Organic Synthesis. An siRNA can be made by separately synthesizing a single stranded RNA molecule, or each respective strand of a double-stranded RNA molecule, after which the component strands can then be annealed.

A large bioreactor, e.g., the OligoPilot II from Pharmacia Biotec AB (Uppsala Sweden), can be used to produce a large amount of a particular RNA strand for a given siRNA. The OligoPilot II reactor can efficiently couple a nucleotide using only a 1.5 molar excess of a phosphoramidite nucleotide. To make an RNA strand, ribonucleotides amidites are used. Standard cycles of monomer addition can be used to synthesize the 21 to 23 nucleotide strand for the siRNA. Typically, the two complementary strands are produced separately and then annealed, e.g., after release from the solid support and deprotection.

Organic synthesis can be used to produce a discrete siRNA species. The complementary of the species to a particular target gene can be precisely specified. For example, the species may be complementary to a region that includes a polymorphism, e.g., a single nucleotide polymorphism. Further the location of the polymorphism can be precisely defined. In some embodiments, the polymorphism is located in an internal region, e.g., at least 4, 5, 7, or 9 nucleotides from one or both of the termini.

dsiRNA Cleavage. siRNAs can also be made by cleaving a larger siRNA. The cleavage can be mediated in vitro or in vivo. For example, to produce iRNAs by cleavage in vitro, the following method can be used:

In vitro transcription. dsiRNA is produced by transcribing a nucleic acid (DNA) segment in both directions. For example, the HiScribe™ RNAi transcription kit (New England Biolabs) provides a vector and a method for producing a dsiRNA for a nucleic acid segment that is cloned into the vector at a position flanked on either side by a T7 promoter. Separate templates are generated for T7 transcription of the two complementary strands for the dsiRNA. The templates are transcribed in vitro by addition of T7 RNA polymerase and dsiRNA is produced. Similar methods using PCR and/or other RNA polymerases (e.g., T3 or SP6 polymerase) can also be dotoxins that may contaminate preparations of the recombinant enzymes.

In Vitro Cleavage. In one embodiment, RNA generated by this method is carefully purified to remove endsiRNA is cleaved in vitro into siRNAs, for example, using a Dicer or comparable RNAse III-based activity. For example, the dsiRNA can be incubated in an in vitro extract from Drosophila or using purified components, e.g., a purified RNAse or RISC complex (RNA-induced silencing complex). See, e.g., Ketting et al. Genes Dev 2001 October 15; 15(20):2654-9; and Hammond Science 2001 August 10; 293(5532):1146-50.

dsiRNA cleavage generally produces a plurality of siRNA species, each being a particular 21 to 23 nt fragment of a source dsiRNA molecule. For example, siRNAs that include sequences complementary to overlapping regions and adjacent regions of a source dsiRNA molecule may be present.

Regardless of the method of synthesis, the siRNA preparation can be prepared in a solution (e.g., an aqueous and/or organic solution) that is appropriate for formulation. For example, the siRNA preparation can be precipitated and redissolved in pure double-distilled water, and lyophilized. The dried siRNA can then be resuspended in a solution appropriate for the intended formulation process.

Making iRNA Agents Conjugated to a Targeting Ligand

In some embodiments, the targeting ligand conjugated to the iRNA agent via a nucleobase, sugar moiety, or internucleosidic linkage.

Conjugation to purine nucleobases or derivatives thereof can occur at any position including, endocyclic and exocyclic atoms. In some embodiments, the 2-, 6-, 7-, or 8-positions of a purine nucleobase are attached to a conjugate moiety. Conjugation to pyrimidine nucleobases or derivatives thereof can also occur at any position. In some embodiments, the 2-, 5-, and 6-positions of a pyrimidine nucleobase can be substituted with a conjugate moiety. When a targeting ligand is conjugated to a nucleobase, the preferred position is one that does not interfere with hybridization, i.e., does not interfere with the hydrogen bonding interactions needed for base pairing. In one embodiment, the targeting ligand may be conjugated to a nucleobase via a linker containing an alkyl, alkenyl or amide linkage.

Conjugation to sugar moieties of nucleosides can occur at any carbon atom. Exemplary carbon atoms of a sugar moiety that a targeting ligand can be attached to include the 2′, 3′, and 5′ carbon atoms. A targeting ligand can also be attached to the 1′ position, such as in an abasic residue. In one embodiment, the targeting ligand may be conjugated to a sugar moiety, via a 2′-O modification, with or without a linker.

Internucleosidic linkages can also bear targeting ligands. For phosphorus-containing linkages (e.g., phosphodiester, phosphorothioate, phosphorodithioate, phosphoroamidate, and the like), the targeting ligand can be attached directly to the phosphorus atom or to an O, N, or S atom bound to the phosphorus atom. For amine- or amide-containing internucleosidic linkages (e.g., PNA), the targeting ligand can be attached to the nitrogen atom of the amine or amide or to an adjacent carbon atom.

There are numerous methods for preparing conjugates of oligonucleotides. Generally, an oligonucleotide is attached to a conjugate moiety by contacting a reactive group (e.g., OH, SH, amine, carboxyl, aldehyde, and the like) on the oligonucleotide with a reactive group on the conjugate moiety. In some embodiments, one reactive group is electrophilic and the other is nucleophilic.

For example, an electrophilic group can be a carbonyl-containing functionality and a nucleophilic group can be an amine or thiol. Methods for conjugation of nucleic acids and related oligonucleotides with and without linking groups are well described in the literature such as, for example, in Manoharan in Antisense Research and Applications, Crooke and LeBleu, eds., CRC Press, Boca Raton, Fla., 1993, Chapter 17, which is incorporated herein by reference in its entirety.

In one embodiment, a first (complementary) RNA strand and a second (sense) RNA strand can be synthesized separately, wherein one of the RNA strands comprises a pendant targeting ligand, and the first and second RNA strands can be mixed to form a dsRNA. The step of synthesizing the RNA strand preferably involves solid-phase synthesis, wherein individual nucleotides are joined end to end through the formation of internucleotide 3′-5′ phosphodiester bonds in consecutive synthesis cycles.

In one embodiment, a targeting ligand having a phosphoramidite group is coupled to the 3′-end or 5′-end of either the first (complementary) or second (sense) RNA strand in the last synthesis cycle. In the solid-phase synthesis of an RNA, the nucleotides are initially in the form of nucleoside phosphoramidites. In each synthesis cycle, a further nucleoside phosphoramidite is linked to the —OH group of the previously incorporated nucleotide. If the targeting ligand has a phosphoramidite group, it can be coupled in a manner similar to a nucleoside phosphoramidite to the free OH end of the RNA synthesized previously in the solid-phase synthesis. The synthesis can take place in an automated and standardized manner using a conventional RNA synthesizer. Synthesis of the targeting ligand having the phosphoramidite group may include phosphitylation of a free hydroxyl to generate the phosphoramidite group.

In general, the oligonucleotides can be synthesized using protocols known in the art, for example, as described in Caruthers et al., Methods in Enzymology (1992) 211:3-19; WO 99/54459; Wincott et al., Nucl. Acids Res. (1995) 23:2677-2684; Wincott et al., Methods Mol. Bio., (1997) 74:59; Brennan et al., Biotechnol. Bioeng. (1998) 61:33-45; and U.S. Pat. No. 6,001,311; each of which is hereby incorporated by reference in its entirety. In general, the synthesis of oligonucleotides involves conventional nucleic acid protecting and coupling groups, such as dimethoxytrityl at the 5′-end, and phosphoramidites at the 3′-end. In a non-limiting example, small scale syntheses are conducted on a Expedite 8909 RNA synthesizer sold by Applied Biosystems, Inc. (Weiterstadt, Germany), using ribonucleoside phosphoramidites sold by ChemGenes Corporation (Ashland, Mass.). Alternatively, syntheses can be performed on a 96-well plate synthesizer, such as the instrument produced by Protogene (Palo Alto, Calif), or by methods such as those described in Usman et al., J. Am. Chem. Soc. (1987) 109:7845; Scaringe, et al., Nucl. Acids Res. (1990) 18:5433; Wincott, et al., Nucl. Acids Res. (1990) 23:2677-2684; and Wincott, et al., Methods Mol. Bio. (1997) 74:59, each of which is hereby incorporated by reference in its entirety.

The nucleic acid molecules of the present invention may be synthesized separately and joined together post-synthetically, for example, by ligation (Moore et al., Science (1992) 256:9923; WO 93/23569; Shabarova et al., Nucl. Acids Res. (1991) 19:4247; Bellon et al., Nucleosides & Nucleotides (1997) 16:951; Bellon et al., Bioconjugate Chem. (1997) 8:204; or by hybridization following synthesis and/or deprotection. The nucleic acid molecules can be purified by gel electrophoresis using conventional methods or can be purified by high pressure liquid chromatography (HPLC; see Wincott et al., supra, the totality of which is hereby incorporated herein by reference) and re-suspended in water.

### Pharmaceutical Compositions

In one aspect, the invention features a pharmaceutical composition that includes an iRNA (an siRNA), e.g., a double-stranded siRNA, or ssiRNA, (e.g., a precursor, e.g., a larger siRNA which can be processed into a ssiRNA, or a DNA which encodes an siRNA, e.g., a double-stranded siRNA, or ssiRNA, or precursor thereof) including a nucleotide sequence complementary to a target RNA, e.g., substantially and/or exactly complementary. The target RNA can be a transcript of an endogenous human gene. In one embodiment, the siRNA (a) is 19-25 nucleotides long, for example, 21-23 nucleotides, (b) is complementary to an endogenous target RNA, and, optionally, (c) includes at least one 3′ overhang 1-5 nt long. In one embodiment, the pharmaceutical composition can be an emulsion, microemulsion, cream, jelly, or liposome.

In one example the pharmaceutical composition includes an iRNA (an siRNA) mixed with a topical delivery agent. The topical delivery agent can be a plurality of microscopic vesicles. The microscopic vesicles can be liposomes. In some embodiments the liposomes are cationic liposomes.

In another aspect, the pharmaceutical composition includes an iRNA (an siRNA), e.g., a double-stranded siRNA, or ssiRNA (e.g., a precursor, e.g., a larger siRNA which can be processed into a ssiRNA, or a DNA which encodes an siRNA, e.g., a double-stranded siRNA, or ssiRNA, or precursor thereof) admixed with a topical penetration enhancer. In one embodiment, the topical penetration enhancer is a fatty acid. The fatty acid can be arachidonic acid, oleic acid, lauric acid, caprylic acid, capric acid, myristic acid, palmitic acid, stearic acid, linoleic acid, linolenic acid, dicaprate, tricaprate, monolein, dilaurin, glyceryl 1-monocaprate, 1-dodecylazacycloheptan-2-one, an acylcarnitine, an acylcholine, or a C₁₋₁₀ alkyl ester, monoglyceride, diglyceride or pharmaceutically acceptable salt thereof.

In another embodiment, the topical penetration enhancer is a bile salt. The bile salt can be cholic acid, dehydrocholic acid, deoxycholic acid, glucholic acid, glycholic acid, glycodeoxycholic acid, taurocholic acid, taurodeoxycholic acid, chenodeoxycholic acid, ursodeoxycholic acid, sodium tauro-24,25-dihydro-fusidate, sodium glycodihydrofusidate, polyoxyethylene-9-lauryl ether or a pharmaceutically acceptable salt thereof.

In another embodiment, the penetration enhancer is a chelating agent. The chelating agent can be EDTA, citric acid, a salicyclate, a N-acyl derivative of collagen, laureth-9, an N-amino acyl derivative of a beta-diketone or a mixture thereof.

In another embodiment, the penetration enhancer is a surfactant, e.g., an ionic or nonionic surfactant. The surfactant can be sodium lauryl sulfate, polyoxyethylene-9-lauryl ether, polyoxyethylene-20-cetyl ether, a perfluorchemical emulsion or mixture thereof.

In another embodiment, the penetration enhancer can be selected from a group consisting of unsaturated cyclic ureas, 1-alkyl-alkones, 1-alkenylazacyclo-alakanones, steroidal anti-inflammatory agents and mixtures thereof. In yet another embodiment the penetration enhancer can be a glycol, a pyrrol, an azone, or a terpenes.

In one aspect, the invention features a pharmaceutical composition including an iRNA (an siRNA), e.g., a double-stranded siRNA, or ssiRNA, (e.g., a precursor, e.g., a larger siRNA which can be processed into a ssiRNA, or a DNA which encodes an siRNA, e.g., a double-stranded siRNA, or ssiRNA, or precursor thereof) in a form suitable for oral delivery. In one embodiment, oral delivery can be used to deliver an siRNA composition to a cell or a region of the gastro-intestinal tract, e.g., small intestine, colon (e.g., to treat a colon cancer), and so forth. The oral delivery form can be tablets, capsules or gel capsules. In one embodiment, the siRNA of the pharmaceutical composition modulates expression of a cellular adhesion protein, modulates a rate of cellular proliferation, or has biological activity against eukaryotic pathogens or retroviruses. In another embodiment, the pharmaceutical composition includes an enteric material that substantially prevents dissolution of the tablets, capsules or gel capsules in a mammalian stomach. In some embodiments the enteric material is a coating. The coating can be acetate phthalate, propylene glycol, sorbitan monoleate, cellulose acetate trimellitate, hydroxy propyl methylcellulose phthalate or cellulose acetate phthalate.

In another embodiment, the oral dosage form of the pharmaceutical composition includes a penetration enhancer. The penetration enhancer can be a bile salt or a fatty acid. The bile salt can be ursodeoxycholic acid, chenodeoxycholic acid, and salts thereof. The fatty acid can be capric acid, lauric acid, and salts thereof.

In another embodiment, the oral dosage form of the pharmaceutical composition includes an excipient. In one example the excipient is polyethyleneglycol. In another example the excipient is precirol.

In another embodiment, the oral dosage form of the pharmaceutical composition includes a plasticizer. The plasticizer can be diethyl phthalate, triacetin dibutyl sebacate, dibutyl phthalate or triethyl citrate.

In one aspect, the invention features a pharmaceutical composition including an iRNA (an siRNA) and a delivery vehicle. In one embodiment, the siRNA is (a) is 19-25 nucleotides long, for example, 21-23 nucleotides, (b) is complementary to an endogenous target RNA, and, optionally, (c) includes at least one 3′ overhang 1-5 nucleotides long.

In one embodiment, the delivery vehicle can deliver an iRNA (an siRNA), e.g., a double-stranded siRNA, or ssiRNA, (e.g., a precursor, e.g., a larger siRNA which can be processed into a ssiRNA, or a DNA which encodes an siRNA, e.g., a double-stranded siRNA, or ssiRNA, or precursor thereof) to a cell by a topical route of administration. The delivery vehicle can be microscopic vesicles. In one example the microscopic vesicles are liposomes. In some embodiments the liposomes are cationic liposomes. In another example the microscopic vesicles are micelles. In one aspect, the invention features a pharmaceutical composition including an siRNA, e.g., a double-stranded siRNA, or ssiRNA, (e.g., a precursor, e.g., a larger siRNA which can be processed into a ssiRNA, or a DNA which encodes an siRNA, e.g., a double-stranded siRNA, or ssiRNA, or precursor thereof) in an injectable dosage form. In one embodiment, the injectable dosage form of the pharmaceutical composition includes sterile aqueous solutions or dispersions and sterile powders. In some embodiments the sterile solution can include a diluent such as water; saline solution; fixed oils, polyethylene glycols, glycerin, or propylene glycol.

In one aspect, the invention features a pharmaceutical composition including an iRNA (an siRNA), e.g., a double-stranded siRNA, or ssiRNA, (e.g., a precursor, e.g., a larger siRNA which can be processed into a ssiRNA, or a DNA which encodes an siRNA, e.g., a double-stranded siRNA, or ssiRNA, or precursor thereof) in oral dosage form. In one embodiment, the oral dosage form is selected from the group consisting of tablets, capsules and gel capsules. In another embodiment, the pharmaceutical composition includes an enteric material that substantially prevents dissolution of the tablets, capsules or gel capsules in a mammalian stomach. In some embodiments the enteric material is a coating. The coating can be acetate phthalate, propylene glycol, sorbitan monoleate, cellulose acetate trimellitate, hydroxy propyl methyl cellulose phthalate or cellulose acetate phthalate. In one embodiment, the oral dosage form of the pharmaceutical composition includes a penetration enhancer, e.g., a penetration enhancer described herein.

In another embodiment, the oral dosage form of the pharmaceutical composition includes an excipient. In one example the excipient is polyethyleneglycol. In another example the excipient is precirol.

In another embodiment, the oral dosage form of the pharmaceutical composition includes a plasticizer. The plasticizer can be diethyl phthalate, triacetin dibutyl sebacate, dibutyl phthalate or triethyl citrate.

In one aspect, the invention features a pharmaceutical composition including an iRNA (an siRNA), e.g., a double-stranded siRNA, or ssiRNA, (e.g., a precursor, e.g., a larger siRNA which can be processed into a ssiRNA, or a DNA which encodes an siRNA, e.g., a double-stranded siRNA, or ssiRNA, or precursor thereof) in a rectal dosage form. In one embodiment, the rectal dosage form is an enema. In another embodiment, the rectal dosage form is a suppository.

In one aspect, the invention features a pharmaceutical composition including an iRNA (an siRNA), e.g., a double-stranded siRNA, or ssiRNA, (e.g., a precursor, e.g., a larger siRNA which can be processed into a ssiRNA, or a DNA which encodes an siRNA, e.g., a double-stranded siRNA, or ssiRNA, or precursor thereof) in a vaginal dosage form. In one embodiment, the vaginal dosage form is a suppository. In another embodiment, the vaginal dosage form is a foam, cream, or gel.

In one aspect, the invention features a pharmaceutical composition including an iRNA (an siRNA), e.g., a double-stranded siRNA, or ssiRNA, (e.g., a precursor, e.g., a larger siRNA which can be processed into a ssiRNA, or a DNA which encodes an siRNA, e.g., a double-stranded siRNA, or ssiRNA, or precursor thereof) in a pulmonary or nasal dosage form. In one embodiment, the siRNA is incorporated into a particle, e.g., a macroparticle, e.g., a microsphere. The particle can be produced by spray drying, lyophilization, evaporation, fluid bed drying, vacuum drying, or a combination thereof. The microsphere can be formulated as a suspension, a powder, or an implantable solid.

### Treatment Methods and Routes of Delivery

Another aspect of the invention relates to a method of reducing the expression of a target gene in a cell, comprising contacting said cell with the oligonucleotide. In one embodiment, the cell is an extrahepatic cell.

Another aspect of the invention relates to a method of reducing the expression of a target gene in a subject, comprising administering to the subject the oligonucleotide.

Another aspect of the invention relates to a method of treating a subject having a CNS disorder, comprising administering to the subject a therapeutically effective amount of the double-stranded iRNA agent of the invention, thereby treating the subject. Exemplary CNS disorders that can be treated by the method of the invention include Alzheimer, amyotrophic lateral sclerosis (ALS), frontotemporal dementia, Huntington, Parkinson, spinocerebellar, prion, and lafora.

The oligonucleotide can be delivered to a subject by a variety of routes, depending on the type of genes targeted and the type of disorders to be treated. In some embodiments, the oligonucleotide is administered extrahepatically, such as an ocular administration (e.g., intravitreal administration) or an intrathecal or intracerebroventricular administration.

In one embodiment, the oligonucleotide is administered intrathecally or intracerebroventricularly. By intrathecal or intracerebroventricular administration of the double-stranded iRNA agent, the method can reduce the expression of a target gene in a brain or spine tissue, for instance, cortex, cerebellum, cervical spine, lumbar spine, and thoracic spine.

In some embodiments, exemplary target genes are APP, ATXN2, C9orf72, TARDBP, MAPT(Tau), HTT, SNCA, FUS, ATXN3, ATXN1, SCA1, SCA7, SCA8, MeCP2, PRNP, SOD1, DMPK, and TTR. To reduce the expression of these target genes in the subject, the oligonucleotide can be administered to the eye(s) directly (e.g., intravitreally). By intravitreal administration of the double-stranded iRNA agent, the method can reduce the expression of the target gene in an ocular tissue.

For ease of exposition the formulations, compositions and methods in this section are discussed largely with regard to modified siRNAs. It may be understood, however, that these formulations, compositions and methods can be practiced with other siRNAs, e.g., unmodified siRNAs, and such practice is within the invention. A composition that includes a iRNA can be delivered to a subject by a variety of routes. Exemplary routes include: intravenous, topical, rectal, anal, vaginal, nasal, pulmonary, ocular.

The iRNA molecules of the invention can be incorporated into pharmaceutical compositions suitable for administration. Such compositions typically include one or more species of iRNA and a pharmaceutically acceptable carrier. As used herein the language “pharmaceutically acceptable carrier” is intended to include any and all solvents, dispersion media, coatings, antibacterial and antifungal agents, isotonic and absorption delaying agents, and the like, compatible with pharmaceutical administration. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

The pharmaceutical compositions of the present invention may be administered in a number of ways depending upon whether local or systemic treatment is desired and upon the area to be treated. Administration may be topical (including ophthalmic, vaginal, rectal, intranasal, transdermal), oral or parenteral. Parenteral administration includes intravenous drip, subcutaneous, intraperitoneal or intramuscular injection, or intrathecal or intraventricular or intracerebroventricular administration.

The route and site of administration may be chosen to enhance targeting. For example, to target muscle cells, intramuscular injection into the muscles of interest would be a logical choice. Lung cells might be targeted by administering the iRNA in aerosol form. The vascular endothelial cells could be targeted by coating a balloon catheter with the iRNA and mechanically introducing the DNA.

Formulations for topical administration may include transdermal patches, ointments, lotions, creams, gels, drops, suppositories, sprays, liquids and powders. Conventional pharmaceutical carriers, aqueous, powder or oily bases, thickeners and the like may be necessary or desirable. Coated condoms, gloves and the like may also be useful.

Compositions for oral administration include powders or granules, suspensions or solutions in water, syrups, elixirs or non-aqueous media, tablets, capsules, lozenges, or troches. In the case of tablets, carriers that can be used include lactose, sodium citrate and salts of phosphoric acid. Various disintegrants such as starch, and lubricating agents such as magnesium stearate, sodium lauryl sulfate and talc, are commonly used in tablets. For oral administration in capsule form, useful diluents are lactose and high molecular weight polyethylene glycols. When aqueous suspensions are required for oral use, the nucleic acid compositions can be combined with emulsifying and suspending agents. If desired, certain sweetening and/or flavoring agents can be added.

Compositions for intrathecal or intraventricular or intracerebroventricular administration may include sterile aqueous solutions which may also contain buffers, diluents and other suitable additives.

Formulations for parenteral administration may include sterile aqueous solutions which may also contain buffers, diluents and other suitable additives. Intraventricular injection may be facilitated by an intraventricular catheter, for example, attached to a reservoir. For intravenous use, the total concentration of solutes may be controlled to render the preparation isotonic.

For ocular administration, ointments or droppable liquids may be delivered by ocular delivery systems known to the art such as applicators or eye droppers. Such compositions can include mucomimetics such as hyaluronic acid, chondroitin sulfate, hydroxypropyl methylcellulose or poly(vinyl alcohol), preservatives such as sorbic acid, EDTA or benzylchronium chloride, and the usual quantities of diluents and/or carriers.

In one embodiment, the administration of the iRNA (siRNA), e.g., a double-stranded siRNA, or ssiRNA, composition is parenteral, e.g., intravenous (e.g., as a bolus or as a diffusible infusion), intradermal, intraperitoneal, intramuscular, intrathecal, intraventricular, intracerebroventricular, intracranial, subcutaneous, transmucosal, buccal, sublingual, endoscopic, rectal, oral, vaginal, topical, pulmonary, intranasal, urethral or ocular. Administration can be provided by the subject or by another person, e.g., a health care provider. The medication can be provided in measured doses or in a dispenser which delivers a metered dose. Selected modes of delivery are discussed in more detail below.

Intrathecal Administration. In one embodiment, the is delivered by intrathecal injection (i.e. injection into the spinal fluid which bathes the brain and spinal cord tissue). Intrathecal injection of iRNA agents into the spinal fluid can be performed as a bolus injection or via minipumps which can be implanted beneath the skin, providing a regular and constant delivery of siRNA into the spinal fluid. The circulation of the spinal fluid from the choroid plexus, where it is produced, down around the spinal cord and dorsal root ganglia and subsequently up past the cerebellum and over the cortex to the arachnoid granulations, where the fluid can exit the CNS, that, depending upon size, stability, and solubility of the compounds injected, molecules delivered intrathecally could hit targets throughout the entire CNS.

In some embodiments, the intrathecal administration is via a pump. The pump may be a surgically implanted osmotic pump. In one embodiment, the osmotic pump is implanted into the subarachnoid space of the spinal canal to facilitate intrathecal administration.

In some embodiments, the intrathecal administration is via an intrathecal delivery system for a pharmaceutical including a reservoir containing a volume of the pharmaceutical agent, and a pump configured to deliver a portion of the pharmaceutical agent contained in the reservoir. More details about this intrathecal delivery system may be found in PCT/US2015/013253, filed on Jan. 28, 2015, which is incorporated by reference in its entirety.

The amount of intrathecally or intracerebroventricularly injected iRNA agents may vary from one target gene to another target gene and the appropriate amount that has to be applied may have to be determined individually for each target gene. Typically, this amount ranges between 10 μg to 2 mg, preferably 50 μg to 1500 μg, more preferably 100 μg to 1000 μg.

Rectal Administration. The invention also provides methods, compositions, and kits, for rectal administration or delivery of siRNAs described herein.

Accordingly, an iRNA (an siRNA), e.g., a double-stranded siRNA, or ssiRNA, (e.g., a precursor, e.g., a larger siRNA which can be processed into a ssiRNA, or a DNA which encodes a an siRNA, e.g., a double-stranded siRNA, or ssiRNA, or precursor thereof) described herein, e.g., a therapeutically effective amount of a siRNA described herein, e.g., a siRNA having a double stranded region of less than 40, and, for example, less than 30 nucleotides and having one or two 1-3 nucleotide single strand 3′ overhangs can be administered rectally, e.g., introduced through the rectum into the lower or upper colon. This approach is particularly useful in the treatment of, inflammatory disorders, disorders characterized by unwanted cell proliferation, e.g., polyps, or colon cancer.

The medication can be delivered to a site in the colon by introducing a dispensing device, e.g., a flexible, camera-guided device similar to that used for inspection of the colon or removal of polyps, which includes means for delivery of the medication.

The rectal administration of the siRNA is by means of an enema. The siRNA of the enema can be dissolved in a saline or buffered solution. The rectal administration can also by means of a suppository, which can include other ingredients, e.g., an excipient, e.g., cocoa butter or hydropropylmethylcellulose.

Ocular Delivery. The iRNA agents described herein can be administered to an ocular tissue. For example, the medications can be applied to the surface of the eye or nearby tissue, e.g., the inside of the eyelid. They can be applied topically, e.g., by spraying, in drops, as an eyewash, or an ointment. Administration can be provided by the subject or by another person, e.g., a health care provider. The medication can be provided in measured doses or in a dispenser which delivers a metered dose. The medication can also be administered to the interior of the eye, and can be introduced by a needle or other delivery device which can introduce it to a selected area or structure. Ocular treatment is particularly desirable for treating inflammation of the eye or nearby tissue.

In certain embodiments, the double-stranded iRNA agents may be delivered directly to the eye by ocular tissue injection such as periocular, conjunctival, subtenon, intracameral, intravitreal, intraocular, anterior or posterior juxtascleral, subretinal, subconjunctival, retrobulbar, or intracanalicular injections; by direct application to the eye using a catheter or other placement device such as a retinal pellet, intraocular insert, suppository or an implant comprising a porous, non-porous, or gelatinous material; by topical ocular drops or ointments; or by a slow release device in the cul-de-sac or implanted adjacent to the sclera (transscleral) or in the sclera (intrascleral) or within the eye. Intracameral injection may be through the cornea into the anterior chamber to allow the agent to reach the trabecular meshwork. Intracanalicular injection may be into the venous collector channels draining Schlemm's canal or into Schlemm's canal.

In one embodiment, the double-stranded iRNA agents may be administered into the eye, for example the vitreous chamber of the eye, by intravitreal injection, such as with pre-filled syringes in ready-to-inject form for use by medical personnel.

For ophthalmic delivery, the double-stranded iRNA agents may be combined with ophthalmologically acceptable preservatives, co-solvents, surfactants, viscosity enhancers, penetration enhancers, buffers, sodium chloride, or water to form an aqueous, sterile ophthalmic suspension or solution. Solution formulations may be prepared by dissolving the conjugate in a physiologically acceptable isotonic aqueous buffer. Further, the solution may include an acceptable surfactant to assist in dissolving the double-stranded iRNA agents. Viscosity building agents, such as hydroxymethyl cellulose, hydroxyethyl cellulose, methylcellulose, polyvinylpyrrolidone, or the like may be added to the pharmaceutical compositions to improve the retention of the double-stranded iRNA agents.

To prepare a sterile ophthalmic ointment formulation, the double-stranded iRNA agents is combined with a preservative in an appropriate vehicle, such as mineral oil, liquid lanolin, or white petrolatum. Sterile ophthalmic gel formulations may be prepared by suspending the double-stranded iRNA agents in a hydrophilic base prepared from the combination of, for example, CARBOPOL®-940 (BF Goodrich, Charlotte, N.C.), or the like, according to methods known in the art.

Topical Delivery. Any of the siRNAs described herein can be administered directly to the skin. For example, the medication can be applied topically or delivered in a layer of the skin, e.g., by the use of a microneedle or a battery of microneedles which penetrate into the skin, but, for example, not into the underlying muscle tissue. Administration of the siRNA composition can be topical. Topical applications can, for example, deliver the composition to the dermis or epidermis of a subject. Topical administration can be in the form of transdermal patches, ointments, lotions, creams, gels, drops, suppositories, sprays, liquids or powders. A composition for topical administration can be formulated as a liposome, micelle, emulsion, or other lipophilic molecular assembly. The transdermal administration can be applied with at least one penetration enhancer, such as iontophoresis, phonophoresis, and sonophoresis.

For ease of exposition the formulations, compositions and methods in this section are discussed largely with regard to modified siRNAs. It may be understood, however, that these formulations, compositions and methods can be practiced with other siRNAs, e.g., unmodified siRNAs, and such practice is within the invention. In some embodiments, an siRNA, e.g., a double-stranded siRNA, or ssiRNA, (e.g., a precursor, e.g., a larger siRNA which can be processed into a ssiRNA, or a DNA which encodes an siRNA, e.g., a double-stranded siRNA, or ssiRNA, or precursor thereof) is delivered to a subject via topical administration. “Topical administration” refers to the delivery to a subject by contacting the formulation directly to a surface of the subject. The most common form of topical delivery is to the skin, but a composition disclosed herein can also be directly applied to other surfaces of the body, e.g., to the eye, a mucous membrane, to surfaces of a body cavity or to an internal surface. As mentioned above, the most common topical delivery is to the skin. The term encompasses several routes of administration including, but not limited to, topical and transdermal. These modes of administration typically include penetration of the skin's permeability barrier and efficient delivery to the target tissue or stratum. Topical administration can be used as a means to penetrate the epidermis and dermis and ultimately achieve systemic delivery of the composition. Topical administration can also be used as a means to selectively deliver oligonucleotides to the epidermis or dermis of a subject, or to specific strata thereof, or to an underlying tissue.

The term “skin,” as used herein, refers to the epidermis and/or dermis of an animal. Mammalian skin consists of two major, distinct layers. The outer layer of the skin is called the epidermis. The epidermis is comprised of the stratum corneum, the stratum granulosum, the stratum spinosum, and the stratum basale, with the stratum corneum being at the surface of the skin and the stratum basale being the deepest portion of the epidermis. The epidermis is between 50 μm and 0.2 mm thick, depending on its location on the body.

Beneath the epidermis is the dermis, which is significantly thicker than the epidermis. The dermis is primarily composed of collagen in the form of fibrous bundles. The collagenous bundles provide support for, inter alia, blood vessels, lymph capillaries, glands, nerve endings and immunologically active cells.

One of the major functions of the skin as an organ is to regulate the entry of substances into the body. The principal permeability barrier of the skin is provided by the stratum corneum, which is formed from many layers of cells in various states of differentiation. The spaces between cells in the stratum corneum is filled with different lipids arranged in lattice-like formations that provide seals to further enhance the skins permeability barrier.

The permeability barrier provided by the skin is such that it is largely impermeable to molecules having molecular weight greater than about 750 Da. For larger molecules to cross the skin's permeability barrier, mechanisms other than normal osmosis must be used.

Several factors determine the permeability of the skin to administered agents. These factors include the characteristics of the treated skin, the characteristics of the delivery agent, interactions between both the drug and delivery agent and the drug and skin, the dosage of the drug applied, the form of treatment, and the post treatment regimen. To selectively target the epidermis and dermis, it is sometimes possible to formulate a composition that comprises one or more penetration enhancers that will enable penetration of the drug to a preselected stratum.

Transdermal delivery is a valuable route for the administration of lipid soluble therapeutics. The dermis is more permeable than the epidermis and therefore absorption is much more rapid through abraded, burned or denuded skin. Inflammation and other physiologic conditions that increase blood flow to the skin also enhance transdermal adsorption. Absorption via this route may be enhanced by the use of an oily vehicle (inunction) or through the use of one or more penetration enhancers. Other effective ways to deliver a composition disclosed herein via the transdermal route include hydration of the skin and the use of controlled release topical patches. The transdermal route provides a potentially effective means to deliver a composition disclosed herein for systemic and/or local therapy.

In addition, iontophoresis (transfer of ionic solutes through biological membranes under the influence of an electric field) (Lee et al., Critical Reviews in Therapeutic Drug Carrier Systems, 1991, p. 163), phonophoresis or sonophoresis (use of ultrasound to enhance the absorption of various therapeutic agents across biological membranes, notably the skin and the cornea) (Lee et al., Critical Reviews in Therapeutic Drug Carrier Systems, 1991, p. 166), and optimization of vehicle characteristics relative to dose position and retention at the site of administration (Lee et al., Critical Reviews in Therapeutic Drug Carrier Systems, 1991, p. 168) may be useful methods for enhancing the transport of topically applied compositions across skin and mucosal sites.

The compositions and methods provided may also be used to examine the function of various proteins and genes in vitro in cultured or preserved dermal tissues and in animals. The invention can be thus applied to examine the function of any gene. The methods of the invention can also be used therapeutically or prophylactically. For example, for the treatment of animals that are known or suspected to suffer from diseases such as psoriasis, lichen planus, toxic epidermal necrolysis, ertythema multiforme, basal cell carcinoma, squamous cell carcinoma, malignant melanoma, Paget's disease, Kaposi's sarcoma, pulmonary fibrosis, Lyme disease and viral, fungal and bacterial infections of the skin.

Pulmonary Delivery. Any of the siRNAs described herein can be administered to the pulmonary system. Pulmonary administration can be achieved by inhalation or by the introduction of a delivery device into the pulmonary system, e.g., by introducing a delivery device which can dispense the medication. Certain embodiments may use a method of pulmonary delivery by inhalation. The medication can be provided in a dispenser which delivers the medication, e.g., wet or dry, in a form sufficiently small such that it can be inhaled. The device can deliver a metered dose of medication. The subject, or another person, can administer the medication. Pulmonary delivery is effective not only for disorders which directly affect pulmonary tissue, but also for disorders which affect other tissue. siRNAs can be formulated as a liquid or nonliquid, e.g., a powder, crystal, or aerosol for pulmonary delivery.

For ease of exposition the formulations, compositions and methods in this section are discussed largely with regard to modified siRNAs. It may be understood, however, that these formulations, compositions and methods can be practiced with other siRNAs, e.g., unmodified siRNAs, and such practice is within the invention. A composition that includes an siRNA, e.g., a double-stranded siRNA, or ssiRNA, (e.g., a precursor, e.g., a larger siRNA which can be processed into a ssiRNA, or a DNA which encodes an siRNA, e.g., a double-stranded siRNA, or ssiRNA, or precursor thereof) can be administered to a subject by pulmonary delivery. Pulmonary delivery compositions can be delivered by inhalation by the patient of a dispersion so that the composition, for example, iRNA, within the dispersion can reach the lung where it can be readily absorbed through the alveolar region directly into blood circulation. Pulmonary delivery can be effective both for systemic delivery and for localized delivery to treat diseases of the lungs.

Pulmonary delivery can be achieved by different approaches, including the use of nebulized, aerosolized, micellular and dry powder-based formulations. Delivery can be achieved with liquid nebulizers, aerosol-based inhalers, and dry powder dispersion devices. Metered-dose devices are may be used. One of the benefits of using an atomizer or inhaler is that the potential for contamination is minimized because the devices are self contained. Dry powder dispersion devices, for example, deliver drugs that may be readily formulated as dry powders. A iRNA composition may be stably stored as lyophilized or spray-dried powders by itself or in combination with suitable powder carriers. The delivery of a composition for inhalation can be mediated by a dosing timing element which can include a timer, a dose counter, time measuring device, or a time indicator which when incorporated into the device enables dose tracking, compliance monitoring, and/or dose triggering to a patient during administration of the aerosol medicament.

The term “powder” means a composition that consists of finely dispersed solid particles that are free flowing and capable of being readily dispersed in an inhalation device and subsequently inhaled by a subject so that the particles reach the lungs to permit penetration into the alveoli. Thus, the powder is said to be “respirable.” For example, the average particle size is less than about 10 μm in diameter with a relatively uniform spheroidal shape distribution. In some embodiments, the diameter is less than about 7.5 μm and in some embodiments less than about 5.0 μm. Usually the particle size distribution is between about 0.1 μm and about 5 μm in diameter, sometimes about 0.3 μm to about 5 μm.

The term “dry” means that the composition has a moisture content below about 10% by weight (% w) water, usually below about 5% w and in some cases less it than about 3% w. A dry composition can be such that the particles are readily dispersible in an inhalation device to form an aerosol.

The term “therapeutically effective amount” is the amount present in the composition that is needed to provide the desired level of drug in the subject to be treated to give the anticipated physiological response.

The term “physiologically effective amount” is that amount delivered to a subject to give the desired palliative or curative effect.

The term “pharmaceutically acceptable carrier” means that the carrier can be taken into the lungs with no significant adverse toxicological effects on the lungs.

The types of pharmaceutical excipients that are useful as carrier include stabilizers such as human serum albumin (HSA), bulking agents such as carbohydrates, amino acids and polypeptides; pH adjusters or buffers; salts such as sodium chloride; and the like. These carriers may be in a crystalline or amorphous form or may be a mixture of the two.

Bulking agents that are particularly valuable include compatible carbohydrates, polypeptides, amino acids or combinations thereof. Suitable carbohydrates include monosaccharides such as galactose, D-mannose, sorbose, and the like; disaccharides, such as lactose, trehalose, and the like; cyclodextrins, such as 2-hydroxypropyl-.beta.-cyclodextrin; and polysaccharides, such as raffinose, maltodextrins, dextrans, and the like; alditols, such as mannitol, xylitol, and the like. A group of carbohydrates may include lactose, threhalose, raffinose maltodextrins, and mannitol. Suitable polypeptides include aspartame. Amino acids include alanine and glycine, with glycine being used in some embodiments.

Additives, which are minor components of the composition of this invention, may be included for conformational stability during spray drying and for improving dispersibility of the powder. These additives include hydrophobic amino acids such as tryptophan, tyrosine, leucine, phenylalanine, and the like.

Suitable pH adjusters or buffers include organic salts prepared from organic acids and bases, such as sodium citrate, sodium ascorbate, and the like; sodium citrate may be used in some embodiments.

Pulmonary administration of a micellar iRNA formulation may be achieved through metered dose spray devices with propellants such as tetrafluoroethane, heptafluoroethane, dimethylfluoropropane, tetrafluoropropane, butane, isobutane, dimethyl ether and other non-CFC and CFC propellants.

Oral or Nasal Delivery. Any of the siRNAs described herein can be administered orally, e.g., in the form of tablets, capsules, gel capsules, lozenges, troches or liquid syrups. Further, the composition can be applied topically to a surface of the oral cavity.

Any of the siRNAs described herein can be administered nasally. Nasal administration can be achieved by introduction of a delivery device into the nose, e.g., by introducing a delivery device which can dispense the medication. Methods of nasal delivery include spray, aerosol, liquid, e.g., by drops, or by topical administration to a surface of the nasal cavity. The medication can be provided in a dispenser with delivery of the medication, e.g., wet or dry, in a form sufficiently small such that it can be inhaled. The device can deliver a metered dose of medication. The subject, or another person, can administer the medication.

Nasal delivery is effective not only for disorders which directly affect nasal tissue, but also for disorders which affect other tissue siRNAs can be formulated as a liquid or nonliquid, e.g., a powder, crystal, or for nasal delivery. As used herein, the term “crystalline” describes a solid having the structure or characteristics of a crystal, i.e., particles of three-dimensional structure in which the plane faces intersect at definite angles and in which there is a regular internal structure. The compositions of the invention may have different crystalline forms. Crystalline forms can be prepared by a variety of methods, including, for example, spray drying.

For ease of exposition the formulations, compositions and methods in this section are discussed largely with regard to modified siRNAs. It may be understood, however, that these formulations, compositions and methods can be practiced with other siRNAs, e.g., unmodified siRNAs, and such practice is within the invention. Both the oral and nasal membranes offer advantages over other routes of administration. For example, drugs administered through these membranes have a rapid onset of action, provide therapeutic plasma levels, avoid first pass effect of hepatic metabolism, and avoid exposure of the drug to the hostile gastrointestinal (GI) environment. Additional advantages include easy access to the membrane sites so that the drug can be applied, localized and removed easily.

In oral delivery, compositions can be targeted to a surface of the oral cavity, e.g., to sublingual mucosa which includes the membrane of ventral surface of the tongue and the floor of the mouth or the buccal mucosa which constitutes the lining of the cheek. The sublingual mucosa is relatively permeable thus giving rapid absorption and acceptable bioavailability of many drugs. Further, the sublingual mucosa is convenient, acceptable and easily accessible.

The ability of molecules to permeate through the oral mucosa appears to be related to molecular size, lipid solubility and peptide protein ionization. Small molecules, less than 1000 daltons appear to cross mucosa rapidly. As molecular size increases, the permeability decreases rapidly. Lipid soluble compounds are more permeable than non-lipid soluble molecules. Maximum absorption occurs when molecules are un-ionized or neutral in electrical charges. Therefore charged molecules present the biggest challenges to absorption through the oral mucosae.

A pharmaceutical composition of iRNA may also be administered to the buccal cavity of a human being by spraying into the cavity, without inhalation, from a metered dose spray dispenser, a mixed micellar pharmaceutical formulation as described above and a propellant. In one embodiment, the dispenser is first shaken prior to spraying the pharmaceutical formulation and propellant into the buccal cavity. For example, the medication can be sprayed into the buccal cavity or applied directly, e.g., in a liquid, solid, or gel form to a surface in the buccal cavity. This administration is particularly desirable for the treatment of inflammations of the buccal cavity, e.g., the gums or tongue, e.g., in one embodiment, the buccal administration is by spraying into the cavity, e.g., without inhalation, from a dispenser, e.g., a metered dose spray dispenser that dispenses the pharmaceutical composition and a propellant.

An aspect of the invention also relates to a method of delivering an oligonucleotide into the CNS by intrathecal or intracerebroventricular delivery, or into an ocular tissue by ocular delivery, e.g., an intravitreal delivery.

Some embodiments relates to a method of reducing the expression of a target gene in a subject, comprising administering to the subject the oligonucleotide described herein. In one embodiment, the oligonucleotide is administered intrathecally or intracerebroventricularly (to reduce the expression of a target gene in a brain or spine tissue). In one embodiment, the oligonucleotide is administered ocularly, e.g., intravitreally, (to reduce the expression of a target gene in an ocular tissue).

Another aspect of the invention relates to a method of bioactivating an oligonucleotide that comprises one or more 2′-modified nucleosides, wherein the 2′-position of the nucleoside is modified by a bio-cleavable linking group optionally connected to a ligand. The method comprises the step of: exposing the oligonucleotide to a physiological condition that causes the bio-cleavable linking group to be cleaved from the 2′-modified nucleoside, thereby regenerating the 2′—OH group of the nucleoside. The bio-cleavable linking group comprises acetal, disulfide, carbamate, amide, sulfonamide, a biocleavable carbohydrate linker, or combinations thereof.

All the above embodiments relating to the oligonucleotide and structural formula at the 2′-position of the nucleoside in the above aspect of the invention relating to the oligonucleotide are suitable in this aspect of the invention relating to a method of bioactivating an oligonucleotide.

The method is further illustrated in Examples 1-2 below.

The invention is further illustrated by the following examples, which should not be construed as further limiting. The contents of all references, pending patent applications and published patents, cited throughout this application are hereby expressly incorporated by reference.

### EXAMPLES

The invention now being generally described, it will be more readily understood by reference to the following examples which are included merely for purposes of illustration of certain aspects and embodiments of the present invention and are not intended to limit the invention.

### Example 1. Design and Synthesis of Novel Cleavable Linkers at 2′-Position for Improved siRNA Delivery

General synthetic approaches of nucleoside building blocks containing 2′-cleavable linkers and the examples of linker structures are shown below. Briefly, under specific cellular environments and certain conditions such as oxidative and/or reductive conditions, enzyme-triggered degradation can unmask and release the 2′-cleavable linkers form the nucleosides and regenerate 2′-hydroxy group at specific position of siRNA. The linkers possess structural units at which specific cellular enzyme can react as its substrate. The linkers are also connected to self-immolative linker units, which are also released spontaneously at physiological conditions.

The linkers are introduced into nucleoside ribose 2′-position and the phosphoramidite or H-phosphonate building blocks are used for oligonucleotide synthesis. Even though the examples shown here are based on uridine, the chemistries are applicable to other nucleosides, such as adenosine, guanosine, cytidine, etc., in a similar synthetic manner.

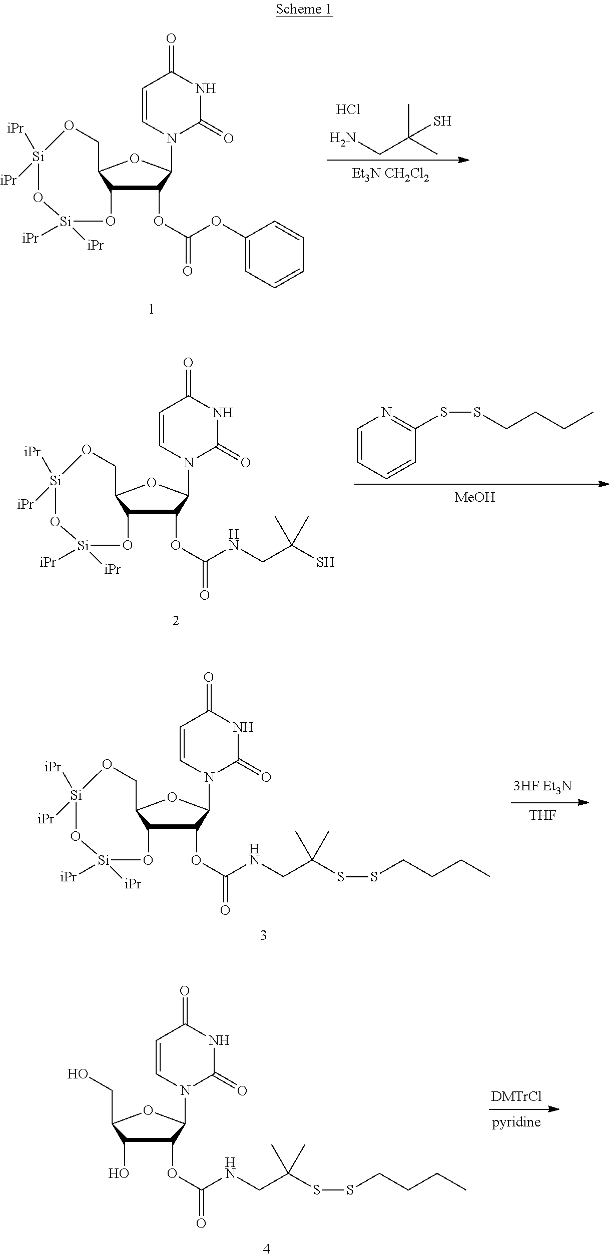

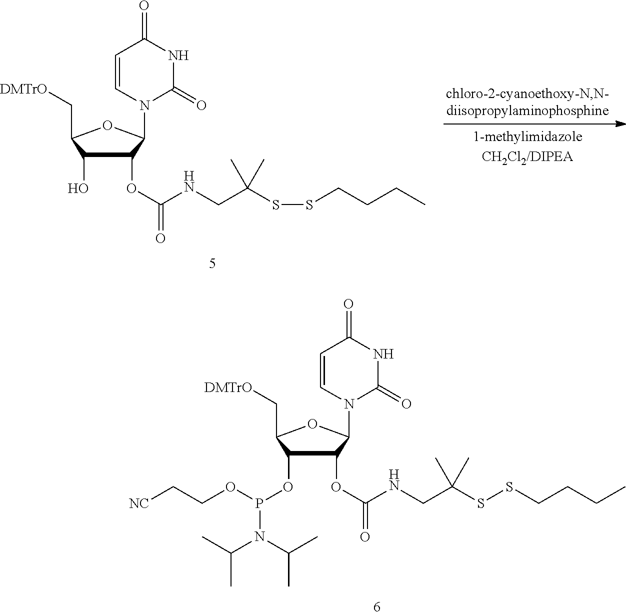

Compound 2: Compound 1 (5.00 g, 8.24 mmol) was dissolved in anhydrous dichloromethane (DCM) (20 mL), and 1-amino-2-methylpropane-2-thiol hydrochloride (6.24 g, 19.78 mmol, 2.4 equivalent) was added. The reaction was cooled to 0° C. on ice bath, then N, N-diisopropylethylamine (DIPEA) (5.74 mL, 41.20 mmol, 5 equivalent) was added dropwise. The reaction was removed from ice, warmed to room temperature, and stirred overnight. After TLC confirmed reaction completion, the reaction was diluted with DCM and standard aqueous workup was performed with saturated aqueous NaHCO₃ solution. The organic layers were pooled, washed with sat brine solution, dried over anhydrous Na₂SO₄, and concentrated under reduced pressure. The crude residue was dried in vacuo before resuspending in DCM and pre-absorbed to silica gel. The crude material was purified via flash chromatography, and eluted with gradient 0-35% ethyl acetate (EtOAc) in hexanes. After fractional analysis via TLC, the fractions containing the desired compound was combined then concentrated under reduced pressure. The desired compound was dried in vacuo and isolated as a white foam (4.18 g, 6.76 mmol, 82%). ¹H NMR (400 MHz, DMSO-d₆) δ 11.41 (d, J=2.2 Hz, 1H), 7.70 (d, J=8.1 Hz, 1H), 7.59 (d, J=6.5 Hz, 1H), 5.67 (s, 1H), 5.59 (dd, J=8.1, 2.1 Hz, 1H), 5.37 (dd, J=5.6, 1.7 Hz, 1H), 4.49 (dd, J=8.4, 5.6 Hz, 1H), 4.09 (dd, J=12.8, 3.8 Hz, 1H), 3.98-3.85 (m, 2H), 3.13 (qd, J=13.7, 6.4 Hz, 2H), 2.73 (s, 1H), 1.23 (d, J=3.8 Hz, 5H), 1.10-0.93 (m, 26H), 0.92-0.82 (m, 1H).

Compound 3: In a round bottom flask with magnetic stir bar, Compound 2 (4.18 g, 6.76 mmol) was dissolved in methanol (35 mL). In a separate vial, 2-(butyldisulfanyl) pyridine (2.70 g, 13.53 mmol, 2 equivalent) was dissolved in methanol (25 mL) then added dropwise to the reaction flask. The vial was rinsed with additional methanol (7 mL). The reaction was stirred overnight at ambient temperature. After TLC confirmed lack of starting material, the reaction mixture was removed from stir, diluted with methanol, and concentrated under reduced pressure. The crude residue was resuspended in dichloromethane, pre-absorbed to silica gel and dried in vacuo for 1 hour. The crude was purified via flash chromatography, and eluted with gradient 0-27% EtOAc in hexanes. Fractional analysis was completed via TLC and the fractions containing desired compound were combined, concentrated, and dried in vacuo overnight. Compound 3 was isolated as a white foam (4.56 g, 6.46 mmol, 95%). ¹H NMR (500 MHz, DMSO-d₆) δ 11.41 (s, 1H), 7.69 (d, J=8.0 Hz, 1H), 7.56 (t, J=6.3 Hz, 1H), 5.66 (d, J=1.8 Hz, 1H), 5.59 (d, J=8.1 Hz, 1H), 5.36 (dd, J=5.6, 1.8 Hz, 1H), 4.49 (dd, J=8.4, 5.6 Hz, 1H), 4.09 (dd, J=12.9, 3.9 Hz, 1H), 3.94 (dd, J=12.9, 2.9 Hz, 1H), 3.91-3.84 (m, 1H), 3.14 (qd, J=13.7, 6.4 Hz, 2H), 2.70 (t, J=7.3 Hz, 2H), 1.56 (p, J=7.3 Hz, 2H), 1.34 (h, J=7.4 Hz, 2H), 1.23 (d, J=4.9 Hz, 1H), 1.18 (d, J=2.9 Hz, 5H), 1.10-0.94 (m, 20H), 0.87 (t, J=7.4 Hz, 4H).

Compound 4: Compound 3 (4.56 g, 6.46 mmol) was dissolved in anhydrous tetrahydrofuran (32 mL) at room temperature. The reaction was cooled to 0° C. on ice. Triethylamine trihydrofluoride, 18 M in THF, (1.79 mL, 32.39, 5 equivalent) was added slowly to the reaction. After 10 minutes, the reaction flask was removed from ice and continued to stir at ambient temperature for 2.5 hours. Once TLC confirmed lack of starting material, the reaction was taken off stir, concentrated under reduced pressure, and dried in vacuo overnight. The crude residue was resuspended in DCM, pre-absorbed to silica gel, then purified via flash chromatography, and eluted with gradient 0-8% MeOH in DCM. Fractional analysis was done via TLC and the fractions containing desired compound were combined, concentrated, and dried in vacuo overnight. Compound 4 was isolated as white chalky solid (2.87 g, 6.91 mmol, 95%). ¹H NMR (400 MHz, DMSO-d₆) δ 11.33 (s, 1H), 7.90 (d, J=8.1 Hz, 1H), 7.48 (t, J=6.3 Hz, 1H), 5.99 (d, J=6.1 Hz, 1H), 5.67 (d, J=8.1 Hz, 1H), 5.48 (d, J=5.2 Hz, 1H), 5.19 (t, J=4.9 Hz, 1H), 5.05 (t, J=5.7 Hz, 1H), 4.20 (td, J=5.3, 3.5 Hz, 1H), 3.89 (q, J=3.3 Hz, 1H), 3.69-3.51 (m, 2H), 3.17 (d, J=5.2 Hz, 1H), 3.11 (dd, J=6.5, 2.4 Hz, 2H), 2.69 (t, J=7.3 Hz, 2H), 1.56 (p, J=7.2 Hz, 2H), 1.34 (h, J=7.3 Hz, 2H), 1.17 (d, J=5.2 Hz, 6H), 0.87 (t, J=7.4 Hz, 3H). ¹³C NMR (126 MHz, d₂) δ 162.99, 155.64, 150.56, 140.65, 102.14, 85.68, 85.46, 74.76, 68.92, 60.88, 51.00, 49.61, 48.59, 40.11, 40.02, 39.95, 39.85, 39.78, 39.69, 39.61, 39.52, 39.44, 39.35, 39.19, 39.02, 30.77, 24.94, 24.84, 20.92, 13.49.

Compound 5: Prior to reaction, Compound 4 (2.87 g, 6.91 mmol) was co-evaporated twice with pyridine then dried in vacuo overnight. Compound 4 was resuspended in anhydrous pyridine (31 mL), cooled to 0° C. on ice and 4,4′-dimethoxytrityl chloride (2.52 g, 7.43 mmol, 1.2 equivalent) was added. After stirring for 10 min at 0° C., the ice bath was removed, and reaction continued to stir at ambient temperature overnight. After confirming the lack of starting material via TLC analysis, the reaction was quenched with methanol (2 mL). The reaction was removed from stir and concentrated under reduced pressure. The crude residue was dried in vacuo to remove residual pyridine. The remaining crude was resuspended in EtOAc and standard aqueous workup was performed with saturated aqueous NaHCO₃ solution. The organic layers were pooled, washed with saturated brine solution, dried over anhydrous Na₂SO₄, concentrated under reduced pressure, then dried in vacuo overnight. The crude residue was resuspended in DCM and pre-absorbed to silica gel (pre-treated with 2% TEA), then purified via flash chromatography, eluted 0-65% EtOAc in Hexanes. TLC fractional analysis was performed and fractions containing desired compound were combined, concentrated, co-evaporated twice with acetonitrile then dried in vacuo overnight. Compound 5 was isolated as a white foam (4.11 g, 5.37 mmol, 86%). ¹H NMR (500 MHz, DMSO-d₆) δ 11.39 (s, 1H), 7.69 (d, J=8.1 Hz, 1H), 7.52 (t, J=6.3 Hz, 1H), 7.41-7.37 (m, 2H), 7.31 (t, J=7.6 Hz, 2H), 7.25 (dd, J=7.5, 5.1 Hz, 5H), 6.93-6.86 (m, 4H), 5.92 (d, J=4.8 Hz, 1H), 5.54 (d, J=5.7 Hz, 1H), 5.38 (d, J=8.0 Hz, 1H), 5.17 (t, J=5.2 Hz, 1H), 4.34 (q, J=5.6 Hz, 1H), 4.01-3.95 (m, 1H), 3.74 (s, 6H), 3.28 (dd, J=10.6, 4.7 Hz, 1H), 3.21 (dd, J=10.6, 2.8 Hz, 1H), 3.18-3.11 (m, 2H), 2.70 (t, J=7.3 Hz, 2H), 1.61-1.51 (m, 2H), 1.34 (q, J=7.4 Hz, 2H), 1.19 (d, J=4.0 Hz, 6H), 0.86 (t, J=7.4 Hz, 3H).

Compound 6: To prepare for phosphitylation, Compound 5 (200 mg, 261 μmol) was co-evaporated with pyridine twice then dried in vacuo. The round bottom flask containing the starting material was flushed with argon several times then fitted with balloon containing argon. Compound 5 was dissolved in anhydrous dichloromethane (31 mL) and DIPEA (90.9 μL, 522 μmol, 2 equivalent) was added drop wise. After few minutes of stirring, 1-methylimdiazole (20.8 μL, 261 μmol, 1.0 equivalent) was added. A fter the reaction was cooled to 0° C. on ice bath, 2-cyanoethyl-N,N-diisopropylchlorophosphoramidite (31 μL, 287 mmol, 1.1 equivalent) was added slowly. The reaction stirred on ice for 10 minutes, then allowed to come to room temperature, and stirred for 2 hours. After TLC analysis confirmed lack of starting material, the reaction was removed from stir, diluted with DCM, and quick standard aqueous workup was performed. The organic layers were pooled, washed with saturated brine solution, dried over anhydrous Na₂SO₄, concentrated under reduced pressure, and then dried in vacuo for several hours. The crude residue was resuspended in minimal DCM, and then the liquid was loaded to a 12 g Gold Teledyne Isco column (pre-treated with bolus of TEA then flushed 5 column volume of hexanes), purified via flash chromatography, and eluted with gradient 70% EtOAc in hexanes. Fractional analysis was completed by TLC and the fractions containing desired compound were combined, concentrated, co-evaporated several times with acetonitrile, and then dried in vacuo overnight. The desired phosphoramidite Compound 6 was isolated as white foam (181 mg, 187 μmol, 72%) and stored at −20° C. under argon atmosphere. ¹H NMR (400 MHz, acetonitrile-d₃) δ 8.96 (s, 1H), 7.62 (t, J=8.7 Hz, 1H), 7.48-7.42 (m, 2H), 7.37-7.28 (m, 6H), 7.28-7.22 (m, 1H), 6.88 (dd, J=8.9, 3.6 Hz, 4H), 6.00 (dd, J=13.0, 5.7 Hz, 1H), 5.85 (dt, J=23.2, 6.4 Hz, 1H), 5.41 (t, J=5.5 Hz, 1H), 5.33 (dd, J=7.8, 5.5 Hz, 1H), 4.60 (d, J=6.5 Hz, 1H), 4.20 (dd, J=23.3, 3.8 Hz, 1H), 3.77 (d, J=1.6 Hz, 6H), 3.59 (tdd, J=13.7, 6.3, 3.1 Hz, 2H), 3.41 (d, J=3.1 Hz, 1H), 3.38-3.24 (m, 2H), 2.77-2.65 (m, 3H), 2.50 (t, J=6.1 Hz, 1H), 1.61 (qt, J=7.6, 4.6 Hz, 2H), 1.43-1.33 (m, 2H), 1.29-1.21 (m, 6H), 1.21-1.09 (m, 10H), 1.05 (d, J=6.8 Hz, 3H), 0.90 (t, J=7.3 Hz, 3H). ³¹P NMR (202 MHz, CD₃CN) δ 151.06, 150.88.

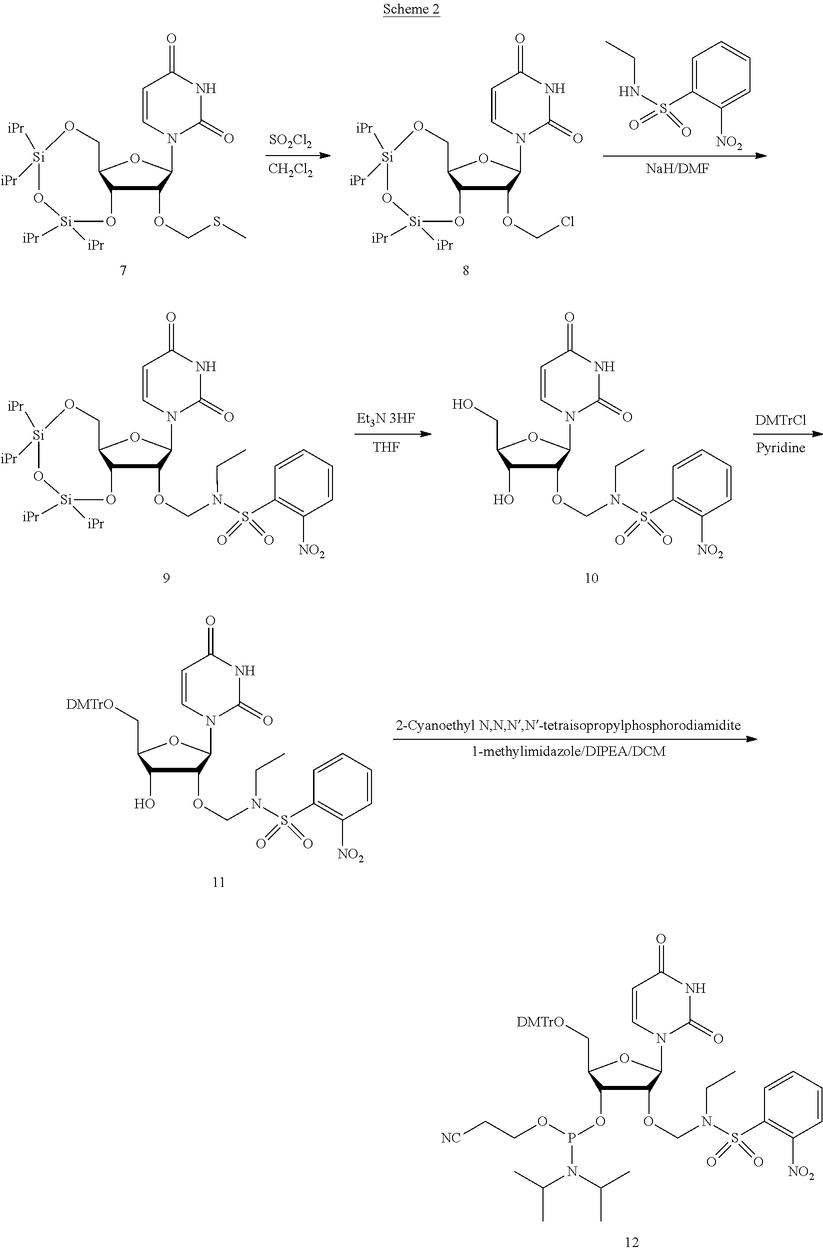

Compound 9: To a solution of Compound 7 (5.10 g, 9.33 mmol) in dichloromethane (75 mL) was added sulfuryl chloride (0.983 mL, 12.1 mmol) in CH₂Cl₂ (10 mL) dropwise at room temperature. The reaction mixture was stirred for 4 hours, and then the solvent was evaporated to give Compound 8 as white foam. To a solution of N-Ethyl-2-nitrobenzene-1-sulfonamide (3.24 g, 14.1 mmol) in DMF 50 mL was added NaH (405 mg, 16.9 mmol) was added at 0° C. The mixture was stirred at room temperature for 45 minutes then a solution of the crude Compound 8 dissolved in DMF (50 mL) was dropwise added at room temperature. The reaction mixture was stirred overnight, and then evaporated. The residue was extracted with EtOAc (300 mL) and saturated aqueous NaCl solution (150 mL). The organic layer was separated, dried over anhydrous Na₂SO₄, filtered, and evaporated. The crude material was purified via flash chromatography, and eluted with 0-60% EtOAc in hexanes to give Compound 9 as a white foam (3.55 g, 4.87 mmol, 52% from Compound 7). ¹H NMR (400 MHz, DMSO-d₆) δ 11.43 (s, 1H), 8.15 (d, J=7.8 Hz, 1H), 7.97 (d, J=8.2 Hz, 2H), 7.93-7.79 (m, 3H), 7.68-7.59 (m, 2H), 5.61-5.49 (m, 5H), 5.17-4.98 (m, 2H), 4.36-4.22 (m, 3H), 4.12 (q, J=11.7 Hz, 2H), 3.97-3.85 (m, 5H), 3.36 (q, J=7.1 Hz, 3H), 1.11 (q, J=6.9 Hz, 4H), 1.08-0.89 (m, 71H).

Compound 10: To a solution of Compound 9 (3.54 g, 4.86 mmol) was dissolved in anhydrous THE (25 mL) at room temperature then cooled to 0° C. on ice. After triethylamine trihydrofluoride (1.50 mL, 27.5 mmol) was carefully added dropwise to reaction, mixture was brought to room temperature and stirred for 4 hours. After TLC analysis showed lack of starting material, the reaction was removed from stir and concentrated under reduced pressure. The crude residue was dissolved in DCM and pre-absorbed to silica gel to purify via flash chromatography, and eluted with 0-8% MeOH in CH₂Cl₂, to give Compound 10 as a white foam (613 mg, 1.26 mmol, 25%). ¹H NMR (500 MHz, DMSO-d₆) δ 11.35 (d, J=1.7 Hz, 1H), 8.10 (dd, J=7.9, 1.4 Hz, 1H), 7.96 (dd, J=7.8, 1.4 Hz, 1H), 7.91-7.85 (m, 2H), 7.82 (td, J=7.7, 1.4 Hz, 1H), 5.87 (d, J=4.8 Hz, 1H), 5.65 (dd, J=8.1, 1.5 Hz, 1H), 5.39 (d, J=5.6 Hz, 1H), 5.13 (t, J=4.9 Hz, 1H), 5.02-4.91 (m, 2H), 4.14 (dq, J=11.5, 5.1 Hz, 2H), 4.09 (q, J=5.3 Hz, 1H), 3.60 (dddd, J=42.4, 12.0, 4.9, 3.1 Hz, 2H), 3.28 (qd, J=7.2, 4.1 Hz, 2H), 3.17 (d, J=5.2 Hz, 1H), 1.04 (t, J=7.1 Hz, 3H).

Compound 11: Compound 10 (610 mg, 1.25 mmol) was co-evaporated in anhydrous pyridine twice (10 mL each), and then dried in vacuo. The starting material was re-suspended in anhydrous pyridine (6 mL), cooled to 0° C. on ice, and then 4,4′-dimethoxytrityl chloride (5.08 mg, 1.50 mmol) was added to the reaction. The reaction mixture was removed from ice bath after 5 minutes, and then stirred for 14 hours at ambient temperature. After TLC analysis confirmed lack of starting materials, the reaction mixture was removed from stir, concentrated under reduced pressure, and dried in vacuo for few hours. The crude residue was resuspended in EtOAc, and standard aqueous work-up was performed. The organic extracts were combined, concentrated under reduced pressure, and then dried in vacuo. The crude was purified via flash chromatography, and eluted with 04% MeOH in CH₂Cl₂ to give compound 11 as a white foam (710 mg, 0.900 mmol, 72%). ¹H NMR (400 MHz, DMSO-d₆) δ 11.40 (d, J=2.2 Hz, 1H), 8.10 (dd, J=7.7, 1.5 Hz, 1H), 7.97 (dd, J=7.8, 1.5 Hz, 1H), 7.86 (dtd, J=20.3, 7.5, 1.4 Hz, 2H), 7.71 (d, J=8.1 Hz, 1H), 7.42-7.20 (m, 11H), 6.93-6.87 (m, 4H), 5.84 (d, J=4.2 Hz, 1H), 5.52 (d, J=6.3 Hz, 1H), 5.25 (dd, J=8.1, 2.1 Hz, 1H), 5.03 (s, 2H), 4.33 (q, J=5.6 Hz, 1H), 4.21 (t, J=4.9 Hz, 1H), 3.74 (s, 6H), 3.41-3.25 (m, 4H), 3.20 (dd, J=10.7, 2.8 Hz, 1H), 1.07 (t, J=7.1 Hz, 3H).

Compound 12: To a solution of Compound 11 (1.00 g, 1.27 mmol) in DCM (10 ml) and DIPEA (0.664 ml, 3.81 mmol) was added 2-cyanoethyl N,N-diisopropylchlorophosphoramidite (0.370 ml, 1.65 mmol) and i-methylimidazole (0.101 mL, 1.27 mmol) at 0° C. The mixture was stirred at 0° C. for 2 hours. The reaction mixture was diluted with CH₂Cl₂ (100 mL), and then washed with saturated NaHCO₃ aqueous solution (100 mL). The organic layer was separated, dried over anhydrous Na₂SO₄, filtered, and concentrated. The amidite was purified via flash chromatography, and eluted with 0-66% EtOAc in hexane to give Compound 12 as a white foam (930 mg, 0.940 mmol, 74%). ³¹P NMR (202 MHz, CD₃CN) δ 151.50, 150.70.

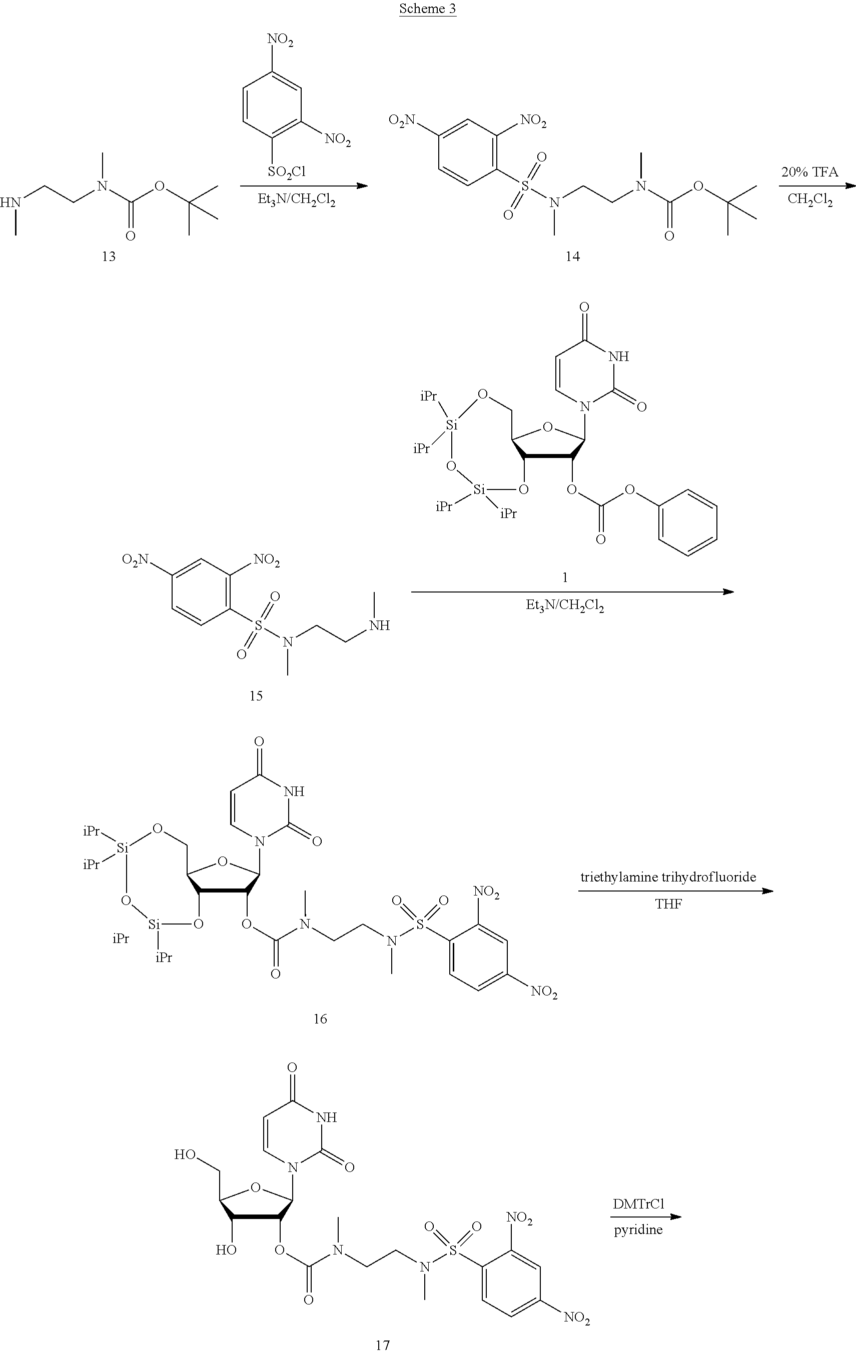

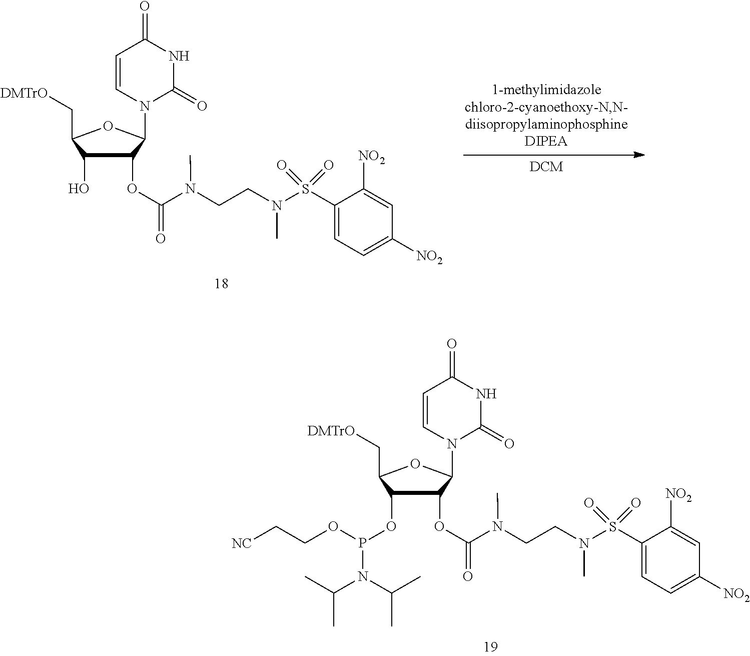

Compound 14: In a 1 L round bottom flask fitted with magnetic stir bar, tert-butyl N-methyl-N-[2-(methylamino) ethyl] carbamate 13 (8.75 g, 46.48 mmol, 1.0 equivalent) and 2,4-dinitrobenzenesulfonyl chloride (13.63 g, 51.12 mmol, 1.1 equivalent) were dissolved in anhydrous dichloromethane (430 mL). The reaction was cooled to 0° C. on ice bath, and triethylamine (12.96 mL, 92.95 mmol, 2.0 equivalent) was added drop wise. After 10 minutes, the ice bath was removed, and the reaction was stirred at ambient temperature overnight. After TLC analysis confirmed lack of starting material, the reaction was diluted with DCM, and standard aqueous workup was performed. The dried and concentrated crude residue was resuspended in DCM, pre-absorbed to silica gel, then purified via flash chromatography, and eluted with 30% EtOAc in hexanes to give Compound 14 as an orange-brown oil (18.44 g, 44.07 mmol, 94%). ¹H NMR (400 MHz, DMSO-d₆) δ 8.97 (d, J=2.3 Hz, 1H), 8.56 (dd, J=8.7, 2.3 Hz, 1H), 8.24 (d, J=8.7 Hz, 1H), 3.32 (s, 1H), 2.91 (s, 4H), 2.79 (d, J=5.4 Hz, 3H), 1.40 (s, 9H). ¹³C NMR (101 MHz, DMSO) δ 154.51, 149.88, 147.67, 135.57, 131.58, 126.96, 120.04, 78.78, 59.73, 47.74, 46.02, 45.19, 34.81, 34.47, 34.18, 33.99, 27.96, 14.07.

Compound 15: Compound 14 (18.30 g, 43.74 mmol) was dissolved in anhydrous dichloromethane (315 mL), and then cooled to 0° C. on ice. After 10 minutes on ice, 2,2,2-trifluoroacetic acid (69 mL, 895.61 mmol) in 20% v/v DCM was added via addition funnel to the reaction mixture. Additional DCM (30 mL) was used to rinse the addition funnel and added to the reaction. The reaction was slowly warmed to ambient temperature and stirred for 5 hours. After TLC analysis verified lack of starting material, the reaction mixture was removed from stir, diluted with DCM, and then concentrated under reduced pressure. The residue was co-evaporated twice with toluene then dried in vacuo to give Compound 15 as TFA salt (27.04 g, quantitative yield). ¹H NMR (400 MHz, DMSO-d₆) δ 9.01 (d, J=2.3 Hz, 1H), 8.71 (s, 3H), 8.60 (dd, J=8.7, 2.3 Hz, 1H), 8.28 (d, J=8.7 Hz, 1H), 3.51 (t, J=6.3 Hz, 2H), 3.19 (p, J=6.1 Hz, 3H), 2.95 (s, 4H), 2.61 (t, J=5.2 Hz, 4H).

Compound 16: Compound 15 (1.70 g, 3.94 mmol, TFA salt) was dissolved in anhydrous dichloromethane (8.21 mL). To the reaction flask, Compound 1 (3.59 g, 5.91 mmol, 1.5 equivalent) was added and stirred at room temperature to dissolve. Once both starting materials were in solution, the reaction was cooled to 0° C. on ice. Triethylamine (1.65 mL, 11.82 mmol, 3.0 equivalent) was added drop wise to the reaction. The reaction was brought to ambient temperature, then was heated at reflux, and continued to stir. After heating overnight, TLC analysis showed both starting material and formation of new spot. Additional reagents, including Compound 15 (0.5 equivalent), DCM (4 mL) and TEA (1 mL), were added to the reaction mixture at ambient temperature. The reaction was heated at reflux. After stirring for additional 48 hours, the reaction was removed from stir, diluted with DCM, and then standard aqueous workup was performed. The crude residue was purified via silica gel flash chromatography, and the desired compound was eluted with 80% EtOAc in hexanes to give Compound 16 as a yellow foam (1.13 g, 1.35 mmol, 34%). ¹H NMR (400 MHz, DMSO-d₆) δ 11.43 (s, 1H), 8.57 (dd, J=7.9, 2.8 Hz, 1H), 8.20 (ddd, J=13.3, 9.6, 2.9 Hz, 1H), 7.65 (d, J=8.1 Hz, 1H), 7.40 (t, J=10.5 Hz, 1H), 5.70-5.57 (m, 2H), 5.35 (dd, J=5.8, 2.0 Hz, 1H), 4.55 (dt, J=9.8, 4.8 Hz, 1H), 4.09-3.99 (m, 1H), 3.93 (dd, J=12.6, 2.8 Hz, 1H), 3.82 (ddt, J=19.9, 8.0, 3.9 Hz, 1H), 3.69 (q, J=10.5, 10.0 Hz, 2H), 3.59 (d, J=5.2 Hz, 1H), 2.97-2.80 (m, 6H), 1.12-0.89 (m, 28H), 0.89-0.78 (m, 1H).

Compound 17: Compound 16 (870 mg, 1.05 mmol) was dissolved in anhydrous THF (5 mL) then cooled to 0° C. on ice. Triethylamine trihydrofluoride (18 M, 290.81 μL, 5.23 mmol) was carefully added dropwise to reaction. After stirring for 10 minutes, the ice bath was removed, and reaction was stirred at ambient temperature for 2.5 hours. The reaction was removed from stir, concentrated under reduced pressure, and then dried in vacuo overnight. The crude residue was purified via flash chromatography, and eluted with 10% MeOH in DCM to give Compound 17 as yellow oily solid (510 mg, 0.867 mmol, 83%). ¹H NMR (500 MHz, DMSO-d₆) δ 11.33 (d, J=21.1 Hz, 1H), 8.56 (dd, J=9.5, 2.8 Hz, 1H), 8.20 (td, J=9.4, 2.8 Hz, 1H), 7.90 (dd, J=14.7, 8.1 Hz, 1H), 7.40 (dd, J=26.9, 9.6 Hz, 1H), 6.07 (d, J=6.2 Hz, 1H), 5.67 (dd, J=13.6, 6.9 Hz, 2H), 5.21 (dt, J=21.5, 4.9 Hz, 1H), 5.03 (dt, J=36.7, 5.6 Hz, 1H), 4.27-4.14 (m, 1H), 3.91 (dd, J=19.0, 3.3 Hz, 1H), 3.73-3.51 (m, 3H), 3.43 (ddd, J=22.0, 14.1, 6.7 Hz, 1H), 2.92-2.80 (m, 6H).

Compound 18: Compound 17 (616 mg, 1.05 mmol) was co-evaporated twice with pyridine then dried in vacuo overnight. Compound 17 was resuspended in anhydrous pyridine (9 mL), cooled to 0° C. on ice and 4,4′-dimethoxytrityl chloride (426 mg, 1.26 mmol) was added. After stirring for 10 minutes at 0° C., the ice bath was removed, and the reaction was stirred at ambient temperature overnight. After confirming the lack of starting material via TLC analysis, the reaction was quenched with methanol (1 mL). The reaction was removed from stir and concentrated under reduced pressure. The crude was resuspended in EtOAc and standard aqueous workup was performed with saturated aqueous NaHCO₃ solution. The organic layers were pooled, washed with saturated brine solution, dried over anhydrous Na₂SO₄, and concentrated under reduced pressure. The crude residue was purified via flash chromatography, and eluted 0-4% MeOH in DCM to give Compound 18 as a yellow foam (780 mg, 0.876 mmol, 83%). ¹H NMR (400 MHz, DMSO-d₆) δ 11.39 (d, 1H), 8.55 (dd, J=10.6, 2.8 Hz, 1H), 8.23-8.15 (m, 1H), 7.68 (t, J=8.3 Hz, 1H), 7.46-7.35 (m, 3H), 7.31 (dd, J=8.4, 6.7 Hz, 2H), 7.28-7.20 (m, 5H), 6.90 (d, 4H), 6.01-5.46 (m, 2H), 5.41 (td, J=7.9, 1.8 Hz, 1H), 5.18 (dt, J=21.1, 5.2 Hz, 1H), 4.39-4.25 (m, 1H), 3.74 (s, 7H), 3.57-3.40 (m, 1H), 3.21 (d, J=9.6 Hz, 1H), 2.90 (s, 3H), 2.86 (d, J=3.6 Hz, 3H).

Compound 19: Compound 18 (520 mg, 0.584 mmol) was co-evaporated with acetonitrile twice and dried in vacuo for several hours. Compound 18 was resuspended in anhydrous dichloromethane (7 mL), and then flushed with argon. To the reaction flask, DIPEA (406 μL, 2.33 mmol) and 1-methylimidazole (46.5 μL, 0.584 mmol) were added dropwise. The reaction was cooled to 0° C. on ice and 2-cyanoethyl-N,N-diisopropylchlorophosphoramidite (156 μL, 0.700 mmol) was added dropwise. After 5 minutes, the reaction was removed from ice bath and stirred overnight at ambient temperature. After confirming lack of starting material via TLC, the reaction mixture was diluted with CH₂Cl₂ (50 mL) and then washed with saturated NaHCO₃ aqueous solution (50 mL). The organic layer was separated, dried over anhydrous Na₂SO₄, filtered, and concentrated. The amidite was purified via flash chromatography, and eluted with 0-66% EtOAc in hexane to give Compound 19 as yellow foam (530 mg, 0.486 mmol, 83%). ¹H NMR (500 MHz, acetonitrile-d₃) δ 9.00 (s, 1H), 8.53 (dd, J=10.6, 2.8 Hz, 1H), 8.25-8.13 (m, 1H), 7.69-7.59 (m, 1H), 7.51-7.42 (m, 3H), 7.38-7.20 (m, 8H), 6.88 (dtd, J=9.5, 4.8, 4.3, 2.8 Hz, 4H), 5.43-5.29 (m, 2H), 4.70-4.57 (m, 1H), 4.19-3.98 (m, 2H), 3.81-3.74 (m, 7H), 3.74-3.29 (m, 6H), 2.97-2.86 (m, 5H), 2.75 (t, J=5.9 Hz, 2H), 2.62 (dt, J=22.1, 6.0 Hz, 1H), 2.45 (dtd, J=21.5, 6.0, 2.1 Hz, 1H), 1.23 (t, J=6.7 Hz, 10H), 1.10 (dt, J=23.0, 6.7 Hz, 8H), 1.02 (dd, J=15.4, 6.8 Hz, 3H).

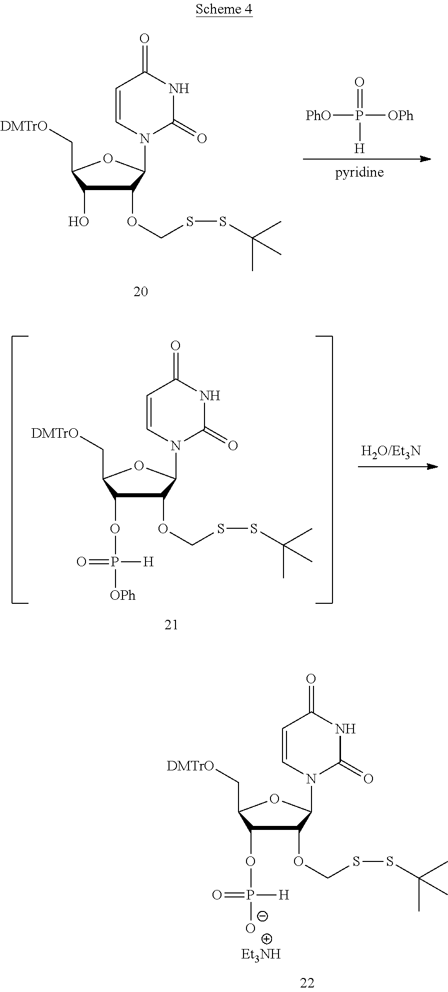

Synthesis of compound 22: Compound 20 (500 mg, 0.734 mmol) was taken in a round bottom flask and co-evaporated with anhydrous pyridine (10 mL) twice and dried in vacuo for 1 hour. The material was dissolved in anhydrous pyridine (18 mL) and diphenyl phosphite (0.565 mL, 2.94 mmol) was added dropwise. The mixture was stirred at room temperature for 45 minutes, then quenched by addition of a mixture of water-triethylamine (1:1, v/v, 4 mL), and left stirring for 1 hour. After removing the solvents under reduced pressure, the residue was partitioned between CH₂Cl₂ (50 mL) and saturated NaHCO₃ aqueous solution (50 mL). The organic layer was additionally washed with saturated NaHCO₃ aqueous solution (50 mL), separated, dried over anhydrous Na₂SO₄, filtered, and concentrated in vacuo. The crude viscous oil was purified by flash column chromatography on silica gel, and eluted with 0-5% MeOH containing 5% Et₃N in CH₂Cl₂ to give Compound 22 as a white foam (210 mg, 0.248 mmol, 34%). ¹H NMR (500 MHz, acetonitrile-d₃) δ 11.74 (s, 1H), 9.94 (s, 1H), 7.73 (d, J=8.1 Hz, 1H), 7.49-7.39 (m, 3H), 7.38-7.29 (m, 6H), 7.26 (t, J=7.3 Hz, 2H), 6.99-6.82 (m, 4H), 5.98 (d, J=4.9 Hz, 1H), 5.19 (d, J=8.1 Hz, 1H), 5.08 (d, J=11.3 Hz, 1H), 4.99 (d, J=11.4 Hz, 1H), 4.91 (dt, J=9.6, 5.1 Hz, 1H), 4.50 (t, J=5.0 Hz, 1H), 4.24 (dt, J=5.2, 2.8 Hz, 1H), 3.77 (s, 6H), 3.41 (dd, J=11.0, 3.0 Hz, 1H), 3.38-3.31 (m, 1H), 2.98 (q, J=7.3 Hz, 8H), 1.30 (s, 9H), 1.22 (t, J=7.3 Hz, 11H). ³¹P NMR (202 MHz, acetonitrile-d3) δ 2.17.

This triethylammonium salt was dissolved in CH₂Cl₂ then converted to its DBU salt by extracted with 0.5 M aqueous DBU solution for the oligonucleotide syntheses.

### Synthesis of 2′-Modified Nucleoside Building Blocks with 2′-Carbamate Linker

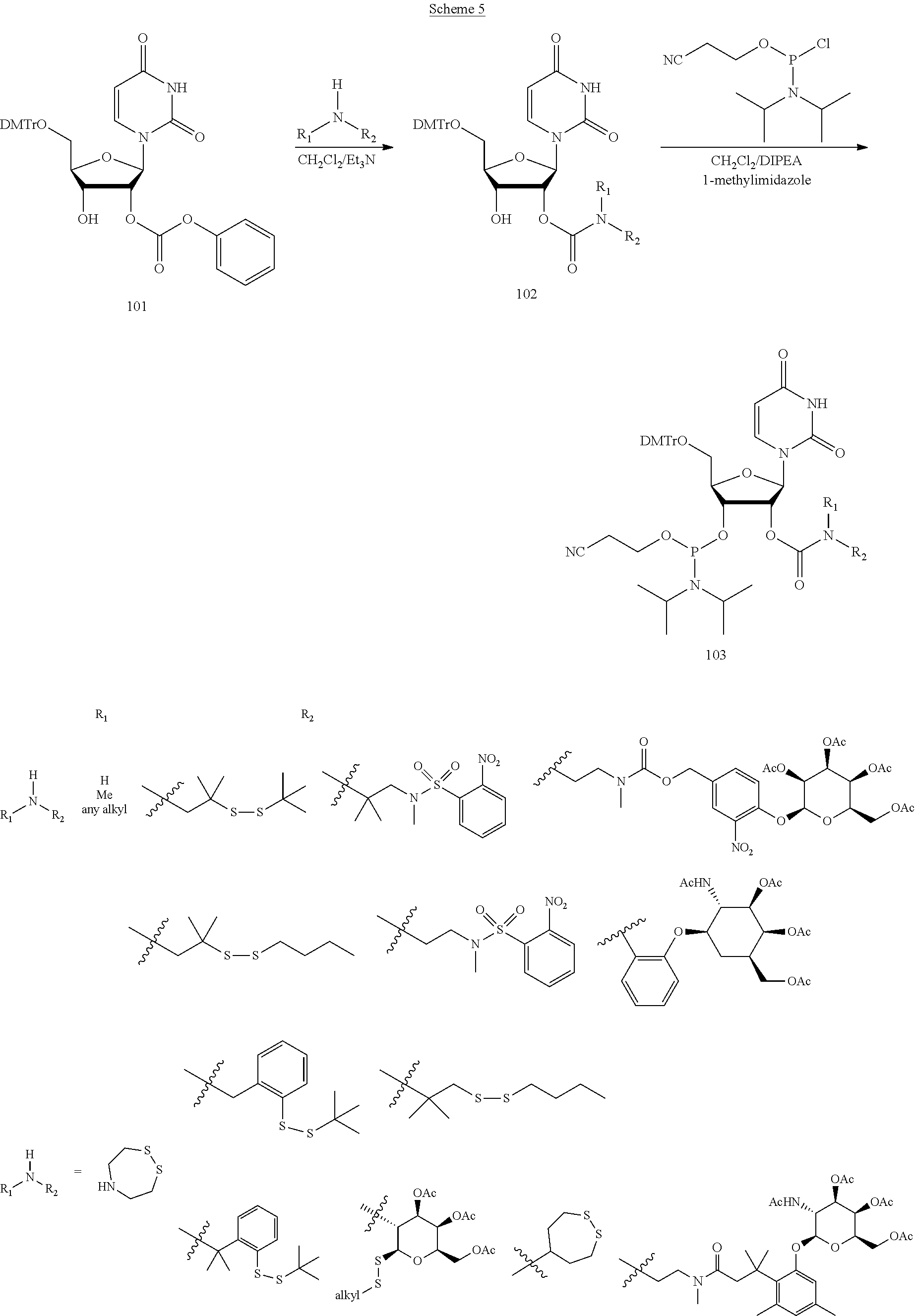

### Synthesis of 2′-Modified Nucleoside Building Blocks with 2′-Acetal Linker

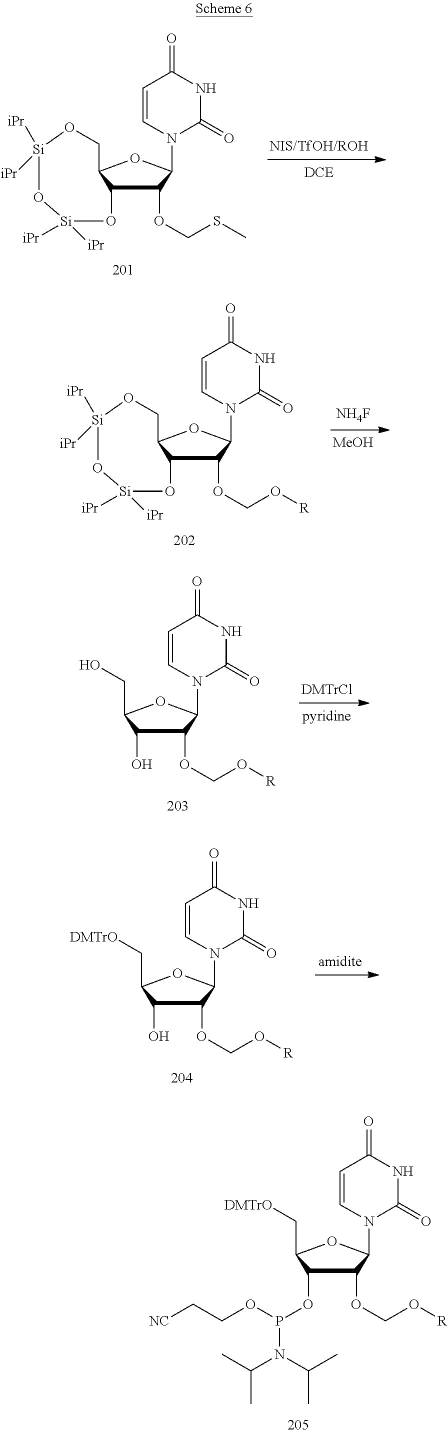

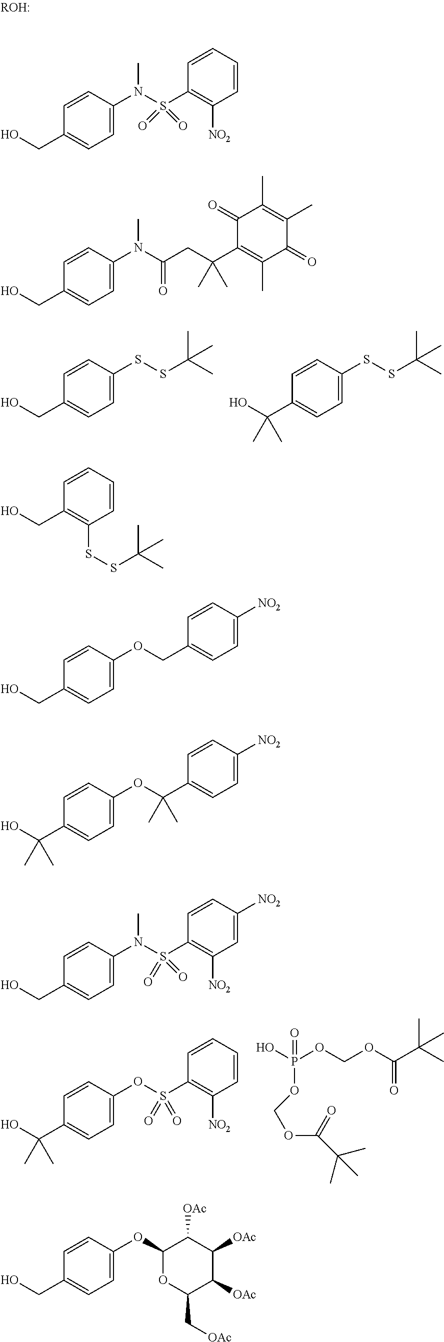

### Synthesis of 2′-Modified Nucleoside Building Blocks with 2′-Methylene Sulfonamide Linker

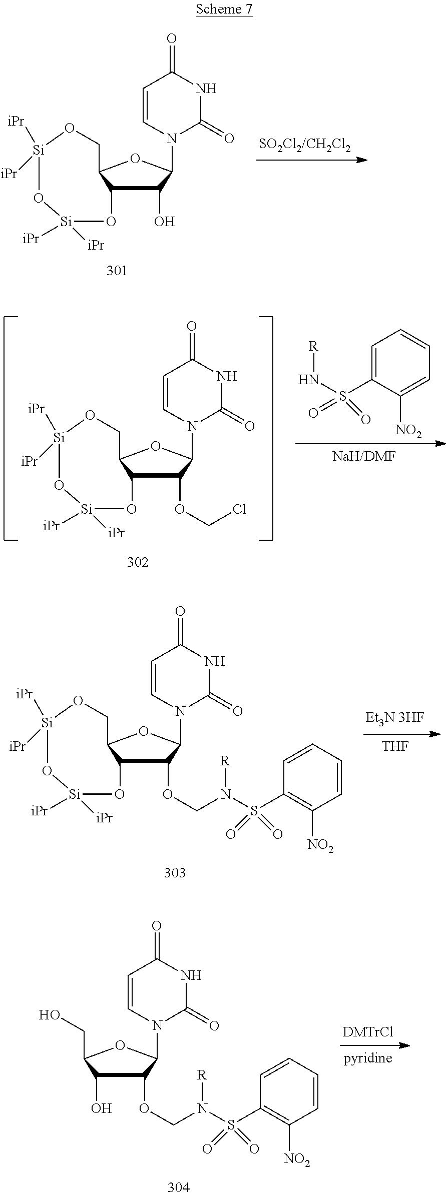

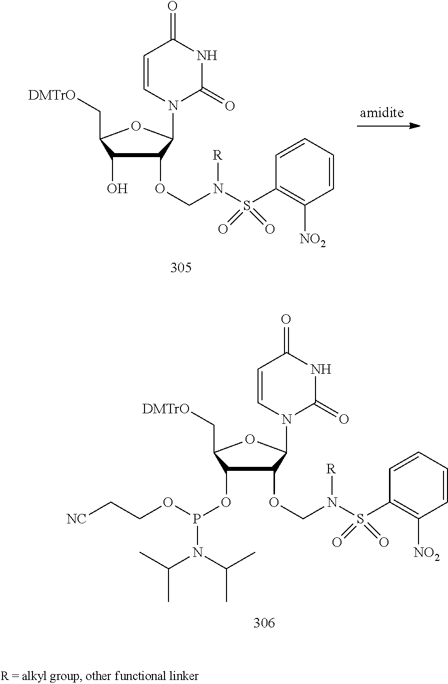

### Synthesis of 2′-Modified Nucleoside Building Blocks with 2′-Methylene Carbamate Linker

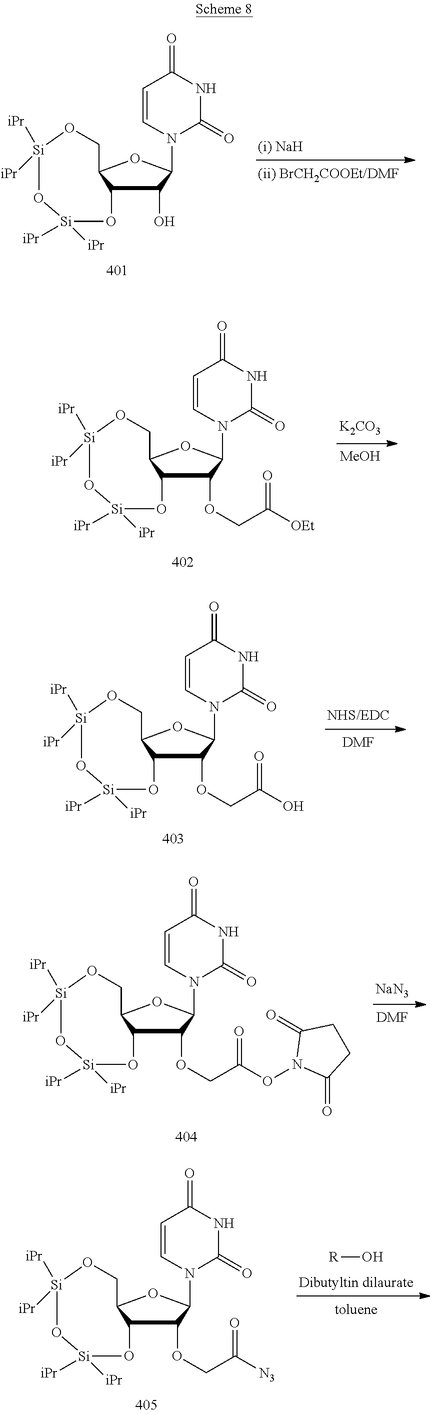

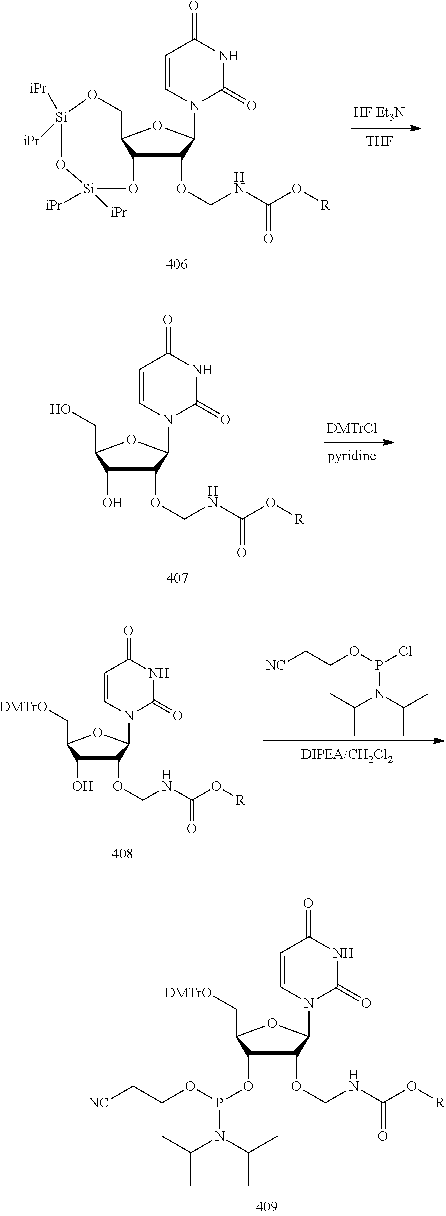

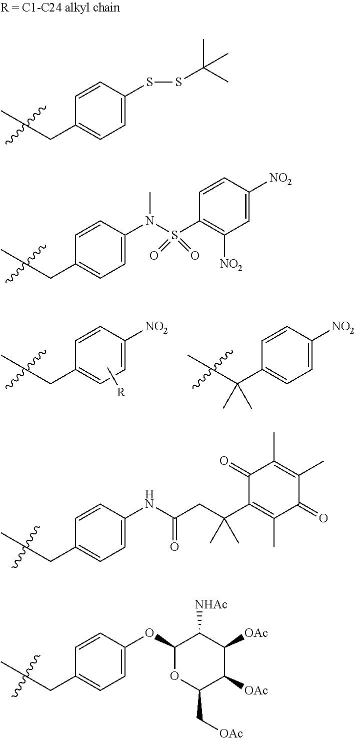

### Synthesis of 2′-Modified Nucleoside Building Blocks with 2′-Methylene Amide Linker

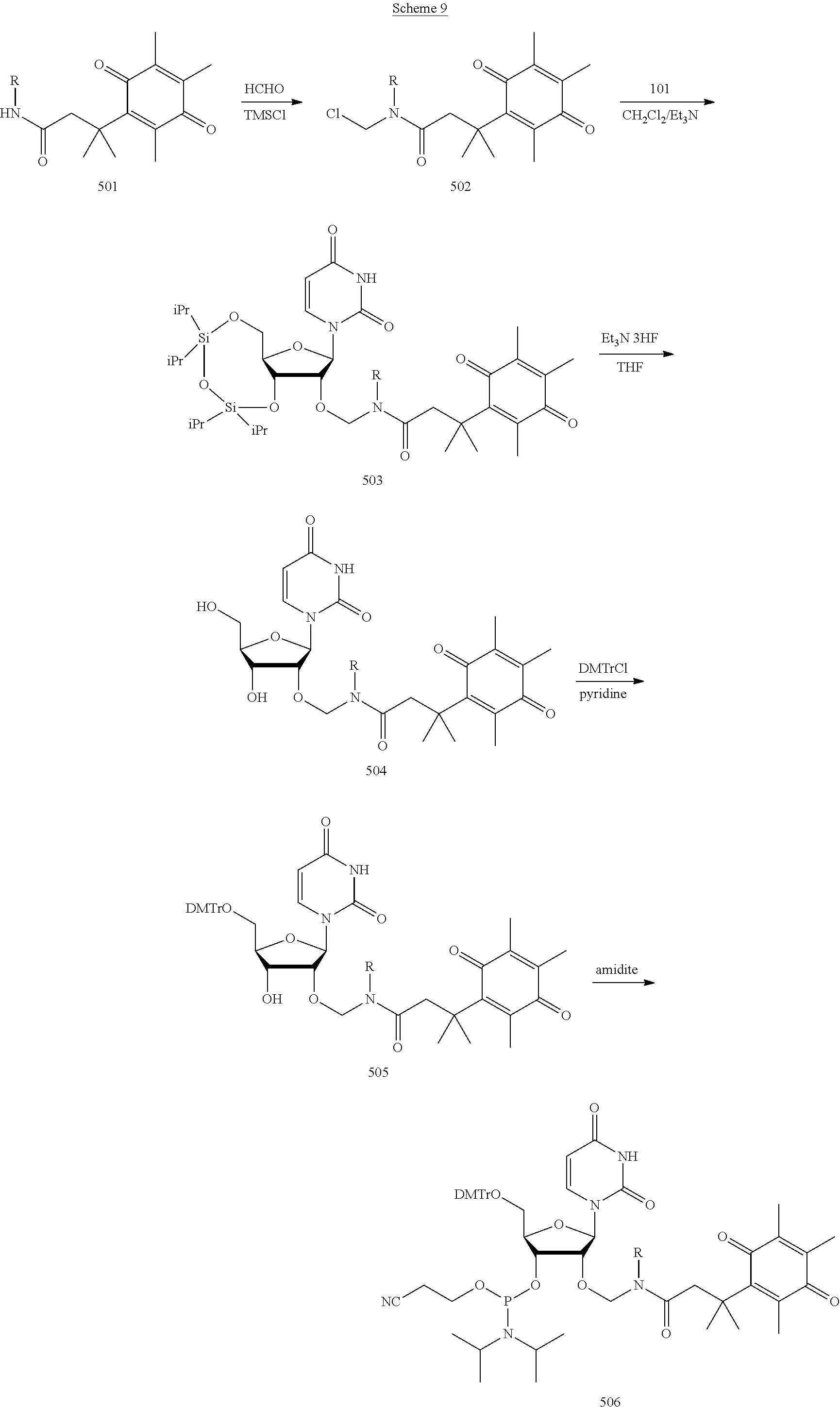

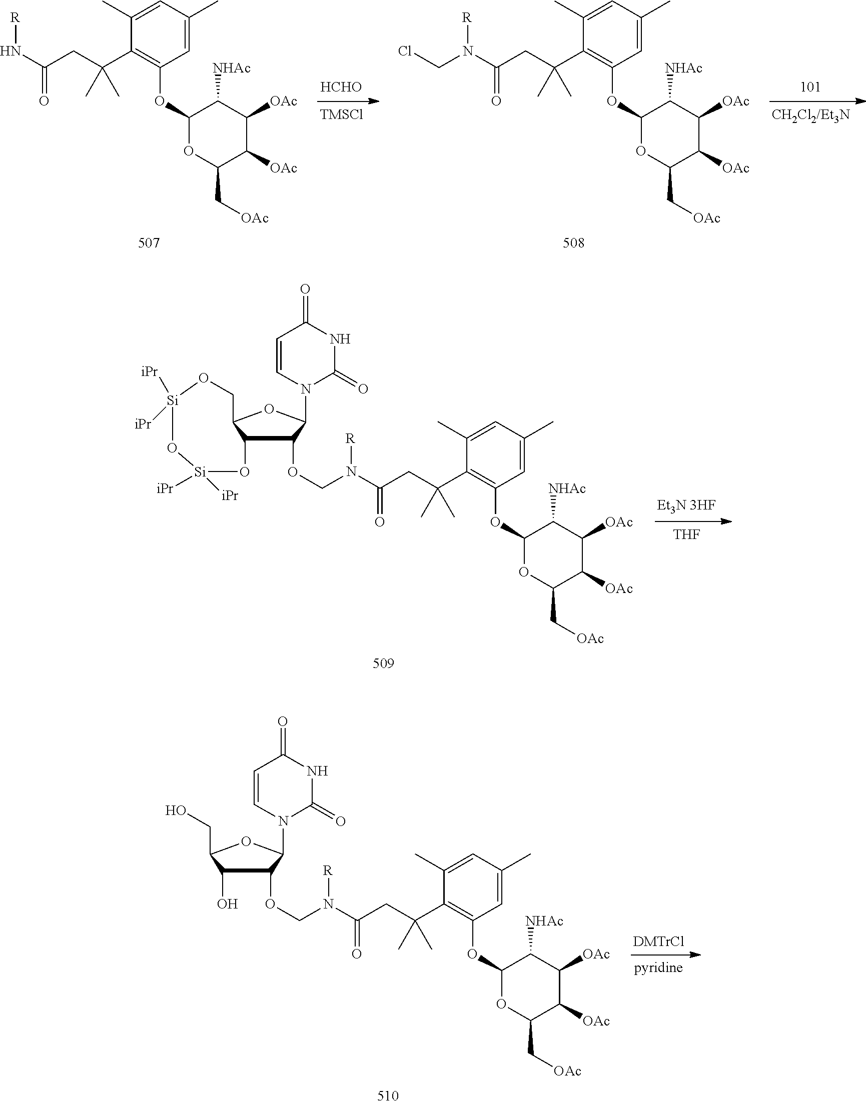

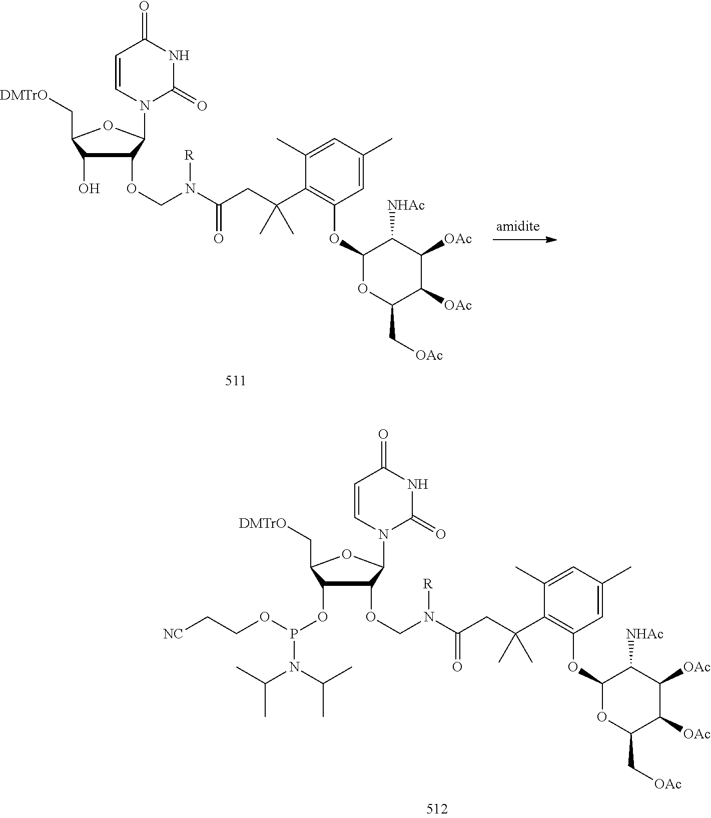

### Synthesis of 2′-Modified Nucleoside Building Blocks with 2′-Carbohydrate Linker

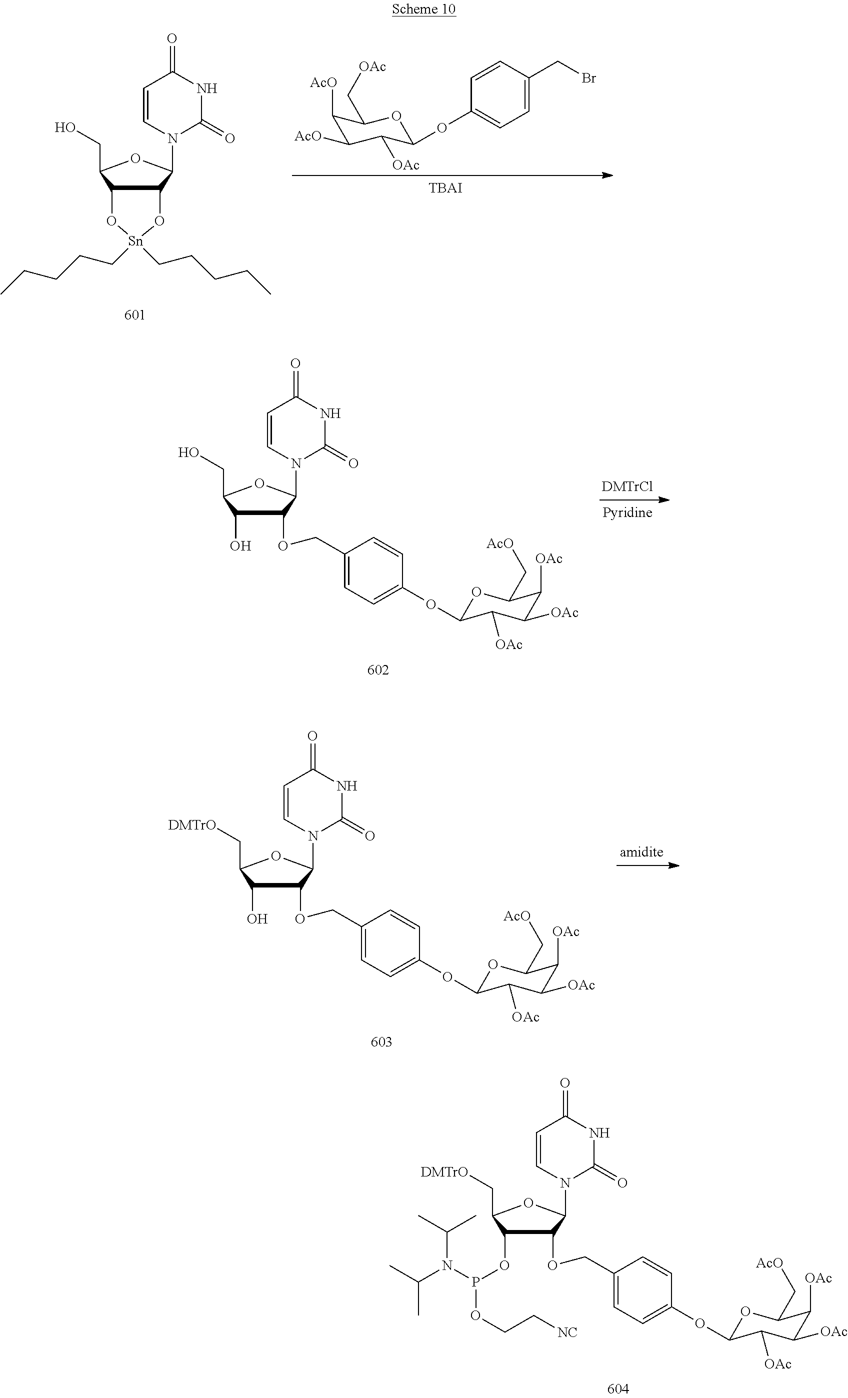

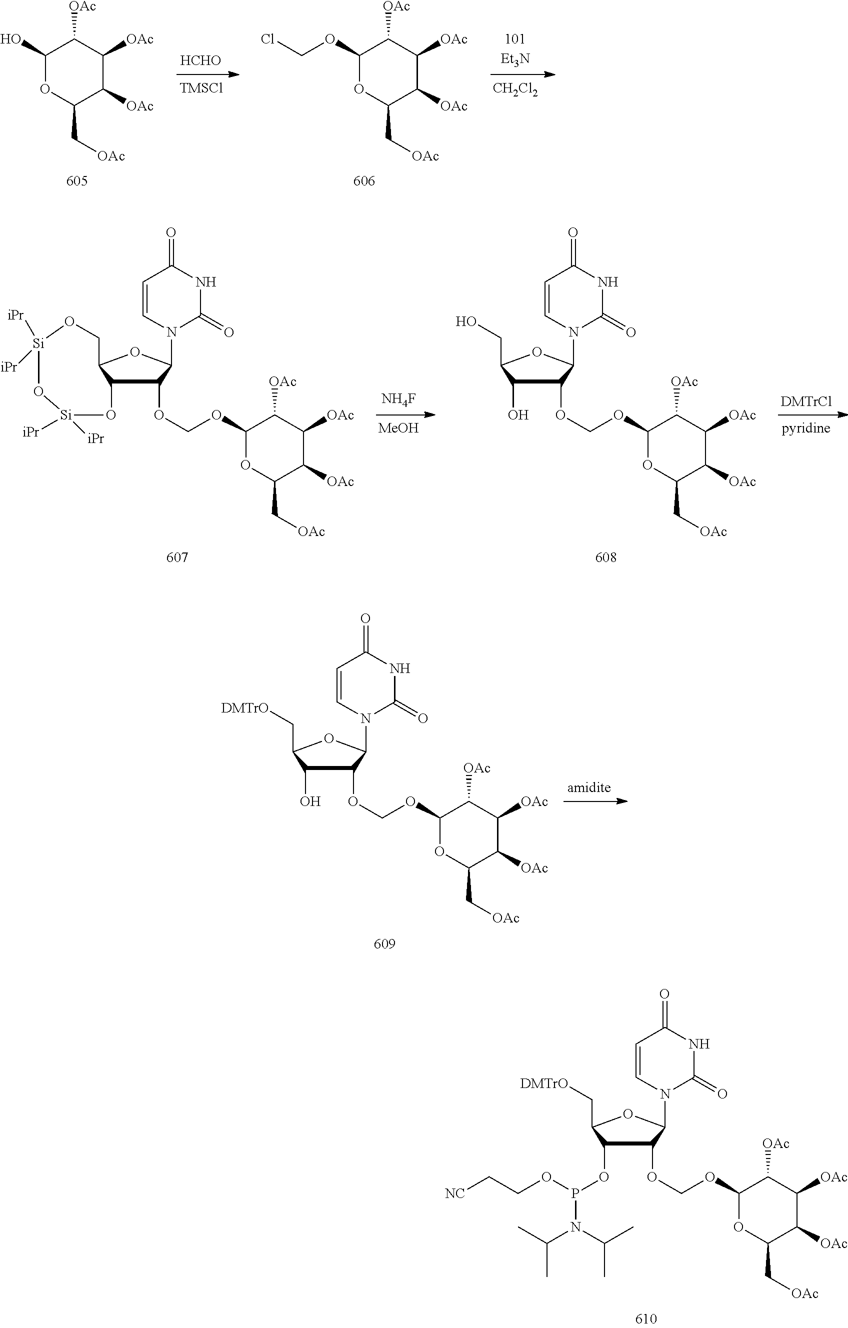

### Synthesis of 2′-Modified Nucleoside Building Blocks (H-Phosphonate/Phosphoramidite) with 2′-Methylene Disulfide Linker

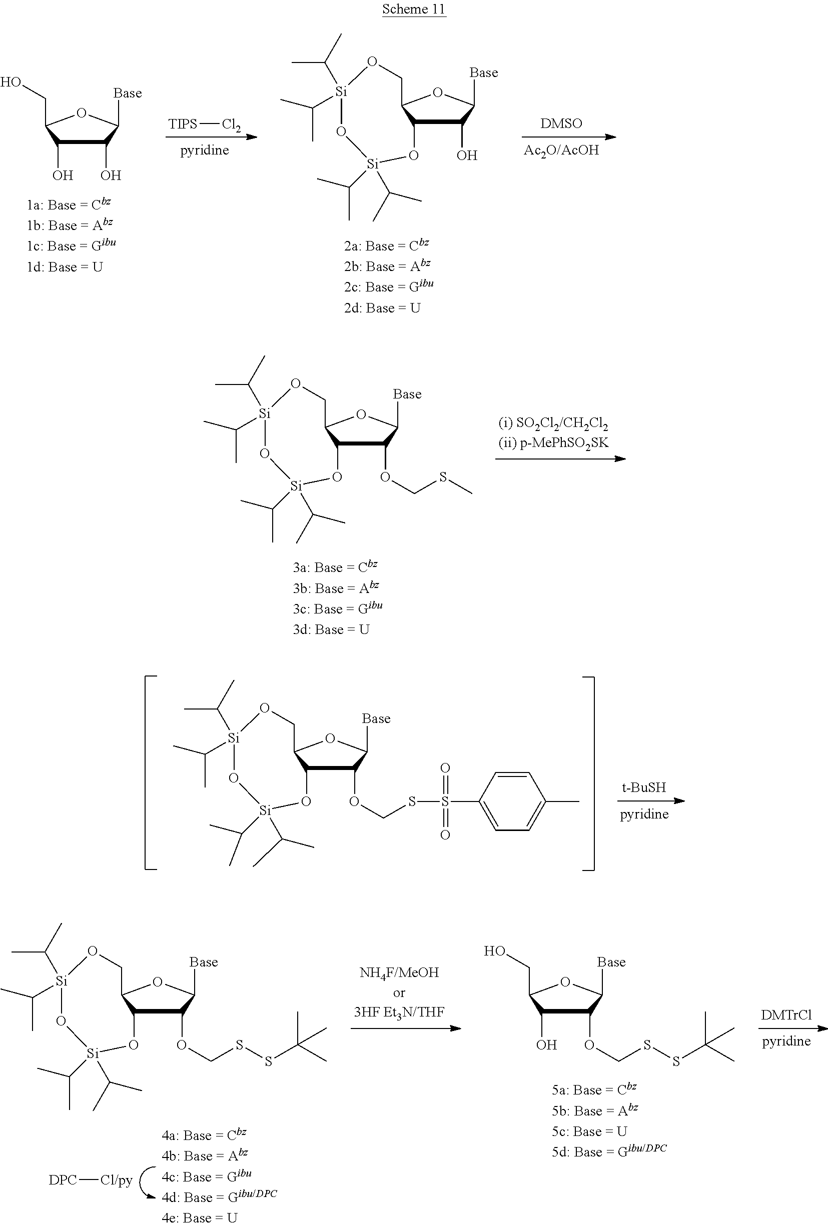

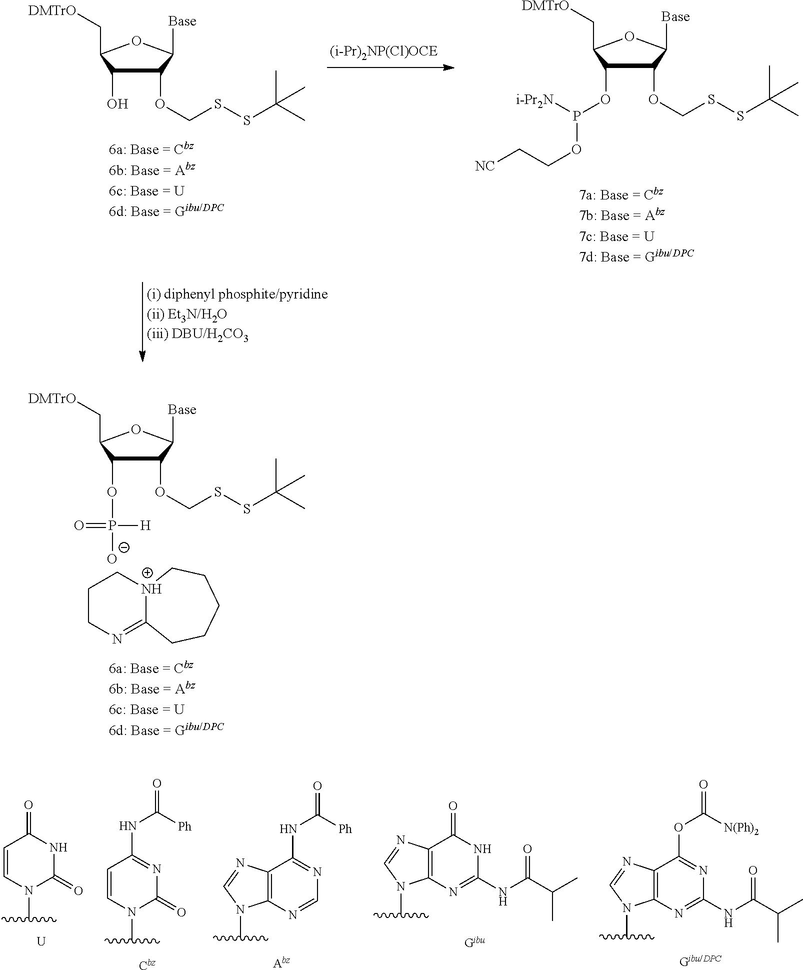

Details for phosphoramidite synthesis can be found in Semenyuk et al., J. Am. Chem. Soc. 128(38): 12356-57 (2006), which is incorporated herein by reference in its entirety.

### Synthesis of chirally pure 2′-PivOM-5′-(S)-Me-uridine-2′-fluorouridine phosphorothioate dinucleotide (URsUfm)

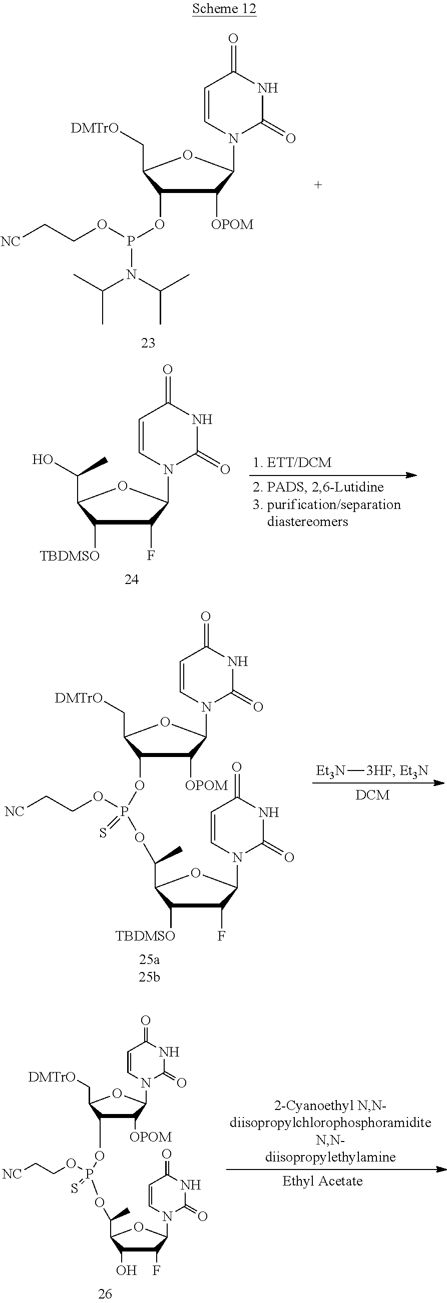

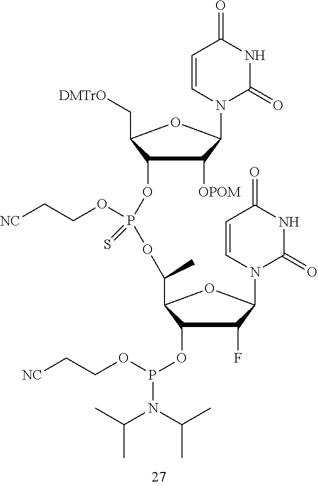

Compound 25: Compound 23 (1.24 g, 3.32 mmol) was dissolved in anhydrous PGP166,C3 dichloromethane (20 mL). To this solution was added a solution of compound 24 (3.00 g, 3.49 mmol, 1.05 equiv.) in anhydrous dichloromethane (15 mL), followed by ETT (26.6 mL, 6.6 mmol, 2 equiv.). The reaction mixture was stirred at room temperature under argon for 2.5 hours. Phenylacetyl disulfide (1.51 g, 5.0 mmol, 1.5 equiv.) and 2,6-lutidine (0.58 mL, 5.0 mmol, 1.5 equiv.) was added and the reaction mixture was stirred for an additional 2.5 hours. The reaction mixture was diluted with dichloromethane (200 mL) and washed with water (2×75 mL). The organic layer was dried over anhydrous Na₂SO₄, filtered, and concentrated to an oil. The diastereomers were separated by flash column chromatography on silica gel (220 g), and eluted with EtOAc: hexane (1:1 to 5:1). Compound 25a R_{f} (top spot)=0.50, Compound 25b R_{f} (bottom spot)=0.45 in 2:1, EtOAc: hexane. Yield: Compound 25a (1.75 g, 45%, white foam) and Compound 25b (1.09 g, 28%, white foam). Compound 25a ¹HNMR: (500 MHz, acetonitrile-d₃) δ 9.41 (d, J=33.5 Hz, 2H), 7.61 (d, J=8.1 Hz, 1H), 7.51 (d, J=8.2 Hz, 1H), 7.47-7.40 (m, 2H), 7.38-7.29 (m, 7H), 7.29-7.22 (m, 1H), 6.93-6.84 (m, 4H), 5.93-5.82 (m, 2H), 5.59 (d, J=8.1 Hz, 1H), 5.41 (dd, J=7.4, 3.5 Hz, 2H), 5.28 (d, J=6.6 Hz, 1H), 5.11-5.01 (m, 2H), 4.98 (dd, J=4.7, 2.3 Hz, 1H), 4.91-4.80 (m, 1H), 4.63 (t, J=5.2 Hz, 1H), 4.40-4.25 (m, 2H), 4.22-4.06 (m, 2H), 3.98 (t, J=6.4 Hz, 1H), 3.77 (s, 7H), 3.47-3.37 (m, 2H), 2.76-2.65 (m, 2H), 1.44 (d, J=6.5 Hz, 3H), 1.13 (s, 9H), 0.91 (s, 9H), 0.13 (d, J=7.2 Hz, 6H). ¹³C NMR (101 MHz, acetonitrile-d₃) δ 178.53, 164.08, 163.90, 159.84, 151.38, 151.35, 145.46, 141.50, 141.23, 136.35, 136.27, 131.14, 131.12, 129.10, 129.07, 129.02, 128.14, 118.36, 114.27, 103.37, 103.04, 94.59, 92.71, 90.03, 89.68, 89.49, 88.41, 88.07, 86.40, 86.32, 82.94, 82.88, 80.22, 80.17, 78.54, 78.49, 75.94, 75.89, 71.68, 71.53, 64.34, 64.29, 63.27, 56.01, 55.99, 39.44, 27.29, 26.06, 20.11, 20.02, 18.63, 18.35, −4.19, −4.73. ³¹P NMR (202 MHz, acetonitrile-d₃) S 68.20. Compound 25b ¹H NMR: (500 MHz, acetonitrile-d₃) δ 9.30 (s, 1H), 7.61 (d, J=8.2 Hz, 1H), 7.45 (dd, J=8.4, 6.8 Hz 4H), 7.33 (d, J=8.9 Hz, 7H), 7.25 (t, J=7.4 Hz, 1H), 6.89 (d, J=8.6 Hz, 5H), 5.96 (d, J=6.0 Hz, 1H), 5.82 (dd, J=19.1, 2.0 Hz, 1H), 5.60 (d, J=8.1 Hz, 1H), 5.46-5.36 (m, 2H), 5.31 (d, J=6.7 Hz, 1H), 5.18-5.07 (m, 2H), 4.99 (dd, J=4.7, 2.0 Hz, 1H), 4.81-4.72 (m, 1H), 4.64 (t, J=5.6 Hz, 1H), 4.39-4.30 (m, 2H), 4.30-4.18 (m, 2H), 3.95-3.89 (m, 1H), 3.77 (s, 7H), 3.45-3.36 (m, 2H), 2.86-2.76 (m, 2H), 1.34 (d, J=6.5 Hz, 3H), 1.13 (s, 9H), 0.90 (s, 9H), 0.13 (d, J=9.3 Hz, 6H). ¹³C NMR (101 MHz, acetonitrile-d₃) δ 178.54, 164.20, 163.84, 159.87, 151.48, 151.26, 145.47, 141.91, 141.04, 136.27, 136.20, 131.17, 131.15, 129.09, 129.07, 128.15, 121.82, 118.68, 118.37, 114.29, 103.57, 102.97, 94.63, 92.76, 90.69, 90.33, 89.61, 88.18, 87.70, 86.19, 86.11, 83.27, 83.22, 80.50, 80.46, 77.66, 77.60, 76.99, 76.95, 71.22, 71.06, 64.37, 64.33, 63.55, 56.02, 56.00, 39.46, 27.30, 26.08, 20.15, 20.06, 18.78, 18.63, −4.19, −4.64. ³¹P NMR (202 MHz, acetonitrile-d₃) S 67.97.

Compound 26: Compound 25a (1.46 g, 1.25 mmol) was dissolved in anhydrous dichloromethane (10 mL). Et₃N·3HF (1.02 mL, 6.3 mmol, 5 equiv.) and Et₃N (0.35 mL, 2.5 mmol, 2 equiv.) were added to the solution. The reaction mixture was stirred at room temperature under argon for 68 hours. The reaction mixture was diluted with dichloromethane (10 mL) and washed with 5% NaHCO₃ (2×15 mL), 5% NaCl (1×25 mL), and saturated NaCl (1×25 mL). The organic layer was dried over anhydrous Na₂SO₄, filtered, and concentrated to a white solid. The product was purified by flash column chromatography on silica gel (40 g), and eluted with 6% methanol: methanol/dichloromethane, R_{f}=0.25. Yield: Compound 26 (1.0 g, 76%, white solid). ¹H NMR: (500 MHz, acetonitrile-d₃) δ 9.32 (d, J=15.0 Hz, 2H), 7.57 (dd, J=8.2, 6.3 Hz, 2H), 7.49-7.36 (m, 2H), 7.36-7.26 (m, 6H), 7.26-7.20 (m, 1H), 6.93-6.81 (m, 4H), 5.88 (d, J=6.3 Hz, 1H), 5.82 (dd, J=18.3, 1.5 Hz, 1H), 5.58 (d, J=8.1 Hz, 1H), 5.43 (d, J=8.1 Hz, 1H), 5.37 (d, J=6.8 Hz, 1H), 5.22 (d, J=6.8 Hz, 1H), 5.09-4.94 (m, 2H), 4.89-4.80 (m, 1H), 4.56 (t, J=5.8 Hz, 1H), 4.36-4.25 (m, 2H), 4.19-4.03 (m, 2H), 3.97-3.91 (m, 1H), 3.75 (s, 6H), 3.72 (d, J=7.1 Hz, 1H), 3.47-3.38 (m, 2H), 2.74-2.61 (m, 2H), 1.47 (d, J=6.5 Hz, 3H), 1.09 (s, 9H). ¹³C NMR (101 MHz, acetonitrile-d₃) δ 178.84, 164.16, 163.74, 159.87, 151.46, 151.26, 145.47, 141.24, 140.91, 136.27, 131.15, 131.11, 129.11, 129.05, 128.16, 118.55, 118.38, 114.32, 103.66, 102.70, 95.55, 93.71, 90.08, 89.73, 89.56, 88.23, 87.26, 85.17, 85.09, 83.29, 83.23, 80.25, 80.21, 77.24, 77.19, 76.90, 76.86, 70.01, 69.85, 64.15, 64.10, 63.50, 56.02, 56.00, 39.46, 27.25, 20.13, 20.04, 18.53, 2.01, 1.80, 1.60, 1.39, 1.18, 0.98, 0.77. ³¹P NMR (202 MHz, acetonitrile-d₃) δ 68.23.

Compound 27: Compound 26 (0.40 g, 0.38 mmol) was dried overnight under high vacuum and dissolved in anhydrous ethyl acetate (2 mL). N,N-diisopropylethylamine (0.1 mL, 0.57 mmol, 1.5 equiv.), and 2-cyanoethyl N,N-diisopropylchlorophosphoramidite (0.13 mL, 0.57 mmol, 1.5 equiv.) were added to the solution and stirred at room temperature under argon for 2 hours. Triethanolamine (0.35 mL, 0.95 mmol, 2.5 equiv, 2.7 M solution in acetonitrile:toluene (4:9)) was added to the reaction mixture and stirred for 5 minutes. The reaction mixture was diluted with ethyl acetate (20 mL) and washed with 5% NaCl (3×20 mL) and saturated NaCl (1×20 mL). The organic layer was dried over Na₂SO₄, filtered, and concentrated to a white foam. The product was purified by flash column chromatography on silica gel (24 g), and eluted with EtOAc: hexane (2:1 to 3:1). R_{f}=0.3 in EtOAc: hexane (2:1). Yield Compound 27 (0.43 g, 91%, white foam). ¹H NMR: (500 MHz, acetonitrile-d₃) δ 9.21 (s, 2H), 7.59 (t, J=8.2 Hz, 1H), 7.50 (dd, J=8.1, 6.8 Hz, 1H), 7.44 (d, J=7.4 Hz, 2H), 7.38-7.28 (m, 6H), 7.25 (t, J=7.2 Hz, 1H), 6.96-6.83 (m, 4H), 5.95-5.79 (m, 2H), 5.54 (dd, J=9.3, 8.1 Hz, 1H), 5.50-5.37 (m, 2H), 5.31-4.99 (m, 3H), 4.94-4.83 (m, 1H), 4.63 (q, J=5.8 Hz, 1H), 4.55-4.34 (m, 1H), 4.29 (d, J=3.7 Hz, 1H), 4.22-4.03 (m, 3H), 3.93-3.58 (m, 10H), 3.49-3.33 (m, 2H), 2.76-2.60 (m, 4H), 1.47 (dd, J=15.7, 6.5 Hz, 3H), 1.33-1.15 (m, 12H), 1.13 (d, J=1.8 Hz, 9H). ³¹P NMR (202 MHz, acetonitrile-d₃) δ 152.15, 152.09, 151.96, 151.92, 68.21, 68.15.

### Synthesis of 2′-Tert-Butyl Disulfide Methylene-U Building Block for Chirally Pure Phosphorothioate Oligonucleotide

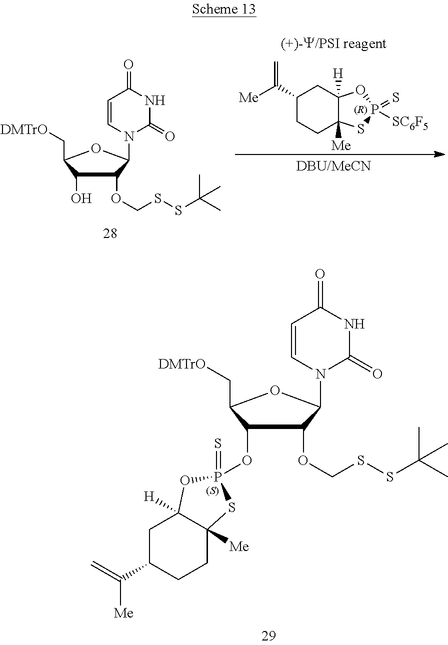

Compound 29: Compound 28 (2.13 g, 3.13 mmol) was dried overnight under high vacuum and dissolved in anhydrous acetonitrile (30 mL). (+)-Ψ/PSI reagent (1.82 g, 4.07 mmol, 1.3 equiv.), and DBU (1.02 g, 4.07 mmol, 1.3 equiv.) were added to the solution and stirred at room temperature under argon for 30 min at room temperature. The reaction mixture was diluted with EtOAC and extracted with aqueous NaHCO₃ and then brine. The organic layer was dried over Na₂SO₄ and concentrated. Silica gel column purification eluted with 0-50% EtOAc in hexane gave compound 29 (2.60 g, 2.80 mmol) of white foam product, with 90% yield. ¹H NMR (500 MHz, CD₃CN) δ 9.05-8.95 (m, 1H), 7.58 (d, J=8.2 Hz, 1H), 7.46-7.37 (m, 2H), 7.35-7.19 (m, 7H), 6.90-6.82 (m, 4H), 5.94 (d, J=6.8 Hz, 1H), 5.40-5.26 (m, 2H), 4.95 (q, J=1.5 Hz, 1H), 4.88 (s, 2H), 4.87-4.80 (m, 1H), 4.55 (ddd, J=6.4, 5.2, 0.9 Hz, 1H), 4.41 (ddd, J=12.8, 3.7, 2.6 Hz, 1H), 4.25 (q, J=2.9 Hz, 1H), 3.74 (s, 6H), 3.42 (dd, J=11.1, 3.1 Hz, 1H), 3.32 (dd, J=11.1, 3.1 Hz, 1H), 2.57 (s, 1H), 2.25-2.15 (m, 1H), 2.04 (td, J=13.4, 4.2 Hz, 1H), 1.93-1.82 (m, 4H), 1.72 (s, 3H), 1.62 (s, 3H), 1.25 (s, 9H). ³¹P NMR (202 MHz, CD₃CN) δ 103.47. MS-ESI 949.3 [M+Na], 925.2 [M-H]

### Example 2. In Vivo/In Vitro Fluorescence Probe for RNAi Activity & Cellular Trafficking

An exemplary in vivo in vitro fluorence probe incorporated into 2′-modified nucleoside and reaction mechanism for RNAi activity & cellular trafficking are shown in Scheme 14.

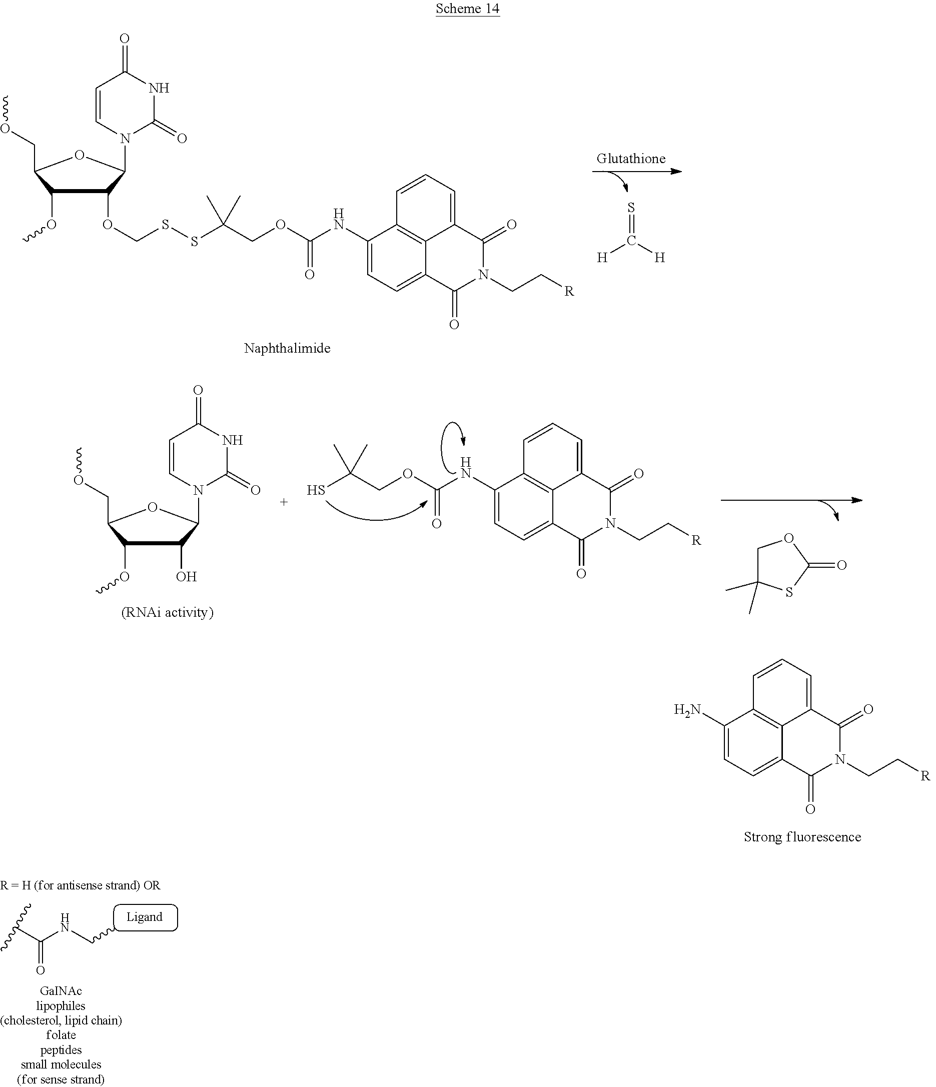

Exemplary 2′-fluorescence probe linker nucleoside building blocks for in vivo turn-on probe for RNAi activity and cellular trafficking are shown in Scheme 15.

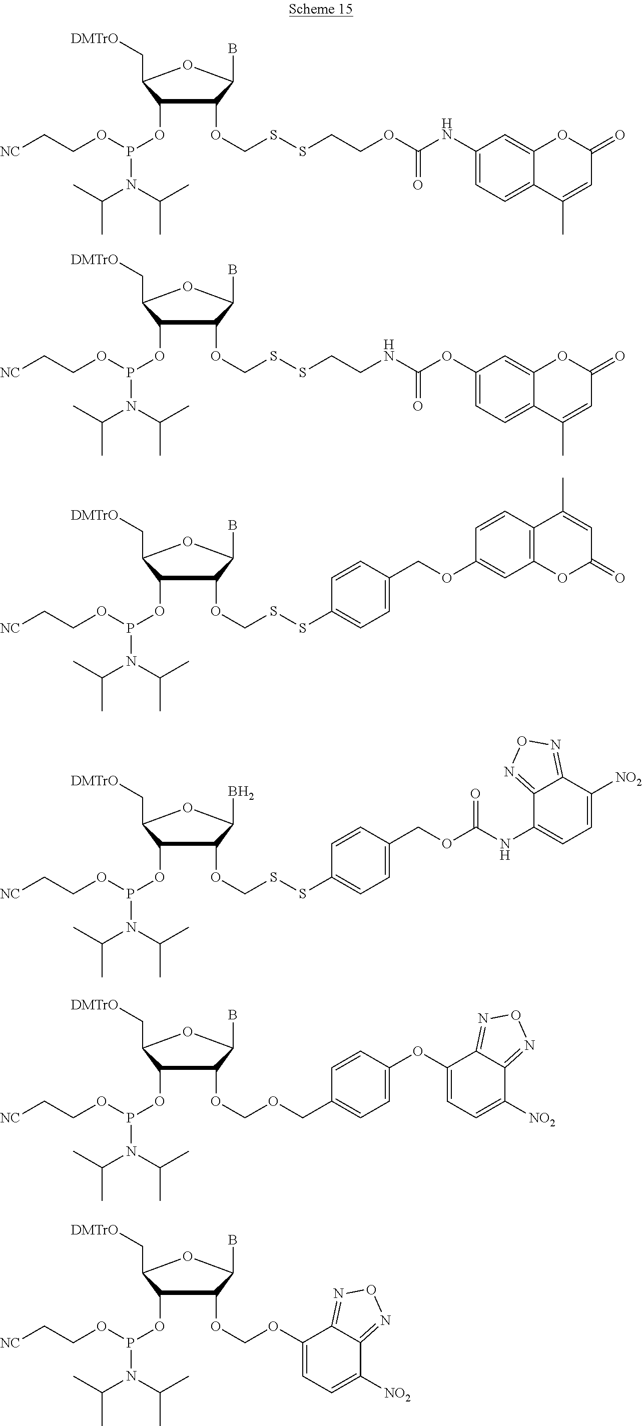

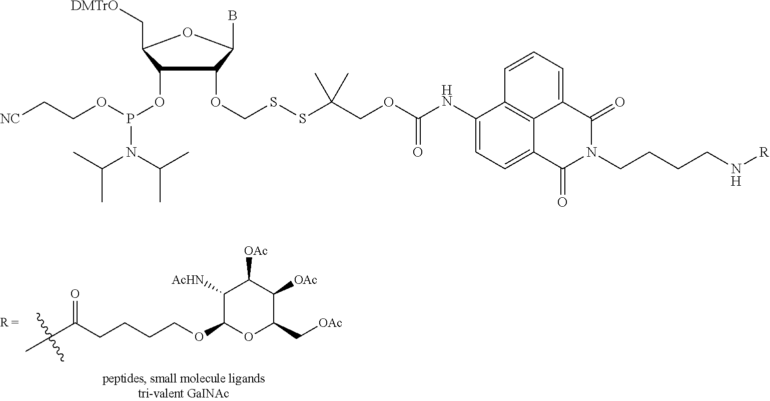

In above scheme, R can also be H, linear or branched C₁-C₂₄ alkyl (e.g., Me), C≡CH, OH, O-alkyl (e.g., OMe, O-nPr), O-alkylamino, O—(CH₂)₂OMe, SMe, Cl, F, NMe₂, or NH₂. B is A^{Bz}; C^{Ac}; 5-Me-C^{Ac}; G^{iBu}; I; U; T; 2-thiouridine; 4-thiouridine; C5-modified pyrimidines; C2-modified purines; N8-modifed purines; phenoxazine; G-clamp; non-canonical mono, bi and tricyclic heterocycles; pseudouracil; isoC; isoG; 2,6-diamninopurine; pseudocytosine; 2-aminopurine; xanthosine; N6-alkyl-A; O6-alkyl-G; or unnatural nucleobase.

Any other fluorescence probe can also be installed.

### Example 3. Synthesis of Oligonucleotides Containing 2′-Modified Nucleoside

### Synthesis of Y137-Containing Oligonucleotide Using General Synthesis Procedures

General synthesis procedures are illustrated in Scheme 16 below, using Y137 as an example.

General Synthesis Procedures for Y137:
1. Deblocking:
   I. Single strand oligonucleotide (0.60 g, 0.04 mmol) loaded on CPG support in Merrifield peptide synthesis flask was washed with deblock solution four times (300 TCA in DCM) with 10 mL each.
   II. The support was washed with anhydrous acetonitrile, followed by 1:1 ACN:Pyridine solution (100 mL each), then dried under argon.
2. Coupling/condensation:
   I. H-Phosphonate 22 (73.2 mg, 0.08 mmol, 2 equivalent) dissolved in 1:1 ACN:Pyr solution (2 mL, 40 mM) and DPPC activator (2 mL, 120 mM solution (50.7 μL, 0.24 mmol, 3 equivalent)) were added simultaneously to the support, then vortex for 10 minutes (as a nice swirl, not splash).
   II. The support was dried under argon, and then washed with dry 1:1 ACN:Pyr solution and dry ACN (100 mL each). The support was dried by passing argon.
3. Sulfurization
   I. Elemental sulfur (320 mg) was dissolved in pyridine (32 mL) to get a 0.5 M solution. The sulfur solution (10 mL) was added to the support and vortex for 30 minutes.
   II. Support was washed twice with dry ACN (100 mL).
   III. The support was transferred to vacuum chamber and dried under high vacuum.
4. De-tritylation and capping:
   I. After drying, the support was packed into 40 μmol column and put on the ABI synthesizer for deblock and capping using the standard protocol.
   II. Y137 at the position 1 of the antisense strand was only de-tritylated, and Y137 at the position 2 of the antisense strand, was treated with de-block solution, and then coupled with 2′-OMe U and sulfurized as PS linkage.
5. Deprotection and purification
   I. Standard oligonucleotide deprotection and purification procedure was used to generating the single strands containing Y137 monomers.

### Synthesis of Y87-Containing Oligonucleotide Using Post-Synthetic Modifications

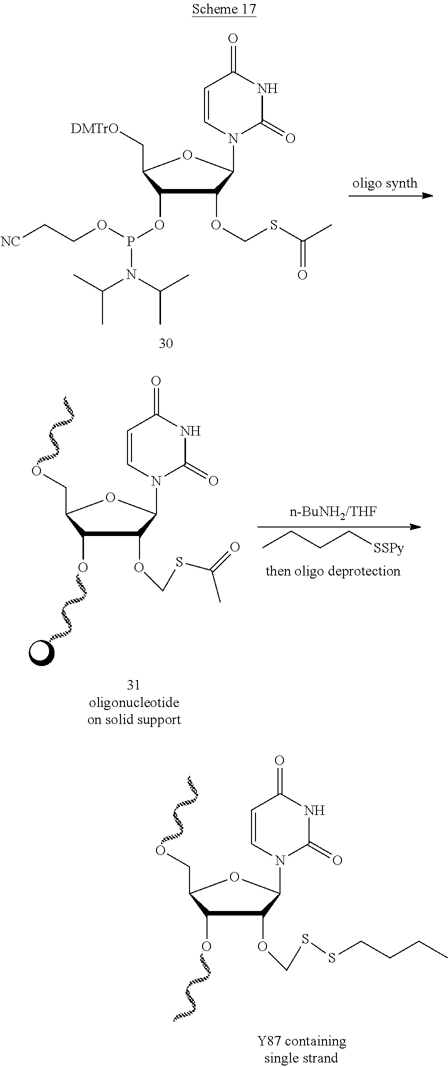

Y87-containing oligonucleotide was synthesized using procedures described in Biscans et al., Org. Biomol. Chem. 14: 7010 (2016), which is incorporated herein by reference in its entirety. Briefly, building block 23 was synthesized as reported in Biscans et al., and then introduced into oligonucleotides on solid support. The precursor oligonucleotide on the solid support cartridge were treated with n-buthylamine in THF and n-butyl pyridyl disulfide in THE using two syringes for the introduction of butyl disulfide at 2′-position.

TABLE 1
siRNA single strands synthesized for in vitro studies

| | | | | | Molecular |
|---|---|---|---|---|---|
| Oligo | | | | Molecular | Weight |
| ID | Strand | Target | Oligo Seq | Weight | Found |
| A- | antis | TTR | usY87saudAgadGcaadGadAcacugu | 7704.36 | 7700.21 |
| 816093 | | | ususu | | |
| A- | antis | TTR | usY137saudAgadGcaadGadAcacug | 7704.37 | 7700.21 |
| 1690793 | | | uususu | | |
| A- | antis | TTR | usY138saudAgadGcaadGadAcacug | 7789.48 | 7785.26 |
| 1690794 | | | uususu | | |
| A- | antis | TTR | usY139saudAgadGcaadGadAcacug | 7812.37 | 7808.23 |
| 1690795 | | | uususu | | |
| A- | antis | TTR | usY 140saudAgadGcaadGadAcacug | 7854.45 | 7850.28 |
| 1690796 | | | uususu | | |
| A- | antis | TTR | Y137sdTsaudAgadGcaadGadAcacu | 7688.37 | 7684.22 |
| 1690798 | | | guususu | | |
| A- | antis | TTR | Y138sdTsaudAgadGcaadGadAcacu | 7773.48 | 7769.27 |
| 1690799 | | | guususu | | |
| A- | antis | TTR | Y139sdTsaudAgadGcaadGadAcacu | 7796.37 | 7792.23 |
| 1690800 | | | guususu | | |
| A- | antis | TTR | Y140sdTsaudAgadGcaadGadAcacu | 7838.45 | 7834.29 |
| 1690801 | | | guususu | | |
| A- | antis | TTR | Y87sdTsaudAgadGcaadGadAcacug | 7688.36 | 7684.21 |
| 1690802 | | | uususu | | |
| A- | sense | TTR | asascaguguY87cY87ugcucuauaaL96 | 8975.03 | 8973.44 |
| 816092 | | | | | |
| A- | sense | TTR | asascagugudTcY87ugcucuauaaL96 | 8838.82 | 8838.31 |
| 2483638 | | | | | |
| A- | sense | TTR | asascagugu Y87cdTugcucuauaaL96 | 8838.82 | 8838.58 |
| 2483637 | | | | | |
| A- | sense | TTR | asascagugudTcY139ugcucuauaaL96 | 8946.82 | 8946.59 |
| 2483634 | | | | | |
| A- | sense | TTR | asascagugu Y139cdTugcucuauaaL96 | 8946.82 | 8946.60 |
| 2483633 | | | | | |
| A- | sense | TTR | asascagugu Y139cY139ugcucuauaaL | 9191.03 | 9190.71 |
| 2483632 | | | 96 | | |
| A- | sense | TTR | asascagugudTcY137ugcucuauaaL96 | 8838.83 | 8839.15 |
| 2483631 | | | | | |
| A- | sense | TTR | asascagugu Y137cdTugcucuauaaL96 | 8838.83 | 8838.52 |
| 2483630 | | | | | |
| A- | sense | TTR | asascagugu Y137c Y 137ugcucuauaaL | 8975.05 | 8974.39 |
| 2483629 | | | 96 | | |
| A- | Antis | SOD1 | Y137sUfsuagAfgUfGfaggaUfuAfaa | 7895.38 | 7895.40 |
| 2483639 | | | augsasg | | |
| A- | Antis | SOD1 | Y137s(Ufms)uagAfgUfGfaggaUfuA | 7909.40 | 7908.44 |
| 2483641 | | | faaaugsasg | | |
| A- | Antis | SOD1 | (Y137Rs)UfsuagAfgUfGfaggaUfuA | 7895.38 | 7894.74 |
| 2872600 | | | faaaugsasg | | |

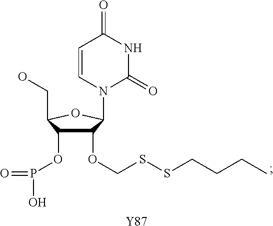

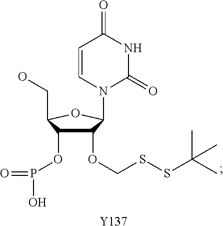

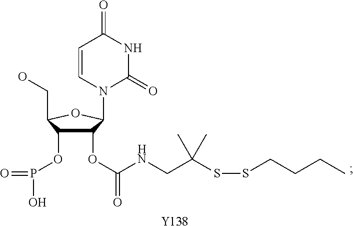

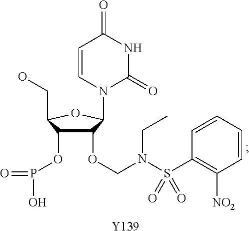

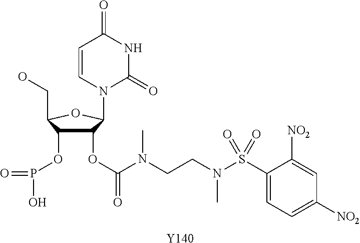

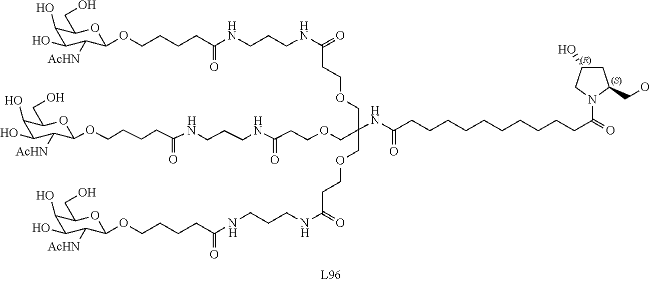

d followed with upper case letter-2′-deoxy sugar modification; upper case letter followed
with f-2′-F sugar modification; lower case letter-2′-O-methyl (2′-OMe) sugar
modification; s-phosphorothioate (PS) linkage.

TABLE 2
siRNA duplexes synthesized for in vitro and in vivo studies

| | | | | | | Molecular |
|---|---|---|---|---|---|---|
| Duplex | Oligo | | | | Molecular | Weight |
| ID | ID | Strand | Target | Oligo Seq | Weight | found |
| AD- | A- | sense | TTR | asascaguGfuUfCfUfugcucua | 8686.46 | 8681.99 |
| 64958 | 128009 | | | uaaL96 | | |
| | A- | antis | TTR | usUfsauaGfagcaagaAfcAfcu | 7656.12 | 7652.17 |
| | 126312 | | | guususu | | |
| AD- | A- | sense | TTR | asascagugudTcdTugcucuaua | 8702.60 | 8698.08 |
| 238841 | 128292 | | | aL96 | | |
| | A- | antis | TTR | usdTsaudAgdAgcaadGadAc | 7568.15 | 7564.21 |
| | 463190 | | | acuguususu | | |
| AD- | A- | sense | TTR | asascagugudTcdTugcucuaua | 8702.60 | 8698.08 |
| 266701 | 128292 | | | aL96 | | |
| | A- | antis | TTR | usUsaudAgadGcaadGadAca | 7570.13 | 7566.19 |
| | 515454 | | | cuguususu | | |
| AD- | A- | sense | TTR | asascagugudTcdTugcucuaua | 8702.60 | 8698.08 |
| 266719 | 128292 | | | aL96 | | |
| | A- | antis | TTR | UsdTsaudAgadGcaadGadAc | 7554.13 | 7550.19 |
| | 515472 | | | acuguususu | | |
| AD- | A- | sense | TTR | asascagugudTcdTugcucuaua | 8702.60 | 8698.08 |
| 511258 | 128292 | | | aL96 | | |
| | A- | antis | TTR | us Y87saudAgadGcaadGadA | 7704.37 | 7700.21 |
| | 816093 | | | cacuguususu | | |
| AD- | A- | sense | TTR | asascagugudTcdTugcucuaua | 8702.60 | 8698.08 |
| 1025231 | 128292 | | | aL96 | | |
| | A- | antis | TTR | usY137saudAgadGcaadGad | 7704.38 | 7700.21 |
| | 169079 | | | Acacuguususu | | |
| | 3 | | | | | |
| AD- | A- | sense | TTR | asascagugudTcdTugcucuaua | 8702.60 | 8698.08 |
| 1020661 | 128292 | | | aL96 | | |
| | A- | antis | TTR | usY138saudAgadGcaadGad | 7789.49 | 7785.26 |
| | 169079 | | | Acacuguususu | | |
| | 4 | | | | | |
| AD- | A- | sense | TTR | asascagugudTcdTugcucuaua | 8702.60 | 8698.08 |
| 1020662 | 128292 | | | aL96 | | |
| | A- | antis | TTR | usY139saudAgadGcaadGad | 7812.38 | 7808.22 |
| | 169079 | | | Acacuguususu | | |
| | 5 | | | | | |
| AD- | A- | sense | TTR | asascagugudTcdTugcucuaua | 8702.60 | 8698.08 |
| 1020663 | 128292 | | | aL96 | | |
| | A- | antis | TTR | usY140saudAgadGcaadGad | 7854.46 | 7850.28 |
| | 169079 | | | Acacuguususu | | |
| | 6 | | | | | |
| AD- | A- | sense | TTR | asascagugudTcdTugcucuaua | 8702.60 | 8698.08 |
| 1025232 | 128292 | | | aL96 | | |
| | A- | antis | TTR | Y137sdTsaudAgadGcaadGa | 7688.38 | 7684.22 |
| | 169079 | | | dAcacuguususu | | |
| | 8 | | | | | |
| AD- | A- | sense | TTR | asascagugudTcdTugcucuaua | 8702.60 | 8698.08 |
| 1020665 | 128292 | | | aL96 | | |
| | A- | antis | TTR | Y138sdTsaudAgadGcaadGa | 7773.49 | 7769.27 |
| | 169079 | | | dAcacuguususu | | |
| | 9 | | | | | |
| AD- | A- | sense | TTR | asascagugudTcdTugcucuaua | 8702.60 | 8698.08 |
| 1020666 | 128292 | | | aL96 | | |
| | A- | antis | TTR | Y139sdTsaudAgadGcaadGa | 7796.38 | 7792.23 |
| | 169080 | | | dAcacuguususu | | |
| | 0 | | | | | |
| AD- | A- | sense | TTR | asascagugudTcdTugcucuaua | 8702.60 | 8698.08 |
| 1020667 | 128292 | | | aL96 | | |
| | A- | antis | TTR | Y140sdTsaudAgadGcaadGa | 7838.46 | 7834.29 |
| | 169080 | | | dAcacuguususu | | |
| | 1 | | | | | |
| AD- | A- | sense | TTR | asascagugudTcdTugcucuaua | 8702.60 | 8698.08 |
| 1020664 | 128292 | | | aL96 | | |
| | A- | antis | TTR | Y87sdTsaudAgadGcaadGad | 7688.37 | 7684.21 |
| | 169080 | | | Acacuguususu | | |
| | 2 | | | | | |
| AD- | A- | sense | TTR | asascaguguUcdTugcucuauaa | 8704.57 | 8700.06 |
| 266698 | 515451 | | | L96 | | |
| | A- | antis | TTR | usdTsaudAgadGcaadGadAc | 7568.16 | 7564.21 |
| | 432271 | | | acuguususu | | |
| AD- | A- | sense | TTR | asascagugudTcUugcucuauaa | 8704.57 | 8700.06 |
| 266699 | 515452 | | | L96 | | |
| | A- | antis | TTR | usdTsaudAgadGcaadGadAc | 7568.16 | 7564.21 |
| | 432271 | | | acuguususu | | |
| AD- | A- | sense | TTR | asascaguguUcUugcucuauaa | 8706.55 | 8702.04 |
| 266700 | 515453 | | | L96 | | |
| | A- | antis | TTR | usdTsaudAgadGcaadGadAc | 7568.16 | 7564.21 |
| | 432271 | | | acuguususu | | |
| AD- | A- | sense | TTR | asascagugudTcdTugcucuaua | 8702.60 | 8698.08 |
| 266701 | 128292 | | | aL96 | | |
| | A- | antis | TTR | usUsaudAgadGcaadGadAca | 7570.13 | 7566.19 |
| | 515454 | | | cuguususu | | |
| AD- | A- | sense | TTR | asascagugu Y87cY87ugcucu | 8975.03 | 8970.07 |
| 511257 | 816092 | | | auaaL96 | | |
| | A- | antis | TTR | usdTsaudAgadGcaadGadAc | 7568.16 | 7564.21 |
| | 432271 | | | acuguususu | | |
| AD- | A- | sense | TTR | asascagugudTcY87ugcucuau | 8838.82 | 8834.08 |
| 1631313 | 248363 | | | aaL96 | | |
| | 8 | | | | | |
| | A- | antis | TTR | usdTsaudAgadGcaadGadAc | 7568.16 | 7564.21 |
| | 432271 | | | acuguususu | | |
| AD- | A- | sense | TTR | asascagugu Y87cdTugcucuau | 8838.82 | 8834.08 |
| 1631314 | 248363 | | | aaL96 | | |
| | 7 | | | | | |
| | A- | antis | TTR | usdTsaudAgadGcaadGadAc | 7568.16 | 7564.21 |
| | 432271 | | | acuguususu | | |
| AD- | A- | sense | TTR | asascagugudTcY139ugcucua | 8946.82 | 8942.10 |
| 1631315 | 248363 | | | uaaL96 | | |
| | 4 | | | | | |
| | A- | antis | TTR | usdTsaudAgadGcaadGadAc | 7568.16 | 7564.21 |
| | 432271 | | | acuguususu | | |
| AD- | A- | sense | TTR | asascagugu Y139cdTugcucua | 8946.82 | 8942.10 |
| 1631316 | 248363 | | | uaaL96 | | |
| | 3 | | | | | |
| | A- | antis | TTR | usdTsaudAgadGcaadGadAc | 7568.16 | 7564.21 |
| | 432271 | | | acuguususu | | |
| AD- | A- | sense | TTR | asascagugu Y139cY139ugcu | 9191.03 | 9186.11 |
| 1631317 | 248363 | | | cuauaaL96 | | |
| | 2 | | | | | |
| | A- | antis | TTR | usdTsaudAgadGcaadGadAc | 7568.16 | 7564.21 |
| | 432271 | | | acuguususu | | |
| AD- | A- | sense | TTR | asascagugudTcY137ugcucua | 8838.83 | 8834.08 |
| 1631318 | 248363 | | | uaaL96 | | |
| | 1 | | | | | |
| | A- | antis | TTR | usdTsaudAgadGcaadGadAc | 7568.16 | 7564.21 |
| | 432271 | | | acuguususu | | |
| AD- | A- | sense | TTR | asascagugu Y137cdTugcucua | 8838.83 | 8834.08 |
| 1631319 | 248363 | | | uaaL96 | | |
| | 0 | | | | | |
| | A- | antis | TTR | usdTsaudAgadGcaadGadAc | 7568.16 | 7564.21 |
| | 432271 | | | acuguususu | | |
| AD- | A- | sense | TTR | asascagugu Y137cY137ugcu | 8975.05 | 8970.09 |
| 1631320 | 248362 | | | cuauaaL96 | | |
| | 9 | | | | | |
| | A- | antis | TTR | usdTsaudAgadGcaadGadAc | 7568.16 | 7564.21 |
| | 432271 | | | acuguususu | | |
| AD- | A- | sense | TTR | asascagugudTcdTugcucuaua | 8702.60 | 8698.08 |
| 238841 | 128292 | | | aL96 | | |
| | A- | antis | TTR | usdTsaudAgdAgcaadGadAc | 7568.15 | 7564.21 |
| | 463190 | | | acuguususu | | |
| AD- | A- | sense | TTR | asascaguguUcdTugcucuauaa | 8704.57 | 8700.06 |
| 266698 | 515451 | | | L96 | | |
| | A- | antis | TTR | usdTsaudAgadGcaadGadAc | 7568.16 | 7564.21 |
| | 432271 | | | acuguususu | | |
| AD- | A- | sense | TTR | asascagugudTcdTugcucuaua | 8702.60 | 8698.08 |
| 511258 | 128292 | | | aL96 | | |
| | A- | antis | TTR | usY87saudAgadGcaadGadA | 7704.37 | 7700.21 |
| | 816093 | | | cacuguususu | | |
| AD- | A- | sense | SOD1 | csasuuu(Uhd)AfaUfCfCfuca | 7043.98 | 7040.25 |
| 401824 | 637448 | | | cucuasasa | | |
| | A- | antis | SOD1 | VPusUfsuagAfgUfGfaggaUf | 7851.16 | 7847.15 |
| | 444402 | | | uAfaaaugsasg | | |
| AD- | A- | sense | SOD1 | csasuuu(Uhd)AfaUfCfCfuca | 7043.98 | 7040.25 |
| 401825 | 637448 | | | cucuasasa | | |
| | A- | antis | SOD1 | usUfsuagAfgUfGfaggaUfuA | 7775.16 | 7771.18 |
| | 268862 | | | faaaugsasg | | |
| AD- | A- | sense | SOD1 | csasuuu(Uhd)AfaUfCfCfuca | 7043.98 | 7040.25 |
| 454957 | 637448 | | | cucuasasa | | |
| | A- | antis | SOD1 | UsUfsuagAfgUfGfaggaUfu | 7761.13 | 7757.16 |
| | 637454 | | | Afaaaugsasg | | |
| AD- | A- | sense | SOD1 | csasuuu(Uhd)AfaUfCfCfuca | 7043.98 | 7040.25 |
| 1718729 | 637448 | | | cucuasasa | | |
| | A- | antis | SOD1 | Y137sUfsuagAfgUfGfagga | 7895.38 | 7891.18 |
| | 248363 | | | UfuAfaaaugsasg | | |
| | 9 | | | | | |
| AD- | A- | sense | SOD1 | csasuuu(Uhd)AfaUfCfCfuca | 7043.98 | 7040.25 |
| 1571162 | 637448 | | | cucuasasa | | |
| | A- | antis | SOD1 | Us(Ufms)uagAfgUfGfagga | 7775.16 | 7771.18 |
| | 248364 | | | UfuAfaaaugsasg | | |
| | 0 | | | | | |
| AD- | A- | sense | SOD1 | csasuuu(Uhd)AfaUfCfCfuca | 7043.98 | 7040.25 |
| 1720685 | 637448 | | | cucuasasa | | |
| | A- | antis | SOD1 | Y137s(Ufms)uagAfgUfGfag | 7909.40 | 7905.20 |
| | 248364 | | | gaUfuAfaaaugsasg | | |
| | 1 | | | | | |
| AD- | A- | sense | SOD1 | csasuuu(Uhd)AfaUfCfCfuca | 7043.98 | 7040.25 |
| 890110 | 637448 | | | cucuasasa | | |
| | A- | antis | SOD1 | (URs)(Ufms)uagAfgUfGfag | 7775.16 | 7771.18 |
| | 158509 | | | gaUfuAfaaaugsasg | | |
| | 3 | | | | | |
| AD- | A- | sense | SOD1 | csasuuu(Uhd)AfaUfCfCfuca | 7043.98 | 7040.25 |
| 1720686 | 637448 | | | cucuasasa | | |
| | A- | antis | SOD1 | (Y137Rs)UfsuagAfgUfGfag | 7895.38 | 7891.18 |
| | 287260 | | | gaUfuAfaaaugsasg | | |
| | 0 | | | | | |

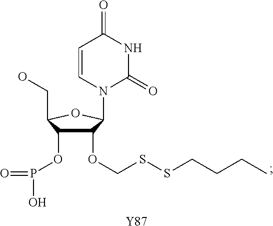

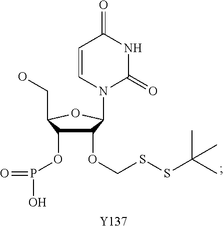

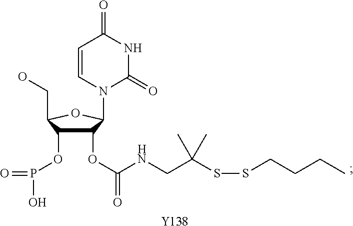

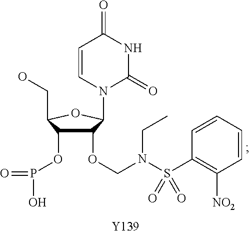

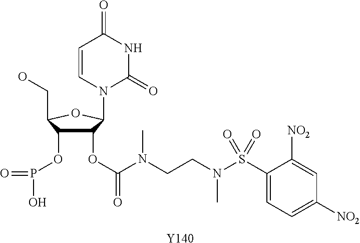

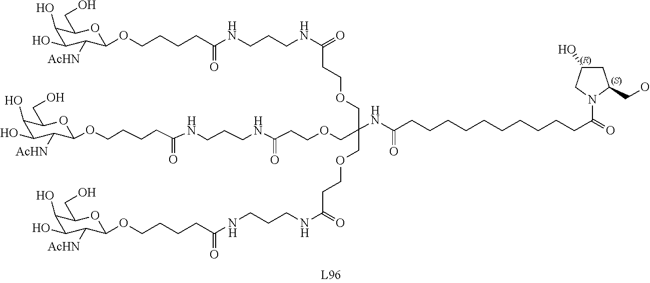

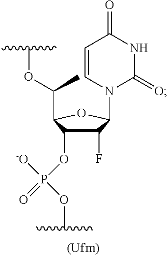

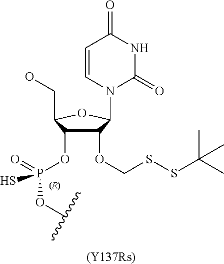

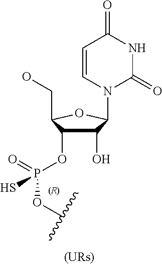

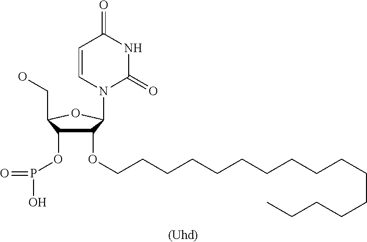

### Example 4. Glutathione Assay to Determine the Half-Life of Single-Strand RNA Containing 2′-Modified Nucleoside

Modified oligonucleotide (23-nt length) was added at 100 μM to a solution of 250 μg (6.25 U/mL) glutathione-S-transferase from equine liver (GST) (Sigma Cat. No. G6511) and 0.1 mg/mL NADPH (Sigma Cat. No. 481973) in 0.1M Tris pH7.2. Glutathione (GSH) (MP Biomedicals, Inc. Cat. No. 101814 #) was added to the mixture for a final concentration of 10 mM. Immediately after addition of GSH, sample was injected onto a Dionex DNAPac PA200 column (4×250 mm) at 30° C. and run on an anion exchange gradient of 35-65% (20 mM Sodium Phosphate, 10-15% CH₃CN, 1M Sodium Bromide pH11) at 1 mL/min for 7.5 minutes.

Glutathione-mediated cleavage kinetics were monitored every hour for 24 hours. The area under the main peak for each hour was normalized to the area from the 0 h time point (first injection). First-order decay kinetics were used to calculate half-lives. A control sequence containing modified oligonucleotide (23-nt length) with 5′ Thiol modifier C6 (Glen Research Cat.No. 10-1936-02) between N6 and N7 was run each day of assay run. A second control sequence containing modified oligonucleotide (23-nt length) with the same 5′ thiol modifier C6 at Ni was also run once per set of sequences. Half-lives were reported relative to half-life of control sequence. Glutathione and GST were prepared as stocks of 100 mM and 10 mg/mL in water, respectively, and aliquoted into 1 mL tubes and stored at −80° C. A new aliquot was used for every day the assay was run.

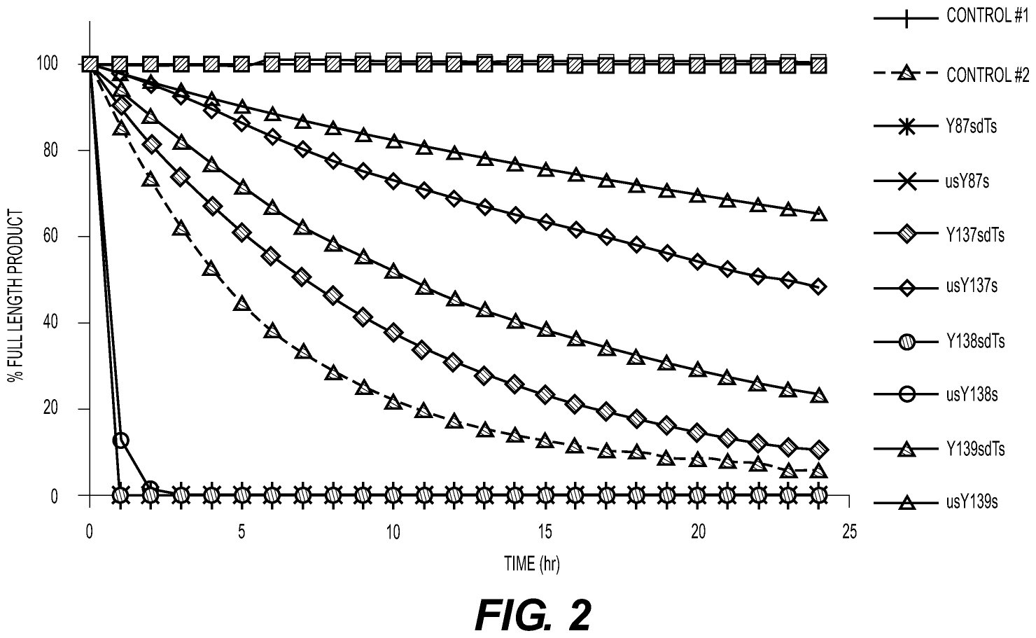

The cleavage profiles of different single-stranded oligonucleotides modified with different chemical modifications to protect 2′—OH group, after treating with glutathione and followed by HPLC over 25 hours, are shown in FIG. 2. The single-stranded oligonucleotides used for the glutathione assay and the results from FIG. 2 are also summarized in Table 3.

TABLE 3
Stability of oligonucleotides containing 2′-modified nucleoside prodrugs after
incubating with glutathione, determined from Figure 2

| | | Half-Life |
|---|---|---|
| Oligo ID | Sequence | (h) |
| A-801703 | Q51uUfaUfaGfaGfcAfagaAfcAfcUfgUfuuu | <1 |
| (Control #1) | | |
| A-801704 | uUfaUfaGfQ51GfcAfagaAfcAfcUfgUfuuu | 5.9 (avg) |
| (Control #2) | | |
| A-1690802 | Y87sdTsaudAgadGcaadGadAcacuguususu | <1 |
| A-816093 | usY87saudAgadGcaadGadAcacuguususu | <1 |
| A-1690798 | Y137sdTsaudAgadGcaadGadAcacuguususu | 7.3 |
| A-1690793 | usY137saudAgadGcaadGadAcacuguususu | 22.6 |
| A-1690799 | Y138sdTsaudAgadGcaadGadAcacuguususu | <1 |
| A-1690794 | usY138saudAgadGcaadGadAcacuguususu | 0.3 |
| A-1690800 | Y139sdTsaudAgadGcaadGadAcacuguususu | 11.4 |
| A-1690795 | usY139saudAgadGcaadGadAcacuguususu | 39.3 |
| A-1690801 | Y140sdTsaudAgadGcaadGadAcacuguususu | 4858.6 |
| A-1690796 | usY140saudAgadGcaadGadAcacuguususu | 3594.4 |

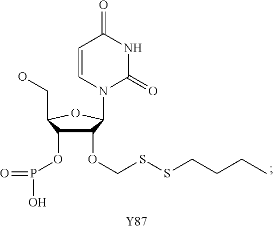

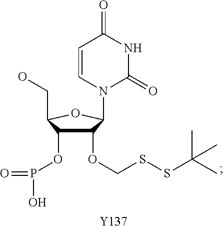

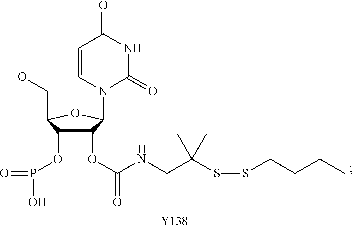

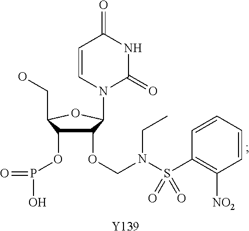

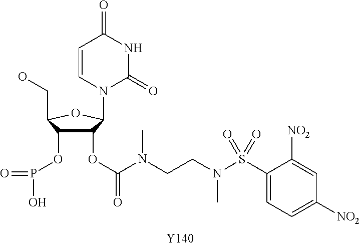

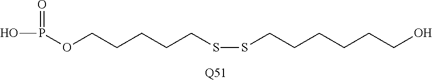

### Example 5. In Vitro Gene Silencing of Oligonucleotides Containing 2′-Modified Nucleotides

Transfection procedure: siRNAs containing 2′-modified nucleoside analogues (Table 2) were transfected in primary mouse hepatocytes with RNAiMAX at 0.1, 1, 10, and 100 nm concentrations and analyzed 24 hours post-transfection. TTR message remaining was determined by qPCR, results are shown in Tables 4 and 5.

Free uptake procedure: siRNAs containing 2′-modifed nucleoside analogous (Table 2) were incubated with primary mouse hepatocytes at 0.1, 1, 10, and 100 nm concentrations in cell culture medium and analyzed after 48 hours. TTR message remaining was determined by qPCR.

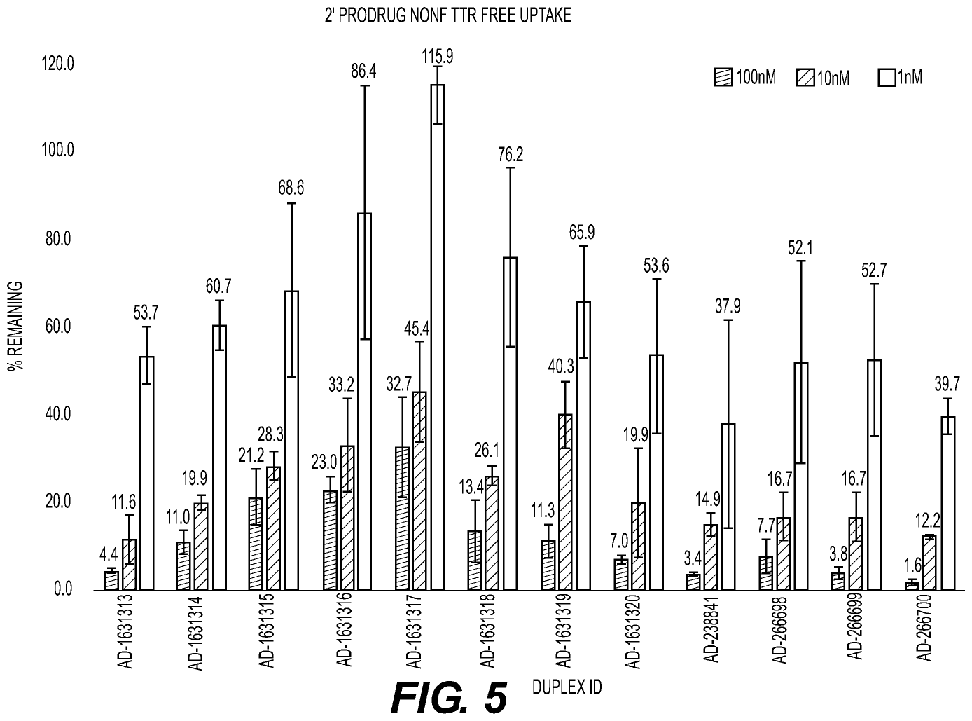

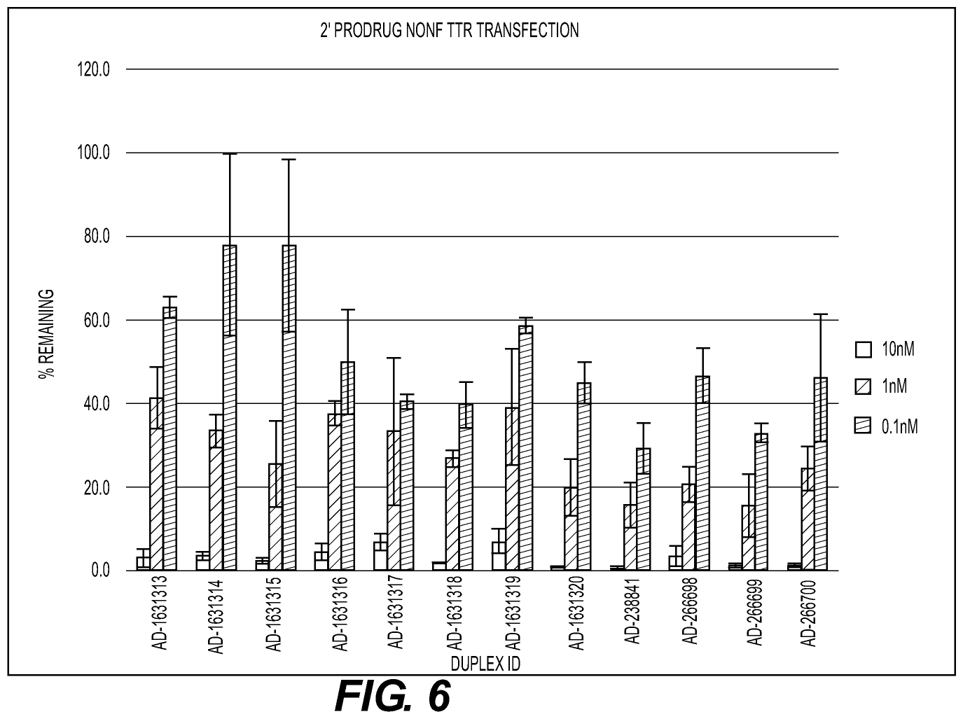

The in vitro gene silencing activities of siRNAs containing 2′-modified nucleoside analogues are shown in FIGS. 5-6.

TABLE 4
In vitro evaluation of 2′-modified siRNAs

| | Free Uptake (TTR | Transfection (TTR | |
|---|---|---|---|
| Sequence ID | remaining) | remaining) | 2′-Modification |
| AD-238841 | 1.37E−03 | 3.68E−05 | Control |
| AD-266719 | 2.33E−04 | 4.23E−05 | 2′-OH @ N1 |
| | | | control |
| AD-1020664 | 7.25E−06 | 3.71E−07 | Y87 |
| AD-1020665 | 6.63E−02 | 1.30E−01 | Y138 |
| AD-1020666 | 2.65E−03 | 4.84E−02 | Y139 |
| AD-1020667 | 1.86E+00 | 3.41E−01 | Y140 |
| AD-1025232 | 6.90E−05 | 9.41E−06 | Y137 |
| AD-266701 | 9.94E−04 | 2.81E−04 | 2′-OH @ N2 |
| | | | control |
| AD-511258 | 1.35E−04 | 7.18E−04 | Y87 |
| AD-1020661 | >100 | >10 | Y138 |
| AD-1020662 | 6.46E−02 | 8.77E−01 | Y139 |
| AD-1020663 | >100 | >10 | Y140 |
| AD-1025231 | 1.80E−04 | 3.87E−03 | Y137 |

TABLE 5
In vitro gene silencing of 2′-modified siRNAs IC₅₀ values (nM)
of Y87-containing 2′-prodrug siRNAs targeting TTR mRNA

| | Transfection, | Free uptake, | |
|---|---|---|---|
| Duplex ID | IC₅₀ (nM) | IC50 (nM) | 2′-Modification |
| AD-64958 | 0.011 | 0.126 | Control |
| AD-266700 | 0.046 | 1.079 | 2′-OH @ N9/N11 sense |
| AD-266701 | 0.022 | 0.520 | 2′-OH @ N1 control |
| AD-238841 | 0.016 | 0.335 | Control |
| AD-511257 | 0.070 | 1.110 | Y87 @ N9/N11 sense |
| AD-511258 | 0.026 | 0.472 | Y87 @ N2 antisense |

### Example 6. In Vivo Evaluation of 2′-Modified siRNAs

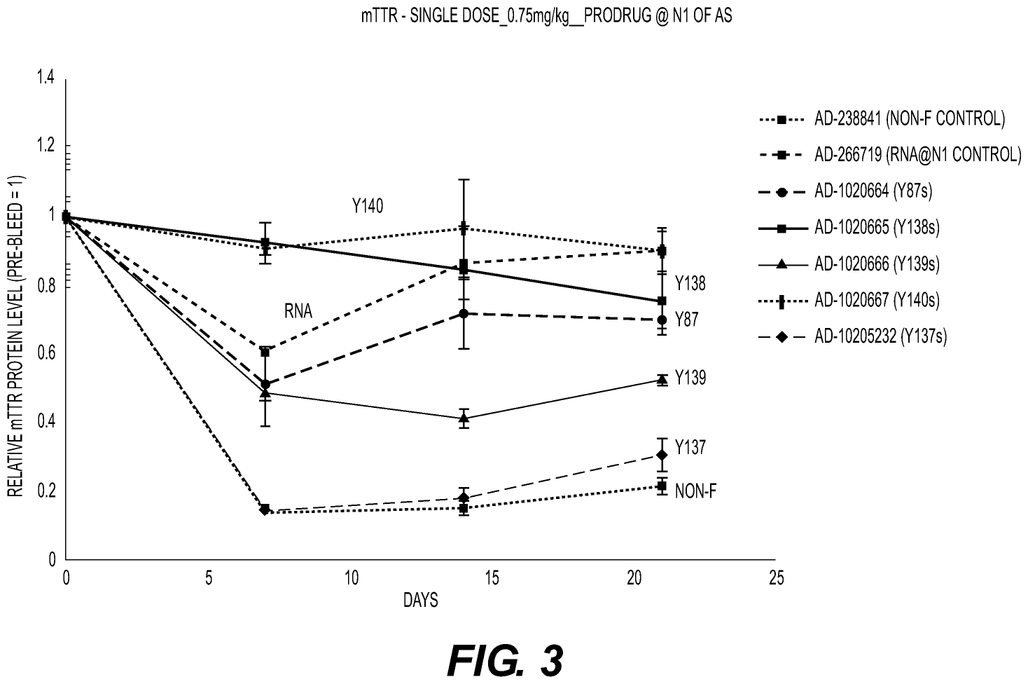

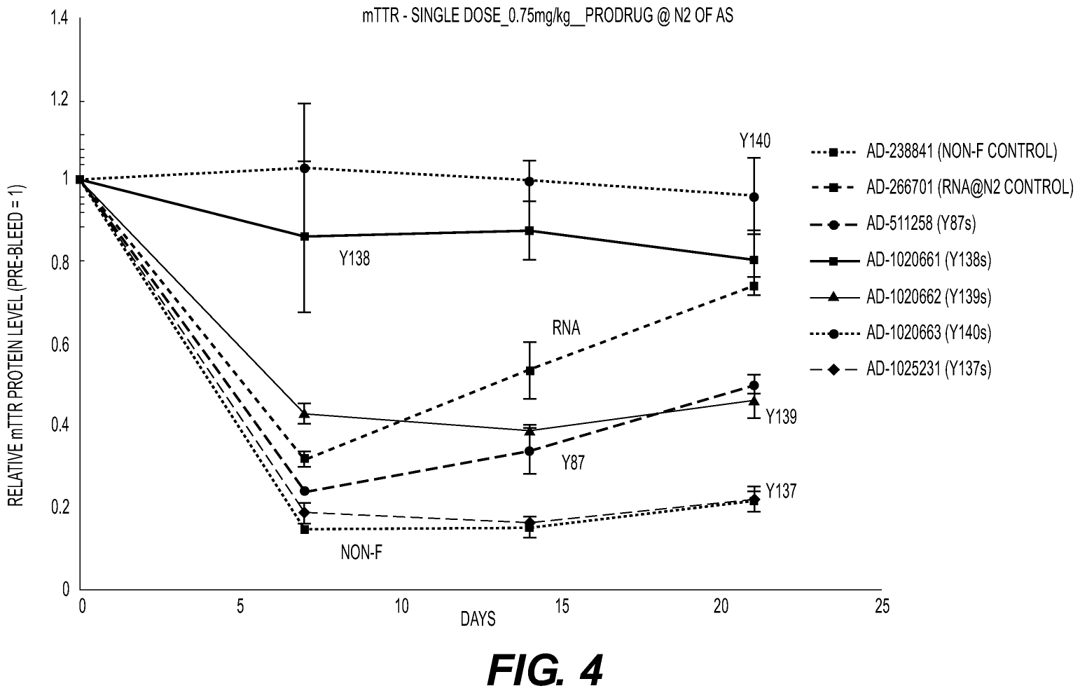

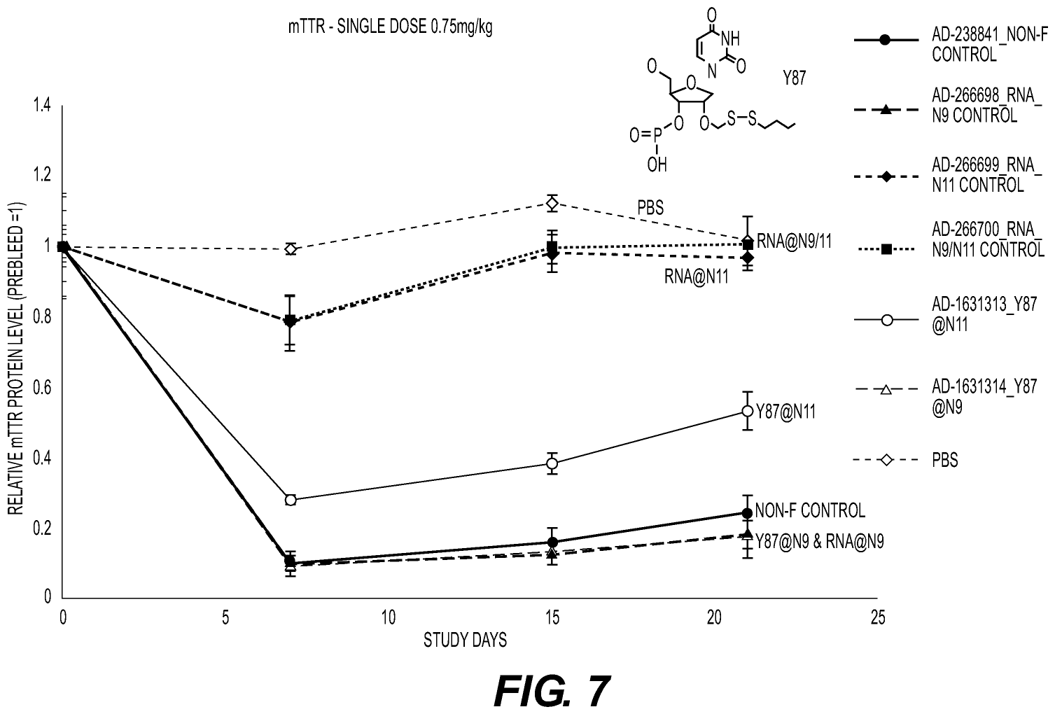

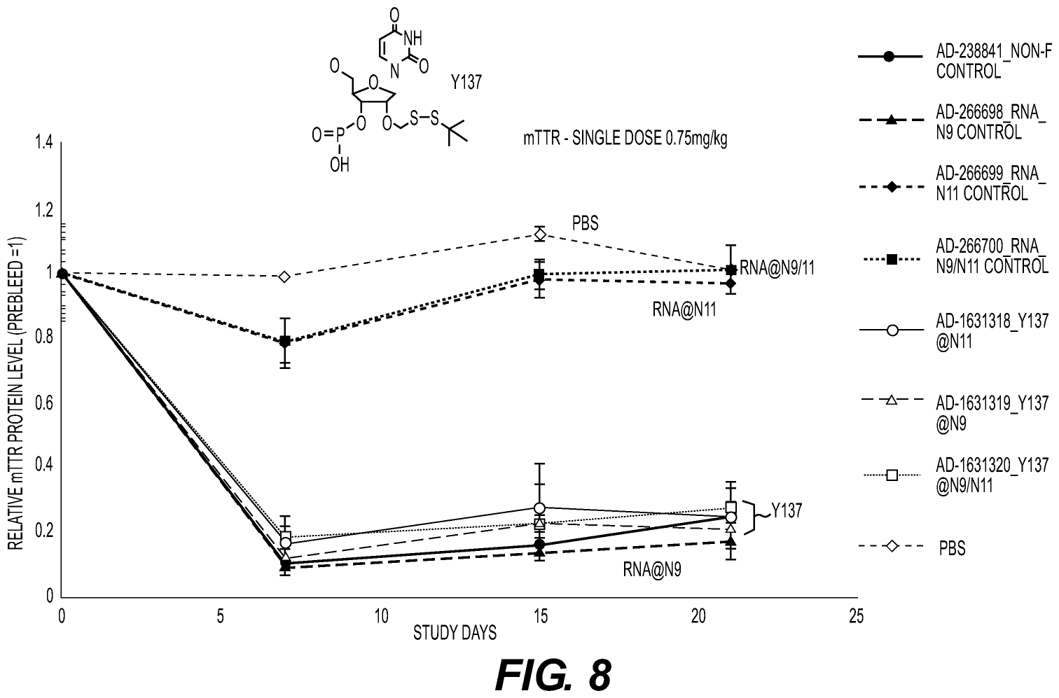

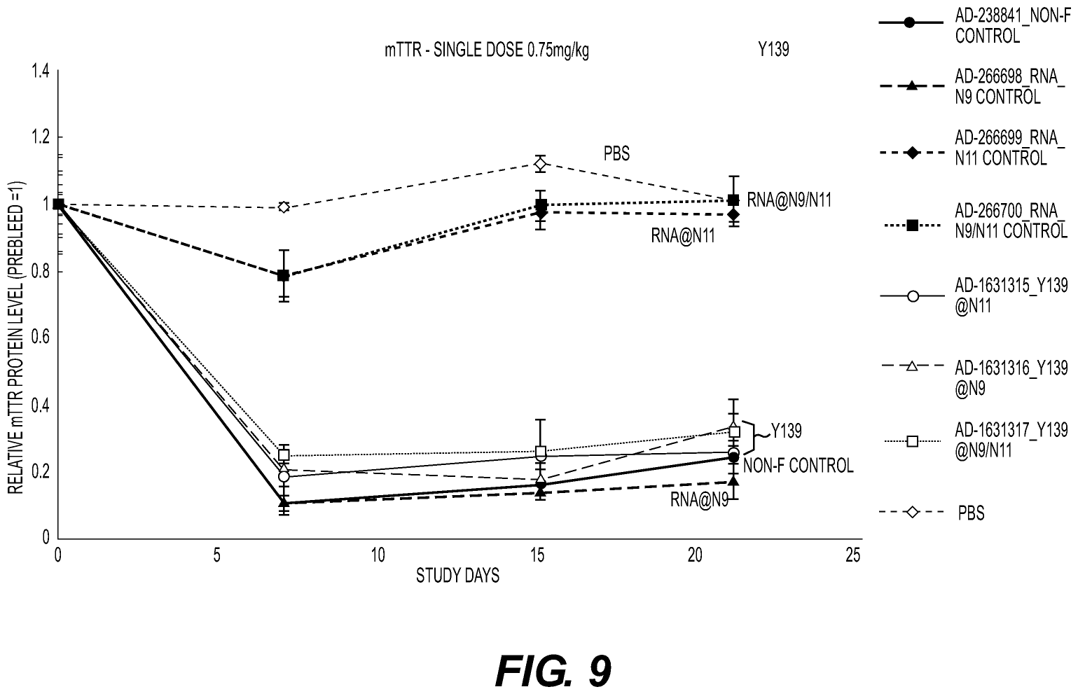

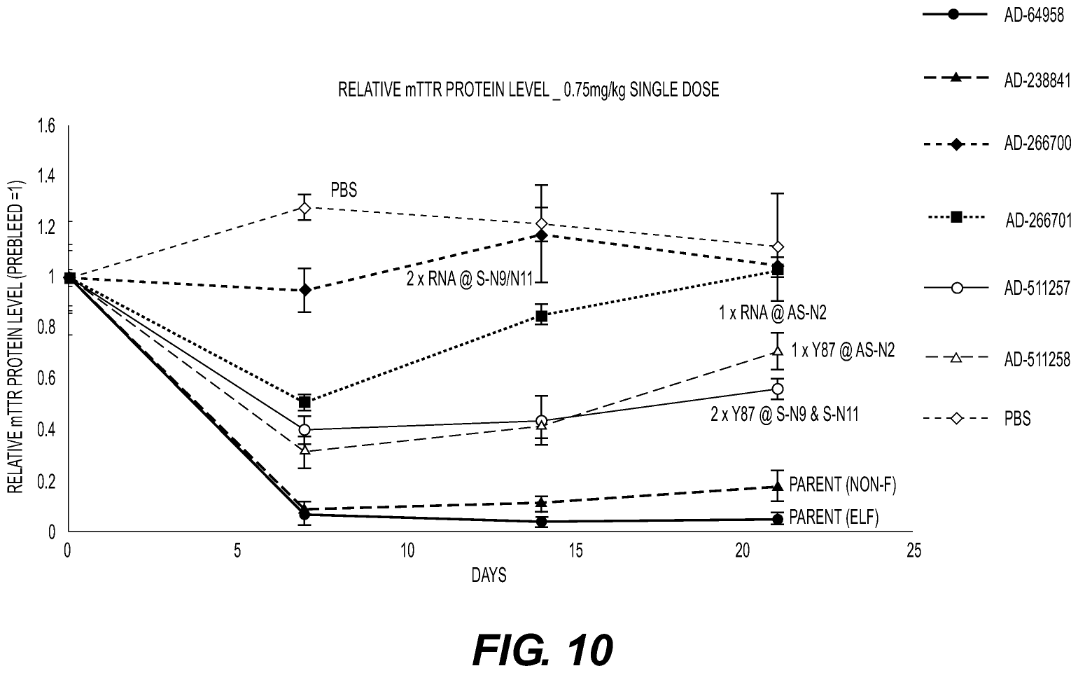

In vivo study procedure: C57b16 female mice (n═3/group) were dosed with either 0.1 mg/mL or 0.3 mg/mL of siRNA duplex containing 2′-modified nucleoside analogues in Table 6. Serum was collected at days 11, 22 and 35 days pos-dose and analyzed via ELISA to determine relative TTR protein levels. The results are shown in FIGS. 3-4 and 7-10.

TABLE 6
siRNA duplexes used in vivo studies in liver

| Duplex ID | Oligo ID | Strand | Target | Oligo Seq |
|---|---|---|---|---|
| AD- | A- | sense | TTR | asascagugudTcdTugcucuauaaL96 |
| 238841 | 128292 | | | |
| | A- | antis | TTR | usdTsaudAgdAgcaadGadAcacuguususu |
| | 463190 | | | |
| AD- | A- | sense | TTR | asascagugudTcdTugcucuauaaL96 |
| 266701 | 128292 | | | |
| | A- | antis | TTR | usUsaudAgadGcaadGadAcacuguususu |
| | 515454 | | | |
| AD- | A- | sense | TTR | asascagugudTcdTugcucuauaaL96 |
| 266719 | 128292 | | | |
| | A- | antis | TTR | UsdTsaudAgadGcaadGadAcacuguususu |
| | 515472 | | | |
| AD- | A- | sense | TTR | asascagugudTcdTugcucuauaaL96 |
| 511258 | 128292 | | | |
| | A- | antis | TTR | usY87saudAgadGcaadGadAcacuguususu |
| | 816093 | | | |
| AD- | A- | sense | TTR | asascagugudTcdTugcucuauaaL96 |
| 1025231 | 128292 | | | |
| | A- | antis | TTR | usY137saudAgadGcaadGadAcacuguususu |
| | 1690793 | | | |
| AD- | A- | sense | TTR | asascagugudTcdTugcucuauaaL96 |
| 1020661 | 128292 | | | |
| | A- | antis | TTR | usY138saudAgadGcaadGadAcacuguususu |
| | 1690794 | | | |
| AD- | A- | sense | TTR | asascagugudTcdTugcucuauaaL96 |
| 1020662 | 128292 | | | |
| | A- | antis | TTR | usY139saudAgadGcaadGadAcacuguususu |
| | 1690795 | | | |
| AD- | A- | sense | TTR | asascagugudTcdTugcucuauaaL96 |
| 1020663 | 128292 | | | |
| | A- | antis | TTR | usY140saudAgadGcaadGadAcacuguususu |
| | 1690796 | | | |
| AD- | A- | sense | TTR | asascagugudTcdTugcucuauaaL96 |
| 1025232 | 128292 | | | |
| | A- | antis | TTR | Y137sdTsaudAgadGcaadGadAcacuguususu |
| | 1690798 | | | |
| AD- | A- | sense | TTR | asascagugudTcdTugcucuauaaL96 |
| 1020665 | 128292 | | | |
| | A- | antis | TTR | Y138sdTsaudAgadGcaadGadAcacuguususu |
| | 1690799 | | | |
| AD- | A- | sense | TTR | asascagugudTcdTugcucuauaaL96 |
| 1020666 | 128292 | | | |
| | A- | antis | TTR | Y139sdTsaudAgadGcaadGadAcacuguususu |
| | 1690800 | | | |
| AD- | A- | sense | TTR | asascagugudTcdTugcucuauaaL96 |
| 1020667 | 128292 | | | |
| | A- | antis | TTR | Y140sdTsaudAgadGcaadGadAcacuguususu |
| | 1690801 | | | |
| AD- | A- | sense | TTR | asascagugudTcdTugcucuauaaL96 |
| 1020664 | 128292 | | | |
| | A- | antis | TTR | Y87sdTsaudAgadGcaadGadAcacuguususu |
| | 1690802 | | | |
| AD- | A- | sense | TTR | asascagugudTcdTugcucuauaaL96 |
| 238841 | 128292 | | | |
| | A- | antis | TTR | usdTsaudAgdAgcaadGadAcacuguususu |
| | 463190 | | | |
| AD- | A- | sense | TTR | asascaguguUcdTugcucuauaaL96 |
| 266698 | 515451 | | | |
| | A- | antis | TTR | usdTsaudAgadGcaadGadAcacuguususu |
| | 432271 | | | |
| AD- | A- | sense | TTR | asascagugudTcUugcucuauaaL96 |
| 266699 | 515452 | | | |
| | A- | antis | TTR | usdTsaudAgadGcaadGadAcacuguususu |
| | 432271 | | | |
| AD- | A- | sense | TTR | asascaguguUcUugcucuauaaL96 |
| 266700 | 515453 | | | |
| | A- | antis | TTR | usdTsaudAgadGcaadGadAcacuguususu |
| | 432271 | | | |
| AD- | A- | sense | TTR | asascagugudTcdTugcucuauaaL96 |
| 266701 | 128292 | | | |
| | A- | antis | TTR | usUsaudAgadGcaadGadAcacuguususu |
| | 515454 | | | |
| AD- | A- | sense | TTR | asascagugu Y87cY87ugcucuauaaL96 |
| 511257 | 816092 | | | |
| | A- | antis | TTR | usdTsaudAgadGcaadGadAcacuguususu |
| | 432271 | | | |
| AD- | A- | sense | TTR | asascagugudTcY87ugcucuauaaL96 |
| 1631313 | 2483638 | | | |
| | A- | antis | TTR | usdTsaudAgadGcaadGadAcacuguususu |
| | 432271 | | | |
| AD- | A- | sense | TTR | asascagugu Y87cdTugcucuauaaL96 |
| 1631314 | 2483637 | | | |
| | A- | antis | TTR | usdTsaudAgadGcaadGadAcacuguususu |
| | 432271 | | | |
| AD- | A- | sense | TTR | asascagugudTcY139ugcucuauaaL96 |
| 1631315 | 2483634 | | | |
| | A- | antis | TTR | usdTsaudAgadGcaadGadAcacuguususu |
| | 432271 | | | |
| AD- | A- | sense | TTR | asascagugu Y139cdTugcucuauaaL96 |
| 1631316 | 2483633 | | | |
| | A- | antis | TTR | usdTsaudAgadGcaadGadAcacuguususu |
| | 432271 | | | |
| AD- | A- | sense | TTR | asascagugu Y139cY139ugcucuauaaL96 |
| 1631317 | 2483632 | | | |
| | A- | antis | TTR | usdTsaudAgadGcaadGadAcacuguususu |
| | 432271 | | | |
| AD- | A- | sense | TTR | asascagugudTcY137ugcucuauaaL96 |
| 1631318 | 2483631 | | | |
| | A- | antis | TTR | usdTsaudAgadGcaadGadAcacuguususu |
| | 432271 | | | |
| AD- | A- | sense | TTR | asascagugu Y137cdTugcucuauaaL96 |
| 1631319 | 2483630 | | | |
| | A- | antis | TTR | usdTsaudAgadGcaadGadAcacuguususu |
| | 432271 | | | |
| AD- | A- | sense | TTR | asascagugu Y137cY 13 7ugcucuauaaL96 |
| 1631320 | 2483629 | | | |
| | A- | antis | TTR | usdTsaudAgadGcaadGadAcacuguususu |
| | 432271 | | | |
| AD- | A- | sense | TTR | asascagugudTcdTugcucuauaaL96 |
| 238841 | 128292 | | | |
| | A- | antis | TTR | usdTsaudAgdAgcaadGadAcacuguususu |
| | 463190 | | | |
| AD- | A- | sense | TTR | asascaguguUcdTugcucuauaaL96 |
| 266698 | 515451 | | | |
| | A- | antis | TTR | usdTsaudAgadGcaadGadAcacuguususu |
| | 432271 | | | |
| AD- | A- | sense | TTR | asascagugudTcdTugcucuauaaL96 |
| 511258 | 128292 | | | |
| | A- | antis | TTR | usY87saudAgadGcaadGadAcacuguususu |
| | 816093 | | | |

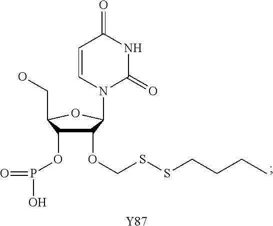

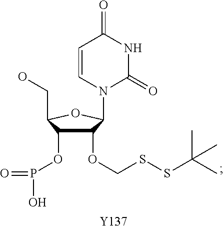

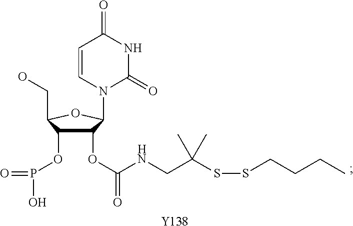

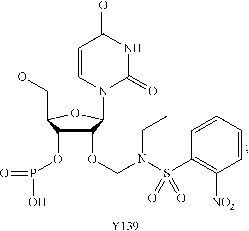

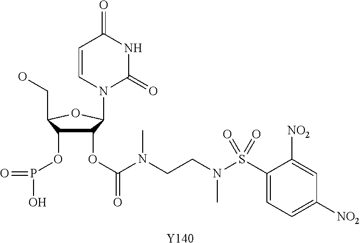

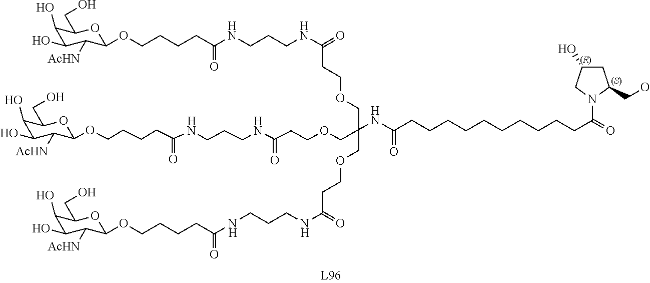

### Example 7. 2′Podrug siRNA for CNS Studies

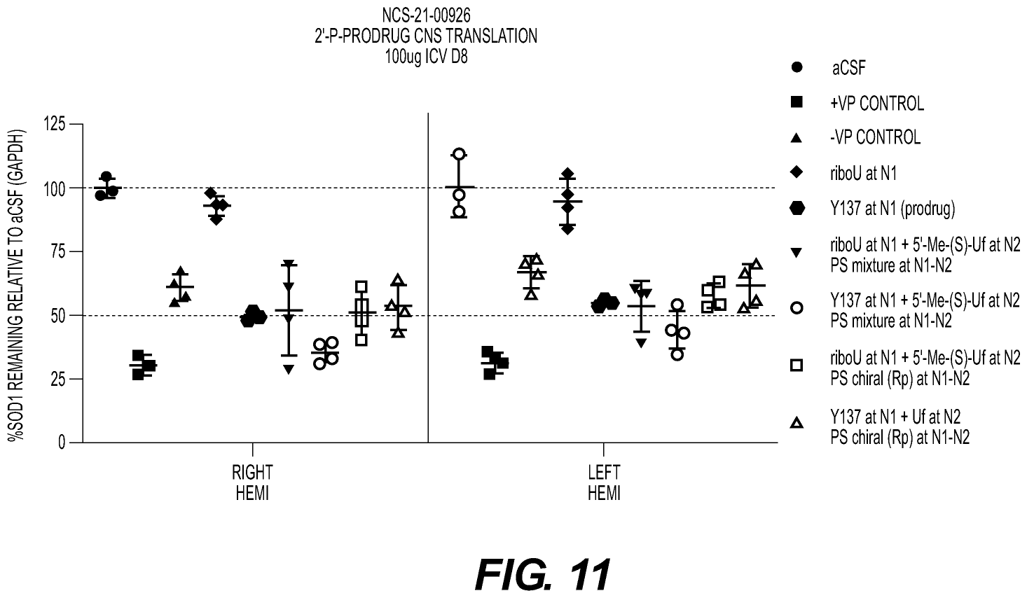

Protocol for Mouse ICV (intracerebroventricular administration) studies: the siRNAs (Table 7) formulated at 20 mg/mL in artificial cerebrospinal fluid (aCSF) were administered as 5 μL freehand ICV injections via the right lateral ventricular space to female C57/BL6 mice. Following anesthesia with isoflurane, the ICV injection site was disinfected with alcohol. siRNA was administered with a glass Hamilton syringe and custom 3 mm length needle to the lateral ventricle approximately 1.0 mm lateral and 0.3 mm rostral from bregma. Once siRNA administration was completed, mice were returned to their home cage and monitored for recovery. The rodents were euthanized 8 days post injection via CO2 exposure. The brain was collected, hemisected, and frozen in liquid nitrogen before being pulverized into powder. RNA was extracted from the tissue powder utilizing a magnetic bead-based process, and the mRNA levels were assessed via RT-qPCR. The results are shown in FIG. 11.

TABLE 7
Duplexes used for CNS studies

| Duplex ID | Oligo ID | Strand | Target | Oligo Seq |
|---|---|---|---|---|
| AD-401824 | A-637448 | sense | SOD1 | csasuuu(Uhd)AfaUfCfCfucacucuasasa |
| | A-444402 | antis | SOD1 | VPusUfsuagAfgUfGfaggaUfuAfaaaugsasg |
| AD-401825 | A-637448 | sense | SOD1 | csasuuu(Uhd)AfaUfCfCfucacucuasasa |
| | A-268862 | antis | SOD1 | usUfsuagAfgUfGfaggaUfuAfaaaugsasg |
| AD-454957 | A-637448 | sense | SOD1 | csasuuu(Uhd)AfaUfCfCfucacucuasasa |
| | A-637454 | antis | SOD1 | UsUfsuagAfgUfGfaggaUfuAfaaaugsasg |
| AD-1718729 | A-637448 | sense | SOD1 | csasuuu(Uhd)AfaUfCfCfucacucuasasa |
| | A-2483639 | antis | SOD1 | Y137sUfsuagAfgUfGfaggaUfuAfaaaugsasg |
| AD-1571162 | A-637448 | sense | SOD1 | csasuuu(Uhd)AfaUfCfCfucacucuasasa |
| | A-2483640 | antis | SOD1 | Us(Ufms)uagAfgUfGfaggaUfuAfaaaugsasg |
| AD-1720685 | A-637448 | sense | SOD1 | csasuuu(Uhd)AfaUfCfCfucacucuasasa |
| | A-2483641 | antis | SOD1 | Y137s(Ufms)uagAfgUfGfaggaUfuAfaaaugsasg |
| AD-890110 | A-637448 | sense | SOD1 | csasuuu(Uhd)AfaUfCfCfucacucuasasa |
| | A-1585093 | antis | SOD1 | (URs)(Ufms)uagAfgUfGfaggaUfuAfaaaugsasg |
| AD-1720686 | A-637448 | sense | SOD1 | csasuuu(Uhd)AfaUfCfCfucacucuasasa |
| | A-2872600 | antis | SOD1 | (Y137Rs)UfsuagAfgUfGfaggaUfuAfaaaugsasg |

### Example 8. Introduction of the 2′-Modified Nucleoside Prodrug Buildings Blocks into siRNAs

Different 2′-modified nucleoside prodrug derivatives can be introduced at specific positions in the sense strand and/or antisense strand of a siRNA duplex, as a temporary protecting group for 2′-hydroxyl group, as shown in Schemes 18-20.

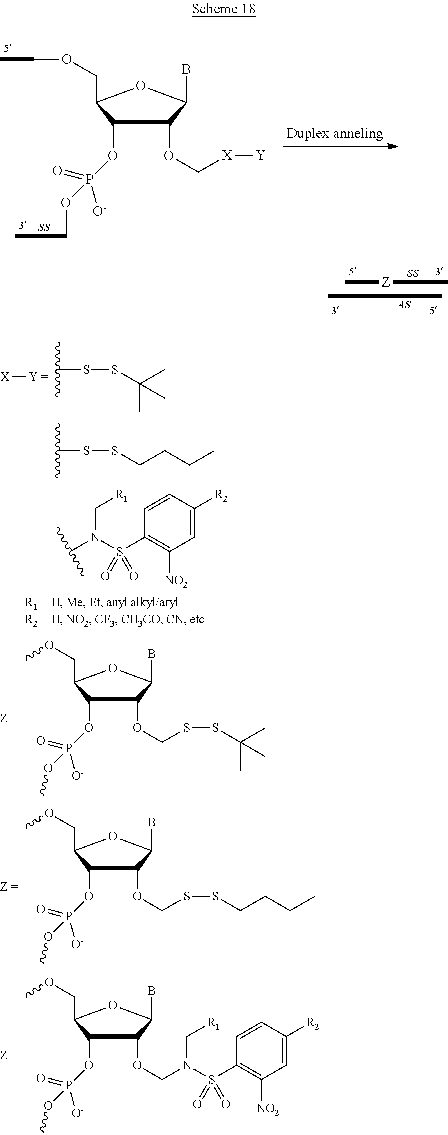

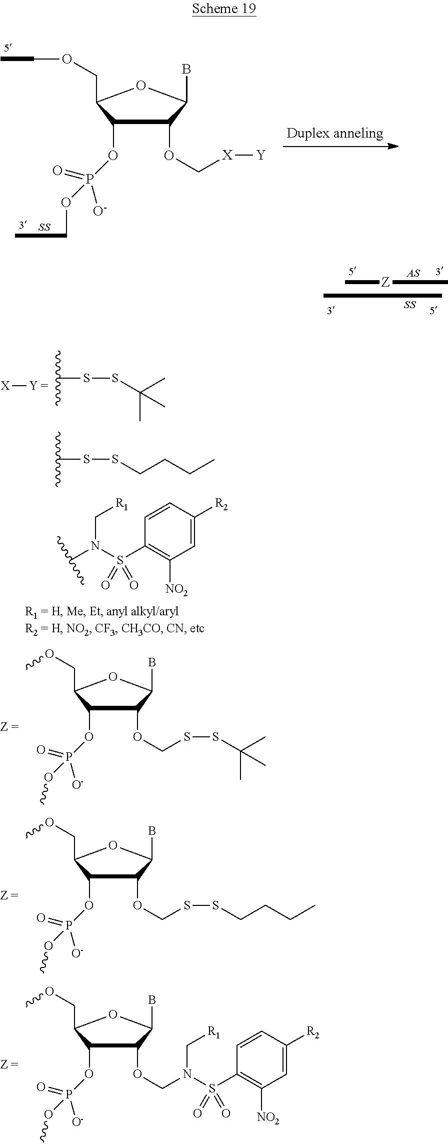

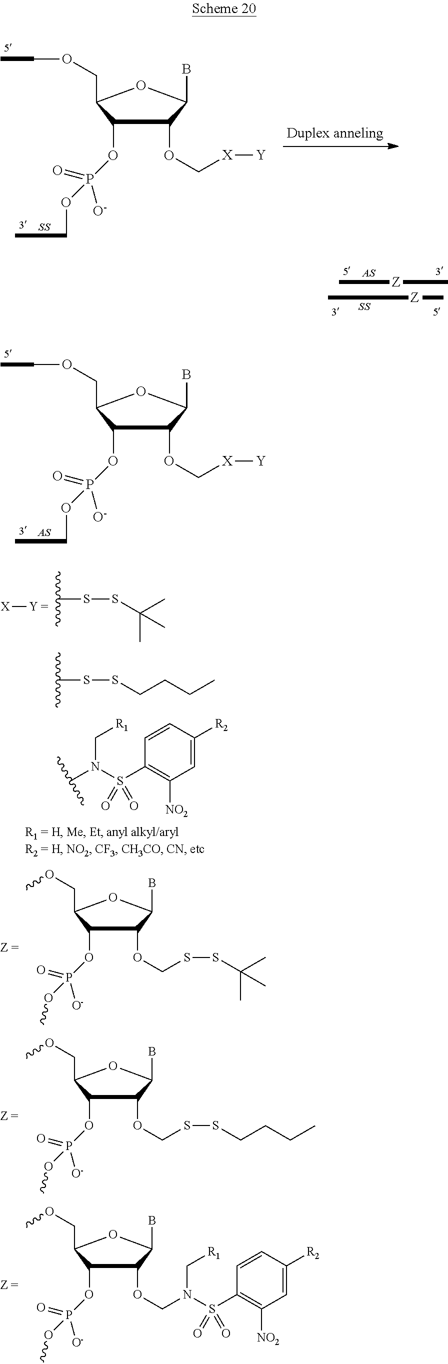

### Example 9. Using the 2′-Modified Nucleoside Prodrugs to Generate Cleavable siRNA Conjugates Having Different Targeting Ligands

Different targeting ligands for specific cell surface receptors can be introduced into a siRNA duplex through the 2′-modified nucleoside prodrug linkers, as shown in Schemes 21-22. These derivatives will be cleaved off after the siRNA enters into cytosol.

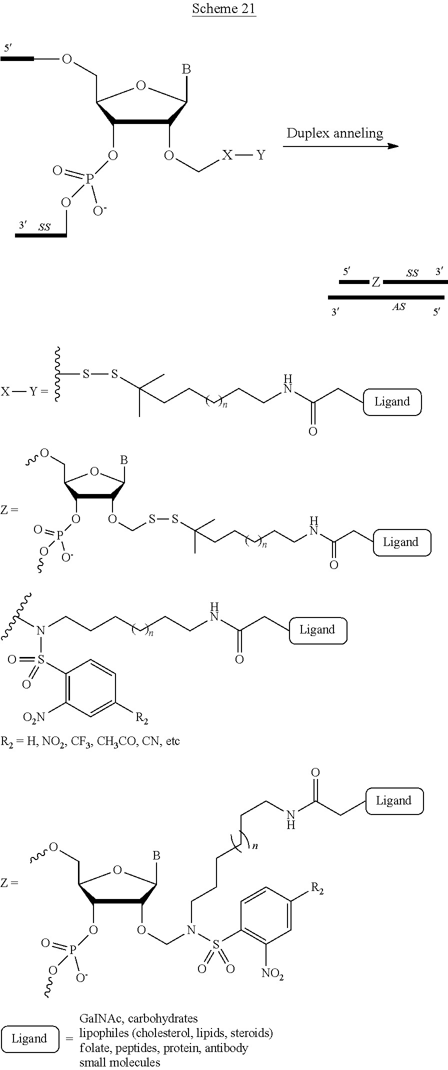

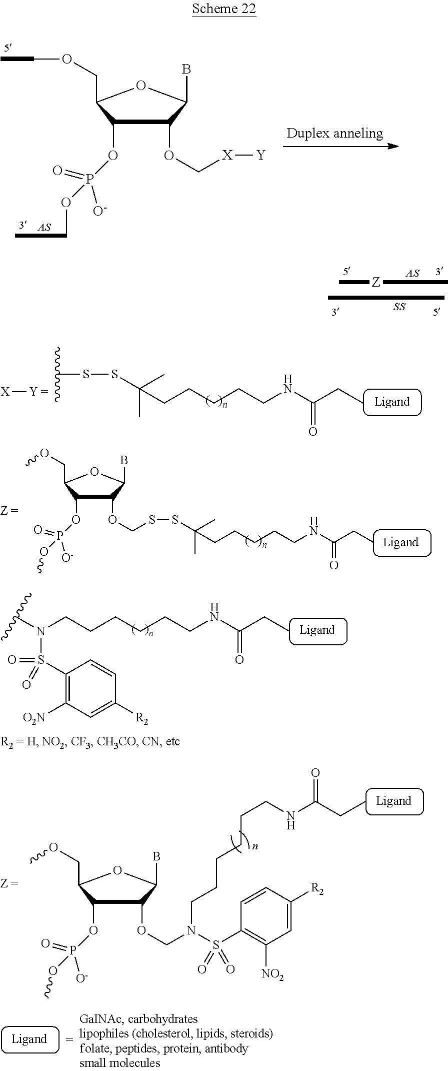

1. An oligonucleotide comprising one or more 2′-modified nucleosides, wherein the 2′-position of the nucleoside has a structure of formula (I): 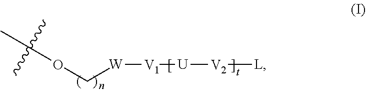
wherein:
W is O, S—S, N(R^{N}), C(O)N(R^{N}), N(R^{N})C(O), N(R^{N})C(O)O, N(R^{N})S(O)₂, or a biocleavable carbohydrate linker;
V₁ and V₂ are each independently for each occurrence absent, alkylene, arylene, heteroarylene, a divalent cycloalkyl, or a divalent heterocyclyl, each of which can be optionally substituted by one or more R²⁰ groups;
U is independently for each occurrence absent, W, O-W, W-O, or a phosphate or modified phosphate;
R^{N} is H, alkyl, or S(O)₂-aryl, each of which can be optionally substituted by one or more R²⁰ groups;
L is H or one or more ligands;
R²⁰ is independently for each occurrence halo, haloalkyl, hydroxyl, alkoxy, methoxyalkoxy, alkylthio, amino, alkylamino, alkynyl, aminoalkyl, aminoalkoxy, nitro, acetyl, or cyano;
n is 0 or 1;
t is an integer of 1-3 representing the number of presence for unit [U-V₂], which can be the same or different for each occurrence; and [symbol]
indicates the attachment of the formula (I) to the 2′-position of the nucleoside.

2. The oligonucleotide of claim 1, wherein the 2′-modified nucleoside has the structure of formula (II): 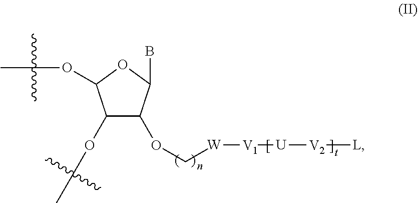
wherein:
each [symbol] indicates the attachment of the formula (II) to an adjacent nucleotide of the oligonucleotide, or hydrogen, or together with the oxygen to which it is attached forms a phosphate, or a modified phosphate, wherein at least one [symbol] attaches to an adjacent nucleotide; and
B is modified or unmodified nucleobase.

3. The oligonucleotide of claim 2, wherein B is independently for each occurrence A, A^{Bz}, C, C^{Ac}, C^{Bz}, 5-Me-C, 5-Me-C^{Ac}, G, G^{iBu}, I, U, T, 2-thiouridine, 4-thiouridine, a C5-modified pyrimidine, C2-modified purine, N8-modifed purine, phenoxazine, G-clamp, non-canonical mono-, bi-, and tri-cyclic heterocycles, a pseudouracil, isoC, isoG, 2,6-diamninopurine, a pseudocytosine, 2-aminopurine, xanthosine, N6-alkyl-A, or O6-alkyl-G.

4. The oligonucleotide of claim 2, wherein the 2′-modified nucleoside has the structure of formula (IIa) or (IIb): 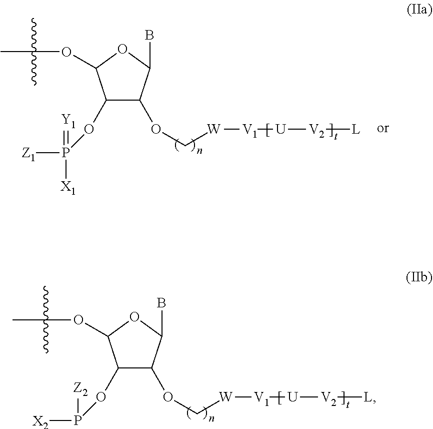
or a salt thereof, wherein: [symbol]
indicates the attachment of the formula (IIa) or (IIb) to the adjacent nucleotide of the oligonucleotide, hydrogen, or together with the oxygen to which it is attached forms a phosphate, or a modified phosphate; and
X₁ and Z₁ are each independently H, OH, OM, OR¹³, SH, SM, SR¹³, C(O)H, S(O)H, or alkyl, each of which can be optionally substituted with one or more R^{sub} groups, N(R′)(R″), B(R¹³)₃, BH₃⁻, Se; or D-Q, wherein D is independently for each occurrence absent, O, S, N(R′), alkylene, each of which can be optionally substituted with one or more R^{sub} groups, and Q is independently for each occurrence a nucleoside or oligonucleotide;
X₂ and Z₂ are each independently N(R′)(R″), OR¹⁸, or D-Q, wherein D is independently for each occurrence absent, O, S, N, N(R′), alkylene, each of which can be optionally substituted with one or more R^{sub} groups, and Q is independently for each occurrence a nucleoside or oligonucleotide;
Y₁ is S, O, or N(R′);
M is an organic or inorganic cation;
R¹³ is independently for each occurrence H, alkyl, alkenyl, alkynyl, cycloalkyl, aryl, aralkyl, ω-amino alkyl, ω-hydroxy alkyl, or ω-hydroxy alkenyl, an oxygen protecting group, each of which can be optionally substituted with one or more R^{sub} groups;
R′ and R″ are each independently H, alkyl, alkenyl, alkynyl, aryl, hydroxy, alkyloxy, ω-amino alkyl, ω-hydroxy alkyl, ω-hydroxy alkenyl, or ω-hydroxy alkynyl, each of which can be optionally substituted with one or more R^{sub} groups;
R¹⁸ is H or alkyl, optionally substituted with one or more R^{sub} groups; and
R^{sub} is independently for each occurrence halo, haloalkyl, alkyl, alkaryl, aryl, aralkyl, hydroxy, alkyloxy, aryloxy, oxo, nitro, amino, acylamino, alkylcarbamoyl, arylcarbamoyl, alkylamino, aminoalkyl, alkoxycarbonyl, carboxy, hydroxyalkyl, alkanesulfonyl, arenesulfonyl, alkanesulfonamido, arenesulfonamido, aralkylsulfonamido, alkylcarbonyl, arylcarbonyl, acyloxy, cyano, or ureido.

5. The oligonucleotide of claim 4, wherein the 2′-modified nucleoside has the structure of formula: 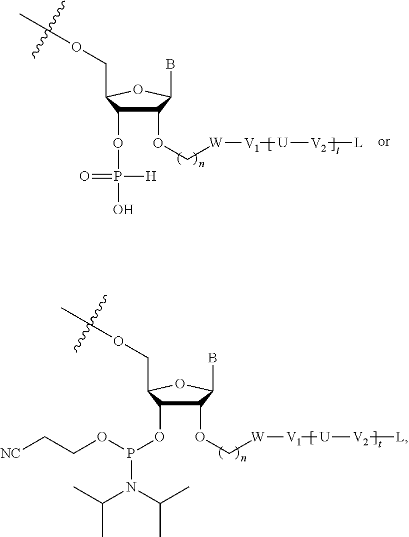
or a salt thereof.

6-9. (canceled)

10. The oligonucleotide of claim 1, wherein n is 1, and W is O, S—S, N(R^{N}), C(O)N(R^{N}), N(R^{N})C(O), N(R^{N})C(O)O, or N(R^{N})S(O)₂.

11. The oligonucleotide of claim 10, wherein the W-V₁-[U-V₂]ₜ-L has the structure of: 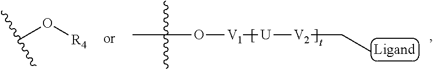
wherein: 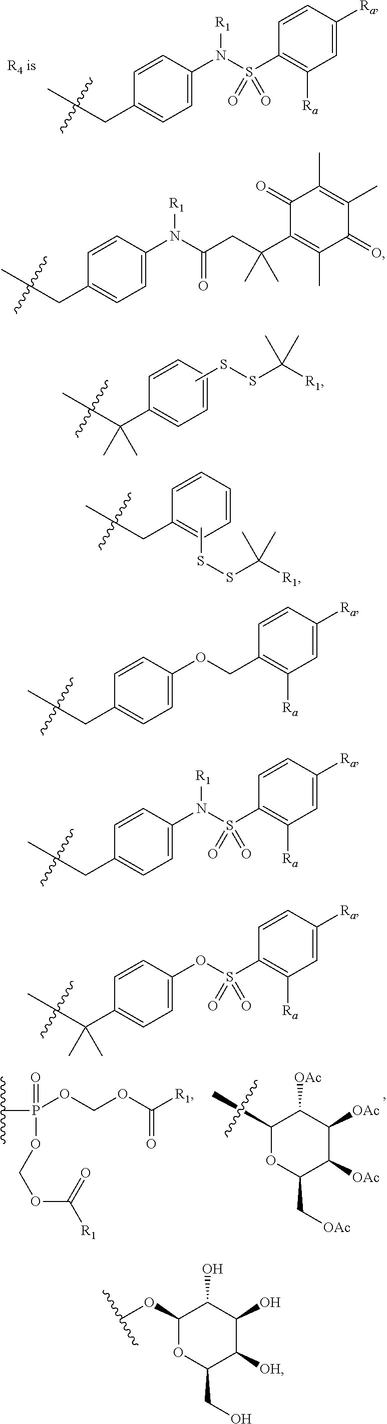 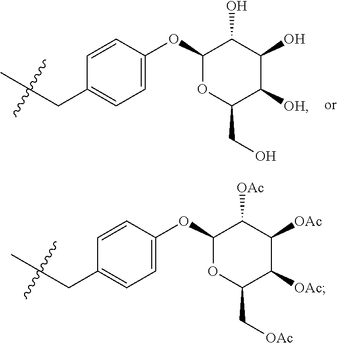
R₁ is independently for each occurrence H, linear or branched C₁-C₂₄ alkyl, aryl, hydroxyl, alkoxy, halo, methoxyalkoxy, alkylthio, amino, alkylamino, alkynyl, aminoalkyl, or aminoalkoxy; and
Rₐ is independently for each occurrence H, NO₂, CF₃, C(O)CH₃, and CN.

12. The oligonucleotide of claim 11, wherein the W-V₁-[U-V₂]ₜ-L has the structure of:

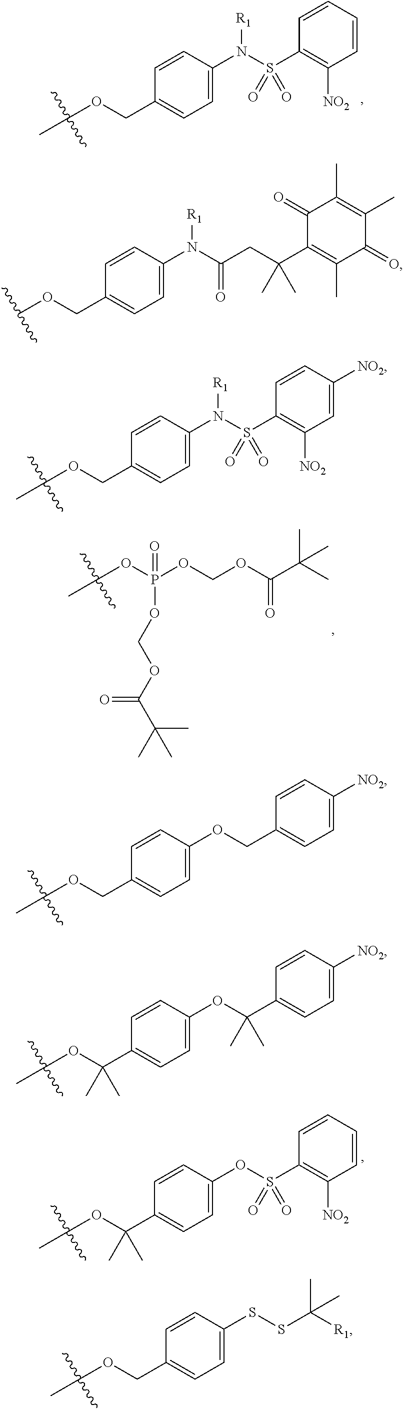

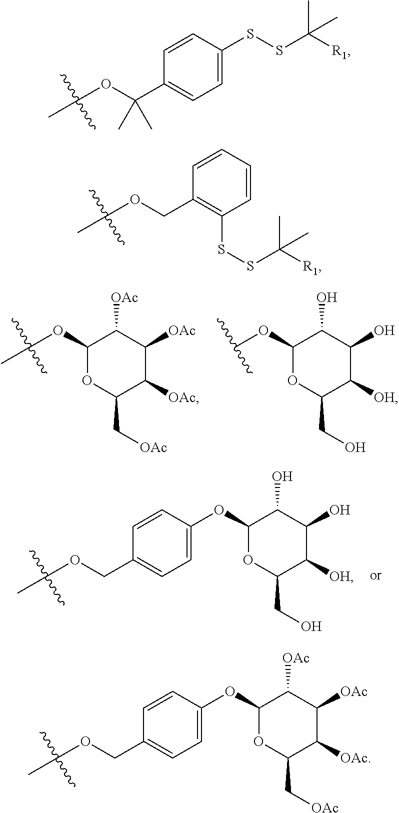

13. The oligonucleotide of claim 10, wherein the W-V₁-[U-V₂]ₜ-L has the structure of: 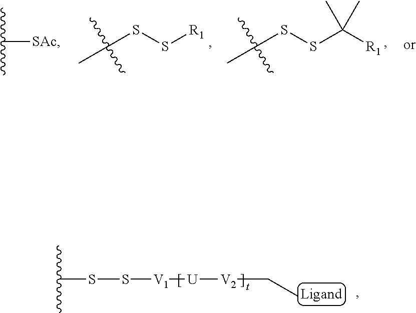
wherein:
R₁ is independently for each occurrence H, linear or branched C₁-C₂₄ alkyl, aryl, hydroxyl, alkoxy, halo, methoxyalkoxy, alkylthio, amino, alkylamino, alkynyl, aminoalkyl, or aminoalkoxy.

14. The oligonucleotide of claim 13, wherein the W-V₁-[U-V₂]ₜ-L has the structure of: 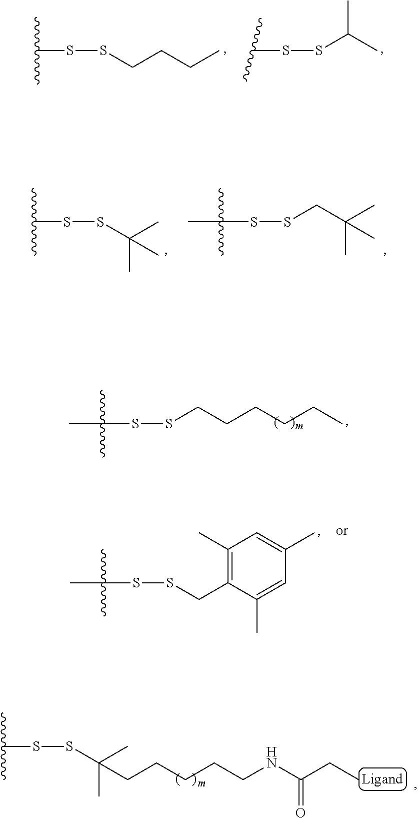
wherein m is an integer of 0-18.

15. The oligonucleotide of claim 10, wherein the W-V₁-[U-V₂]ₜ-L has the structure of: 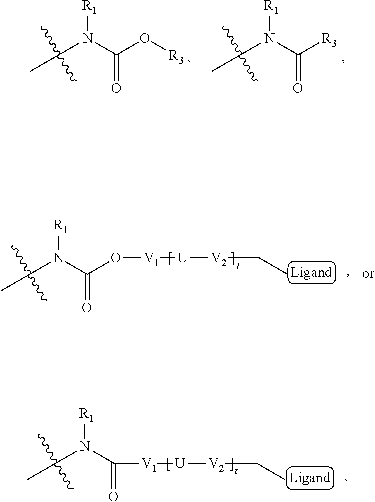
wherein
R₃ is linear or branched alkyl, aryl, 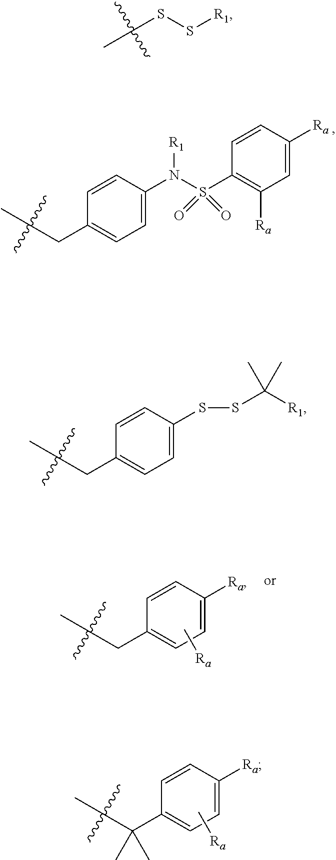
R₁ is independently for each occurrence H, linear or branched C₁-C₂₄ alkyl, aryl, hydroxyl, alkoxy, halo, methoxyalkoxy, alkylthio, amino, alkylamino, alkynyl, aminoalkyl, or aminoalkoxy; and
each Rₐ is independently for each occurrence H, NO₂, CF₃, C(O)CH₃, and CN.

16. The oligonucleotide of claim 15, wherein the W-V₁-[U-V₂]ₜ-L has the structure of:

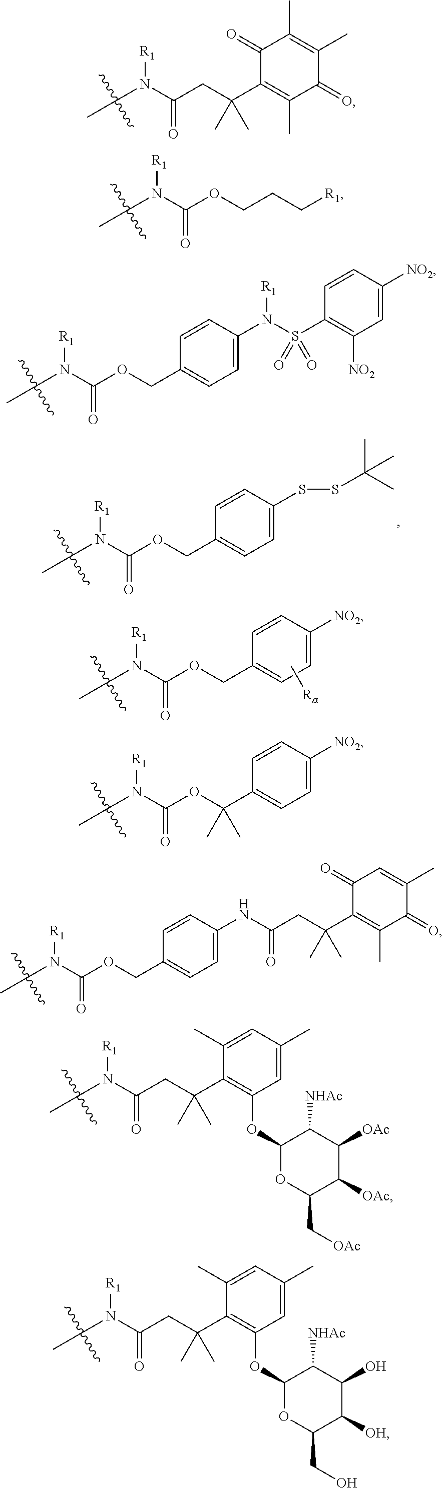

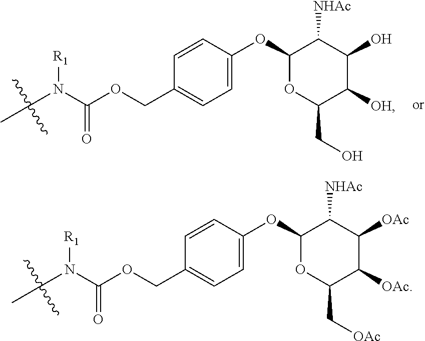

17. The oligonucleotide of claim 10, wherein the W-V₁-U-V₂-L has the structure of: 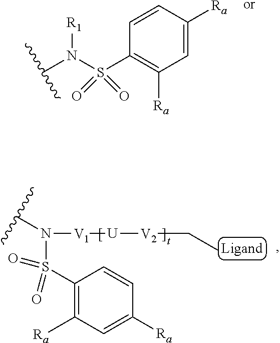
wherein:
R₁ is independently for each occurrence H, linear or branched C₁-C₂₄ alkyl, aryl, hydroxyl, alkoxy, halo, methoxyalkoxy, alkylthio, amino, alkylamino, alkynyl, aminoalkyl, or aminoalkoxy; and
Rₐ is independently for each occurrence H, NO₂, CF₃, C(O)CH₃, and CN.

18. The oligonucleotide of claim 17, wherein the W-V₁-U-V₂-L has the structure of: 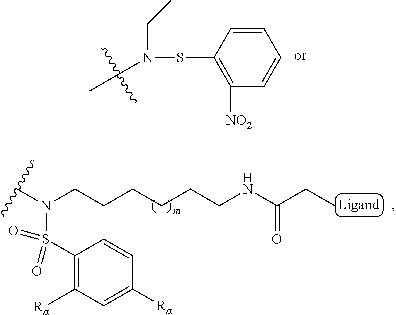
wherein m is an integer of 0-18.

19. The oligonucleotide of claim 1, wherein n is 1, and the W-V₁-U-V₂-L has the structure of: 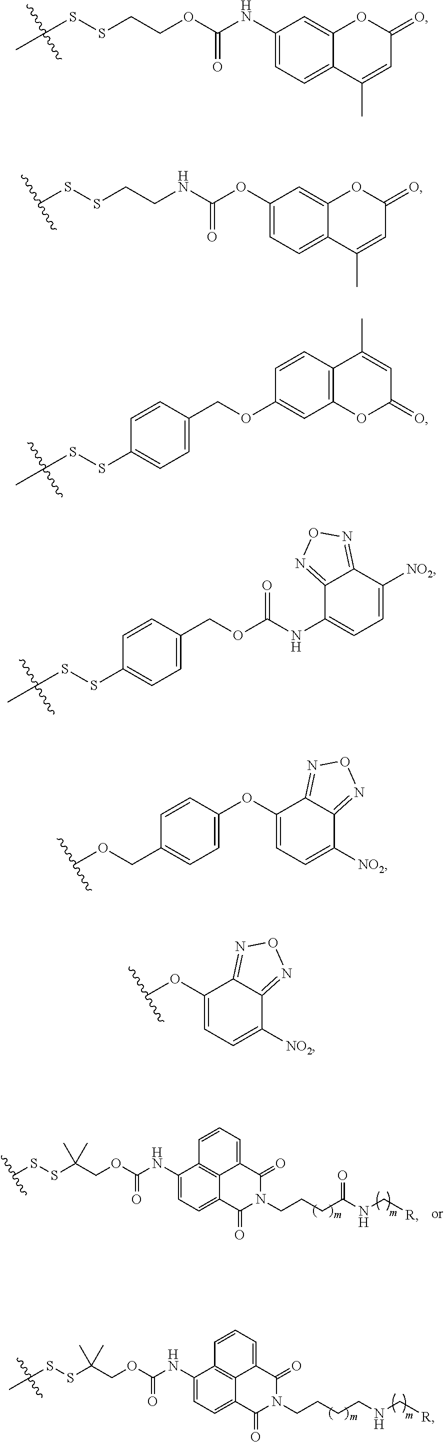
wherein
R is 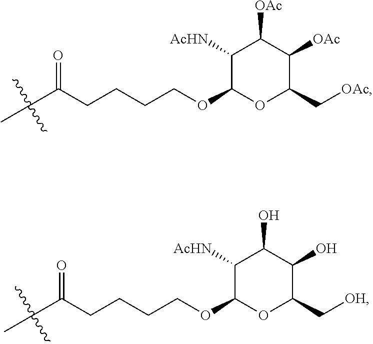 [SMILES: *C(=O)CCCCO[C@@H]1O[C@H](COC(C)=O)[C@H](OC(C)=O)[C@H](OC(C)=O)[C@H]1NC(C)=O ; *C(=O)CCCCO[C@@H]1O[C@H](CO)[C@H](O)[C@H](O)[C@H]1NC(C)=O]
a peptide, a small molecular ligand, GalNAc or multivalent GalNAc, folate or a lipophilic moiety; and
m is independently for each occurrence an integer of 0-18.

20. The oligonucleotide of claim 4, wherein the 2′-modified nucleoside has a structure selected from the group consisting of: 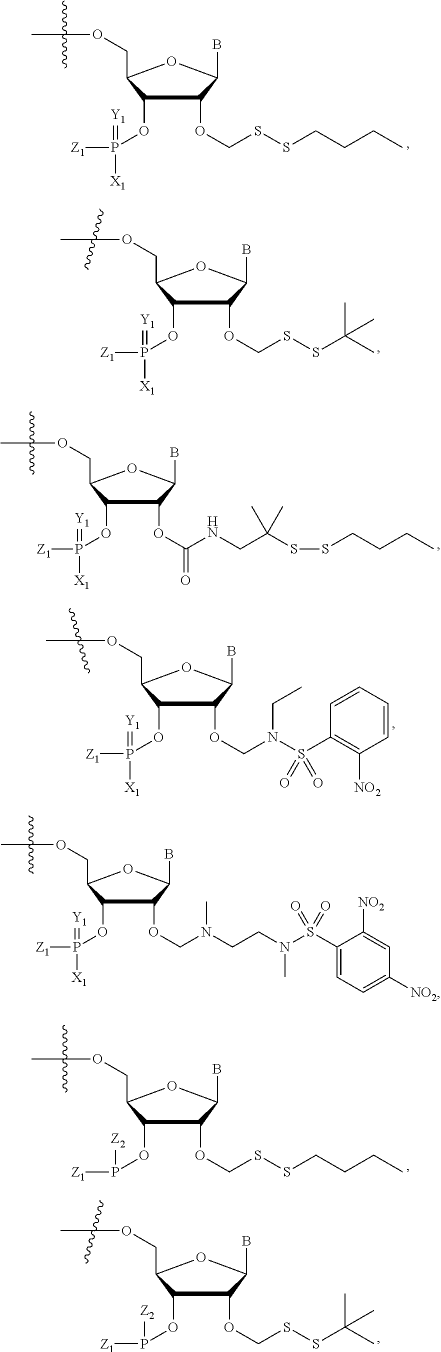 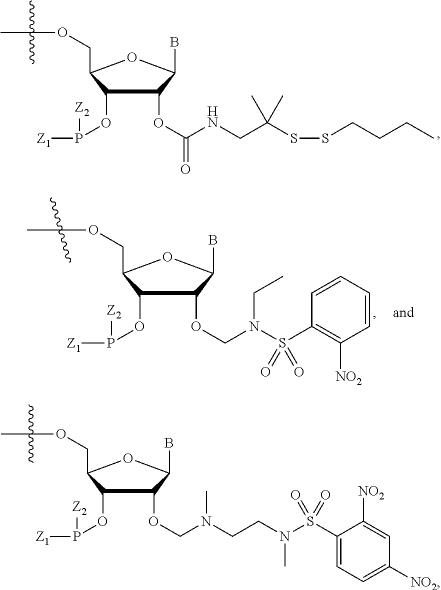
or a salt thereof.

21. The oligonucleotide of claim 20, wherein the 2′-modified nucleoside has a structure selected from the group consisting of: 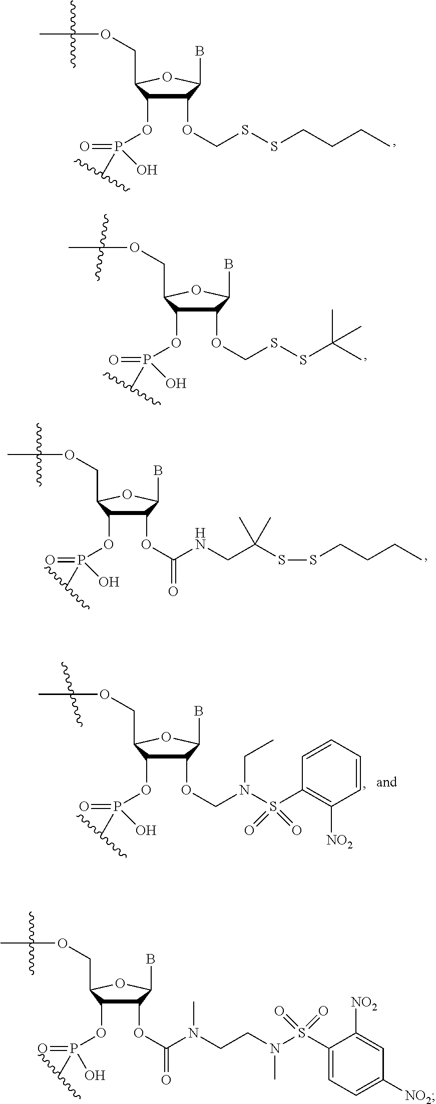
or a salt thereof.

22. The oligonucleotide of claim 1, wherein the L group is one or more ligands, optionally connected via one or more linkers.

23. The oligonucleotide of claim 22, wherein the ligand is selected from the group consisting of an antibody, a ligand-binding portion of a receptor, a ligand for a receptor, an aptamer, a carbohydrate-based ligand, a fatty acid, a lipoprotein, folate, thyrotropin, melanotropin, surfactant protein A, mucin, glycosylated polyaminoacids, transferrin, bisphosphonate, polyglutamate, polyaspartate, a lipophilic moiety, a cholesterol, a steroid, bile acid, vitamin B12, biotin, a fluorophore, and a peptide.

24. The oligonucleotide of claim 1, wherein the oligonucleotide is single-stranded.

25. The oligonucleotide of claim 1, wherein the oligonucleotide is a double-stranded comprising a sense strand and an antisense strand.

26-47. (canceled)


SEQUENCE LISTING (223 sequences)

SEQ ID NO: 1 - DNA, 23 bp, Artificial Sequence
  Description of Artificial Sequence Synthetic oligonucleotide
  Description of Combined DNA/RNA Molecule Synthetic oligonucleotide
uuauagagcaagaacacuguuuu

SEQ ID NO: 2 - DNA, 23 bp, Artificial Sequence
  Description of Artificial Sequence Synthetic oligonucleotide
  Description of Combined DNA/RNA Molecule Synthetic oligonucleotide
uuauagagcaagaacacuguuuu

SEQ ID NO: 3 - DNA, 23 bp, Artificial Sequence
  Description of Artificial Sequence Synthetic oligonucleotide
  Description of Combined DNA/RNA Molecule Synthetic oligonucleotide
uuauagagcaagaacacuguuuu

SEQ ID NO: 4 - DNA, 23 bp, Artificial Sequence
  Description of Artificial Sequence Synthetic oligonucleotide
  Description of Combined DNA/RNA Molecule Synthetic oligonucleotide
uuauagagcaagaacacuguuuu

SEQ ID NO: 5 - DNA, 23 bp, Artificial Sequence
  Description of Artificial Sequence Synthetic oligonucleotide
  Description of Combined DNA/RNA Molecule Synthetic oligonucleotide
uuauagagcaagaacacuguuuu

SEQ ID NO: 6 - DNA, 23 bp, Artificial Sequence
  Description of Artificial Sequence Synthetic oligonucleotide
  Description of Combined DNA/RNA Molecule Synthetic oligonucleotide
utauagagcaagaacacuguuuu

SEQ ID NO: 7 - DNA, 23 bp, Artificial Sequence
  Description of Artificial Sequence Synthetic oligonucleotide
  Description of Combined DNA/RNA Molecule Synthetic oligonucleotide
utauagagcaagaacacuguuuu

SEQ ID NO: 8 - DNA, 23 bp, Artificial Sequence
  Description of Artificial Sequence Synthetic oligonucleotide
  Description of Combined DNA/RNA Molecule Synthetic oligonucleotide
utauagagcaagaacacuguuuu

SEQ ID NO: 9 - DNA, 23 bp, Artificial Sequence
  Description of Artificial Sequence Synthetic oligonucleotide
  Description of Combined DNA/RNA Molecule Synthetic oligonucleotide
utauagagcaagaacacuguuuu

SEQ ID NO: 10 - DNA, 23 bp, Artificial Sequence
  Description of Artificial Sequence Synthetic oligonucleotide
  Description of Combined DNA/RNA Molecule Synthetic oligonucleotide
utauagagcaagaacacuguuuu

SEQ ID NO: 11 - RNA, 21 nt, Artificial Sequence
  Description of Artificial Sequence Synthetic oligonucleotide
aacaguguucuugcucuauaa

SEQ ID NO: 12 - DNA, 21 bp, Artificial Sequence
  Description of Artificial Sequence Synthetic oligonucleotide
  Description of Combined DNA/RNA Molecule Synthetic oligonucleotide
aacagugutcuugcucuauaa

SEQ ID NO: 13 - DNA, 21 bp, Artificial Sequence
  Description of Artificial Sequence Synthetic oligonucleotide
  Description of Combined DNA/RNA Molecule Synthetic oligonucleotide
aacaguguuctugcucuauaa

SEQ ID NO: 14 - DNA, 21 bp, Artificial Sequence
  Description of Artificial Sequence Synthetic oligonucleotide
  Description of Combined DNA/RNA Molecule Synthetic oligonucleotide
aacagugutcuugcucuauaa

SEQ ID NO: 15 - DNA, 21 bp, Artificial Sequence
  Description of Artificial Sequence Synthetic oligonucleotide
  Description of Combined DNA/RNA Molecule Synthetic oligonucleotide
aacaguguuctugcucuauaa

SEQ ID NO: 16 - RNA, 21 nt, Artificial Sequence
  Description of Artificial Sequence Synthetic oligonucleotide
aacaguguucuugcucuauaa

SEQ ID NO: 17 - DNA, 21 bp, Artificial Sequence
  Description of Artificial Sequence Synthetic oligonucleotide
  Description of Combined DNA/RNA Molecule Synthetic oligonucleotide
aacagugutcuugcucuauaa

SEQ ID NO: 18 - DNA, 21 bp, Artificial Sequence
  Description of Artificial Sequence Synthetic oligonucleotide
  Description of Combined DNA/RNA Molecule Synthetic oligonucleotide
aacaguguuctugcucuauaa

SEQ ID NO: 19 - RNA, 21 nt, Artificial Sequence
  Description of Artificial Sequence Synthetic oligonucleotide
aacaguguucuugcucuauaa

SEQ ID NO: 20 - RNA, 23 nt, Artificial Sequence
  Description of Artificial Sequence Synthetic oligonucleotide
uuuagagugaggauuaaaaugag

SEQ ID NO: 21 - RNA, 23 nt, Artificial Sequence
  Description of Artificial Sequence Synthetic oligonucleotide
uuuagagugaggauuaaaaugag

SEQ ID NO: 22 - RNA, 23 nt, Artificial Sequence
  Description of Artificial Sequence Synthetic oligonucleotide
uuuagagugaggauuaaaaugag

SEQ ID NO: 23 - RNA, 21 nt, Artificial Sequence
  Description of Artificial Sequence Synthetic oligonucleotide
aacaguguucuugcucuauaa

SEQ ID NO: 24 - RNA, 23 nt, Artificial Sequence
  Description of Artificial Sequence Synthetic oligonucleotide
uuauagagcaagaacacuguuuu

SEQ ID NO: 25 - DNA, 21 bp, Artificial Sequence
  Description of Artificial Sequence Synthetic oligonucleotide
  Description of Combined DNA/RNA Molecule Synthetic oligonucleotide
aacagugutctugcucuauaa

SEQ ID NO: 26 - DNA, 23 bp, Artificial Sequence
  Description of Artificial Sequence Synthetic oligonucleotide
  Description of Combined DNA/RNA Molecule Synthetic oligonucleotide
utauagagcaagaacacuguuuu

SEQ ID NO: 27 - DNA, 21 bp, Artificial Sequence
  Description of Artificial Sequence Synthetic oligonucleotide
  Description of Combined DNA/RNA Molecule Synthetic oligonucleotide
aacagugutctugcucuauaa

SEQ ID NO: 28 - DNA, 23 bp, Artificial Sequence
  Description of Artificial Sequence Synthetic oligonucleotide
  Description of Combined DNA/RNA Molecule Synthetic oligonucleotide
uuauagagcaagaacacuguuuu

SEQ ID NO: 29 - DNA, 21 bp, Artificial Sequence
  Description of Artificial Sequence Synthetic oligonucleotide
  Description of Combined DNA/RNA Molecule Synthetic oligonucleotide
aacagugutctugcucuauaa

SEQ ID NO: 30 - DNA, 23 bp, Artificial Sequence
  Description of Artificial Sequence Synthetic oligonucleotide
  Description of Combined DNA/RNA Molecule Synthetic oligonucleotide
utauagagcaagaacacuguuuu

SEQ ID NO: 31 - DNA, 21 bp, Artificial Sequence
  Description of Artificial Sequence Synthetic oligonucleotide
  Description of Combined DNA/RNA Molecule Synthetic oligonucleotide
aacagugutctugcucuauaa

SEQ ID NO: 32 - DNA, 23 bp, Artificial Sequence
  Description of Artificial Sequence Synthetic oligonucleotide
  Description of Combined DNA/RNA Molecule Synthetic oligonucleotide
uuauagagcaagaacacuguuuu

SEQ ID NO: 33 - DNA, 21 bp, Artificial Sequence
  Description of Artificial Sequence Synthetic oligonucleotide
  Description of Combined DNA/RNA Molecule Synthetic oligonucleotide
aacagugutctugcucuauaa

SEQ ID NO: 34 - DNA, 23 bp, Artificial Sequence
  Description of Artificial Sequence Synthetic oligonucleotide
  Description of Combined DNA/RNA Molecule Synthetic oligonucleotide
uuauagagcaagaacacuguuuu

SEQ ID NO: 35 - DNA, 21 bp, Artificial Sequence
  Description of Artificial Sequence Synthetic oligonucleotide
  Description of Combined DNA/RNA Molecule Synthetic oligonucleotide
aacagugutctugcucuauaa

SEQ ID NO: 36 - DNA, 23 bp, Artificial Sequence
  Description of Artificial Sequence Synthetic oligonucleotide
  Description of Combined DNA/RNA Molecule Synthetic oligonucleotide
uuauagagcaagaacacuguuuu

SEQ ID NO: 37 - DNA, 21 bp, Artificial Sequence
  Description of Artificial Sequence Synthetic oligonucleotide
  Description of Combined DNA/RNA Molecule Synthetic oligonucleotide
aacagugutctugcucuauaa

SEQ ID NO: 38 - DNA, 23 bp, Artificial Sequence
  Description of Artificial Sequence Synthetic oligonucleotide
  Description of Combined DNA/RNA Molecule Synthetic oligonucleotide
uuauagagcaagaacacuguuuu

SEQ ID NO: 39 - DNA, 21 bp, Artificial Sequence
  Description of Artificial Sequence Synthetic oligonucleotide
  Description of Combined DNA/RNA Molecule Synthetic oligonucleotide
aacagugutctugcucuauaa

SEQ ID NO: 40 - DNA, 23 bp, Artificial Sequence
  Description of Artificial Sequence Synthetic oligonucleotide
  Description of Combined DNA/RNA Molecule Synthetic oligonucleotide
uuauagagcaagaacacuguuuu

SEQ ID NO: 41 - DNA, 21 bp, Artificial Sequence
  Description of Artificial Sequence Synthetic oligonucleotide
  Description of Combined DNA/RNA Molecule Synthetic oligonucleotide
aacagugutctugcucuauaa

SEQ ID NO: 42 - DNA, 23 bp, Artificial Sequence
  Description of Artificial Sequence Synthetic oligonucleotide
  Description of Combined DNA/RNA Molecule Synthetic oligonucleotide
utauagagcaagaacacuguuuu

SEQ ID NO: 43 - DNA, 21 bp, Artificial Sequence
  Description of Artificial Sequence Synthetic oligonucleotide
  Description of Combined DNA/RNA Molecule Synthetic oligonucleotide
aacagugutctugcucuauaa

SEQ ID NO: 44 - DNA, 23 bp, Artificial Sequence
  Description of Artificial Sequence Synthetic oligonucleotide
  Description of Combined DNA/RNA Molecule Synthetic oligonucleotide
utauagagcaagaacacuguuuu

SEQ ID NO: 45 - DNA, 21 bp, Artificial Sequence
  Description of Artificial Sequence Synthetic oligonucleotide
  Description of Combined DNA/RNA Molecule Synthetic oligonucleotide
aacagugutctugcucuauaa

SEQ ID NO: 46 - DNA, 23 bp, Artificial Sequence
  Description of Artificial Sequence Synthetic oligonucleotide
  Description of Combined DNA/RNA Molecule Synthetic oligonucleotide
utauagagcaagaacacuguuuu

SEQ ID NO: 47 - DNA, 21 bp, Artificial Sequence
  Description of Artificial Sequence Synthetic oligonucleotide
  Description of Combined DNA/RNA Molecule Synthetic oligonucleotide
aacagugutctugcucuauaa

SEQ ID NO: 48 - DNA, 23 bp, Artificial Sequence
  Description of Artificial Sequence Synthetic oligonucleotide
  Description of Combined DNA/RNA Molecule Synthetic oligonucleotide
utauagagcaagaacacuguuuu

SEQ ID NO: 49 - DNA, 21 bp, Artificial Sequence
  Description of Artificial Sequence Synthetic oligonucleotide
  Description of Combined DNA/RNA Molecule Synthetic oligonucleotide
aacagugutctugcucuauaa

SEQ ID NO: 50 - DNA, 23 bp, Artificial Sequence
  Description of Artificial Sequence Synthetic oligonucleotide
  Description of Combined DNA/RNA Molecule Synthetic oligonucleotide
utauagagcaagaacacuguuuu

SEQ ID NO: 51 - DNA, 21 bp, Artificial Sequence
  Description of Artificial Sequence Synthetic oligonucleotide
  Description of Combined DNA/RNA Molecule Synthetic oligonucleotide
aacaguguuctugcucuauaa

SEQ ID NO: 52 - DNA, 23 bp, Artificial Sequence
  Description of Artificial Sequence Synthetic oligonucleotide
  Description of Combined DNA/RNA Molecule Synthetic oligonucleotide
utauagagcaagaacacuguuuu

SEQ ID NO: 53 - DNA, 21 bp, Artificial Sequence
  Description of Artificial Sequence Synthetic oligonucleotide
  Description of Combined DNA/RNA Molecule Synthetic oligonucleotide
aacagugutcuugcucuauaa

SEQ ID NO: 54 - DNA, 23 bp, Artificial Sequence
  Description of Artificial Sequence Synthetic oligonucleotide
  Description of Combined DNA/RNA Molecule Synthetic oligonucleotide
utauagagcaagaacacuguuuu

SEQ ID NO: 55 - RNA, 21 nt, Artificial Sequence
  Description of Artificial Sequence Synthetic oligonucleotide
aacaguguucuugcucuauaa

SEQ ID NO: 56 - DNA, 23 bp, Artificial Sequence
  Description of Artificial Sequence Synthetic oligonucleotide
  Description of Combined DNA/RNA Molecule Synthetic oligonucleotide
utauagagcaagaacacuguuuu

SEQ ID NO: 57 - DNA, 21 bp, Artificial Sequence
  Description of Artificial Sequence Synthetic oligonucleotide
  Description of Combined DNA/RNA Molecule Synthetic oligonucleotide
aacagugutctugcucuauaa

SEQ ID NO: 58 - DNA, 23 bp, Artificial Sequence
  Description of Artificial Sequence Synthetic oligonucleotide
  Description of Combined DNA/RNA Molecule Synthetic oligonucleotide
uuauagagcaagaacacuguuuu

SEQ ID NO: 59 - RNA, 21 nt, Artificial Sequence
  Description of Artificial Sequence Synthetic oligonucleotide
aacaguguucuugcucuauaa

SEQ ID NO: 60 - DNA, 23 bp, Artificial Sequence
  Description of Artificial Sequence Synthetic oligonucleotide
  Description of Combined DNA/RNA Molecule Synthetic oligonucleotide
utauagagcaagaacacuguuuu

SEQ ID NO: 61 - DNA, 21 bp, Artificial Sequence
  Description of Artificial Sequence Synthetic oligonucleotide
  Description of Combined DNA/RNA Molecule Synthetic oligonucleotide
aacagugutcuugcucuauaa

SEQ ID NO: 62 - DNA, 23 bp, Artificial Sequence
  Description of Artificial Sequence Synthetic oligonucleotide
  Description of Combined DNA/RNA Molecule Synthetic oligonucleotide
utauagagcaagaacacuguuuu

SEQ ID NO: 63 - DNA, 21 bp, Artificial Sequence
  Description of Artificial Sequence Synthetic oligonucleotide
  Description of Combined DNA/RNA Molecule Synthetic oligonucleotide
aacaguguuctugcucuauaa

SEQ ID NO: 64 - DNA, 23 bp, Artificial Sequence
  Description of Artificial Sequence Synthetic oligonucleotide
  Description of Combined DNA/RNA Molecule Synthetic oligonucleotide
utauagagcaagaacacuguuuu

SEQ ID NO: 65 - DNA, 21 bp, Artificial Sequence
  Description of Artificial Sequence Synthetic oligonucleotide
  Description of Combined DNA/RNA Molecule Synthetic oligonucleotide
aacagugutcuugcucuauaa

SEQ ID NO: 66 - DNA, 23 bp, Artificial Sequence
  Description of Artificial Sequence Synthetic oligonucleotide
  Description of Combined DNA/RNA Molecule Synthetic oligonucleotide
utauagagcaagaacacuguuuu

SEQ ID NO: 67 - DNA, 21 bp, Artificial Sequence
  Description of Artificial Sequence Synthetic oligonucleotide
  Description of Combined DNA/RNA Molecule Synthetic oligonucleotide
aacaguguuctugcucuauaa

SEQ ID NO: 68 - DNA, 23 bp, Artificial Sequence
  Description of Artificial Sequence Synthetic oligonucleotide
  Description of Combined DNA/RNA Molecule Synthetic oligonucleotide
utauagagcaagaacacuguuuu

SEQ ID NO: 69 - RNA, 21 nt, Artificial Sequence
  Description of Artificial Sequence Synthetic oligonucleotide
aacaguguucuugcucuauaa

SEQ ID NO: 70 - DNA, 23 bp, Artificial Sequence
  Description of Artificial Sequence Synthetic oligonucleotide
  Description of Combined DNA/RNA Molecule Synthetic oligonucleotide
utauagagcaagaacacuguuuu

SEQ ID NO: 71 - DNA, 21 bp, Artificial Sequence
  Description of Artificial Sequence Synthetic oligonucleotide
  Description of Combined DNA/RNA Molecule Synthetic oligonucleotide
aacagugutcuugcucuauaa

SEQ ID NO: 72 - DNA, 23 bp, Artificial Sequence
  Description of Artificial Sequence Synthetic oligonucleotide
  Description of Combined DNA/RNA Molecule Synthetic oligonucleotide
utauagagcaagaacacuguuuu

SEQ ID NO: 73 - DNA, 21 bp, Artificial Sequence
  Description of Artificial Sequence Synthetic oligonucleotide
  Description of Combined DNA/RNA Molecule Synthetic oligonucleotide
aacaguguuctugcucuauaa

SEQ ID NO: 74 - DNA, 23 bp, Artificial Sequence
  Description of Artificial Sequence Synthetic oligonucleotide
  Description of Combined DNA/RNA Molecule Synthetic oligonucleotide
utauagagcaagaacacuguuuu

SEQ ID NO: 75 - RNA, 21 nt, Artificial Sequence
  Description of Artificial Sequence Synthetic oligonucleotide
aacaguguucuugcucuauaa

SEQ ID NO: 76 - DNA, 23 bp, Artificial Sequence
  Description of Artificial Sequence Synthetic oligonucleotide
  Description of Combined DNA/RNA Molecule Synthetic oligonucleotide
utauagagcaagaacacuguuuu

SEQ ID NO: 77 - DNA, 21 bp, Artificial Sequence
  Description of Artificial Sequence Synthetic oligonucleotide
  Description of Combined DNA/RNA Molecule Synthetic oligonucleotide
aacagugutctugcucuauaa

SEQ ID NO: 78 - DNA, 23 bp, Artificial Sequence
  Description of Artificial Sequence Synthetic oligonucleotide
  Description of Combined DNA/RNA Molecule Synthetic oligonucleotide
utauagagcaagaacacuguuuu

SEQ ID NO: 79 - DNA, 21 bp, Artificial Sequence
  Description of Artificial Sequence Synthetic oligonucleotide
  Description of Combined DNA/RNA Molecule Synthetic oligonucleotide
aacaguguuctugcucuauaa

SEQ ID NO: 80 - DNA, 23 bp, Artificial Sequence
  Description of Artificial Sequence Synthetic oligonucleotide
  Description of Combined DNA/RNA Molecule Synthetic oligonucleotide
utauagagcaagaacacuguuuu

SEQ ID NO: 81 - DNA, 21 bp, Artificial Sequence
  Description of Artificial Sequence Synthetic oligonucleotide
  Description of Combined DNA/RNA Molecule Synthetic oligonucleotide
aacagugutctugcucuauaa

SEQ ID NO: 82 - DNA, 23 bp, Artificial Sequence
  Description of Artificial Sequence Synthetic oligonucleotide
  Description of Combined DNA/RNA Molecule Synthetic oligonucleotide
uuauagagcaagaacacuguuuu

SEQ ID NO: 83 - RNA, 21 nt, Artificial Sequence
  Description of Artificial Sequence Synthetic oligonucleotide
cauuuuaauccucacucuaaa

SEQ ID NO: 84 - RNA, 23 nt, Artificial Sequence
  Description of Artificial Sequence Synthetic oligonucleotide
uuuagagugaggauuaaaaugag

SEQ ID NO: 85 - RNA, 21 nt, Artificial Sequence
  Description of Artificial Sequence Synthetic oligonucleotide
cauuuuaauccucacucuaaa

SEQ ID NO: 86 - RNA, 23 nt, Artificial Sequence
  Description of Artificial Sequence Synthetic oligonucleotide
uuuagagugaggauuaaaaugag

SEQ ID NO: 87 - RNA, 21 nt, Artificial Sequence
  Description of Artificial Sequence Synthetic oligonucleotide
cauuuuaauccucacucuaaa

SEQ ID NO: 88 - RNA, 23 nt, Artificial Sequence
  Description of Artificial Sequence Synthetic oligonucleotide
uuuagagugaggauuaaaaugag

SEQ ID NO: 89 - RNA, 21 nt, Artificial Sequence
  Description of Artificial Sequence Synthetic oligonucleotide
cauuuuaauccucacucuaaa

SEQ ID NO: 90 - RNA, 23 nt, Artificial Sequence
  Description of Artificial Sequence Synthetic oligonucleotide
uuuagagugaggauuaaaaugag

SEQ ID NO: 91 - RNA, 21 nt, Artificial Sequence
  Description of Artificial Sequence Synthetic oligonucleotide
cauuuuaauccucacucuaaa

SEQ ID NO: 92 - RNA, 23 nt, Artificial Sequence
  Description of Artificial Sequence Synthetic oligonucleotide
uuuagagugaggauuaaaaugag

SEQ ID NO: 93 - RNA, 21 nt, Artificial Sequence
  Description of Artificial Sequence Synthetic oligonucleotide
cauuuuaauccucacucuaaa

SEQ ID NO: 94 - RNA, 23 nt, Artificial Sequence
  Description of Artificial Sequence Synthetic oligonucleotide
uuuagagugaggauuaaaaugag

SEQ ID NO: 95 - RNA, 21 nt, Artificial Sequence
  Description of Artificial Sequence Synthetic oligonucleotide
cauuuuaauccucacucuaaa

SEQ ID NO: 96 - RNA, 23 nt, Artificial Sequence
  Description of Artificial Sequence Synthetic oligonucleotide
uuuagagugaggauuaaaaugag

SEQ ID NO: 97 - RNA, 21 nt, Artificial Sequence
  Description of Artificial Sequence Synthetic oligonucleotide
cauuuuaauccucacucuaaa

SEQ ID NO: 98 - RNA, 23 nt, Artificial Sequence
  Description of Artificial Sequence Synthetic oligonucleotide
uuuagagugaggauuaaaaugag

SEQ ID NO: 99 - RNA, 23 nt, Artificial Sequence
  Description of Artificial Sequence Synthetic oligonucleotide
uuauagagcaagaacacuguuuu

SEQ ID NO: 100 - RNA, 22 nt, Artificial Sequence
  Description of Artificial Sequence Synthetic oligonucleotide
uuauaggcaagaacacuguuuu

SEQ ID NO: 101 - DNA, 23 bp, Artificial Sequence
  Description of Artificial Sequence Synthetic oligonucleotide
  Description of Combined DNA/RNA Molecule Synthetic oligonucleotide
utauagagcaagaacacuguuuu

SEQ ID NO: 102 - DNA, 23 bp, Artificial Sequence
  Description of Artificial Sequence Synthetic oligonucleotide
  Description of Combined DNA/RNA Molecule Synthetic oligonucleotide
uuauagagcaagaacacuguuuu

SEQ ID NO: 103 - DNA, 23 bp, Artificial Sequence
  Description of Artificial Sequence Synthetic oligonucleotide
  Description of Combined DNA/RNA Molecule Synthetic oligonucleotide
utauagagcaagaacacuguuuu

SEQ ID NO: 104 - DNA, 23 bp, Artificial Sequence
  Description of Artificial Sequence Synthetic oligonucleotide
  Description of Combined DNA/RNA Molecule Synthetic oligonucleotide
uuauagagcaagaacacuguuuu

SEQ ID NO: 105 - DNA, 23 bp, Artificial Sequence
  Description of Artificial Sequence Synthetic oligonucleotide
  Description of Combined DNA/RNA Molecule Synthetic oligonucleotide
utauagagcaagaacacuguuuu

SEQ ID NO: 106 - DNA, 23 bp, Artificial Sequence
  Description of Artificial Sequence Synthetic oligonucleotide
  Description of Combined DNA/RNA Molecule Synthetic oligonucleotide
uuauagagcaagaacacuguuuu

SEQ ID NO: 107 - DNA, 23 bp, Artificial Sequence
  Description of Artificial Sequence Synthetic oligonucleotide
  Description of Combined DNA/RNA Molecule Synthetic oligonucleotide
utauagagcaagaacacuguuuu

SEQ ID NO: 108 - DNA, 23 bp, Artificial Sequence
  Description of Artificial Sequence Synthetic oligonucleotide
  Description of Combined DNA/RNA Molecule Synthetic oligonucleotide
uuauagagcaagaacacuguuuu

SEQ ID NO: 109 - DNA, 23 bp, Artificial Sequence
  Description of Artificial Sequence Synthetic oligonucleotide
  Description of Combined DNA/RNA Molecule Synthetic oligonucleotide
utauagagcaagaacacuguuuu

SEQ ID NO: 110 - DNA, 23 bp, Artificial Sequence
  Description of Artificial Sequence Synthetic oligonucleotide
  Description of Combined DNA/RNA Molecule Synthetic oligonucleotide
uuauagagcaagaacacuguuuu

SEQ ID NO: 111 - DNA, 21 bp, Artificial Sequence
  Description of Artificial Sequence Synthetic oligonucleotide
  Description of Combined DNA/RNA Molecule Synthetic oligonucleotide
aacagugutctugcucuauaa

SEQ ID NO: 112 - DNA, 23 bp, Artificial Sequence
  Description of Artificial Sequence Synthetic oligonucleotide
  Description of Combined DNA/RNA Molecule Synthetic oligonucleotide
utauagagcaagaacacuguuuu

SEQ ID NO: 113 - DNA, 21 bp, Artificial Sequence
  Description of Artificial Sequence Synthetic oligonucleotide
  Description of Combined DNA/RNA Molecule Synthetic oligonucleotide
aacagugutctugcucuauaa

SEQ ID NO: 114 - DNA, 23 bp, Artificial Sequence
  Description of Artificial Sequence Synthetic oligonucleotide
  Description of Combined DNA/RNA Molecule Synthetic oligonucleotide
uuauagagcaagaacacuguuuu

SEQ ID NO: 115 - DNA, 21 bp, Artificial Sequence
  Description of Artificial Sequence Synthetic oligonucleotide
  Description of Combined DNA/RNA Molecule Synthetic oligonucleotide
aacagugutctugcucuauaa

SEQ ID NO: 116 - DNA, 23 bp, Artificial Sequence
  Description of Artificial Sequence Synthetic oligonucleotide
  Description of Combined DNA/RNA Molecule Synthetic oligonucleotide
utauagagcaagaacacuguuuu

SEQ ID NO: 117 - DNA, 21 bp, Artificial Sequence
  Description of Artificial Sequence Synthetic oligonucleotide
  Description of Combined DNA/RNA Molecule Synthetic oligonucleotide
aacagugutctugcucuauaa

SEQ ID NO: 118 - DNA, 23 bp, Artificial Sequence
  Description of Artificial Sequence Synthetic oligonucleotide
  Description of Combined DNA/RNA Molecule Synthetic oligonucleotide
uuauagagcaagaacacuguuuu

SEQ ID NO: 119 - DNA, 21 bp, Artificial Sequence
  Description of Artificial Sequence Synthetic oligonucleotide
  Description of Combined DNA/RNA Molecule Synthetic oligonucleotide
aacagugutctugcucuauaa

SEQ ID NO: 120 - DNA, 23 bp, Artificial Sequence
  Description of Artificial Sequence Synthetic oligonucleotide
  Description of Combined DNA/RNA Molecule Synthetic oligonucleotide
uuauagagcaagaacacuguuuu

SEQ ID NO: 121 - DNA, 21 bp, Artificial Sequence
  Description of Artificial Sequence Synthetic oligonucleotide
  Description of Combined DNA/RNA Molecule Synthetic oligonucleotide
aacagugutctugcucuauaa

SEQ ID NO: 122 - DNA, 23 bp, Artificial Sequence
  Description of Artificial Sequence Synthetic oligonucleotide
  Description of Combined DNA/RNA Molecule Synthetic oligonucleotide
uuauagagcaagaacacuguuuu

SEQ ID NO: 123 - DNA, 21 bp, Artificial Sequence
  Description of Artificial Sequence Synthetic oligonucleotide
  Description of Combined DNA/RNA Molecule Synthetic oligonucleotide
aacagugutctugcucuauaa

SEQ ID NO: 124 - DNA, 23 bp, Artificial Sequence
  Description of Artificial Sequence Synthetic oligonucleotide
  Description of Combined DNA/RNA Molecule Synthetic oligonucleotide
uuauagagcaagaacacuguuuu

SEQ ID NO: 125 - DNA, 21 bp, Artificial Sequence
  Description of Artificial Sequence Synthetic oligonucleotide
  Description of Combined DNA/RNA Molecule Synthetic oligonucleotide
aacagugutctugcucuauaa

SEQ ID NO: 126 - DNA, 23 bp, Artificial Sequence
  Description of Artificial Sequence Synthetic oligonucleotide
  Description of Combined DNA/RNA Molecule Synthetic oligonucleotide
uuauagagcaagaacacuguuuu

SEQ ID NO: 127 - DNA, 21 bp, Artificial Sequence
  Description of Artificial Sequence Synthetic oligonucleotide
  Description of Combined DNA/RNA Molecule Synthetic oligonucleotide
aacagugutctugcucuauaa

SEQ ID NO: 128 - DNA, 23 bp, Artificial Sequence
  Description of Artificial Sequence Synthetic oligonucleotide
  Description of Combined DNA/RNA Molecule Synthetic oligonucleotide
utauagagcaagaacacuguuuu

SEQ ID NO: 129 - DNA, 21 bp, Artificial Sequence
  Description of Artificial Sequence Synthetic oligonucleotide
  Description of Combined DNA/RNA Molecule Synthetic oligonucleotide
aacagugutctugcucuauaa

SEQ ID NO: 130 - DNA, 23 bp, Artificial Sequence
  Description of Artificial Sequence Synthetic oligonucleotide
  Description of Combined DNA/RNA Molecule Synthetic oligonucleotide
utauagagcaagaacacuguuuu

SEQ ID NO: 131 - DNA, 21 bp, Artificial Sequence
  Description of Artificial Sequence Synthetic oligonucleotide
  Description of Combined DNA/RNA Molecule Synthetic oligonucleotide
aacagugutctugcucuauaa

SEQ ID NO: 132 - DNA, 23 bp, Artificial Sequence
  Description of Artificial Sequence Synthetic oligonucleotide
  Description of Combined DNA/RNA Molecule Synthetic oligonucleotide
utauagagcaagaacacuguuuu

SEQ ID NO: 133 - DNA, 21 bp, Artificial Sequence
  Description of Artificial Sequence Synthetic oligonucleotide
  Description of Combined DNA/RNA Molecule Synthetic oligonucleotide
aacagugutctugcucuauaa

SEQ ID NO: 134 - DNA, 23 bp, Artificial Sequence
  Description of Artificial Sequence Synthetic oligonucleotide
  Description of Combined DNA/RNA Molecule Synthetic oligonucleotide
utauagagcaagaacacuguuuu

SEQ ID NO: 135 - DNA, 21 bp, Artificial Sequence
  Description of Artificial Sequence Synthetic oligonucleotide
  Description of Combined DNA/RNA Molecule Synthetic oligonucleotide
aacagugutctugcucuauaa

SEQ ID NO: 136 - DNA, 23 bp, Artificial Sequence
  Description of Artificial Sequence Synthetic oligonucleotide
  Description of Combined DNA/RNA Molecule Synthetic oligonucleotide
utauagagcaagaacacuguuuu

SEQ ID NO: 137 - DNA, 21 bp, Artificial Sequence
  Description of Artificial Sequence Synthetic oligonucleotide
  Description of Combined DNA/RNA Molecule Synthetic oligonucleotide
aacagugutctugcucuauaa

SEQ ID NO: 138 - DNA, 23 bp, Artificial Sequence
  Description of Artificial Sequence Synthetic oligonucleotide
  Description of Combined DNA/RNA Molecule Synthetic oligonucleotide
utauagagcaagaacacuguuuu

SEQ ID NO: 139 - DNA, 21 bp, Artificial Sequence
  Description of Artificial Sequence Synthetic oligonucleotide
  Description of Combined DNA/RNA Molecule Synthetic oligonucleotide
aacaguguuctugcucuauaa

SEQ ID NO: 140 - DNA, 23 bp, Artificial Sequence
  Description of Artificial Sequence Synthetic oligonucleotide
  Description of Combined DNA/RNA Molecule Synthetic oligonucleotide
utauagagcaagaacacuguuuu

SEQ ID NO: 141 - DNA, 21 bp, Artificial Sequence
  Description of Artificial Sequence Synthetic oligonucleotide
  Description of Combined DNA/RNA Molecule Synthetic oligonucleotide
aacagugutcuugcucuauaa

SEQ ID NO: 142 - DNA, 23 bp, Artificial Sequence
  Description of Artificial Sequence Synthetic oligonucleotide
  Description of Combined DNA/RNA Molecule Synthetic oligonucleotide
utauagagcaagaacacuguuuu

SEQ ID NO: 143 - RNA, 21 nt, Artificial Sequence
  Description of Artificial Sequence Synthetic oligonucleotide
aacaguguucuugcucuauaa

SEQ ID NO: 144 - DNA, 23 bp, Artificial Sequence
  Description of Artificial Sequence Synthetic oligonucleotide
  Description of Combined DNA/RNA Molecule Synthetic oligonucleotide
utauagagcaagaacacuguuuu

SEQ ID NO: 145 - DNA, 21 bp, Artificial Sequence
  Description of Artificial Sequence Synthetic oligonucleotide
  Description of Combined DNA/RNA Molecule Synthetic oligonucleotide
aacagugutctugcucuauaa

SEQ ID NO: 146 - DNA, 23 bp, Artificial Sequence
  Description of Artificial Sequence Synthetic oligonucleotide
  Description of Combined DNA/RNA Molecule Synthetic oligonucleotide
uuauagagcaagaacacuguuuu

SEQ ID NO: 147 - RNA, 21 nt, Artificial Sequence
  Description of Artificial Sequence Synthetic oligonucleotide
aacaguguucuugcucuauaa

SEQ ID NO: 148 - DNA, 23 bp, Artificial Sequence
  Description of Artificial Sequence Synthetic oligonucleotide
  Description of Combined DNA/RNA Molecule Synthetic oligonucleotide
utauagagcaagaacacuguuuu

SEQ ID NO: 149 - DNA, 21 bp, Artificial Sequence
  Description of Artificial Sequence Synthetic oligonucleotide
  Description of Combined DNA/RNA Molecule Synthetic oligonucleotide
aacagugutcuugcucuauaa

SEQ ID NO: 150 - DNA, 23 bp, Artificial Sequence
  Description of Artificial Sequence Synthetic oligonucleotide
  Description of Combined DNA/RNA Molecule Synthetic oligonucleotide
utauagagcaagaacacuguuuu

SEQ ID NO: 151 - DNA, 21 bp, Artificial Sequence
  Description of Artificial Sequence Synthetic oligonucleotide
  Description of Combined DNA/RNA Molecule Synthetic oligonucleotide
aacaguguuctugcucuauaa

SEQ ID NO: 152 - DNA, 23 bp, Artificial Sequence
  Description of Artificial Sequence Synthetic oligonucleotide
  Description of Combined DNA/RNA Molecule Synthetic oligonucleotide
utauagagcaagaacacuguuuu

SEQ ID NO: 153 - DNA, 21 bp, Artificial Sequence
  Description of Artificial Sequence Synthetic oligonucleotide
  Description of Combined DNA/RNA Molecule Synthetic oligonucleotide
aacagugutcuugcucuauaa

SEQ ID NO: 154 - DNA, 23 bp, Artificial Sequence
  Description of Artificial Sequence Synthetic oligonucleotide
  Description of Combined DNA/RNA Molecule Synthetic oligonucleotide
utauagagcaagaacacuguuuu

SEQ ID NO: 155 - DNA, 21 bp, Artificial Sequence
  Description of Artificial Sequence Synthetic oligonucleotide
  Description of Combined DNA/RNA Molecule Synthetic oligonucleotide
aacaguguuctugcucuauaa

SEQ ID NO: 156 - DNA, 23 bp, Artificial Sequence
  Description of Artificial Sequence Synthetic oligonucleotide
  Description of Combined DNA/RNA Molecule Synthetic oligonucleotide
utauagagcaagaacacuguuuu

SEQ ID NO: 157 - RNA, 21 nt, Artificial Sequence
  Description of Artificial Sequence Synthetic oligonucleotide
aacaguguucuugcucuauaa

SEQ ID NO: 158 - DNA, 23 bp, Artificial Sequence
  Description of Artificial Sequence Synthetic oligonucleotide
  Description of Combined DNA/RNA Molecule Synthetic oligonucleotide
utauagagcaagaacacuguuuu

SEQ ID NO: 159 - DNA, 21 bp, Artificial Sequence
  Description of Artificial Sequence Synthetic oligonucleotide
  Description of Combined DNA/RNA Molecule Synthetic oligonucleotide
aacagugutcuugcucuauaa

SEQ ID NO: 160 - DNA, 23 bp, Artificial Sequence
  Description of Artificial Sequence Synthetic oligonucleotide
  Description of Combined DNA/RNA Molecule Synthetic oligonucleotide
utauagagcaagaacacuguuuu

SEQ ID NO: 161 - DNA, 21 bp, Artificial Sequence
  Description of Artificial Sequence Synthetic oligonucleotide
  Description of Combined DNA/RNA Molecule Synthetic oligonucleotide
aacaguguuctugcucuauaa

SEQ ID NO: 162 - DNA, 23 bp, Artificial Sequence
  Description of Artificial Sequence Synthetic oligonucleotide
  Description of Combined DNA/RNA Molecule Synthetic oligonucleotide
utauagagcaagaacacuguuuu

SEQ ID NO: 163 - RNA, 21 nt, Artificial Sequence
  Description of Artificial Sequence Synthetic oligonucleotide
aacaguguucuugcucuauaa

SEQ ID NO: 164 - DNA, 23 bp, Artificial Sequence
  Description of Artificial Sequence Synthetic oligonucleotide
  Description of Combined DNA/RNA Molecule Synthetic oligonucleotide
utauagagcaagaacacuguuuu

SEQ ID NO: 165 - DNA, 21 bp, Artificial Sequence
  Description of Artificial Sequence Synthetic oligonucleotide
  Description of Combined DNA/RNA Molecule Synthetic oligonucleotide
aacagugutctugcucuauaa

SEQ ID NO: 166 - DNA, 23 bp, Artificial Sequence
  Description of Artificial Sequence Synthetic oligonucleotide
  Description of Combined DNA/RNA Molecule Synthetic oligonucleotide
utauagagcaagaacacuguuuu

SEQ ID NO: 167 - DNA, 21 bp, Artificial Sequence
  Description of Artificial Sequence Synthetic oligonucleotide
  Description of Combined DNA/RNA Molecule Synthetic oligonucleotide
aacaguguuctugcucuauaa

SEQ ID NO: 168 - DNA, 23 bp, Artificial Sequence
  Description of Artificial Sequence Synthetic oligonucleotide
  Description of Combined DNA/RNA Molecule Synthetic oligonucleotide
utauagagcaagaacacuguuuu

SEQ ID NO: 169 - DNA, 21 bp, Artificial Sequence
  Description of Artificial Sequence Synthetic oligonucleotide
  Description of Combined DNA/RNA Molecule Synthetic oligonucleotide
aacagugutctugcucuauaa

SEQ ID NO: 170 - DNA, 23 bp, Artificial Sequence
  Description of Artificial Sequence Synthetic oligonucleotide
  Description of Combined DNA/RNA Molecule Synthetic oligonucleotide
uuauagagcaagaacacuguuuu

SEQ ID NO: 171 - RNA, 21 nt, Artificial Sequence
  Description of Artificial Sequence Synthetic oligonucleotide
cauuuuaauccucacucuaaa

SEQ ID NO: 172 - RNA, 23 nt, Artificial Sequence
  Description of Artificial Sequence Synthetic oligonucleotide
uuuagagugaggauuaaaaugag

SEQ ID NO: 173 - RNA, 21 nt, Artificial Sequence
  Description of Artificial Sequence Synthetic oligonucleotide
cauuuuaauccucacucuaaa

SEQ ID NO: 174 - RNA, 23 nt, Artificial Sequence
  Description of Artificial Sequence Synthetic oligonucleotide
uuuagagugaggauuaaaaugag

SEQ ID NO: 175 - RNA, 21 nt, Artificial Sequence
  Description of Artificial Sequence Synthetic oligonucleotide
cauuuuaauccucacucuaaa

SEQ ID NO: 176 - RNA, 23 nt, Artificial Sequence
  Description of Artificial Sequence Synthetic oligonucleotide
uuuagagugaggauuaaaaugag

SEQ ID NO: 177 - RNA, 21 nt, Artificial Sequence
  Description of Artificial Sequence Synthetic oligonucleotide
cauuuuaauccucacucuaaa

SEQ ID NO: 178 - RNA, 23 nt, Artificial Sequence
  Description of Artificial Sequence Synthetic oligonucleotide
uuuagagugaggauuaaaaugag

SEQ ID NO: 179 - RNA, 21 nt, Artificial Sequence
  Description of Artificial Sequence Synthetic oligonucleotide
cauuuuaauccucacucuaaa

SEQ ID NO: 180 - RNA, 23 nt, Artificial Sequence
  Description of Artificial Sequence Synthetic oligonucleotide
uuuagagugaggauuaaaaugag

SEQ ID NO: 181 - RNA, 21 nt, Artificial Sequence
  Description of Artificial Sequence Synthetic oligonucleotide
cauuuuaauccucacucuaaa

SEQ ID NO: 182 - RNA, 23 nt, Artificial Sequence
  Description of Artificial Sequence Synthetic oligonucleotide
uuuagagugaggauuaaaaugag

SEQ ID NO: 183 - RNA, 21 nt, Artificial Sequence
  Description of Artificial Sequence Synthetic oligonucleotide
cauuuuaauccucacucuaaa

SEQ ID NO: 184 - RNA, 23 nt, Artificial Sequence
  Description of Artificial Sequence Synthetic oligonucleotide
uuuagagugaggauuaaaaugag

SEQ ID NO: 185 - RNA, 21 nt, Artificial Sequence
  Description of Artificial Sequence Synthetic oligonucleotide
cauuuuaauccucacucuaaa

SEQ ID NO: 186 - RNA, 23 nt, Artificial Sequence
  Description of Artificial Sequence Synthetic oligonucleotide
uuuagagugaggauuaaaaugag

SEQ ID NO: 187 - protein, 8 aa, Unknown
  Description of Unknown RGD peptide sequence
GRGDSPKC

SEQ ID NO: 188 - protein, 5 aa, Unknown
  Description of Unknown RGD peptide sequence
  MOD_RES (4)..(4): D-Phe
RGDFC

SEQ ID NO: 189 - protein, 29 aa, Unknown
  Description of Unknown endosomolytic/fusogenic peptide sequence
AALEALAEALEALAEALEALAEAAAAGGC

SEQ ID NO: 190 - protein, 30 aa, Unknown
  Description of Unknown endosomolytic/fusogenic peptide sequence
AALAEALAEALAEALAEALAEALAAAAGGC

SEQ ID NO: 191 - protein, 15 aa, Unknown
  Description of Unknown endosomolytic/fusogenic peptide sequence
ALEALAEALEALAEA

SEQ ID NO: 192 - protein, 22 aa, Unknown
  Description of Unknown endosomolytic/fusogenic peptide sequence
GLFEAIEGFIENGWEGMIWDYG

SEQ ID NO: 193 - protein, 23 aa, Unknown
  Description of Unknown endosomolytic/fusogenic peptide sequence
GLFGAIAGFIENGWEGMIDGWYG

SEQ ID NO: 194 - protein, 48 aa, Unknown
  Description of Unknown endosomolytic/fusogenic peptide sequence
GLFEAIEGFIENGWEGMIDGWYGCGLFEAIEGFIENGWEGMIDGWYGC

SEQ ID NO: 195 - protein, 44 aa, Unknown
  Description of Unknown endosomolytic/fusogenic peptide sequence
GLFEAIEGFIENGWEGMIDGGCGLFEAIEGFIENGWEGMIDGGC

SEQ ID NO: 196 - protein, 35 aa, Unknown
  Description of Unknown endosomolytic/fusogenic peptide sequence
GLFGALAEALAEALAEHLAEALAEALEALAAGGSC

SEQ ID NO: 197 - protein, 34 aa, Unknown
  Description of Unknown endosomolytic/fusogenic peptide sequence
GLFEAIEGFIENGWEGLAEALAEALEALAAGGSC

SEQ ID NO: 198 - protein, 41 aa, Unknown
  Description of Unknown endosomolytic/fusogenic peptide sequence
  MOD_RES (17)..(17): Norleucine
  MOD_RES (38)..(38): Norleucine
GLFEAIEGFIENGWEGXIDGKGLFEAIEGFIENGWEGXIDG

SEQ ID NO: 199 - protein, 19 aa, Unknown
  Description of Unknown endosomolytic/fusogenic peptide sequence
LFEALLELLESLWELLLEA

SEQ ID NO: 200 - protein, 20 aa, Unknown
  Description of Unknown endosomolytic/fusogenic peptide sequence
GLFKALLKLLKSLWKLLLKA

SEQ ID NO: 201 - protein, 20 aa, Unknown
  Description of Unknown endosomolytic/fusogenic peptide sequence
GLFRALLRLLRSLWRLLLRA

SEQ ID NO: 202 - protein, 30 aa, Unknown
  Description of Unknown endosomolytic/fusogenic peptide sequence
WEAKLAKALAKALAKHLAKALAKALKACEA

SEQ ID NO: 203 - protein, 22 aa, Unknown
  Description of Unknown endosomolytic/fusogenic peptide sequence
GLFFEAIAEFIEGGWEGLIEGC

SEQ ID NO: 204 - protein, 26 aa, Unknown
  Description of Unknown endosomolytic/fusogenic peptide sequence
GIGAVLKVLTTGLPALISWIKRKRQQ

SEQ ID NO: 205 - protein, 8 aa, Unknown
  Description of Unknown endosomolytic/fusogenic peptide sequence
HHHHHWYG

SEQ ID NO: 206 - protein, 10 aa, Unknown
  Description of Unknown endosomolytic/fusogenic peptide sequence
CHKKKKKKHC

SEQ ID NO: 207 - protein, 16 aa, Unknown
  Description of Unknown cell permeation peptide sequence
RQIKIWFQNRRMKWKK

SEQ ID NO: 208 - protein, 14 aa, Unknown
  Description of Unknown cell permeation peptide sequence
GRKKRRQRRRPPQC

SEQ ID NO: 209 - protein, 27 aa, Unknown
  Description of Unknown cell permeation peptide sequence
GALFLGWLGAAGSTMGAWSQPKKKRKV

SEQ ID NO: 210 - protein, 18 aa, Unknown
  Description of Unknown cell permeation peptide sequence
LLIILRRRIRKQAHAHSK

SEQ ID NO: 211 - protein, 26 aa, Unknown
  Description of Unknown cell permeation peptide sequence
GWTLNSAGYLLKINLKALAALAKKIL

SEQ ID NO: 212 - protein, 18 aa, Unknown
  Description of Unknown cell permeation peptide sequence
KLALKLALKALKAALKLA

SEQ ID NO: 213 - protein, 9 aa, Unknown
  Description of Unknown cell permeation peptide sequence
RRRRRRRRR

SEQ ID NO: 214 - protein, 10 aa, Unknown
  Description of Unknown cell permeation peptide sequence
KFFKFFKFFK

SEQ ID NO: 215 - protein, 37 aa, Unknown
  Description of Unknown cell permeation peptide sequence
LLGDFFRKSKEKIGKEFKRIVQRIKDFLRNLVPRTES

SEQ ID NO: 216 - protein, 31 aa, Unknown
  Description of Unknown cell permeation peptide sequence
SWLSKTAKKLENSAKKRISEGIAIAIQGGPR

SEQ ID NO: 217 - protein, 30 aa, Unknown
  Description of Unknown cell permeation peptide sequence
ACYCRIPACIAGERRYGTCIYQGRLWAFCC

SEQ ID NO: 218 - protein, 36 aa, Unknown
  Description of Unknown cell permeation peptide sequence
DHYNCVSSGGQCLYSACPIFTKIQGTCYRGKAKCCK

SEQ ID NO: 219 - protein, 42 aa, Unknown
  Description of Unknown cell permeation peptide sequence
RRRPRPPYLPRPRPPPFFPPRLPPRIPPGFPPRFPPRFPGKR

SEQ ID NO: 220 - protein, 13 aa, Unknown
  Description of Unknown cell permeation peptide sequence
ILPWKWPWWPWRR

SEQ ID NO: 221 - protein, 16 aa, Unknown
  Description of Unknown cell permeation peptide sequence
AAVALLPAVLLALLAP

SEQ ID NO: 222 - protein, 11 aa, Unknown
  Description of Unknown cell permeation peptide sequence
AALLPVLLAAP

SEQ ID NO: 223 - protein, 12 aa, Unknown
  Description of Unknown cell permeation peptide sequence
RKCRIVVIRVCR